(19) United States
(12) Patent Application Publication
(10) Pub. No.: US 2012/0149691 A1
(43) Pub. Date: Jun. 14, 2012

(54) Pyrrolo [1,2-b] Pyridazine Derivatives as Janus Kinase Inhibitors

(75) Inventors: Yarlagadda S. Babu, Durham, NC (US); Yarlagadda S. Babu, Durham, NC (US); Pravin L. Kotian, Durham, NC (US); V. Satish Kumar, Durham, NC (US); Minwan Wu, Durham, NC (US); Tsu-Hsing Lin, Durham, NC (US)

(73) Assignee: BioCryst Pharmaceuticals, Inc., Durham, NC (US)

(21) Appl. No.: 13/387,569

(22) PCT Filed: Jul. 30, 2010
(86) PCT No.: PCT/US2010/043987

§ 371 Date: Mar. 1, 2012

Related U.S. Application Data
(60) Provisional application No. 61/230,490, filed on Jul. 31, 2009.

(51) Int. Cl. A61K 31/5415; A61K 31/5025; A61P 37/06; A61K 31/538; A61P 35/00; C07D 487/04; A61K 31/5377

(52) U.S. Cl. 514/224.2; 544/235; 514/248; 544/119; 514/233.2; 544/105; 544/52; 514/230.5

(57) ABSTRACT

The invention provides compounds of formula I:

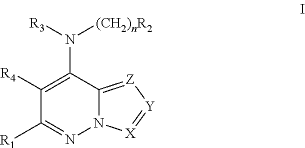

or a salt thereof as described herein. The invention also provides pharmaceutical compositions comprising a compound of formula I, processes for preparing compounds of formula I, intermediates useful for preparing compounds of formula I, and therapeutic methods for suppressing an immune response or treating cancer or a hematologic malignancy using compounds of formula I.

### CROSS-REFERENCE TO RELATED APPLICATION

This patent application claims the benefit of priority of U.S. application Ser. No. 61/230,490, filed Jul. 31, 2009, which application is herein incorporated by reference.

### BACKGROUND OF THE INVENTION

Janus kinase 3 (JAK3) is a cytoplasmic protein tyrosine kinase associated with the common gamma chain (γc), which is an integral component of various cytokine receptors (Elizabeth Kudlacz et al., American Journal of Transplantation, 2004, 4, 51-57).

While effective in the prevention of transplant rejection, commonly used immunosuppressants, such as calcineurin inhibitors, possess a number of significant dose-limiting toxicities, thereby prompting a search for agents with novel mechanisms of action. The inhibition of JAK3 represents an attractive strategy for immunosuppression based upon its limited tissue distribution, lack of constitutive activation and the evidence for its role in immune cell function. JAK3 is a viable target for immunosuppression and transplant rejection. JAK3 specific inhibitors may also be useful for treatment of hematologic and other malignancies that involve pathologic JAK activation.

Currently, there is a need for compounds, compositions and methods that are useful for treating diseases and conditions associated with pathologic JAK activation.

### SUMMARY OF THE INVENTION

In one embodiment, the invention provides a compound of the invention which is a compound of formula I:

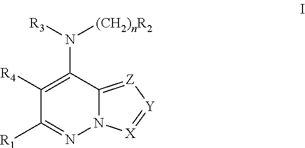

wherein

X is N or CR₅;

Y is N or CR₆;

Z is N or CR₇;

n is 0 or 1;

R₁ is H, halogen, alkyl, cycloalkyl, alkenyl, alkynyl, aryl, heteroaryl, heterocycle, NO₂, —CN, —OH, —OR_{d}, —NR_{b}R_{c}, N₃, SH, —SR_{d}, —C(O)Rₐ, —C(O)ORₐ, —C(O)NR_{b}R_{c}, —C(═NR_{b})NR_{b}R_{c}, NR_{b}COR_{d}, —NR_{b}C(O)OR_{d}, —NR_{b}S(O)₂R_{d}, —NR_{b}CONR_{b}R_{c}, —OC(O)NR_{b}R_{c}, —S(O)R_{d}, —S(O)NR_{b}R_{c}, —S(O)₂R_{d}, —S(O)₂OH, or —S(O)₂NR_{b}R_{c}; wherein any aryl or heteroaryl of R₁ may be optionally substituted with one or more (e.g. 1, 2, 3, 4 or 5) Rₑ groups; and wherein any alkyl, cycloalkyl, alkenyl, alkynyl or heterocycle of R₁ may be optionally substituted with one or more (e.g. 1, 2, 3, 4 or 5) groups selected from Rₑ, oxo and ═NOR_{z};

R₂ is H, alkyl, cycloalkyl, heterocycle, heteroaryl, aryl, —Oalkyl or a bridged ring group; wherein any aryl or heteroaryl of R₂ may be optionally substituted with one or more (e.g. 1, 2, 3, 4 or 5) R_{f} groups; and wherein any alkyl, —Oalkyl, cycloalkyl, heterocycle or bridged ring group of R₂ may be optionally substituted with one or more (e.g. 1, 2, 3, 4 or 5) groups selected from R_{f}, oxo and ═NOR_{z};

R₃ is H, —CN, —C(O)alkyl, —C(O)alkenyl, —C(O)alkynyl, —C(O)cycloalkyl, —C(O)aryl, —C(═O)C(═O)NHlower alkyl, —CONR_{g}Rₕ, alkyl, alkenyl, heterocycle, or heteroaryl; wherein any —C(O)aryl or heteroaryl of R₃ may be optionally substituted with one or more (e.g. 1, 2, 3, 4 or 5) Rᵢ groups; and wherein any alkyl, alkenyl, —C(O)alkyl, —C(O)alkenyl, —C(O)alkynyl, —C(O)cycloalkyl, heterocycle or —C(═O)C(═O)NHlower alkyl of R₃ may be optionally substituted with one or more (e.g. 1, 2, 3, 4 or 5) groups selected from Rᵢ, oxo and ═NOR_{z};

R₄ is halogen, alkyl, cycloalkyl, alkenyl, alkynyl, aryl, heteroaryl, heterocycle, NO₂, —CN, OH, —ORₙ, —NRₖRₘ, N₃, —SH, —SRₙ, —C(O)alkyl, —C(O)alkenyl, —C(O)alkynyl, —C(O)cycloalkyl, —C(O)aryl, —C(O)heteroaryl, —C(O)heterocycle, —C(O)ORⱼ, —C(O)NRₖRₘ, —C(═NRₖ)NRₖRₘ, —NRₖCORₙ, —NRₖC(O)ORₙ, —NRₖS(O)₂Rₙ, —NRₖCONRₖRₘ, —OC(O)NRₖRₘ, —S(O)Rₙ, —S(O)NRₖRₘ, —S(O)₂Rₙ, —S(O)₂OH, —S(O)₂NRₖRₘ, —C(═O)NHNHC(═S)NH₂, —C(═NH)NHOH or —C(═O)C(═O)NHlower alkyl; wherein any aryl, heteroaryl, C(O)aryl or —C(O)heteroaryl of R₄ may be optionally substituted with one or more (e.g. 1, 2, 3, 4 or 5) Rₚ groups and wherein any alkyl, cycloalkyl, alkenyl, alkynyl, heterocycle, C(O)alkyl, —C(O)alkenyl, —C(O)alkynyl, —C(O)cycloalkyl, —C(O)heterocycle or —C(═O)C(═O)NHlower alkyl of R₄ may be optionally substituted with one or more (e.g. 1, 2, 3, 4 or 5) groups selected from Rₚ, oxo and ═NOR_{z};

R₅ is H, OH, NO₂, CO₂H, —NR_{q}Rᵣ, —NHC(O)CF₃, —CONR_{q}Rᵣ, halogen or lower alkyl; which lower alkyl is optionally substituted with one or more (e.g. 1, 2, 3, 4 or 5) Rₛ groups;

R₆ is H, OH, NO₂, CO₂H, —NR_{q}Rᵣ, —CONR_{q}Rᵣ, alkenyl, halogen or lower alkyl; which lower alkyl or alkenyl is optionally substituted with one or more (e.g. 1, 2, 3, 4 or 5) Rₛ groups;

R₇ is H, OH, NO₂, CO₂H, —CONR_{q}Rᵣ, halogen or lower alkyl; which lower alkyl is optionally substituted with one or more (e.g. 1, 2, 3, 4 or 5) Rₛ groups;

each Rₐ is independently selected from H, alkyl, alkenyl, alkynyl, cycloalkyl, heterocycle, heteroaryl and aryl;

R_{b} and R_{c} are each independently selected from H, alkyl, alkenyl, alkynyl, cycloalkyl, heterocycle and heteroaryl; or R_{b} and R_{c} together with the nitrogen to which they are attached form a pyrrolidino, piperidino, piperazino, azetidino, morpholino, or thiomorpholino ring;

each R_{d} is independently selected from alkyl, alkenyl, alkynyl, cycloalkyl, heterocycle, heteroaryl and aryl;

each Rₑ is independently selected from halogen, aryl, heteroaryl, heterocycle, R_{z}, OH, —CN, —OR_{z}, —Oaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z}; wherein any aryl, —Oaryl, —Saryl, —S(O)aryl, —S(O)₂aryl, —NHCOaryl, or NHS(O)₂aryl of Rₑ may be optionally substituted with one or more (e.g. 1, 2, 3, 4 or 5) R_{y} groups;

each R_{f} is independently selected from halogen, aryl, heteroaryl, heterocycle, R_{z}, OH, —CN, —OR_{z}, —Oaryl, —Oheterocycle, —Oheteroaryl, —OC(O)R_{z}, —OC(O)NR_{z1}R_{z2}, SH, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2}, —C(O)heterocycle, —C(O)heteroaryl and —C(O)C(O)R_{z}; wherein any aryl, heteroaryl, —Oaryl, —Oheteroaryl, —Saryl, —Sheteroaryl, —S(O)heteroaryl, —S(O)₂aryl, —S(O)₂heteroaryl, —NHCOaryl, —NHCOheteroaryl, —NHS(O)₂aryl or —C(O)heteroaryl of R_{f} may be optionally substituted with one or more (e.g. 1, 2, 3, 4 or 5) R_{y} groups; and wherein any heterocycle or —C(O)heterocycle of R_{f} may be optionally substituted with one or more (e.g. 1, 2, 3, 4 or 5) groups selected from R_{y}, oxo and ═NOR_{z};

R_{g} and Rₕ are each independently selected from H, alkyl, alkenyl, alkynyl, cycloalkyl, heterocycle and heteroaryl; or R_{g} and Rₕ together with the nitrogen to which they are attached form a pyrrolidino, piperidino, piperazino, azetidino, morpholino, or thiomorpholino ring;

each Rᵢ is independently selected from halogen, aryl, heteroaryl, heterocycle, R_{z}, OH, —CN, —Oaryl, —OC(O)R_{z}, —OC(O)NR_{z1}R_{z2}, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z}; wherein any aryl, —Oaryl, —Saryl, —Sheteroaryl, —S(O)aryl, —S(O)₂aryl, —NHCOaryl, or —NHS(O)₂aryl, of Rᵢ may be optionally substituted with one or more (e.g. 1, 2, 3, 4 or 5) R_{y} groups;

Rⱼ is H, alkyl, alkenyl, alkynyl, cycloalkyl, heterocycle, heteroaryl or aryl;

Rₖ and Rₘ are each independently selected from H, alkyl, alkenyl, alkynyl, cycloalkyl, heterocycle and heteroaryl; or Rₖ and Rₘ together with the nitrogen to which they are attached form a pyrrolidino, piperidino, piperazino, azetidino, morpholino, or thiomorpholino ring;

each Rₙ is independently selected from alkyl, alkenyl, alkynyl, cycloalkyl, heterocycle, heteroaryl and aryl;

each Rₚ is independently selected from halogen, aryl, heteroaryl, heterocycle, R_{z}, OH, —CN, —OR_{z}, —Oaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z}; wherein any aryl, —Oaryl, —Saryl, —S(O)aryl, —S(O)₂aryl, —NHCOaryl, —NHCOheteroaryl, —NHCO₂R_{z}, —NHCONR_{z1}R_{z2} or —NHS(O)₂aryl, of Rₚ may be optionally substituted with one or more (e.g. 1, 2, 3, 4 or 5) R_{y} groups;

R_{q} and Rᵣ are each independently selected from H, alkyl, alkenyl, alkynyl, cycloalkyl, heterocycle and heteroaryl; or R_{q} and Rᵣ together with the nitrogen to which they are attached form a pyrrolidino, piperidino, piperazino, azetidino, morpholino, or thiomorpholino ring;

each Rₛ is independently selected from halogen, aryl, heteroaryl, heterocycle, R_{z}, OH, —CN, —Oaryl, —OC(O)R_{z}, —OC(O)NR_{z1}R_{z2}, oxo, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, ═NOR_{z}, —CHO, —C(O)R_{z}, —C(O)OH, —C(O)OR_{z}, —C(O)NR_{z1}R_{z2} and —C(O)C(O)R_{z}; wherein any aryl, Oaryl, —Saryl, —S(O)aryl, —S(O)₂aryl, —NHCOaryl or —NHS(O)₂aryl of Rₛ may be optionally substituted with one or more (e.g. 1, 2, 3, 4 or 5) R_{y} groups;

each R_{z} is independently lower alkyl or lower cycloalkyl; wherein any lower alkyl or lower cycloalkyl of R_{z} may be optionally substituted with one or more (e.g. 1, 2 or 3) groups selected from halogen, —CN, OH, —Olower alkyl, —NHlower alkyl, —C(O)NHlower alkyl, —C(O)N(lower alkyl)₂, aryl, heterocycle, —Oheterocycle and heteroaryl; wherein aryl, heteroaryl or heterocycle may be optionally substituted with one or more (e.g. 1, 2 or 3) lower alkyl;

R_{z1} and R_{z2} are each independently selected from H, lower alkyl, alkenyl, alkynyl, lower cycloalkyl, heterocycle and heteroaryl; wherein lower alkyl or lower cycloalkyl may be optionally substituted with one or more (e.g. 1, 2 or 3) Rₜ groups; or R_{z1} and R_{z2} together with the nitrogen to which they are attached form a cyclic amino;

each Rₜ is independently selected from halogen, —CN, OH, —Olower alkyl, —NHlower alkyl, —C(O)NHlower alkyl, —C(O)N(lower alkyl)₂, heterocycle and heteroaryl; wherein any heterocycle of Rₜ may be substituted with one or more (e.g. 1, 2 or 3) lower alkyl; and

each R_{y} is independently halogen, aryl, R_{z}, OH, —CN, OR_{z}, —Oaryl, —Oheteroaryl, —OC(O)R_{z}, —OC(O)NR_{z1}R_{z2}, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, CHO, —C(O)R_{z}, —C(O)OH, —C(O)OR_{z}, —C(O)NR_{z1}R_{z2}, —C(O)C(O)R_{z}, heterocycle or heteroaryl;

or a salt thereof.

The invention also provides a pharmaceutical composition comprising a compound of formula I or a pharmaceutically acceptable salt thereof, and a pharmaceutically acceptable diluent or carrier.

The invention also provides method for treating a disease or condition associated with pathologic JAK activation (e.g. a cancer, a hematologic malignancy or other malignancy) in a mammal (e.g. a human), comprising administering a compound of formula I, or a pharmaceutically acceptable salt thereof, to the mammal.

The invention also provides a compound of formula I, or a pharmaceutically acceptable salt thereof, for use in the prophylactic or therapeutic treatment of a disease or condition associated with pathologic JAK activation (e.g. a cancer, a hematologic malignancy or other malignancy).

The invention also provides a compound of formula I, or a pharmaceutically acceptable salt thereof for use in medical therapy (e.g. for use in treating a disease or condition associated with pathologic JAK activation such as cancer, a hematologic malignancy or other malignancy).

The invention also provides a compound of formula I or a pharmaceutically acceptable salt thereof for the manufacture of a medicament for the treatment of a disease or condition associated with pathologic JAK activation (e.g. a cancer, a hematologic malignancy or other malignancy) in a mammal (e.g. a human).

The invention also provides a method for suppressing an immune response in a mammal (e.g. a human), comprising administering a compound of formula I, or a pharmaceutically acceptable salt thereof, to the mammal.

The invention also provides a compound of formula I, or a pharmaceutically acceptable salt thereof, for use in the prophylactic or therapeutic suppression of an immune response.

The invention also provides the use of a compound of formula I, or a pharmaceutically acceptable salt thereof for the manufacture of a medicament for suppressing an immune response in a mammal (e.g. a human).

The invention also provides processes and intermediates disclosed herein that are useful for preparing compounds of formula I or salts thereof.

### DETAILED DESCRIPTION OF THE INVENTION

### Definitions

The term “alkyl” as used herein refers to alkyl groups having from 1 to 10 carbon atoms (i.e. (C₁-C₁₀)alkyl) which are straight or branched monovalent groups. The term “lower alkyl” as used herein refers to alkyl groups having from 1 to 6 carbon atoms which are straight or branched monovalent groups. This term is exemplified by groups such as methyl, ethyl, n-propyl, iso-propyl, n-butyl, t-butyl, isobutyl, n-pentyl, neopentyl, and n-hexyl, and the like.

The terms “alkenyl” or “alkene” as used herein refers to an alkenyl group having from 2 to 10 carbon atoms which are straight or branched monovalent groups and having at least one double bond. Such groups are exemplified by vinyl(ethen-1-yl), allyl, 1-propenyl, 2-propenyl(allyl), 1-methylethen-1-yl, 1-buten-1-yl, 2-buten-1-yl, 3-buten-1-yl, 1-methyl-1-propen-1-yl, 2-methyl-1-propen-1-yl, 1-methyl-2-propen-1-yl, and 2-methyl-2-propen-1-yl, preferably 1-methyl-2-propen-1-yl and the like.

The term “alkynyl” or “alkyne” as used herein refers to an alkynyl group having from 2-10 carbon atoms which are straight or branched monovalent groups and having at least one triple bond. Such groups are exemplified by, but not limited to ethyn-1-yl, propyn-1-yl, propyn-2-yl, 1-methylprop-2-yn-1-yl, butyn-1-yl, butyn-2-yl, butyn-3-yl, and the like.

The term “halogen” as used herein refers to fluoro, chloro, bromo and iodo. In one embodiment halogen is specifically fluoro.

The term “cycloalkyl” as used herein refers to a saturated or partially unsaturated cyclic hydrocarbon ring systems, such as those containing 1 to 3 rings and 3 to 8 carbons per ring wherein multiple ring cycloalkyls can have fused and spiro bonds to one another but not bridging bonds. Therefore, cycloalkyl does not include bridged cyclic hydrocarbons as defined below. Exemplary groups include but are not limited to cyclopropyl, cyclobutyl, cyclopentyl, cyclohexyl, cycloheptyl, cyclooctyl, cyclobutenyl, cyclohexenyl, cyclooctadienyl, decahydronaphthalene and spiro[4.5]decane.

The term “lower cycloalkyl” as used herein refers to a cycloalkyl containing 1 ring and 3-6 carbon atoms. Exemplary groups include cyclopropyl, cyclobutyl, cyclopentyl and cyclohexyl.

The term “aryl” as used herein refers to a monovalent aromatic cyclic group of from 6 to 14 carbon atoms having a single ring (e.g. phenyl) or multiple condensed rings (e.g. naphthyl or anthryl) wherein the condensed rings may be aromatic, saturated or partially saturated provided that at least one of the condensed rings is aromatic. Exemplary aryls include, but are not limited to, phenyl, indanyl, naphthyl, 1,2-dihydronaphthyl and 1,2,3,4-tetrahydronaphthyl.

The term “heteroaryl” as used herein refers to a group of from 1 to 10 carbon atoms and 1 to 4 heteroatoms selected from the group consisting of oxygen, nitrogen and sulfur in the ring. The sulfur and nitrogen heteroatoms atoms may also be present in their oxidized forms. Such heteroaryl groups can have a single aromatic ring with at least one heteroatom (e.g. pyridyl, pyrimidinyl or furyl) or multiple condensed rings (e.g. indolizinyl or benzothienyl) wherein all of the condensed rings may or may not be aromatic and/or contain a heteroatom provided that at least one of the condensed rings is aromatic with at least one heteroatom. Exemplary heteroaryl groups include, but are not limited to pyridyl, pyrrolyl, pyrazinyl, pyrimidinyl, pyridazinyl, pyrazolyl, thienyl, indolyl, thiophenyl, imidazolyl, oxazolyl, thiazolyl, furyl, oxadiazolyl, thiadiazolyl, quinolyl, isoquinolyl, benzothiazolyl, benzoxazolyl, indazolyl, indolyl, quinoxalyl, quinazolyl, 5,6,7,8-tetrahydroisoquinoline and the like.

The term “heterocycle” or “heterocyclic” or “heterocycloalkyl” refers to a group of from 1 to 10 carbon atoms and 1 to 4 heteroatoms selected from the group consisting of oxygen, nitrogen and sulfur in the ring. The sulfur and nitrogen heteroatoms atoms may also be present in their oxidized forms. Such heterocycle groups include a single saturated or partially unsaturated ring with at least one heteroatom (e.g. azetidinyl or piperidinyl). Heterocycle groups also include multiple condensed rings wherein the condensed rings may be aryl, cycloalkyl or heterocycle but not heteroaryl provided that at least one of the condensed rings is a heterocycle (i.e. a saturated or partially unsaturated ring with at least one heteroatom). Heterocycles do not included aza-bridged cyclic hydrocarbons as defined below. Heterocycles may include aziridinyl, azetidinyl, pyrrolizinyl, piperidinyl, homopiperidinyl, morpholinyl, thiomorpholinyl, piperazinyl, tetrahydrofuranyl, tetrahydrothiophenyl, dihydrooxazolyl, tetrahydropyranyl, tetrahydrothiopyranyl, 1,2,3,4-tetrahydroquinolyl, 1,2,3,4-tetrahydroisoquinolyl, benzoxazinyl and dihydrooxazolyl.

The term “cyclic amino” as used herein is a subgroup of heterocycloalkyls and refers to a monovalent 3-membered to 8-membered saturated or partially unsaturated, single, nonaromatic ring which has at least one nitrogen atom, and may have one or more identical or different hetero atoms selected from the group consisting of nitrogen, oxygen, and sulfur wherein the nitrogen or sulfur atoms may be oxidized. Aza-bridged cyclic hydrocarbons are excluded. Cyclic amino includes but is not limited to values such as aziridino, azetidino, pyrrolidino, piperidino, homopiperidino, morpholino, thiomorpholino, and piperazino.

The term “bridged ring group” includes “bridged cyclic hydrocarbon” and “aza-bridged cyclic hydrocarbon.”

The term “bridged cyclic hydrocarbon” is a saturated or partially unsaturated, bicyclic or polycyclic bridged hydrocarbon group having two or three C₃-C₁₀ cycloalkyl rings and at least one bridging group. Bicyclic or polycyclic C₄-C₁₆ bridged hydrocarbon groups are particularly preferable. Bridged cyclic hydrocarbon ring systems include but are not limited to cyclo[2.1.1]hexyl, bicyclo[2.2.1]heptyl, bicyclo[2.2.2]octyl, bicyclo[4.3.1]decyl, bicyclo[3.3.1]nonyl, bornyl, bornenyl, norbornyl, norbornenyl, 6,6-dimethylbicyclo[3.1.1]heptyl, tricyclobutyl, and adamantyl. In one embodiment bridged cyclic hydrocarbon is adamantyl or bicyclo[2.2.1]heptyl.

The term “aza-bridged cyclic hydrocarbon” is a saturated or partially unsaturated, bicyclic or polycyclic bridged hydrocarbon group having two or three rings in which at least one of the atoms is a nitrogen atom. In one embodiment the aza-bridged cyclic hydrocarbon is a bicyclic or polycyclic C₄-C₁₆ aza-bridged cyclic hydrocarbon group. Aza-bridged cyclic hydrocarbons include but are not limited to ring systems such as azanorbornyl, quinuclidinyl, isoquinuclidinyl, tropanyl, 8-azabicyclo[3.2.1]octanyl, azabicyclo[2.2.1]heptanyl, 2-azabicyclo[3.2.1]octanyl, azabicyclo[3.2.2]nonanyl, azabicyclo[3.3.0]nonanyl, and azabicyclo[3.3.1]nonanyl. In one embodiment aza-bridged cyclic hydrocarbon is preferably 8-azabicyclo[3.2.1]octanyl or 2-oxa-5-azabicyclo[2.2.1]hept-5-yl.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Specific values listed below for radicals, substituents, and ranges, are for illustration only; they do not exclude other defined values or other values within defined ranges for the radicals and substituents. The specific values listed below are specific values for compounds of formula I. The specific values listed below are also specific values for compounds of formula Ia, Ib, Ic, Id, Ie, If, Ig, Ih, Ii, Ij, Ik, and Im wherein the values are represented by the formula.

A specific compound of formula I is a compound of formula Ia:

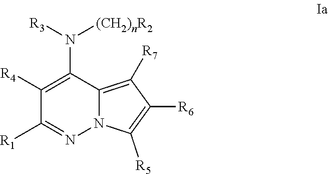

or a salt thereof.

Another specific compound of formula I is a compound of formula Ib:

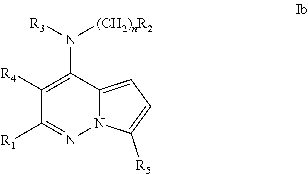

or a salt thereof.

Another specific compound of formula I is a compound of formula Ic:

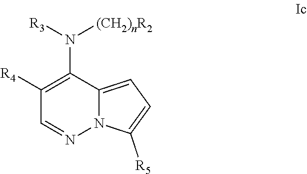

or a salt thereof.

Another specific compound of formula I is a compound of formula Id:

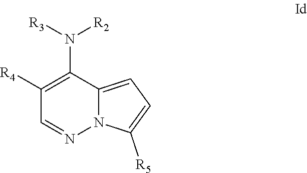

or a salt thereof.

Another specific compound of formula I is a compound of formula Ie:

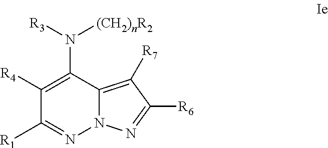

or a salt thereof.

Another specific compound of formula I is a compound of formula If:

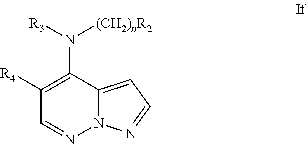

or a salt thereof.

Another specific compound of formula I is a compound of formula Ig:

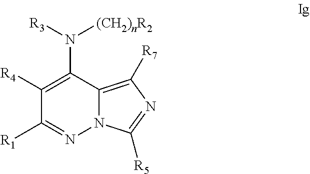

or a salt thereof.

Another specific compound of formula I is a compound of formula Ih:

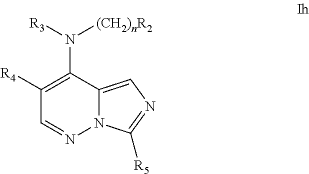

or a salt thereof.

Another specific compound of formula I is a compound of formula Ii:

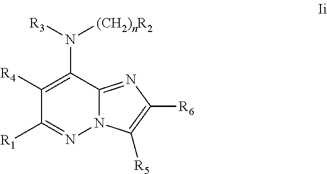

or a salt thereof.

Another specific compound of formula I is a compound of formula Ij:

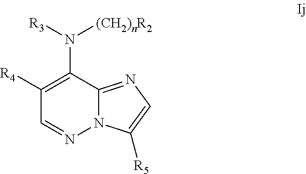

or a salt thereof.

Another specific compound of formula I is a compound of formula Ik:

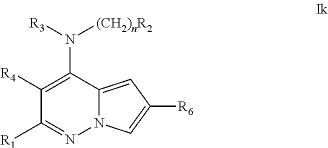

or a salt thereof.

Another specific compound of formula I is a compound of formula Im:

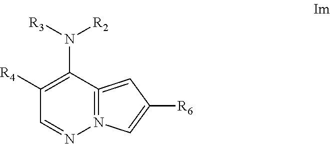

or a salt thereof.

A specific value for X is CR₅.

A specific value for R₅ is H, OH, NO₂, CO₂H, —NR_{q}Rᵣ or —CONH₂.

Another specific value for R₅ is H, NO₂, —NH₂ or —CONH₂.

Another specific value for R₅ is H.

Another specific value for R₅ is NH₂.

Another specific value for R₅ is OH.

Another specific value for R₅ is NO₂.

Another specific value for R₅ is —NHC(O)CF₃.

Another specific value for X is N.

A specific value for Y is CR₆.

A specific value for R₆ is H, OH, NO₂, halogen or NH₂.

Another specific value for R₆ is H.

Another specific value for R₆ is alkenyl.

Another specific value for R₆ is H, NO₂ or NH₂.

Another specific value for Y is N.

A specific value for Z is CR₇.

A specific value for R₇ is H.

Another specific value for Z is N.

A specific group of compounds of formula I are compounds wherein X, Y and Z are each CH.

Another specific group of compounds of formula I are compounds wherein Y and Z are each CH.

Another specific group of compounds of formula I are compounds wherein X is CR₅, Y is CR₆ and Z is CR₇.

Another specific group of compounds of formula I are compounds wherein X is N, Y is CR₆ and Z is CR₇.

Another specific group of compounds of formula I are compounds wherein X is CR₅, Y is N and Z is CR₇.

Another specific group of compounds of formula I are compounds wherein X is CR₅, Y is CR₆ and Z is N.

Another specific group of compounds of formula I are compounds wherein X is N, Y is N and Z is CR₇.

Another specific group of compounds of formula I are compounds wherein X is CR₅, Y is N and Z is N.

Another specific group of compounds of formula I are compounds wherein X is N, Y is CR₆ and Z is N.

Another specific group of compounds of formula I are compounds wherein X is N, Y is N and Z is N.

A specific value for n is 0.

Another specific value for n is 1.

A specific value for R₁ is H.

Another specific value for R₁ is CH₃.

Another specific value for R₁ is Cl.

A specific value for R₃ is alkyl or H.

Another specific value for R₃ is CH₃.

Another specific value for R₃ is H.

A specific group of compounds of formula I are compounds wherein only one of R₁ and R₄ is Cl.

Another specific group of compounds of formula I are compounds wherein only one of R₁ and R₄ is CH₃.

A specific value for R₄ is heteroaryl, —C(O)alkyl, —C(O)NRₖRₘ, —C(O)ORⱼ, —CN, —C(NRₖ)NRₖRₘ or —S(O)₂NRₖRₘ; wherein any heteroaryl of R₄ may be optionally substituted with one or more Rₚ groups; and wherein any alkyl of R₄ may be optionally substituted with one or more groups selected from Rₚ, oxo and ═NOR_{z}.

Another specific value for R₄ is heteroaryl, —C(O)alkyl, —C(O)NRₖRₘ, —C(NRₖ)NRₖRₘ or —S(O)₂NRₖRₘ; wherein any heteroaryl of R₄ may be optionally substituted with one or more Rₚ groups; and wherein any alkyl of R₄ may be optionally substituted with one or more groups selected from Rₚ, oxo and ═NOR_{z}.

Another specific value for R₄ is —C(O)NRₖRₘ, —C(O)ORⱼ or —CN.

Another specific value for R₄ is —C(O)NRₖRₘ.

Another specific value for R₄ is —C(O)NH₂.

Another specific value for R₄ is —S(O)₂NRₖRₘ.

Another specific value for R₄ is —S(O)₂NH₂.

Another specific value for R₄ is —C(═NRₖ)NRₖRₘ.

Another specific value for R₄ is —C(═NH)NH₂.

Another specific value for R₄ is —C(O)alkyl.

Another specific value for R₄ is —C(O)CH₂OH.

Another specific value for R₄ is heteroaryl.

Another specific value for R₄ is heteroaryl substituted with one or more —NH₂ or R_{z} groups.

Another specific value for R₄ is:

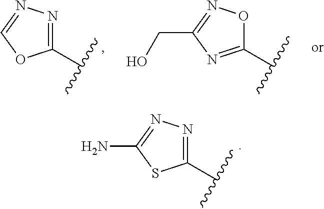

Another specific value for R₄ is:

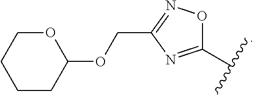

Another specific value for R₄ is —C(O)ORⱼ.

Another specific value for R₄ is —C(O)OH.

Another specific value for R₄ is —C(O)OCH₃.

Another specific value for R₄ is —CN.

Another specific value for R₄ is —C(═O)NHNHC(═S)NH₂ or —C(═NH)NHOH.

A specific value for R₂ is alkyl, cycloalkyl, heterocycle or aryl; wherein any aryl of R₂ may be optionally substituted with one or more R_{f} groups; and wherein any alkyl, cycloalkyl or heterocycle of R₂ may be optionally substituted with one or more groups selected from R_{f}, oxo and ═NOR_{z}.

Another specific value for R₂ is alkyl; wherein alkyl is substituted with one or more R_{f} groups.

Another specific value for R₂ is alkyl; wherein alkyl is substituted with one or two R_{f} groups.

Another specific value for R₂ is aryl; wherein any aryl of R₂ may be optionally substituted with one or more R_{f} groups.

Another specific value for R₂ is phenyl; wherein any phenyl of R₂ may be optionally substituted with one or more R_{f} groups.

Another specific value for R₂ is cycloalkyl or heterocycle; wherein any cycloalkyl or heterocycle of R₂ may be optionally substituted with one or more groups selected from R_{f} and oxo.

Another specific value for R₂ is cyclopropyl, cyclopentyl, cyclohexyl, cycloheptyl, tetrahydropyranyl, tetrahydrofuranyl or piperidinyl; wherein any cyclopropyl, cyclopentyl, cyclohexyl, cycloheptyl, tetrahydropyranyl, tetrahydrofuranyl or piperidinyl of R₂ may be optionally substituted with one or more groups selected from R_{f} and oxo.

Another specific value for R₂ is bridged ring group; wherein any bridged ring group of R₂ may be optionally substituted with one or more groups selected from R_{f} and oxo.

Another specific value for R₂ is bridged cyclic hydrocarbon; wherein any bridged cyclic hydrocarbon of R₂ may be optionally substituted with one or more groups selected from R_{f} and oxo.

Another specific value for R₂ is aza-bridged cyclic hydrocarbon; wherein aza-bridged cyclic hydrocarbon of R₂ may be optionally substituted with one or more groups selected from R_{f} and oxo.

Another specific value for R₂ is adamantyl or 8-azabicyclo[3.2.1]octanyl; wherein any adamantyl or 8-azabicyclo[3.2.1]octanyl of R₂ may be optionally substituted with one or more groups selected from R_{f} and oxo.

Another specific value for R₂ is adamantyl or 8-azabicyclo[3.2.1]octanyl substituted with one or more —OH.

A specific value for R_{f} is halogen, aryl, heteroaryl, heterocycle, R_{z}, OH, —CN, —OR_{z}, —Oaryl, —Oheterocycle, —Oheteroaryl, —NR_{z1}R_{z2}, —NHCOR_{z}, —NHCO₂R_{z}, —C(O)R_{z} and —C(O)NR_{z1}R_{z2}; wherein any aryl, heteroaryl, —Oaryl or —Oheteroaryl of R_{f} may be optionally substituted with one or more R_{y} groups; and wherein any heterocycle of R_{f} may be optionally substituted with one or more groups selected from R_{y} and oxo.

Another specific value for R_{f} is halogen, aryl, heteroaryl, heterocycle, R_{z}, OH, —CN, —OR_{z}, —NR_{z1}R_{z2}, —NHCOR_{z}, —NHCO₂R_{z}, —C(O)R_{z} and —C(O)NR_{z1}R_{z2}; wherein any aryl, heteroaryl or heterocycle of R_{f} may be optionally substituted with one or more R_{y} groups.

Another specific value for R_{f} is aryl, heteroaryl, heterocycle or —NR_{z1}R_{z2}; wherein any aryl, heteroaryl or heterocycle of R_{f} may be optionally substituted with one or more R_{y} groups.

Another specific value for R_{f} phenyl, thiazolyl, morpholinyl, piperizinyl, furanyl, imidazolyl or —NR_{z1}R_{z2}; wherein any phenyl, thiazolyl, morpholinyl, piperizinyl, furanyl or imidazolyl of R_{f} may be optionally substituted with one or more R_{y} groups.

Another specific value for R_{f} is aryl, R_{z}, OH, —NR_{z1}R_{z2}, —NHCOR_{z}, —NHCO₂R_{z}, and —C(O)R_{z}; wherein any aryl, of R_{f} may be optionally substituted with one or more R_{y} groups.

Another specific value for R_{f} is R_{z}.

A specific value for R_{z} is independently a lower alkyl; wherein any lower of alkyl R_{z} may be optionally substituted with one or more groups selected from —CN and aryl.

A specific value for R_{y} is halogen, R_{z}, OH, —CN, —OR_{z}, —NR_{z1}R_{z2}, —NHCOR_{z}, NO₂, —C(O)R_{z} or —C(O)NR_{z1}R_{z2}.

Another specific value for R_{y} is halogen, R_{z}, or —OR_{z}.

Another specific value for R₂ is:

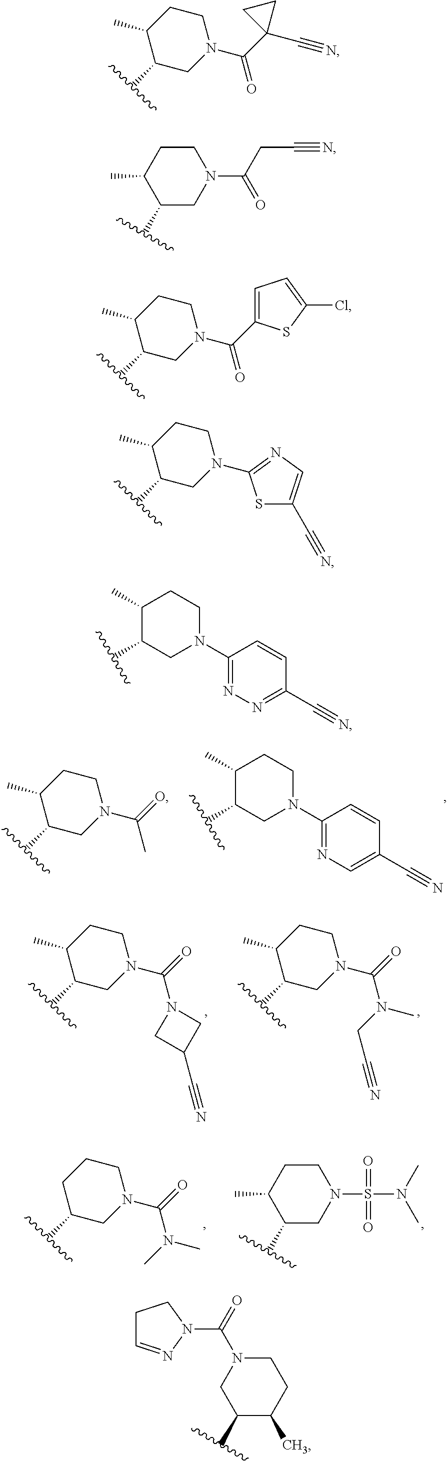

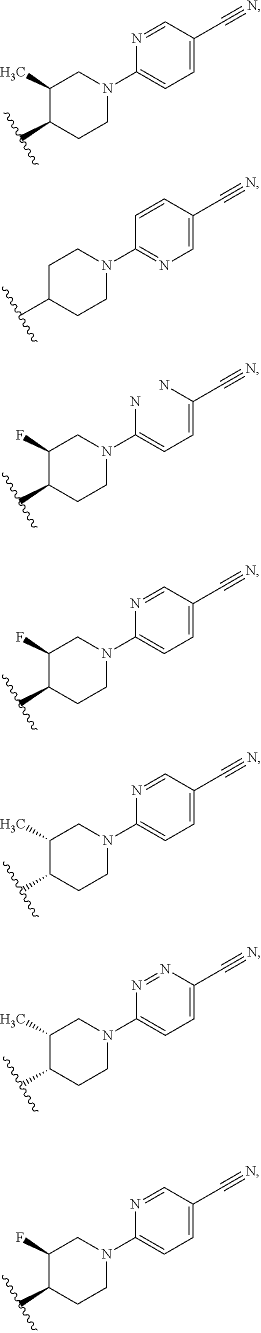

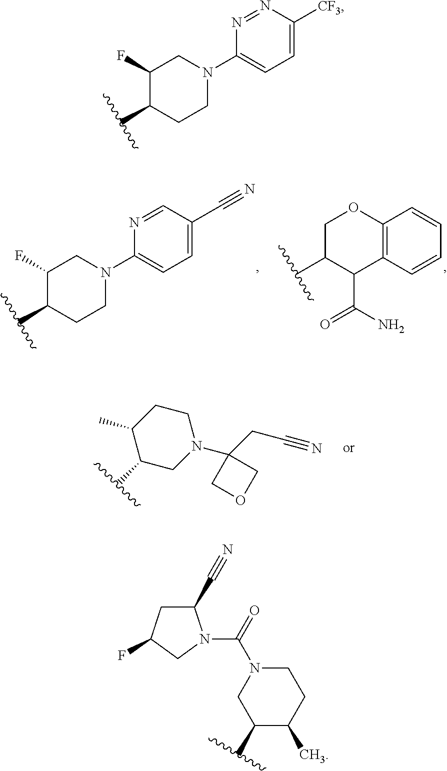

Another specific value for R₂ is:

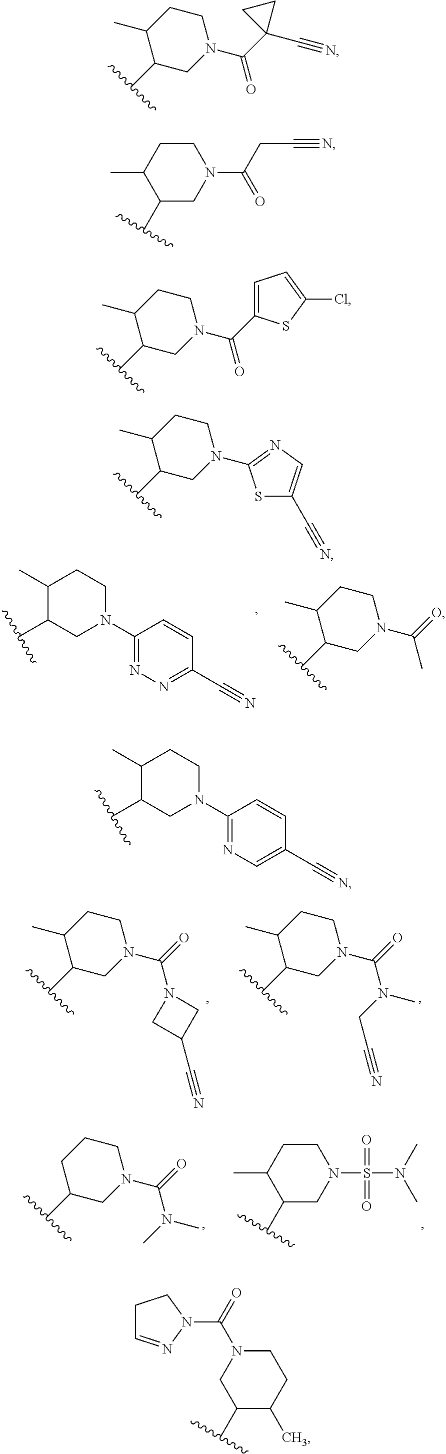

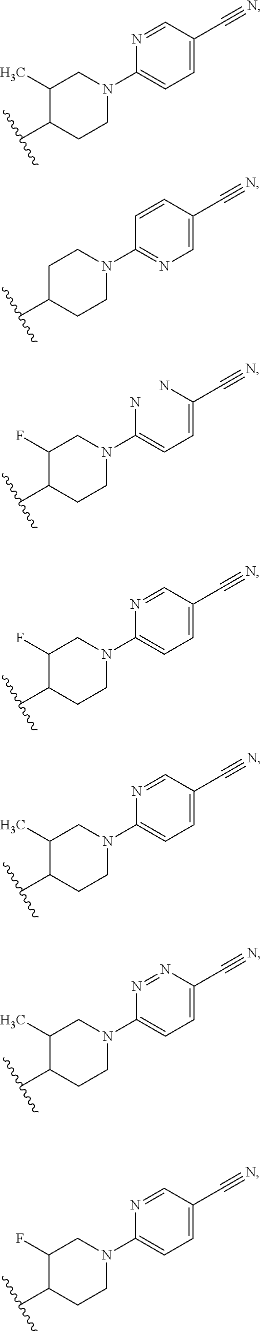

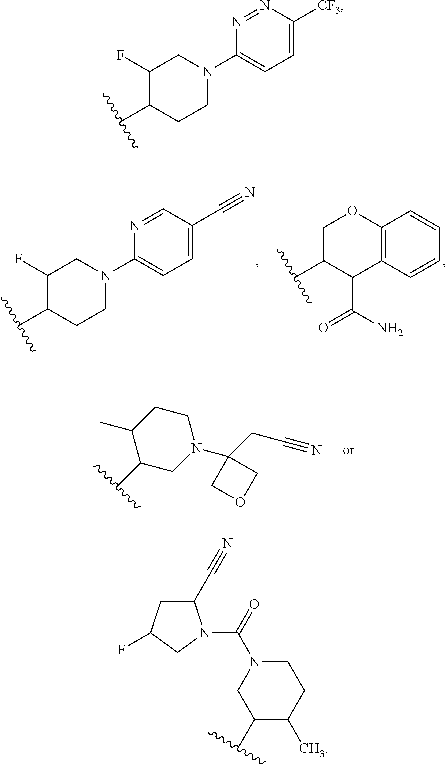

Another specific value for R₂ is:

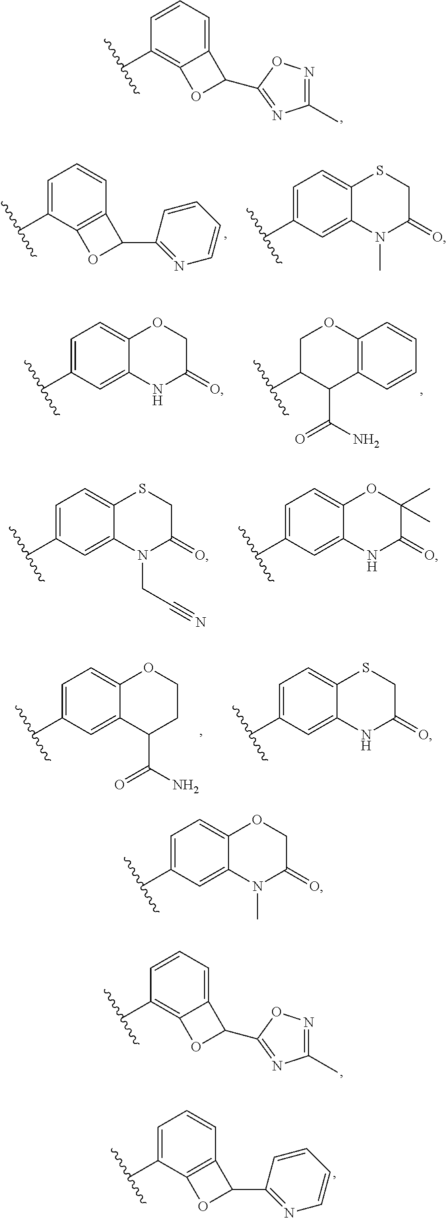

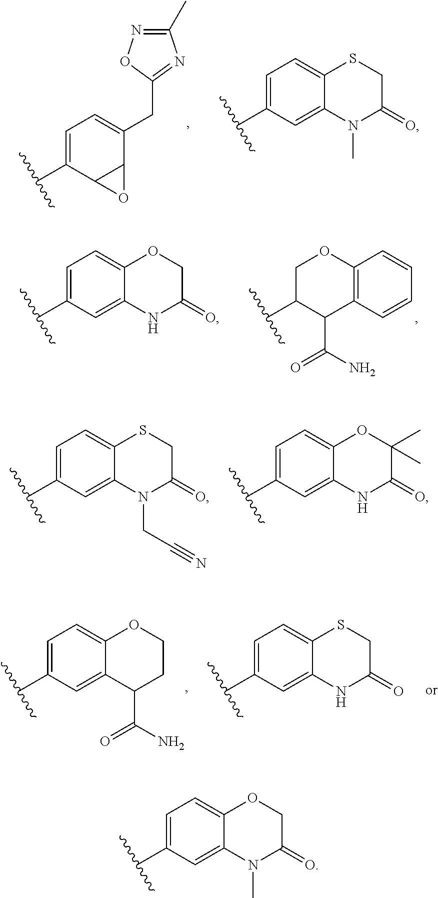

Another specific value for R₂ is:

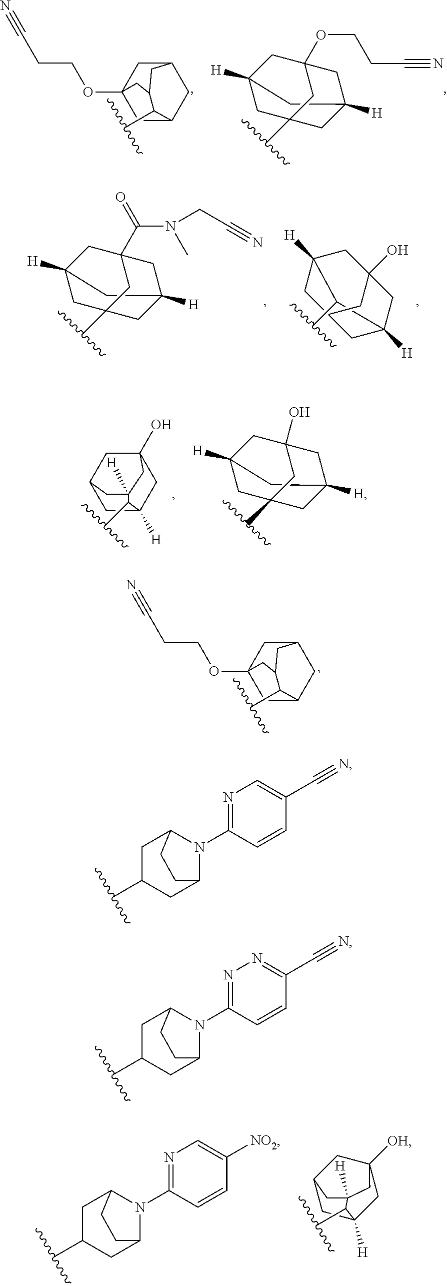

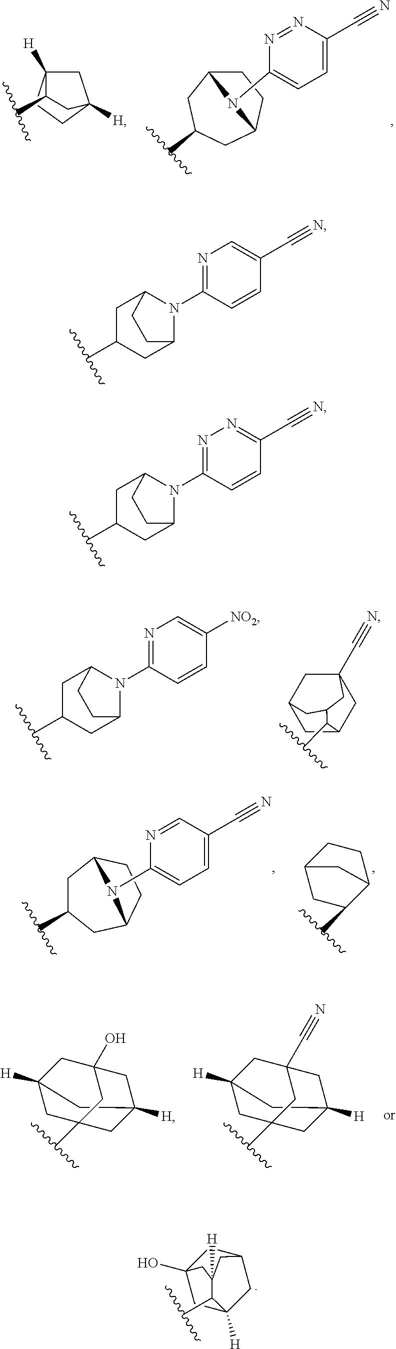

Another specific value for R₂ is:

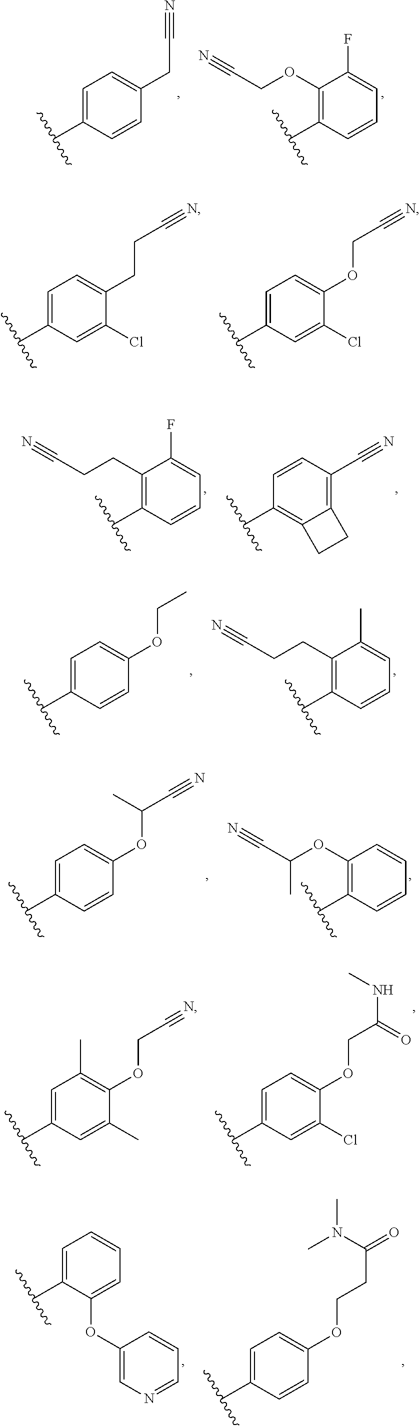

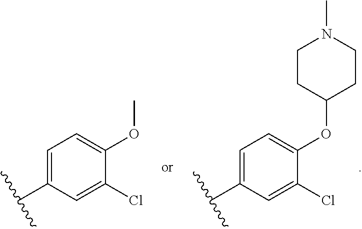

Another specific value for R₂ is:

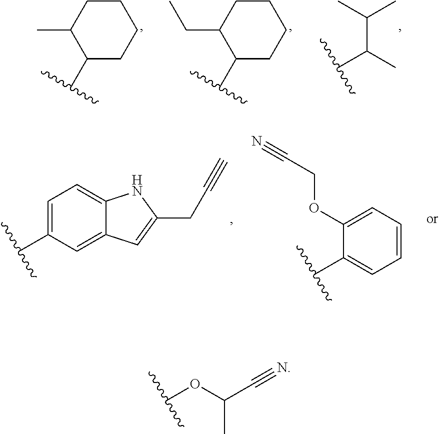

Another specific value for R₂ is:

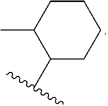

Another specific value for R₂ is:

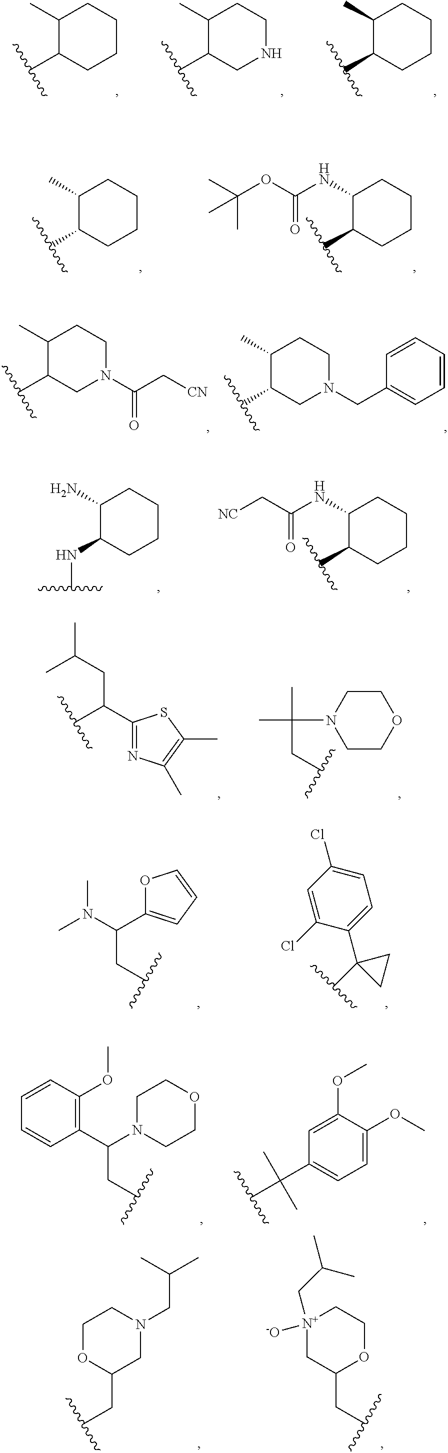

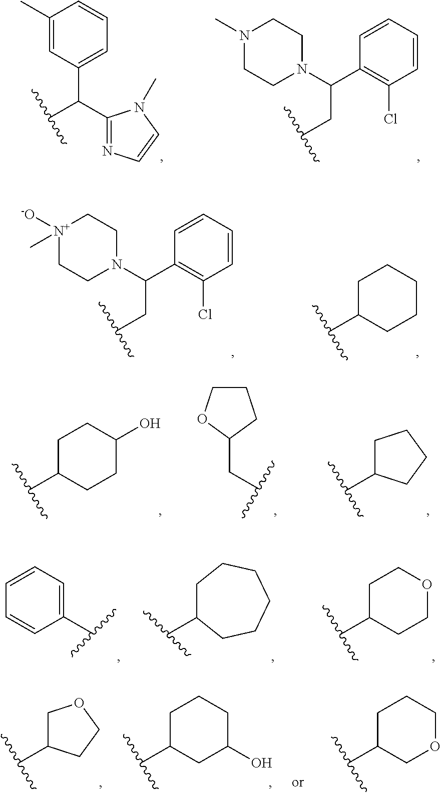

A specific compound of formula I is:

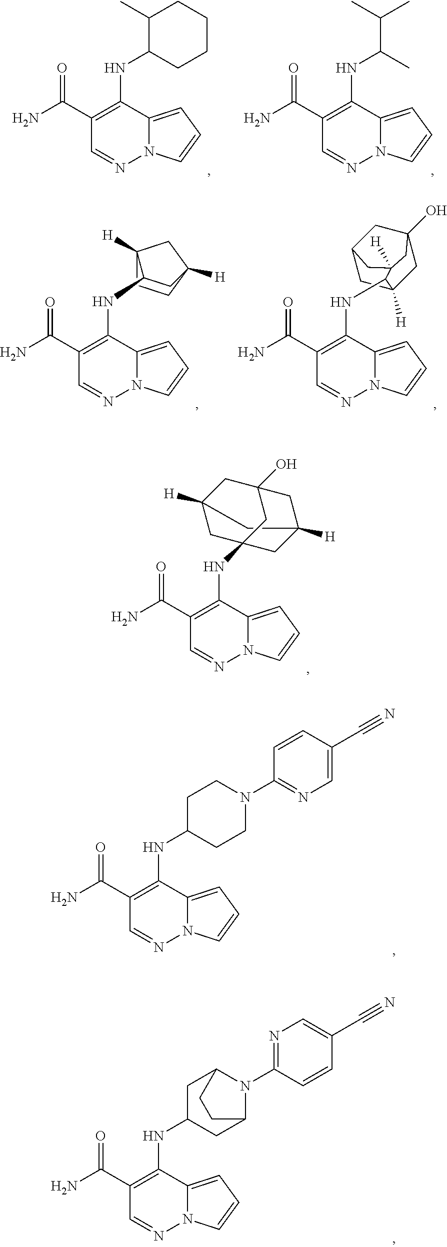

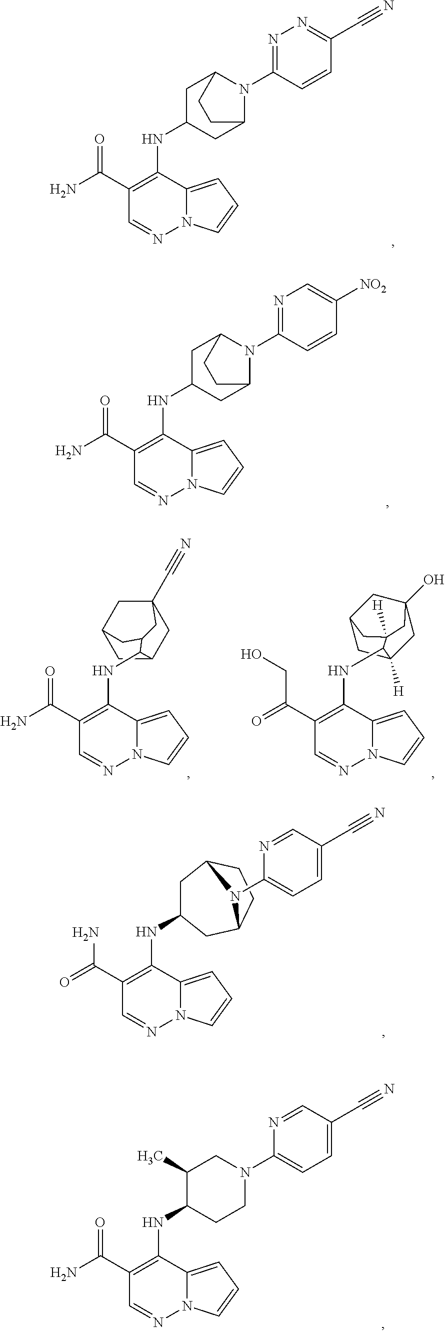

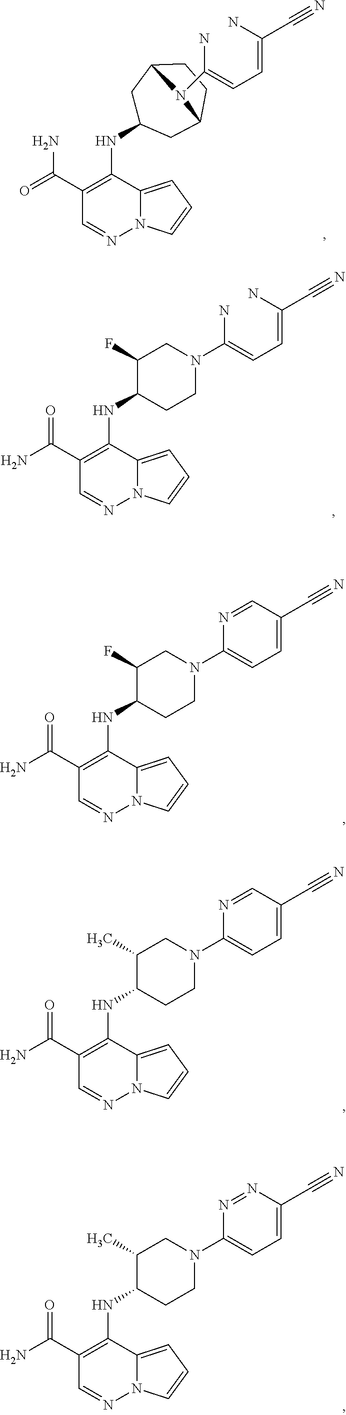

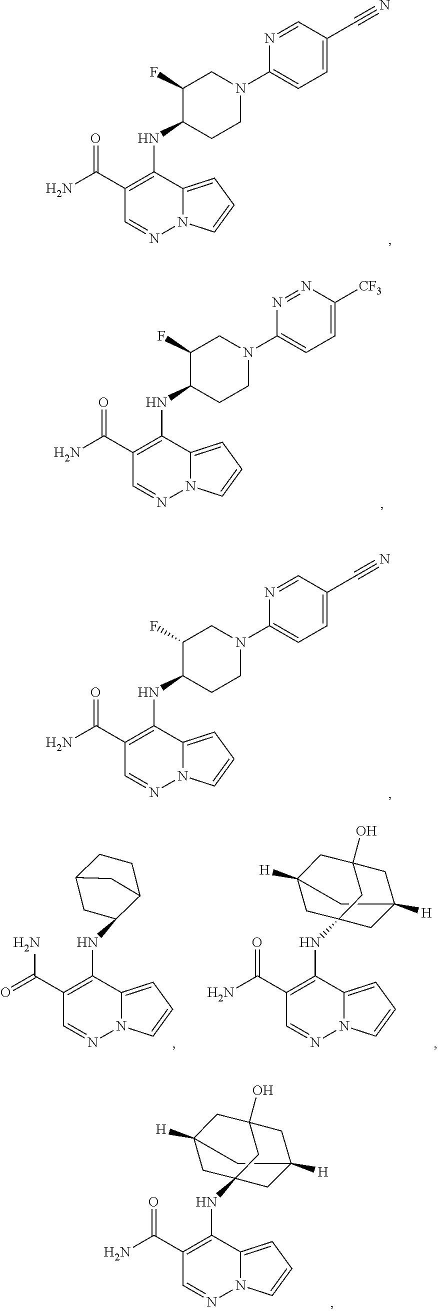

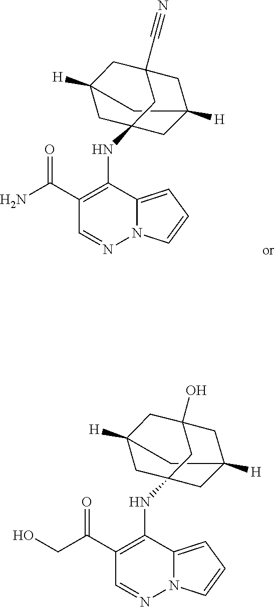

or a salt thereof.

Another specific compound of formula I is:

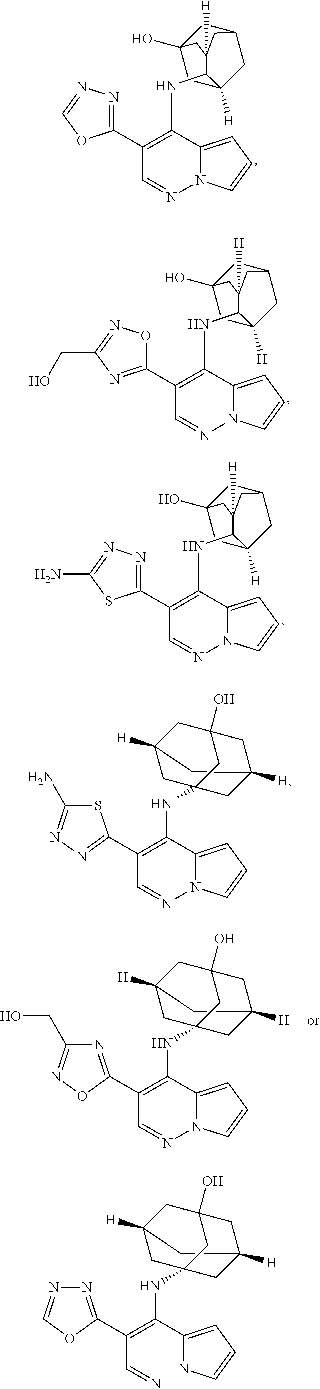

or a salt thereof.

Another specific compound of formula I is:

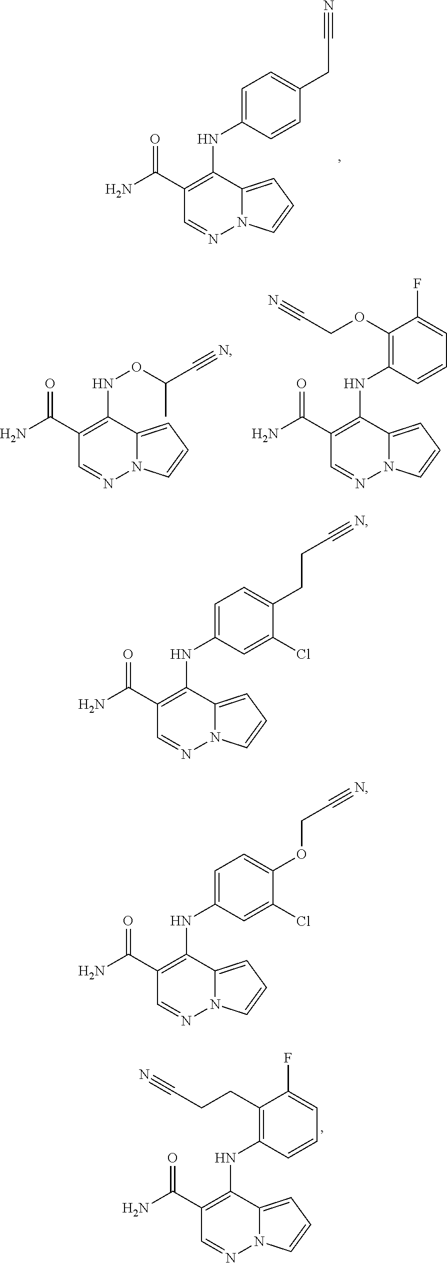

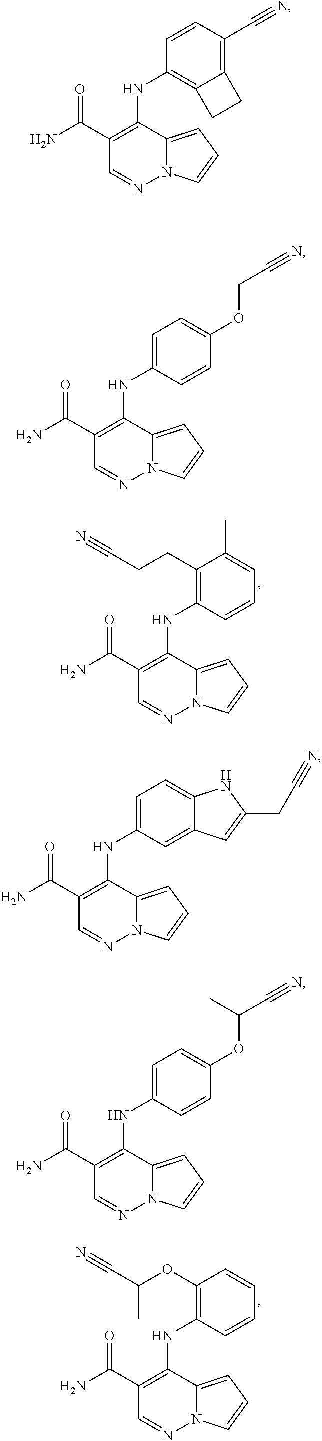

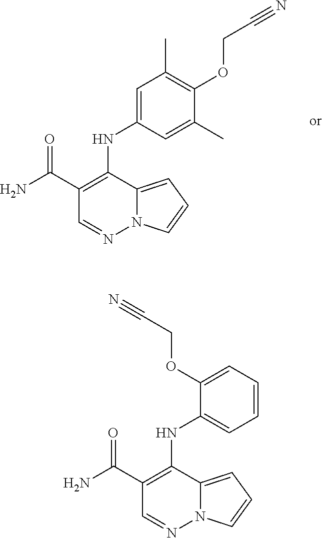

or a salt thereof.

Another specific compound of formula I is:

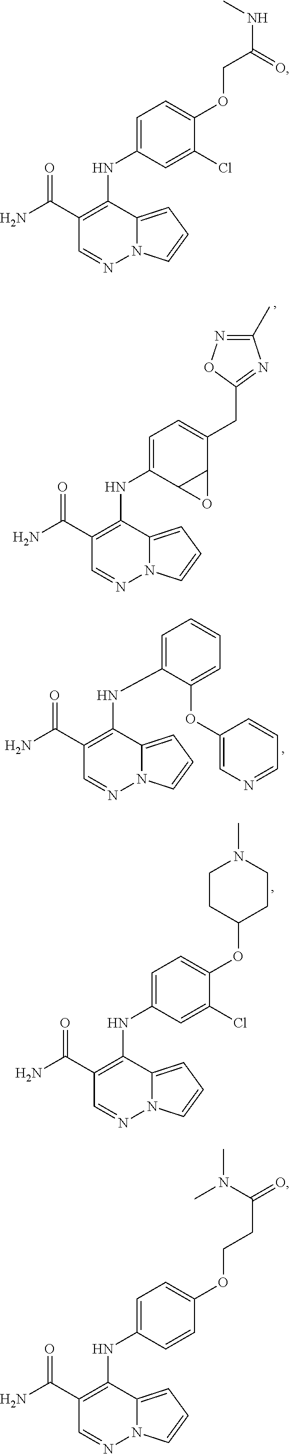

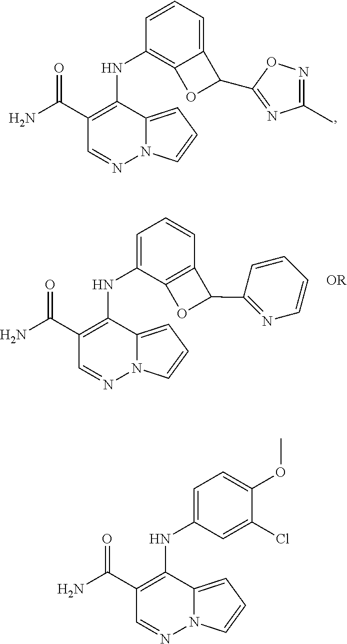

or a salt thereof.

Another specific compound of formula I is:

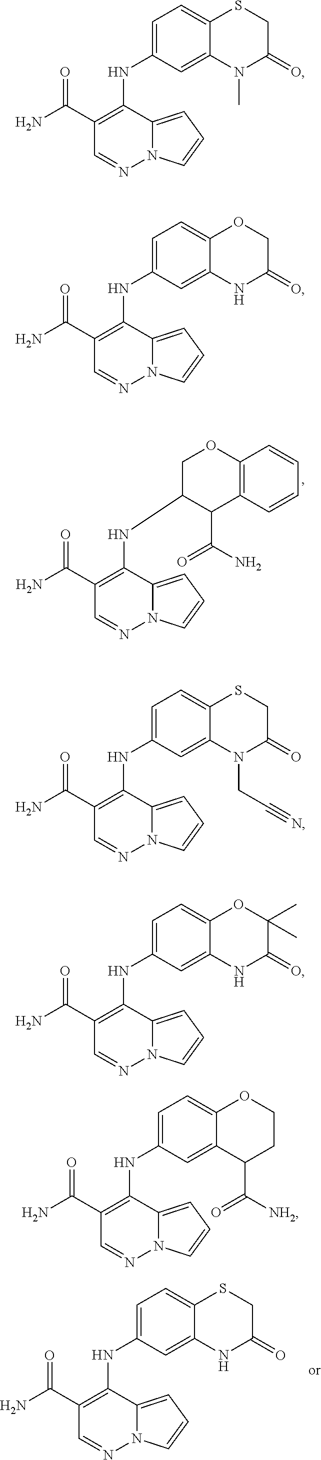

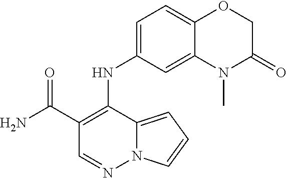

or a salt thereof.

Another specific compound of formula I is:

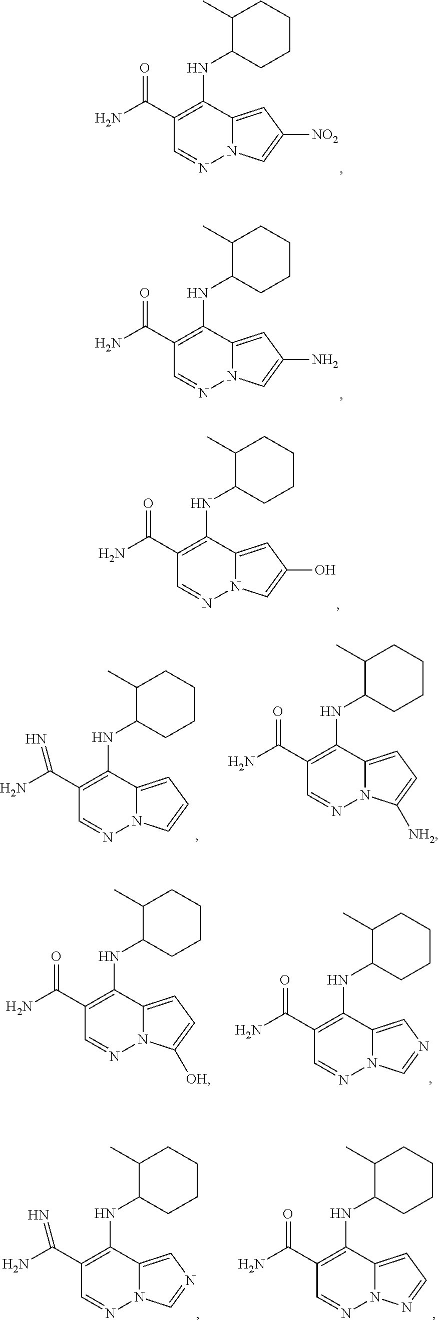

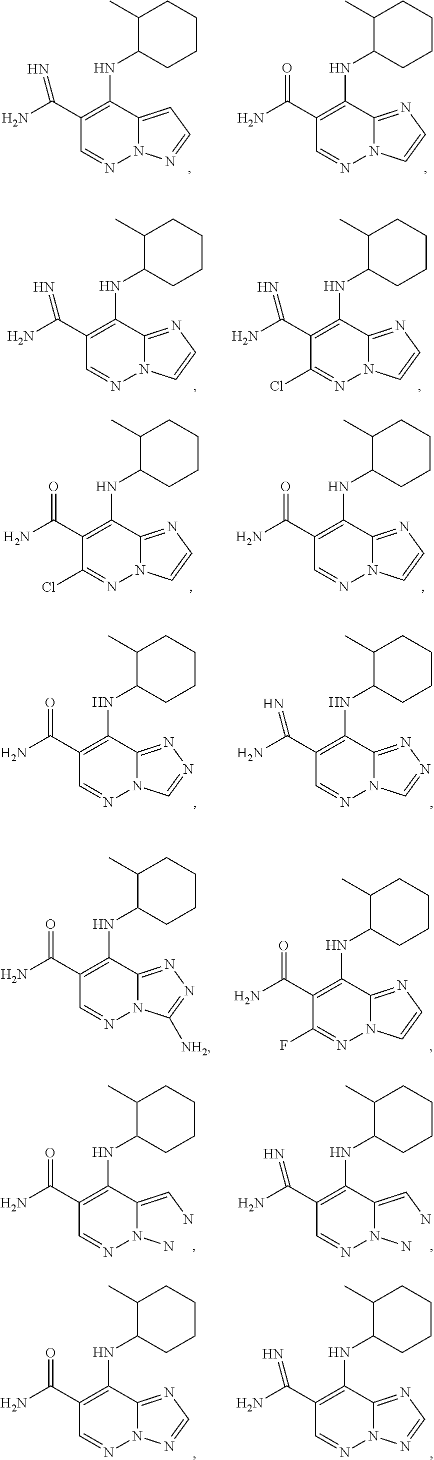

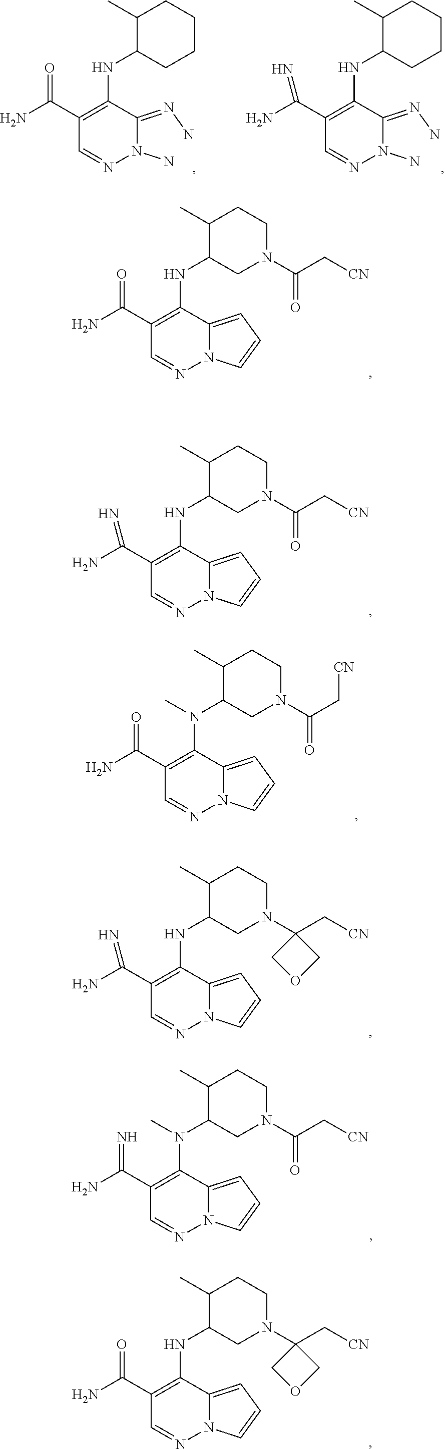

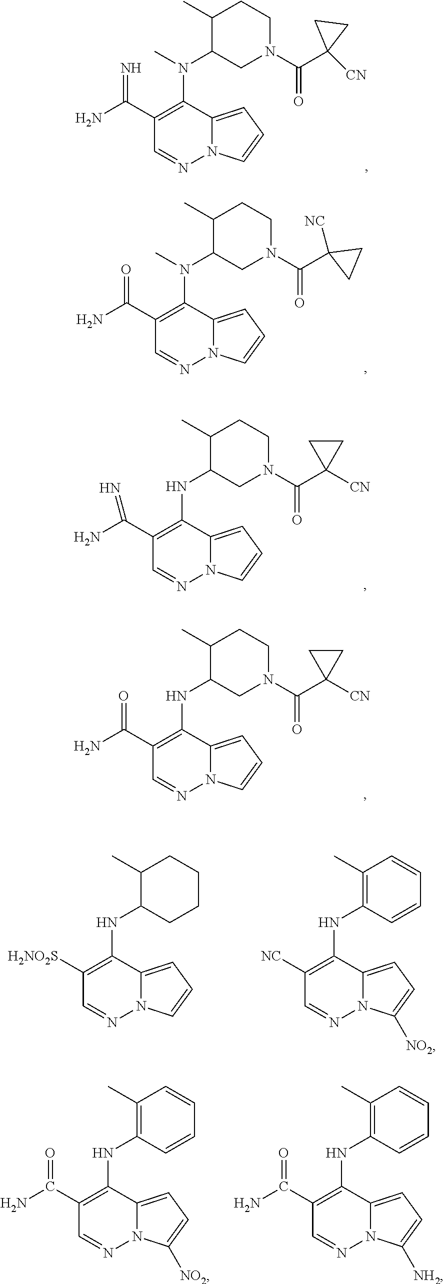

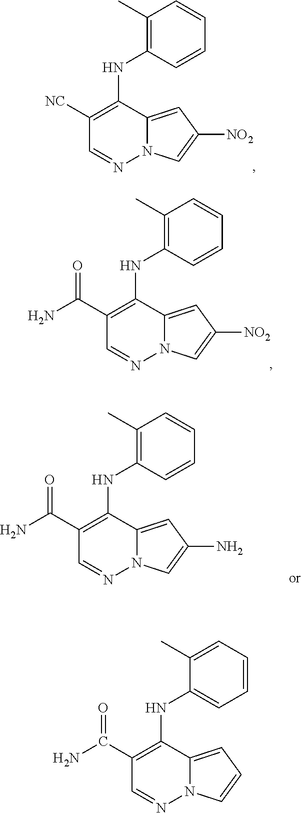

or a salt thereof.

Another specific compound of formula I is:

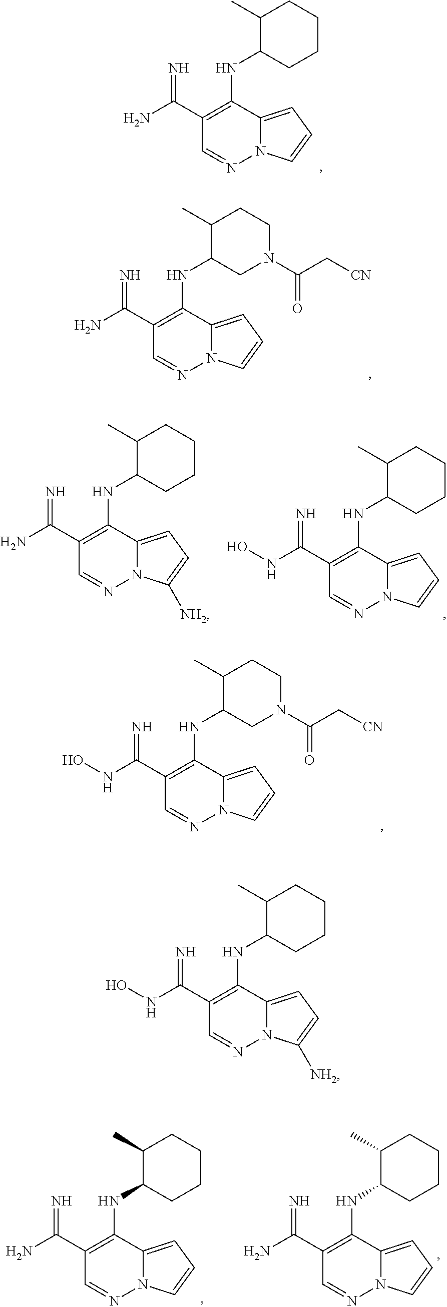

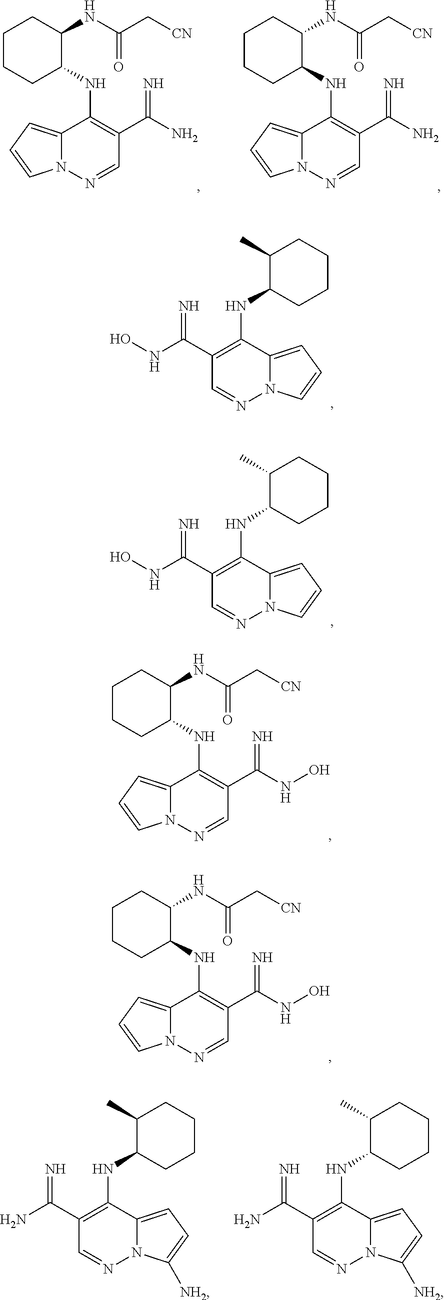

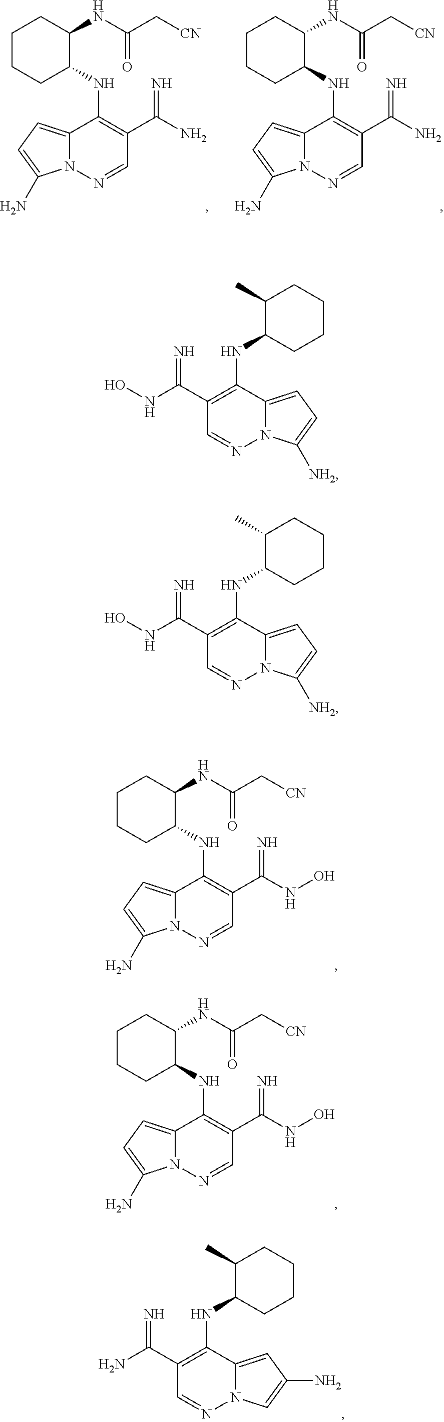

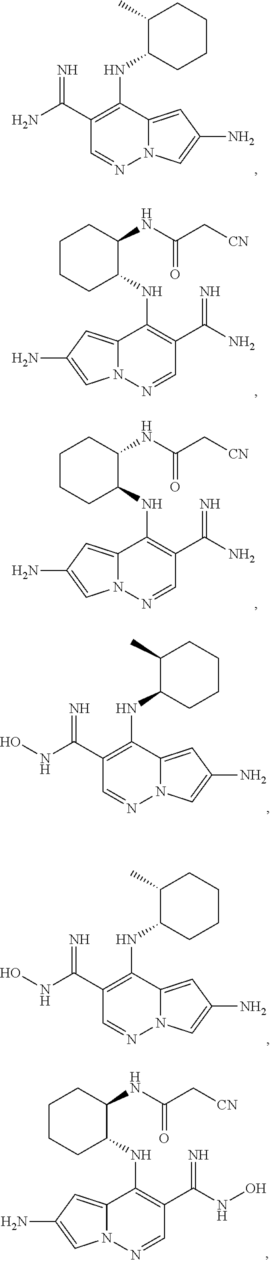

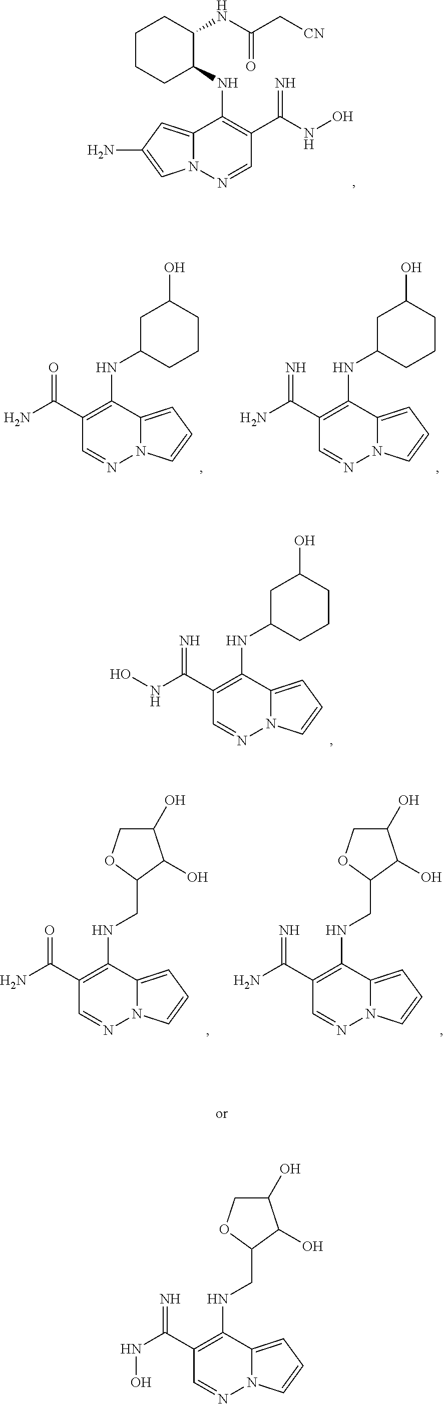

or a salt thereof.

Another specific compound of formula I is

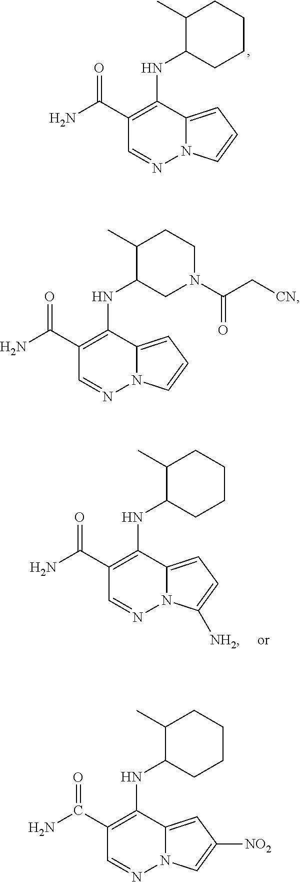

or a salt thereof.

Another specific compound of formula I is

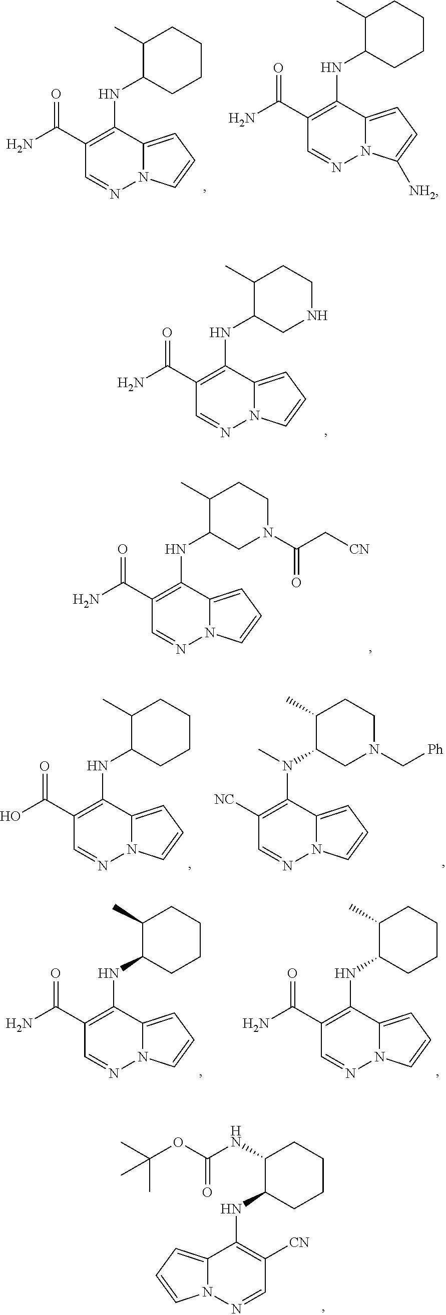

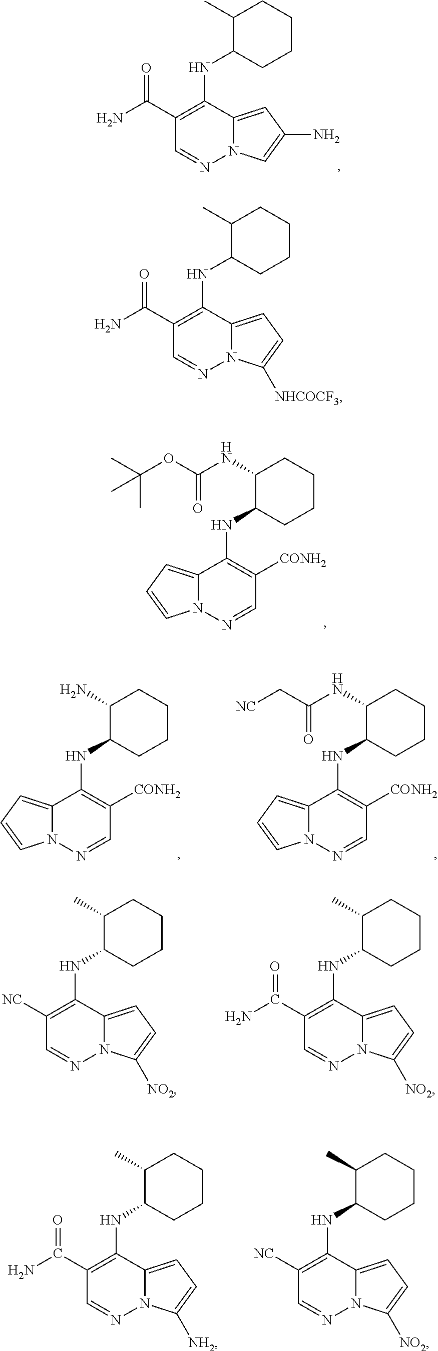

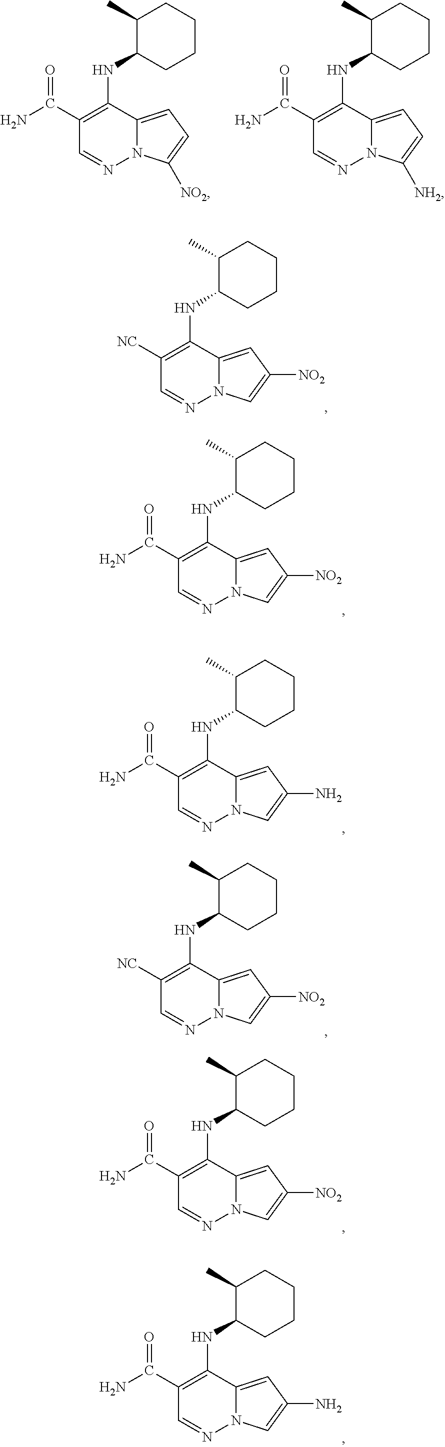

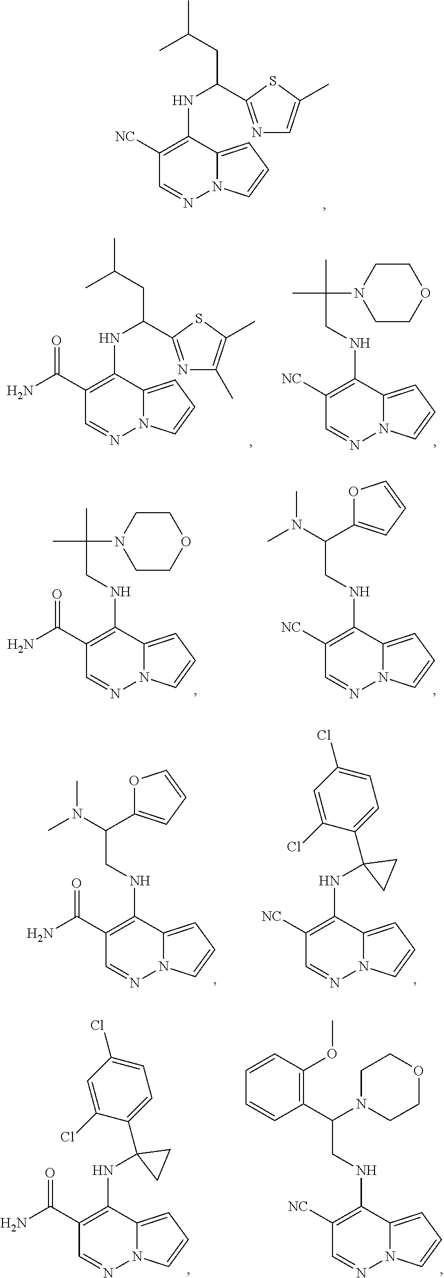

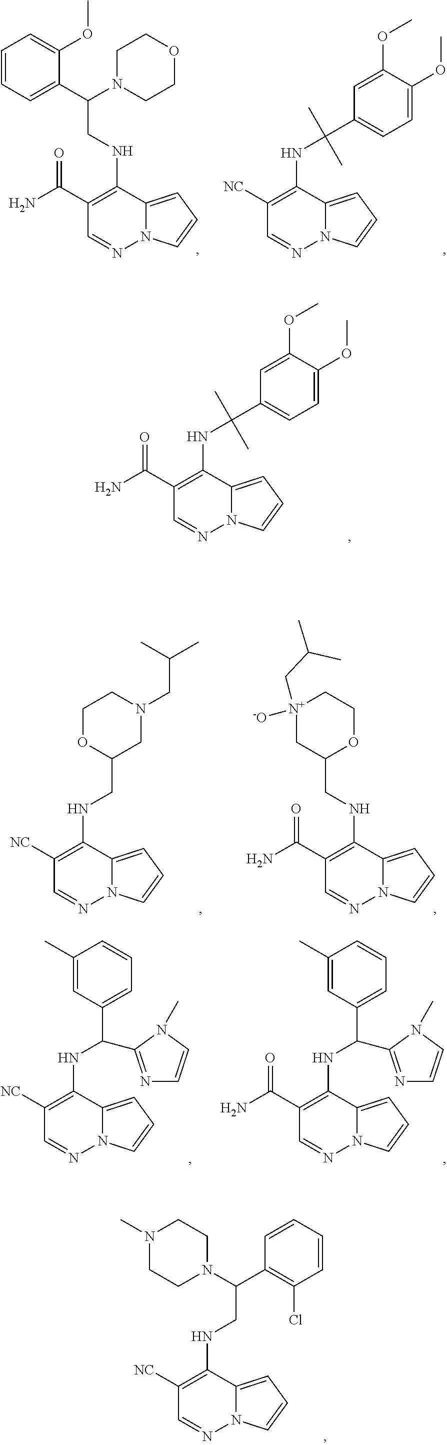

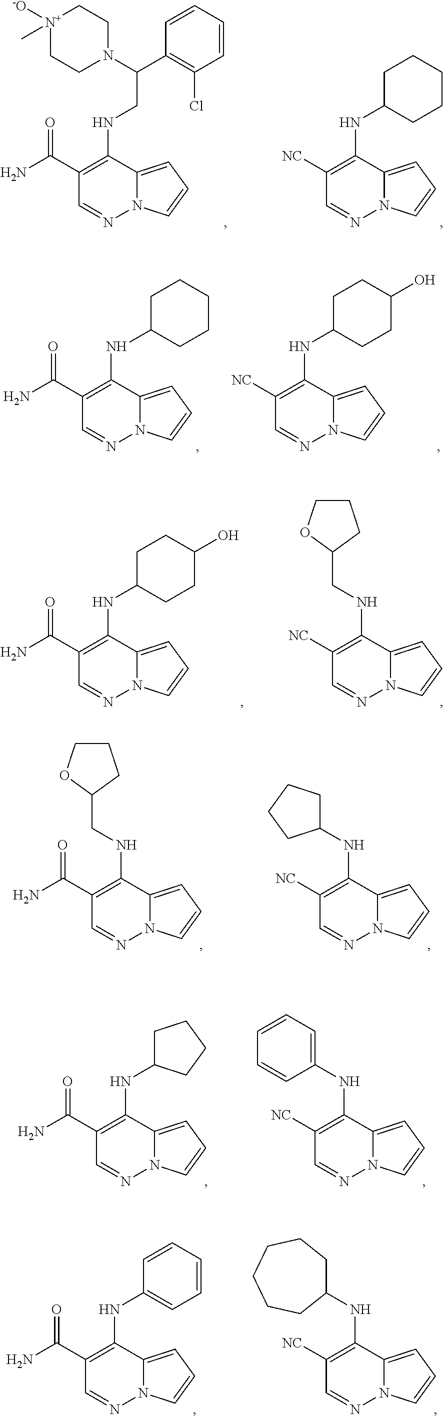

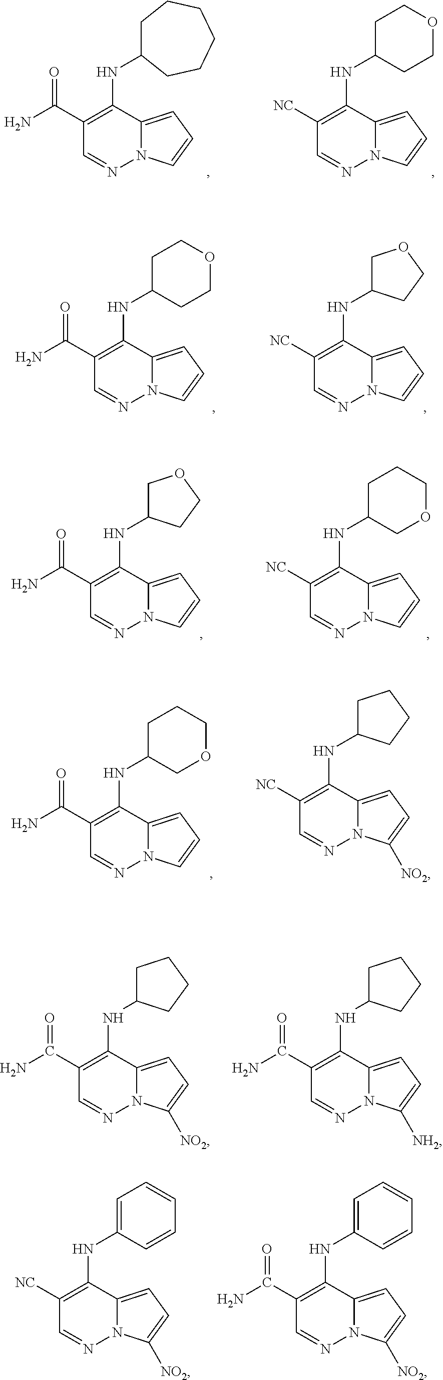

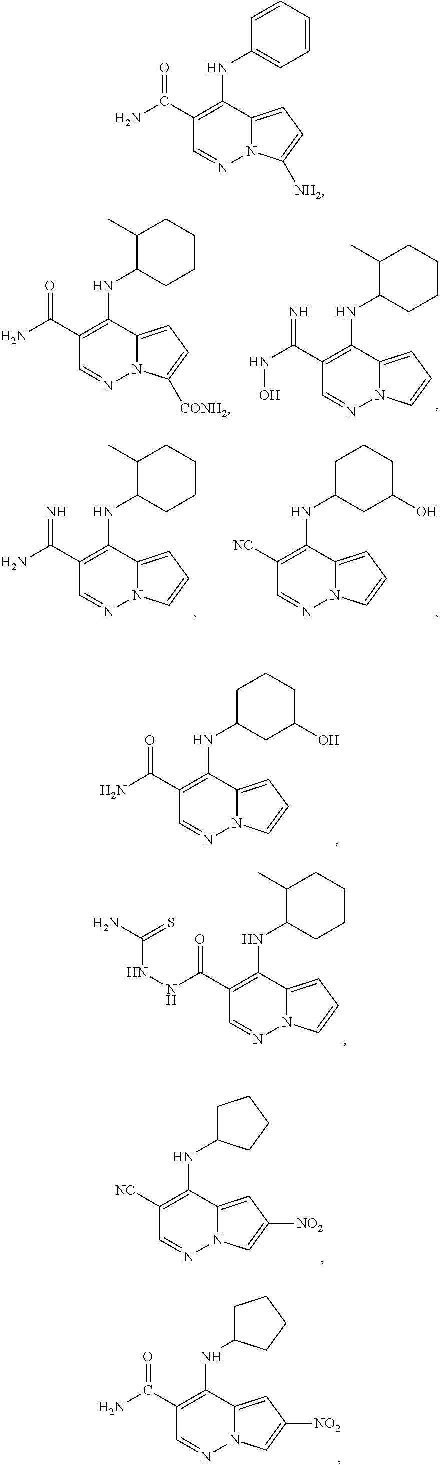

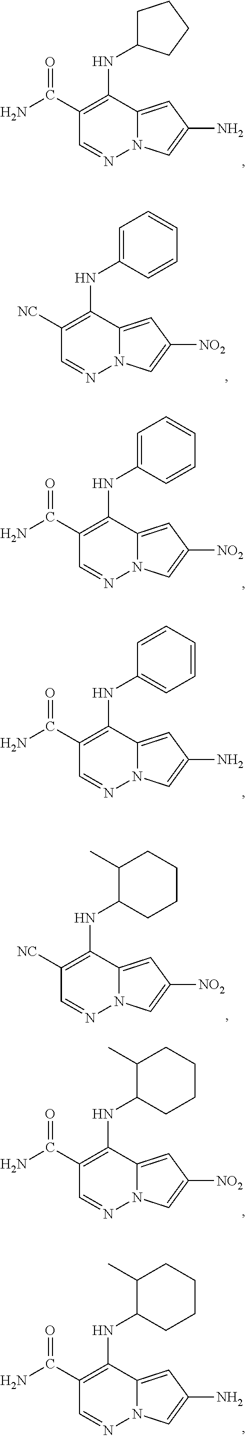

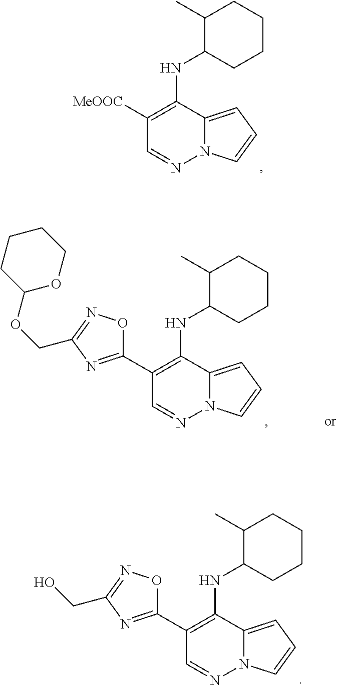

or a salt thereof.

In one embodiment the invention provides a compound of the invention which is a compound of formula I:

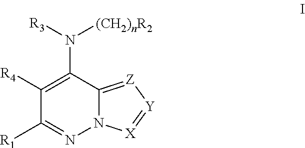

wherein

X is N or CR₅;

Y is N or CR₆;

Z is N or CR₇;

n is 0 or 1;

R₁ is H, halogen, alkyl, cycloalkyl, alkenyl, alkynyl, aryl, heteroaryl, heterocycle, NO₂, —CN, —OH, —OR_{d}, —NR_{b}R_{c}, N₃, SH, —C(O)Rₐ, —C(O)ORₐ, —C(O)NR_{b}R_{c}, —C(═NR_{b})NR_{b}R_{c}, —NR_{b}COR_{d}, —NR_{b}C(O)OR_{d}, —NR_{b}S(O)₂R_{d}, —NR_{b}CONR_{b}R_{c}, —OC(O)NR_{b}R_{c}, —S(O)R_{d}, —S(O)NR_{b}R_{c}, —S(O)₂R_{d}, —S(O)₂OH, or —S(O)₂NR_{b}R_{c} wherein any aryl or heteroaryl of R₁ may be optionally substituted with one or more (e.g. 1, 2, 3, 4 or 5) Rₑ groups and wherein any alkyl, cycloalkyl, alkenyl, alkynyl or heterocycle of R₁ may be optionally substituted with one or more (e.g. 1, 2, 3, 4 or 5) groups selected from Rₑ, oxo and ═NOR_{z};

R₂ is H, alkyl, cycloalkyl, heterocycle, heteroaryl, aryl, —Oalkyl or a bridged ring group wherein any aryl or heteroaryl of R₂ may be optionally substituted with one or more (e.g. 1, 2, 3, 4 or 5) R_{f} groups and wherein any alkyl, cycloalkyl, heterocycle or bridged ring group of R₂ may be optionally substituted with one or more (e.g. 1, 2, 3, 4 or 5) groups selected from R_{f}, oxo and ═NOR_{z};

R₃ is H, —CN, —C(O)alkyl, —C(O)alkenyl, —C(O)alkynyl, —C(O)cycloalkyl, —C(O)aryl, —C(═O)C(═O)NHlower alkyl, —CONR_{g}Rₕ, alkyl, alkenyl, heterocycle, or heteroaryl, wherein any aryl or heteroaryl of R₃ may be optionally substituted with one or more (e.g. 1, 2, 3, 4 or 5) Rᵢ groups and wherein any alkyl, alkenyl, alkynyl, cycloalkyl, heterocycle or lower alkyl of R₃ may be optionally substituted with one or more (e.g. 1, 2, 3, 4 or 5) groups selected from Rᵢ, oxo and ═NOR_{z};

R₄ is halogen, alkyl, cycloalkyl, alkenyl, alkynyl, aryl, heteroaryl, heterocycle, NO₂, —CN, OH, —ORₙ, —NRₖRₘ, N₃, —SH, —SRₙ, —C(O)alkyl, —C(O)alkenyl, —C(O)alkynyl, —C(O)cycloalkyl, —C(O)aryl, —C(O)heteroaryl, —C(O)heterocycle, —C(O)ORⱼ, —C(O)NRₖRₘ, —C(═NRₖ)NRₖRₘ, —NRₖCORₙ, —NRₖC(O)ORₙ, —NRₖS(O)₂Rₙ, —NRₖCONRₖRₘ, —OC(O)NRₖRₘ, —S(O)Rₙ, —S(O)NRₖRₘ, —S(O)₂Rₙ, —S(O)₂OH, —S(O)₂NRₖRₘ or —C(═O)C(═O)NHlower alkyl wherein any aryl or heteroaryl of R₄ may be optionally substituted with one or more (e.g. 1, 2, 3, 4 or 5) Rₚ groups and wherein any alkyl, lower alkyl, cycloalkyl, alkenyl, alkynyl or heterocycle of R₄ may be optionally substituted with one or more (e.g. 1, 2, 3, 4 or 5) groups selected from Rₚ, oxo and ═NOR_{z};

R₅ is H, OH, NO₂, CO₂H, —NR_{q}Rᵣ, halogen or lower alkyl which lower alkyl is optionally substituted with one or more (e.g. 1, 2, 3, 4 or 5) Rₛ groups;

R₆ is H, OH, NO₂, CO₂H, —NR_{q}Rᵣ, halogen or lower alkyl which lower alkyl is optionally substituted with one or more (e.g. 1, 2, 3, 4 or 5) Rₛ groups;

R₇ is H, OH, NO₂, CO₂H, —NR_{q}Rᵣ, halogen or lower alkyl which lower alkyl is optionally substituted with one or more (e.g. 1, 2, 3, 4 or 5) Rₛ groups;

Rₐ is H, alkyl, alkenyl, alkynyl, cycloalkyl, heterocycle, heteroaryl or aryl;

R_{b} and R_{c} are each independently selected from H, alkyl, alkenyl, alkynyl, cycloalkyl, heterocycle and heteroaryl; or R_{b} and R_{c} together with the nitrogen to which they are attached form a pyrrolidino, piperidino, piperazino, azetidino, morpholino, or thiomorpholino ring;

R_{d} is alkyl, alkenyl, alkynyl, cycloalkyl, heterocycle, heteroaryl or aryl;

Rₑ is independently selected from halogen, aryl, heteroaryl, heterocycle, R_{z}, OH, —CN, —OR_{z}, —Oaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z} and wherein any aryl of Rₑ may be optionally substituted with one or more (e.g. 1, 2, 3, 4 or 5) R_{y} groups;

R_{f} is independently selected from halogen, aryl, heteroaryl, heterocycle, R_{z}, OH, —CN, —OR_{z}, —Oaryl, —Oheterocycle, —Oheteroaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2}, —C(O)heterocycle, —C(O)heteroaryl and —C(O)C(O)R_{z} and wherein any aryl, heteroaryl, or heterocycle of R_{f} may be optionally substituted with one or more (e.g. 1, 2, 3, 4 or 5) R_{y} groups;

R_{g} and Rₕ are each independently selected from H, alkyl, alkenyl, alkynyl, cycloalkyl, heterocycle and heteroaryl; or R_{g} and Rₕ together with the nitrogen to which they are attached form a pyrrolidino, piperidino, piperazino, azetidino, morpholino, or thiomorpholino ring;

R₁ is independently selected from halogen, aryl, heteroaryl, heterocycle, R_{z}, OH, —CN, —OR_{z}, —Oaryl, —OC(O)R_{z}, —OC(O)NR_{z1}R_{z2}, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z} and wherein any aryl of Rᵢ may be optionally substituted with one or more (e.g. 1, 2, 3, 4 or 5) R_{y} groups;

Rⱼ is H, alkyl, alkenyl, alkynyl, cycloalkyl, heterocycle, heteroaryl or aryl;

Rₖ and Rₘ are each independently selected from H, alkyl, alkenyl, alkynyl, cycloalkyl, heterocycle and heteroaryl; or Rₖ and Rₘ together with the nitrogen to which they are attached form a pyrrolidino, piperidino, piperazino, azetidino, morpholino, or thiomorpholino ring;

Rₙ is alkyl, alkenyl, alkynyl, cycloalkyl, heterocycle, heteroaryl or aryl;

Rₚ is independently selected from halogen, aryl, heteroaryl, heterocycle, R_{z}, OH, —CN, —OR_{z}, —Oaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z} and wherein any aryl of Rₚ may be optionally substituted with one or more (e.g. 1, 2, 3, 4 or 5) R_{y} groups;

R_{q} and Rᵣ are each independently selected from H, alkyl, alkenyl, alkynyl, cycloalkyl, heterocycle and heteroaryl; or R_{q} and Rᵣ together with the nitrogen to which they are attached form a pyrrolidino, piperidino, piperazino, azetidino, morpholino, or thiomorpholino ring;

Rₛ is independently selected from halogen, aryl, heteroaryl, heterocycle, R_{z}, OH, —CN, —OR_{z}, —Oaryl, —OC(O)R_{z}, —OC(O)NR_{z1}R_{z2}, oxo, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, ═NOR_{z}, —CHO, —C(O)R_{z}, —C(O)OH, —C(O)OR_{z}, —C(O)NR_{z1}R_{z2} and —C(O)C(O)R_{z} wherein any aryl of Rₛ may be optionally substituted with one or more (e.g. 1, 2, 3, 4 or 5) R_{y} groups;

R_{z} is independently lower alkyl or lower cycloalkyl wherein lower alkyl or lower cycloalkyl may be optionally substituted with one or more (e.g. 1, 2 or 3) groups selected from halogen, —CN, OH, —Olower alkyl, —NHlower alkyl, —C(O)NHlower alkyl, —C(O)N(lower alkyl)₂, heterocycle and heteroaryl wherein heterocycle may be substituted with one or more (e.g. 1, 2 or 3) lower alkyl;

R_{z1} and R_{z2} are each independently selected from H, lower alkyl, alkenyl, alkynyl, lower cycloalkyl, heterocycle and heteroaryl, wherein lower alkyl or lower cycloalkyl may be optionally substituted with one or more (e.g. 1, 2 or 3) Rₜ groups; or R_{z1} and R_{z2} together with the nitrogen to which they are attached form a cyclic amino;

Rₜ is independently selected from halogen, —CN, OH, —Olower alkyl, —NHlower alkyl, —C(O)NHlower alkyl, —C(O)N(lower alkyl)₂, heterocycle and heteroaryl wherein any heterocycle of Rₜ may be substituted with one or more (e.g. 1, 2 or 3) lower alkyl; and

each R_{y} is independently halogen, aryl, R_{z}, OH, —CN, OR_{z}, —Oaryl, —Oheteroaryl, —OC(O)R_{z}, —OC(O)NR_{z1}R_{z2}, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, CHO, —C(O)R_{z}, —C(O)OH, —C(O)OR_{z}, —C(O)NR_{z1}R_{z2}, —C(O)C(O)R_{z}, heterocycle or heteroaryl;

or a salt thereof.

The invention also provides for compounds of formula 5d and compounds of formula 5d wherein R²² is NH₂. These compounds are useful as inhibitors of JAK (e.g. JAK1, JAK2 or TYK2).

In cases wherein n=0, R₂ is connected to NR₃ by a carbon atom of R₂ (i.e. carbon linked).

Tautomers:

A wide variety of functional groups and other structures exhibit tautomerism and all tautomers of compounds of formula I are within the scope of the present invention.

For example, pyrazoles may exhibit the isomeric forms referred as tautomers. Tautomers are isomeric forms of a compound that are in equilibrium with each other. The concentrations of the isomeric forms will depend on the environment in which the compound is found and may be different depending on if the compound is a solid or is in an organic or aqueous solution.

Processes which were used to prepare compounds of formula I are provided as further embodiments of the invention and are illustrated in Schemes 13, 16, 18, 19, 37, 40, 45-55 and 57. Additional processes which can be used to prepare compounds of formula I or intermediates useful for preparing compounds of formula I are provided in Schemes 1-12, 14, 15, 17, 20-36, 38, 39, 41-44 and 56 and also represent embodiments of the invention.

### General Methods of Preparation of Invention Compounds:

Heterocycles can be prepared from know methods as reported in the literature (a. Ring system handbook, published by American Chemical Society edition 1993 and subsequent supplements. b. The Chemistry of Heterocyclic Compounds; Weissberger, A., Ed.; Wiley: New York, 1962. c. Nesynov, E. P.; Grekov, A. P. The chemistry of 1,3,4-oxadiazole derivatives. Russ. Chem. Rev. 1964, 33, 508-515. d. Advances in Heterocyclic Chemistry; Katritzky, A. R., Boulton, A. J., Eds.; Academic Press: New York, 1966. e. In Comprehensive Heterocyclic Chemistry; Potts, K. T., Ed.; Pergamon Press: Oxford, 1984. f. Eloy, F. A review of the chemistry of 1,2,4-oxadiazoles. Fortschr. Chem. Forsch. 1965, 4, pp 807-876. g. Adv. Heterocycl. Chem. 1976. h. Comprehensive Heterocyclic Chemistry; Potts, K. T., Ed.; Pergamon Press: Oxford, 1984. i. Chem. Rev. 1961 61, 87-127. j. 1,2,4-Triazoles; John Wiley & Sons: New York, 1981; Vol 37). Some of the functional groups during the synthesis may need to be protected and subsequently deprotected. Examples of suitable protecting groups can be found in “Protective groups in organic synthesis” fourth edition edited by Greene and Wuts.

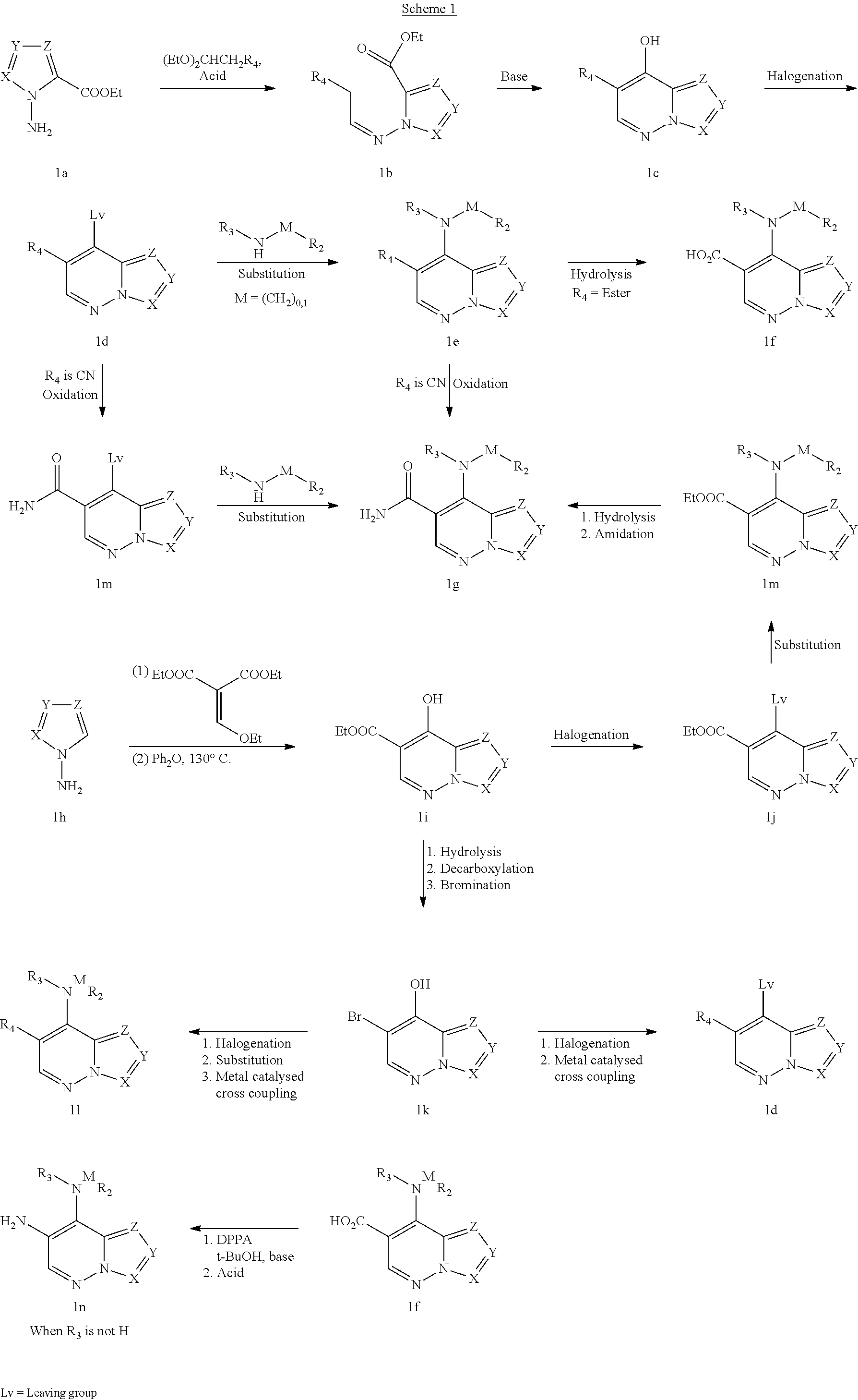

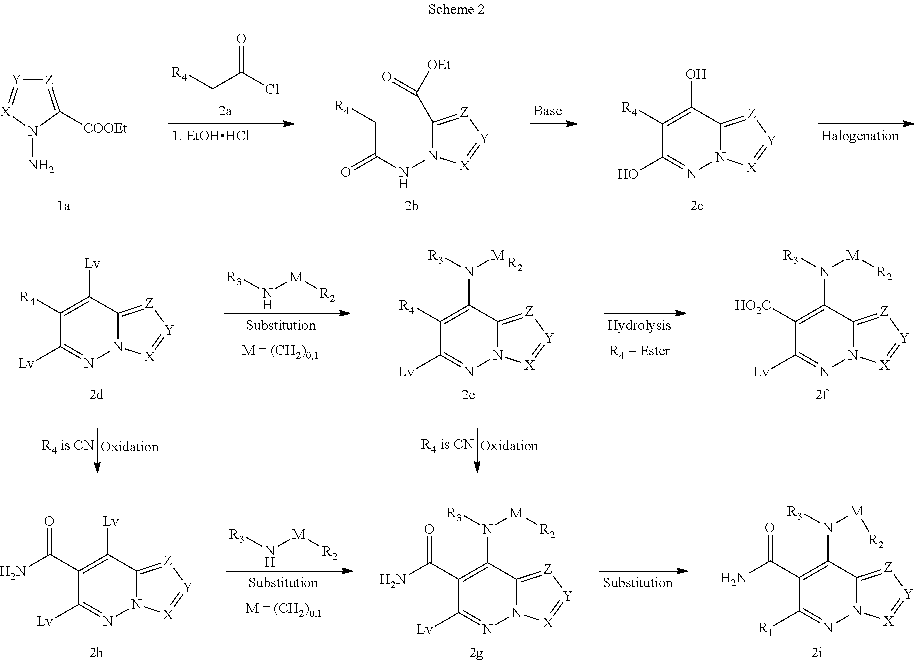

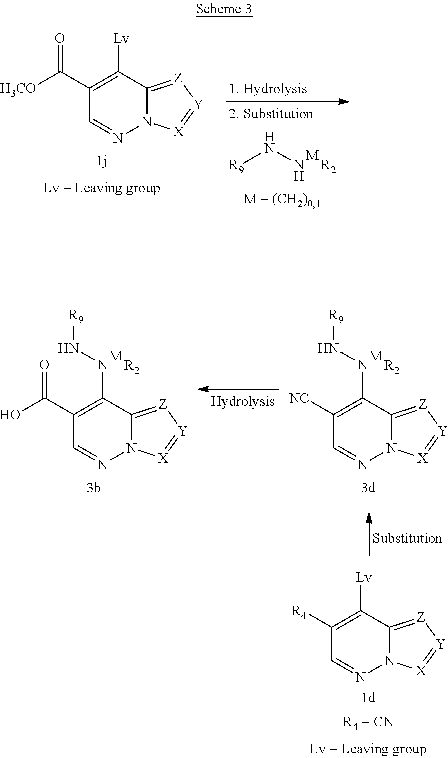

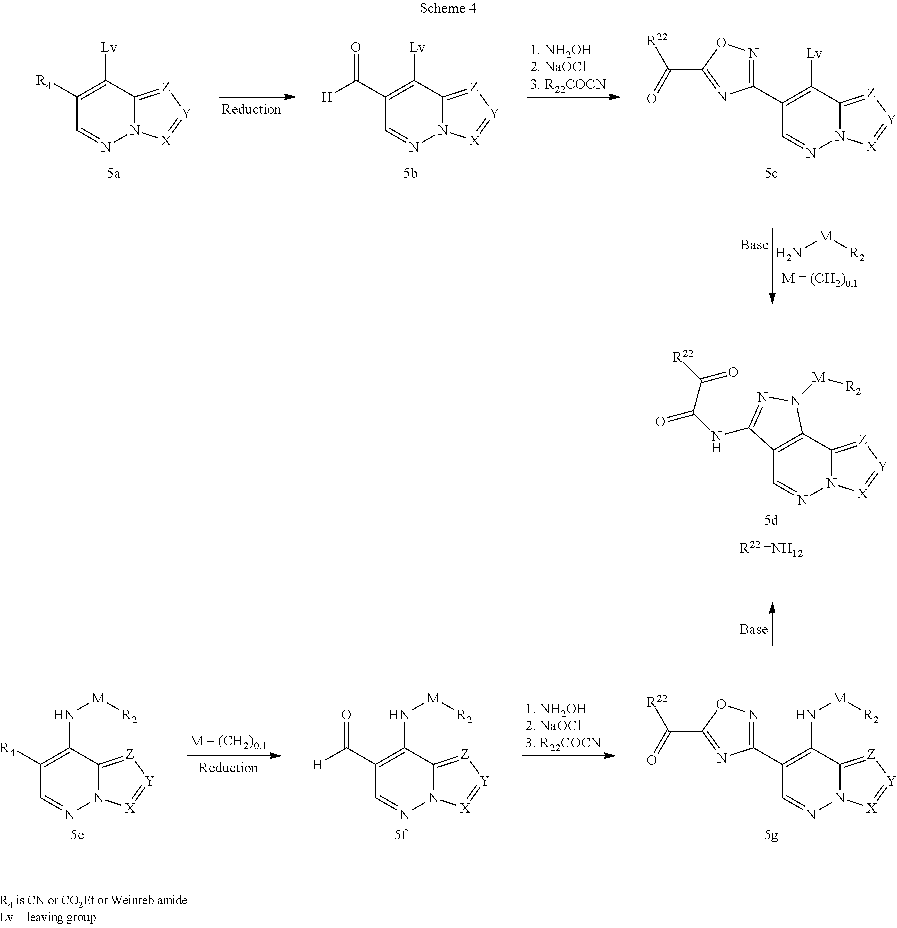

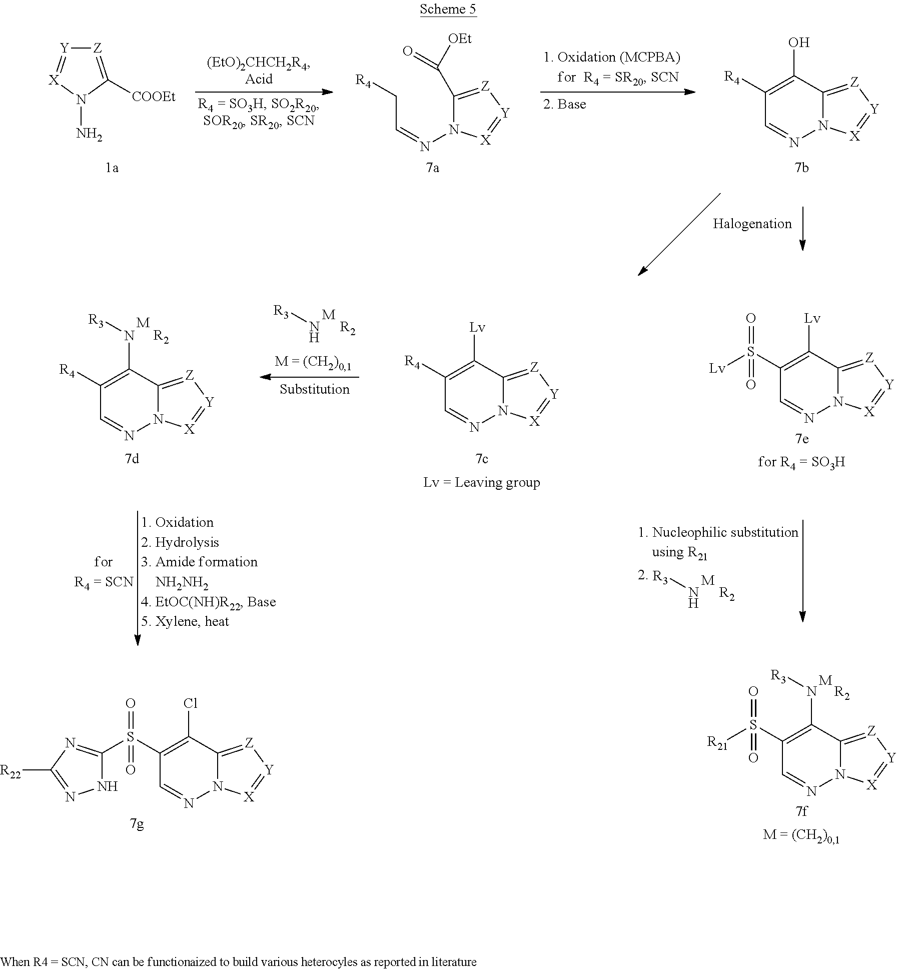

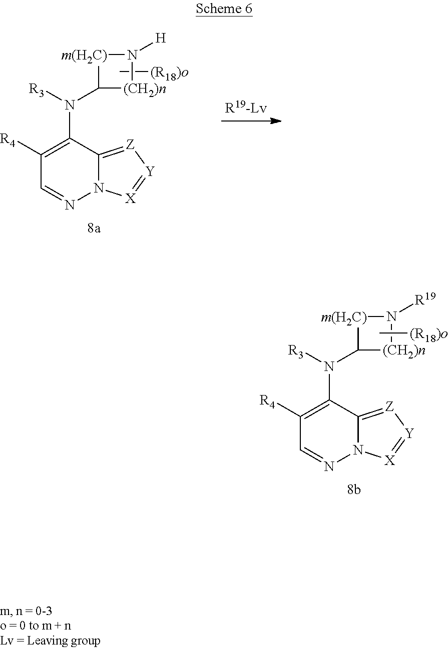

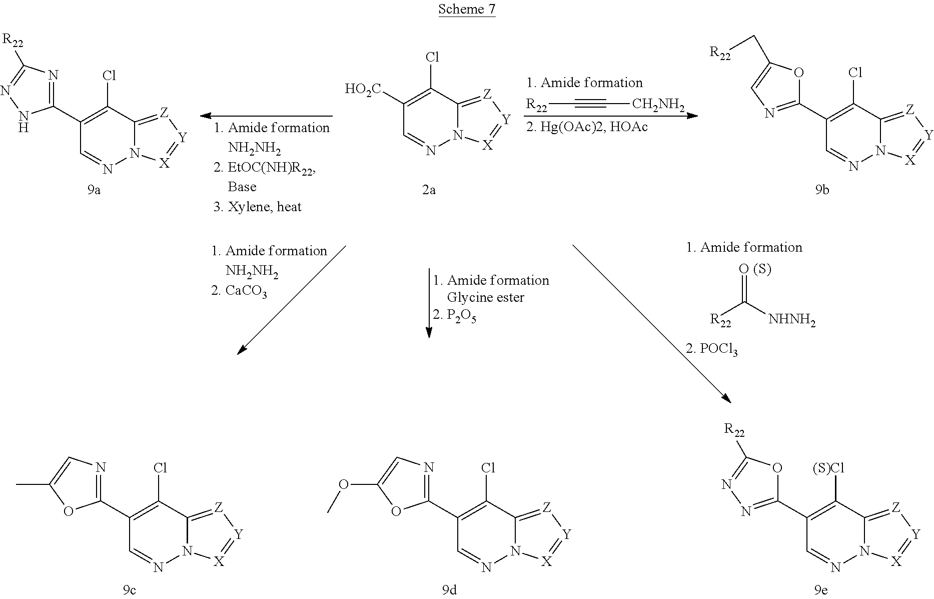

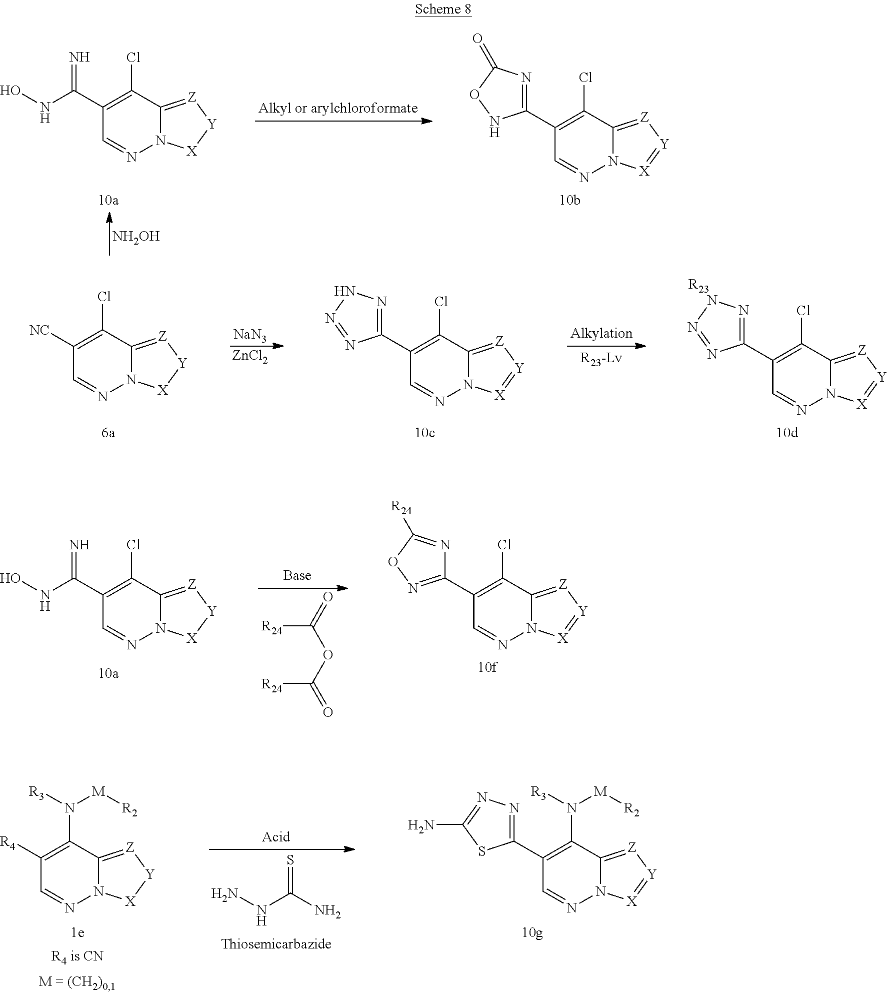

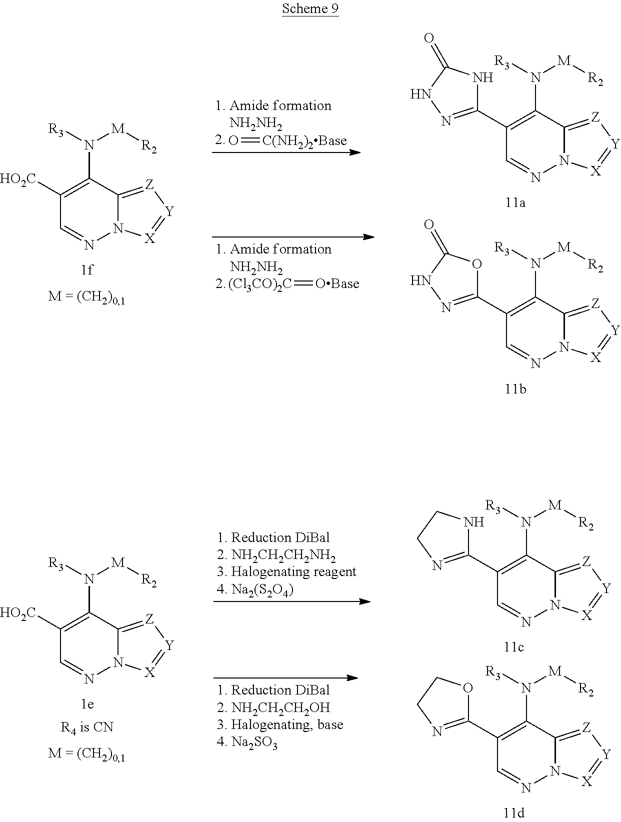

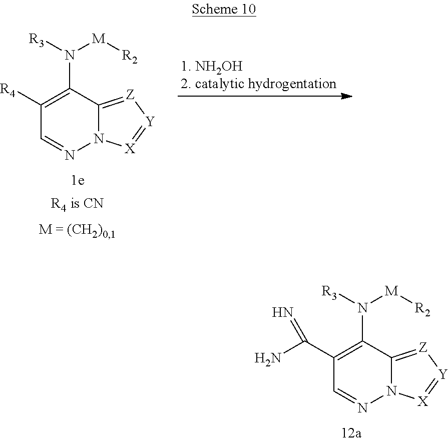

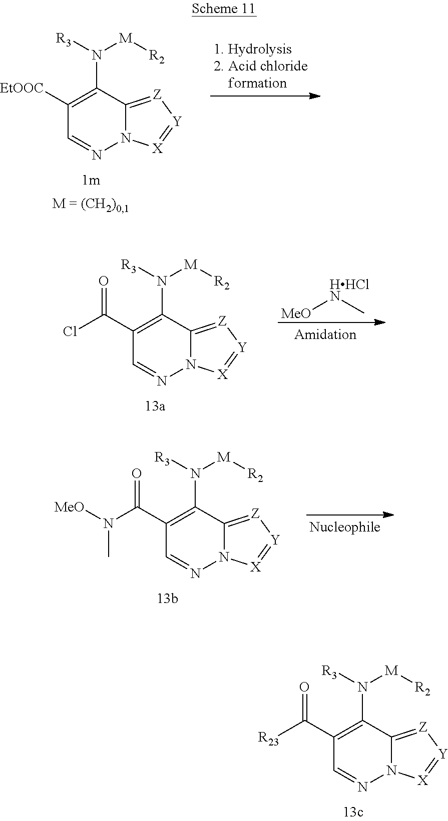

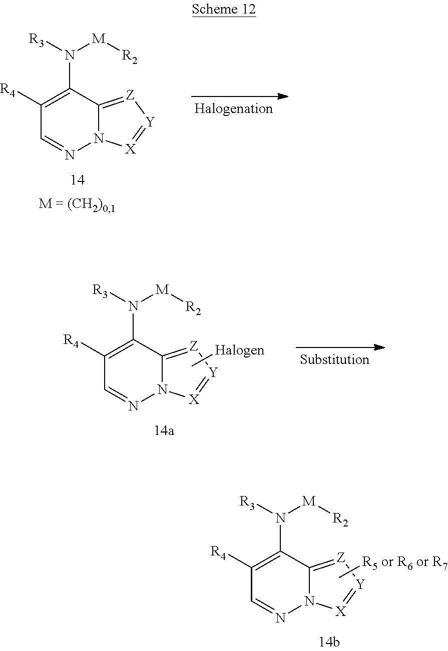

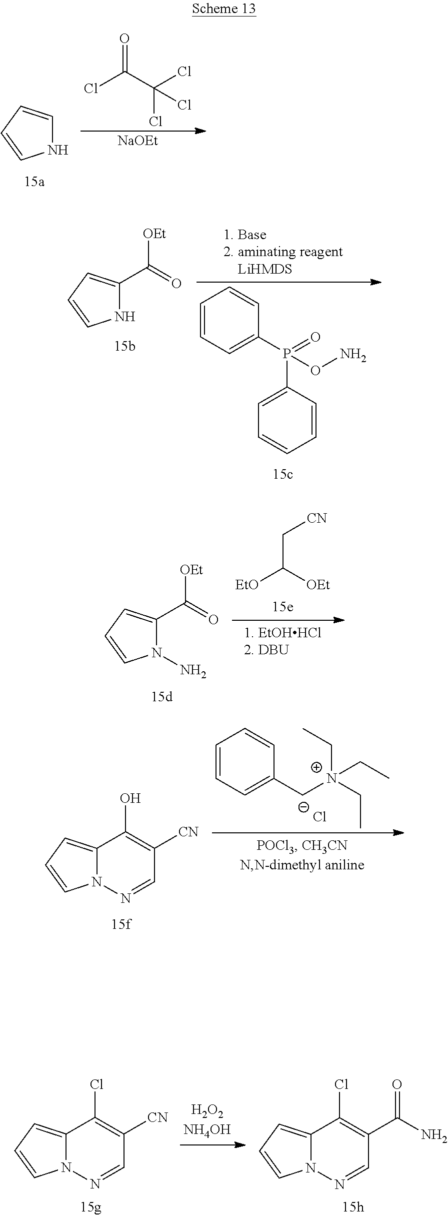

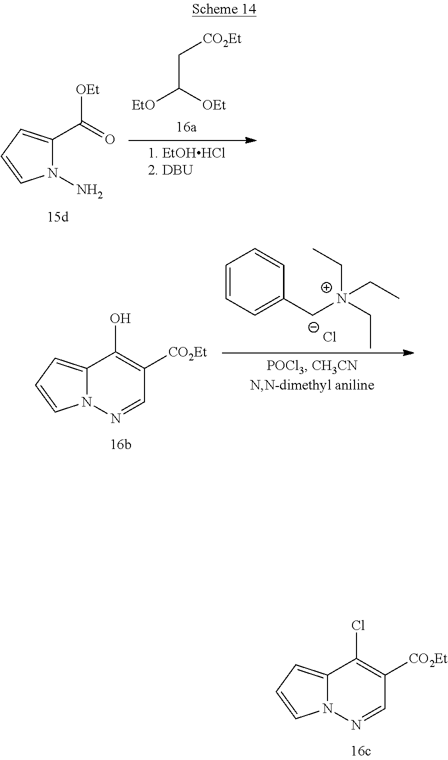

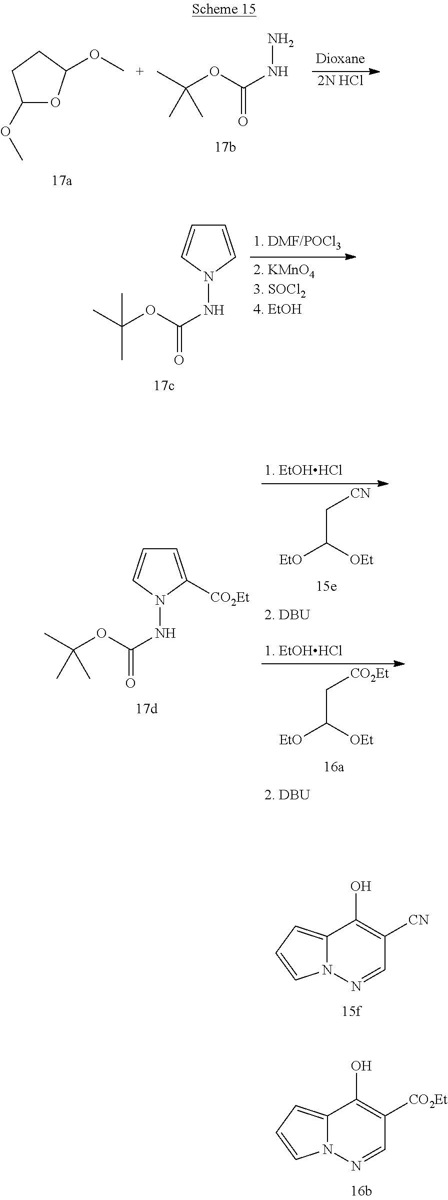

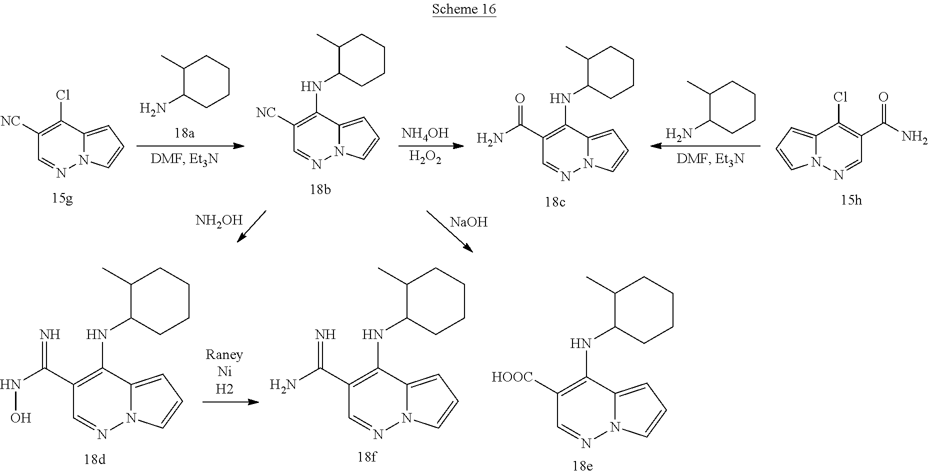

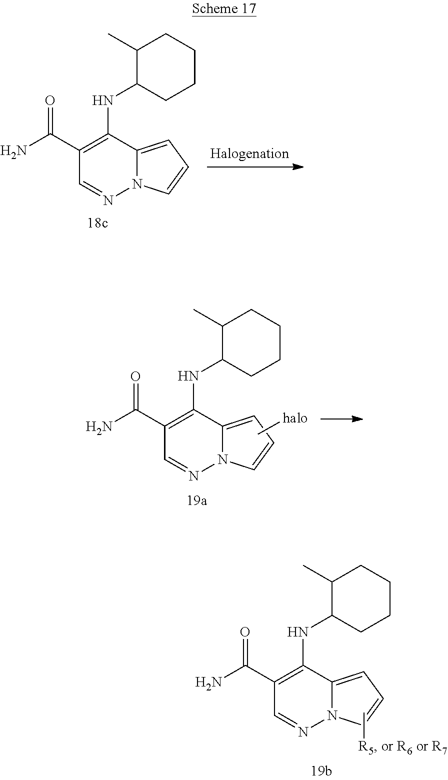

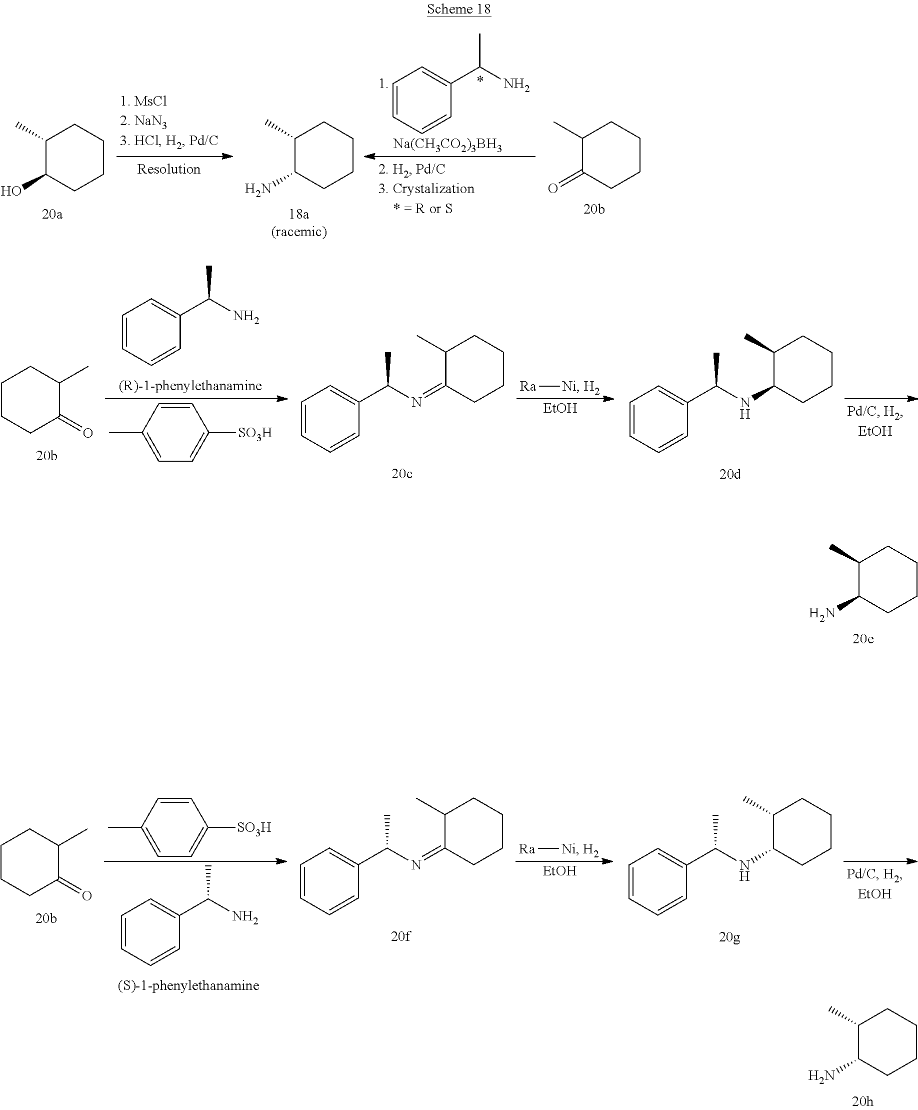

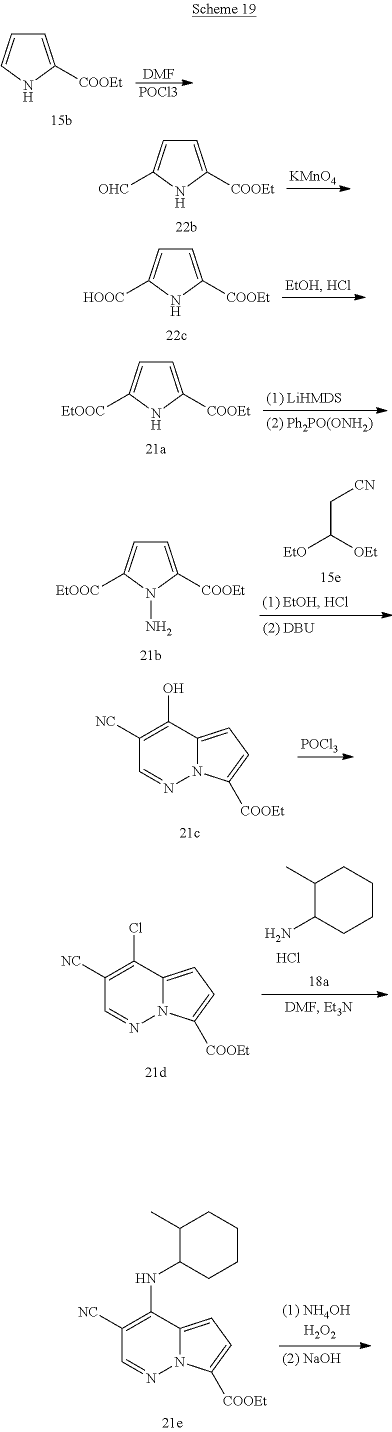

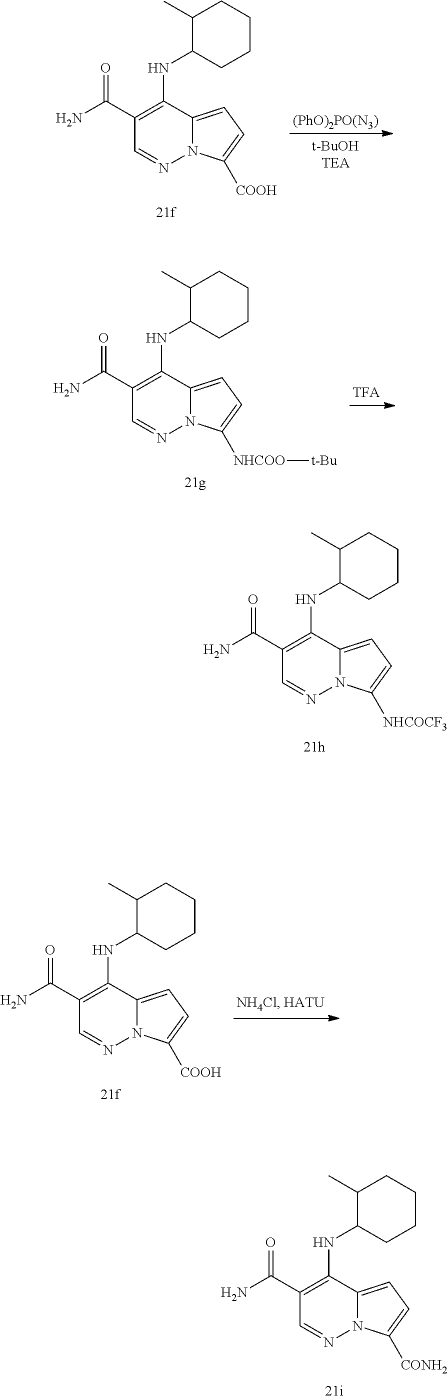

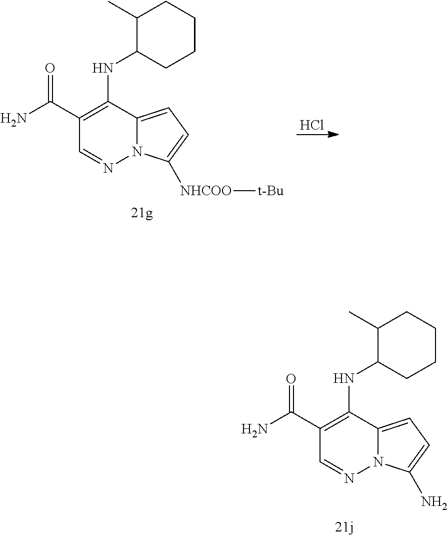

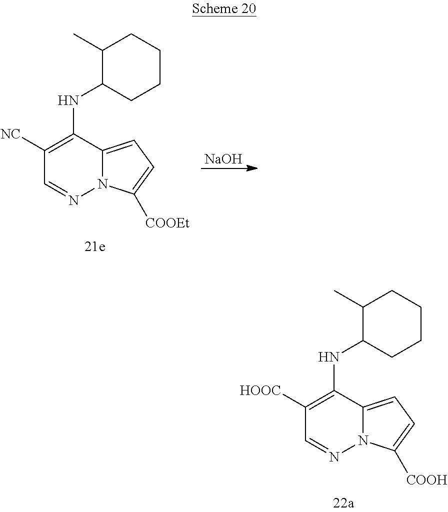

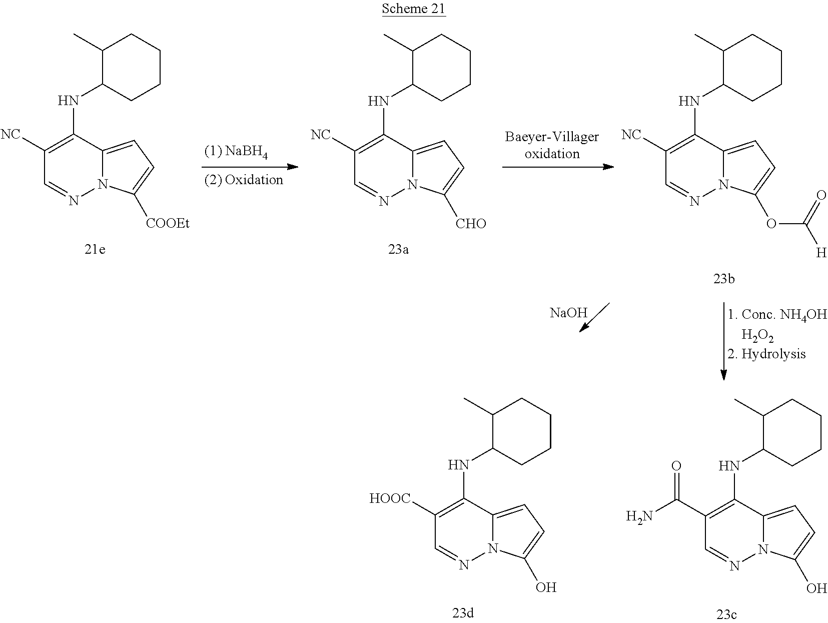

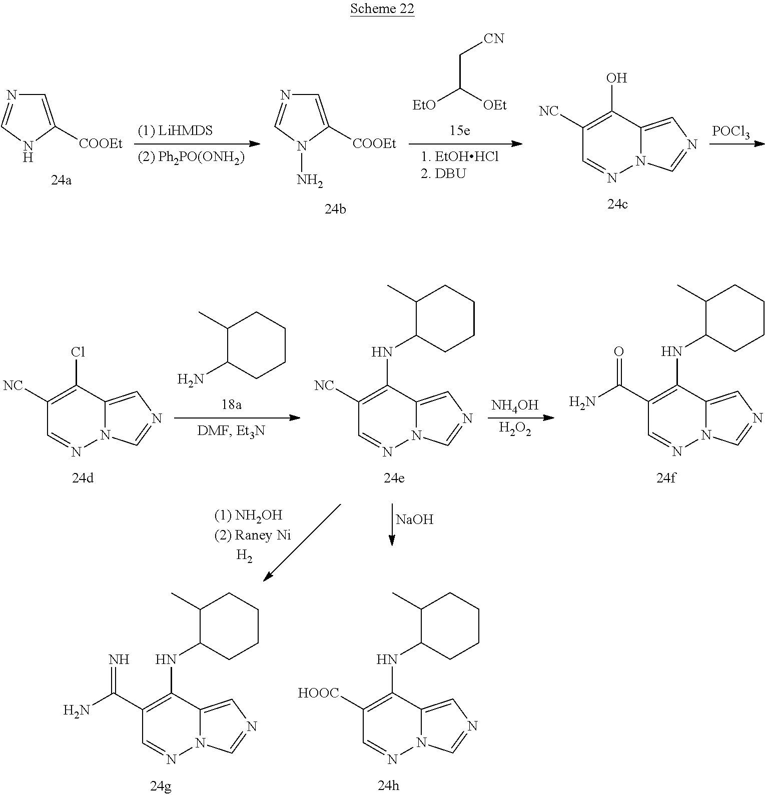

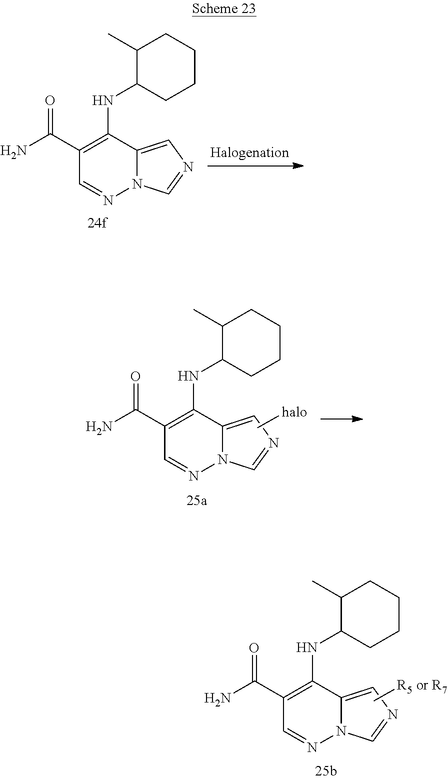

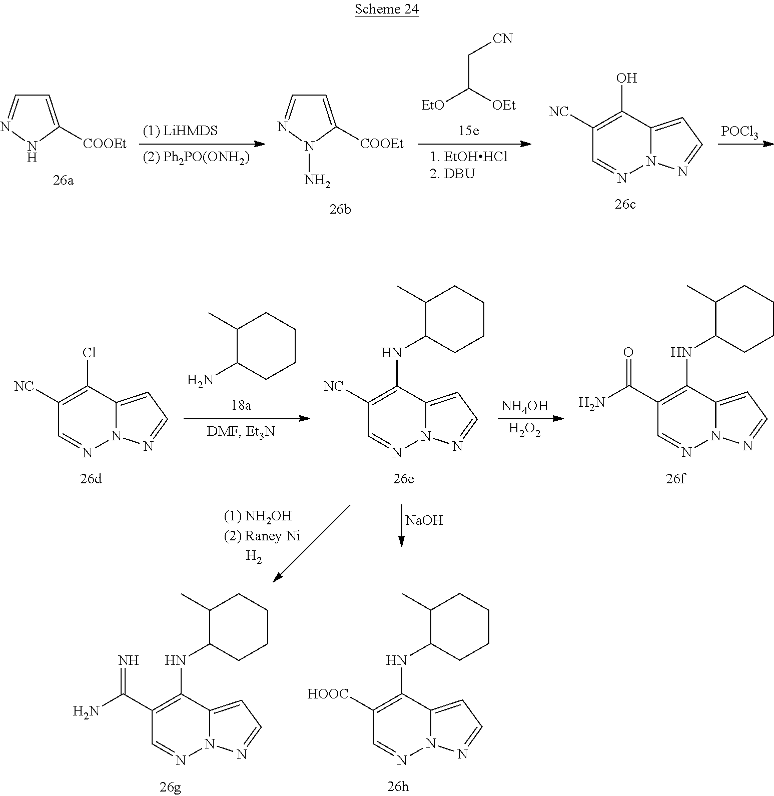

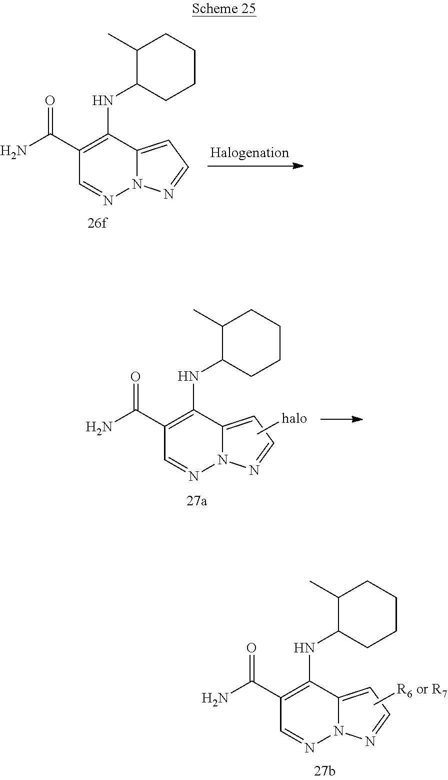

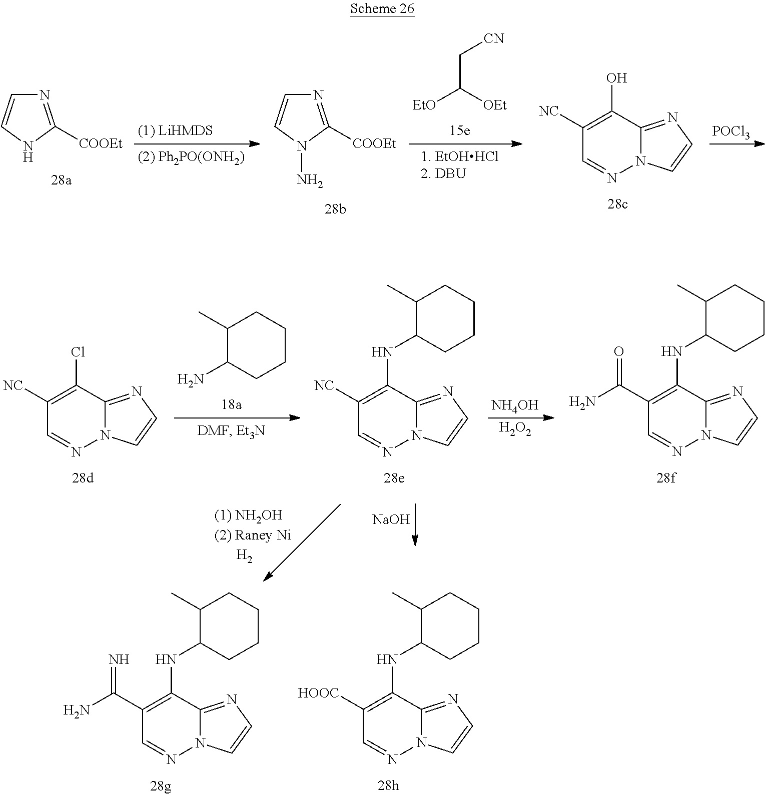

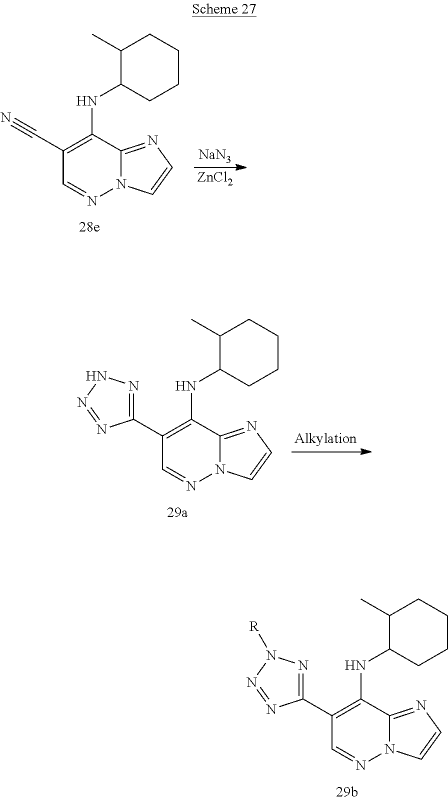

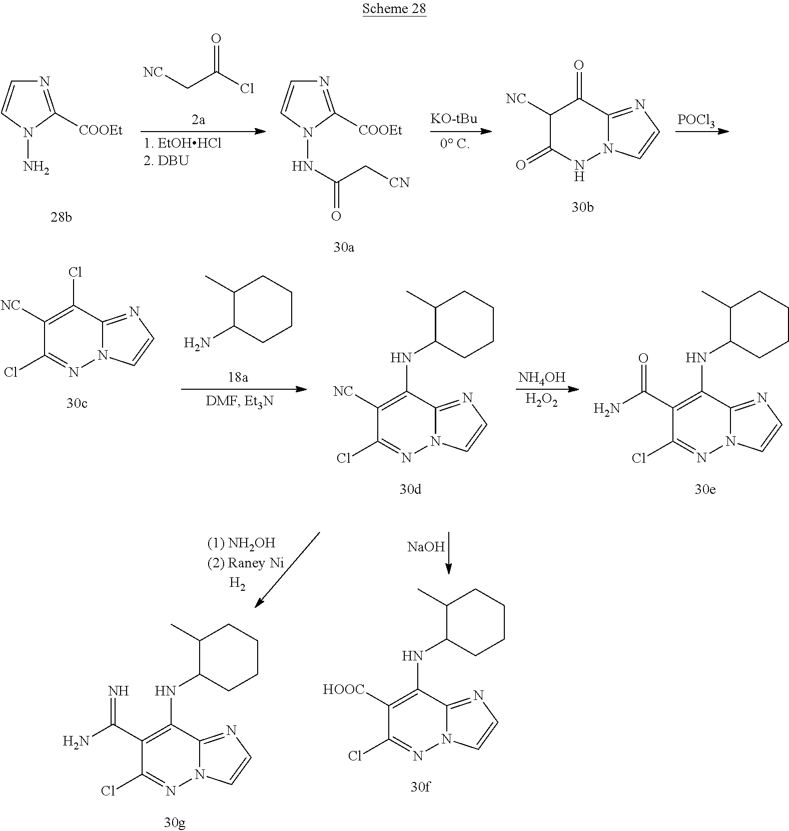

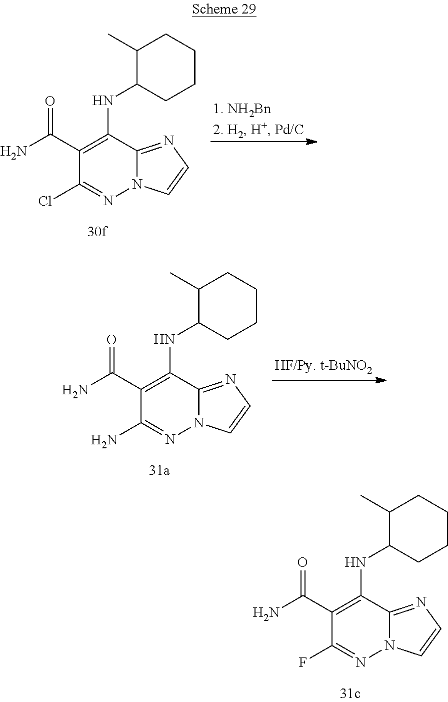

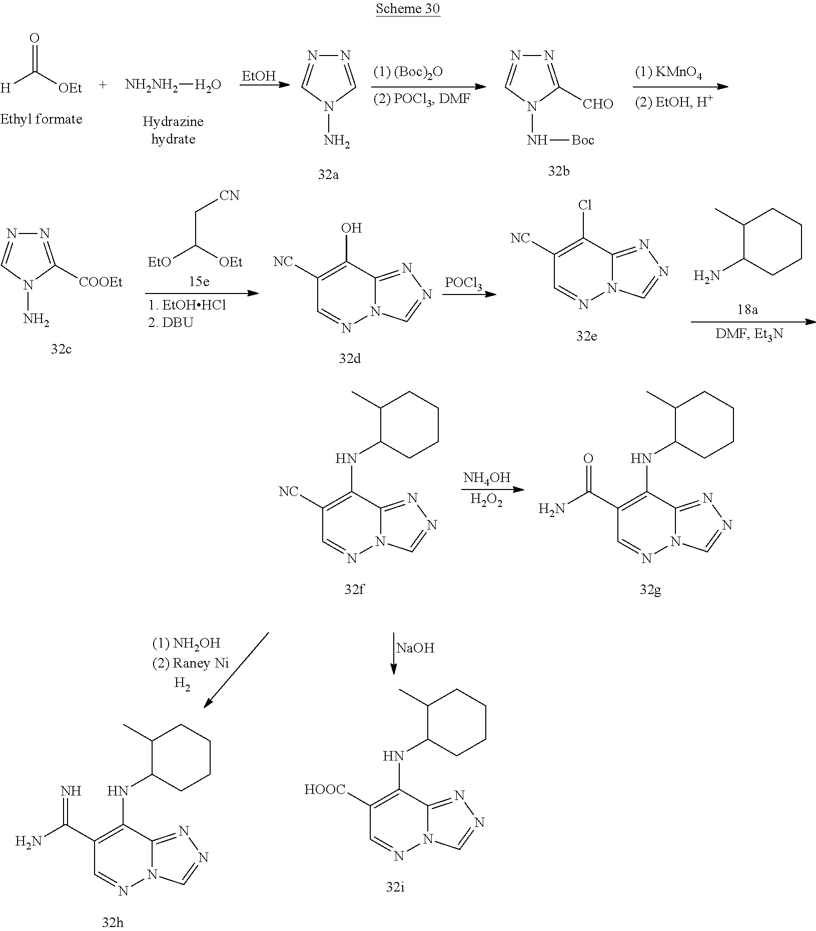

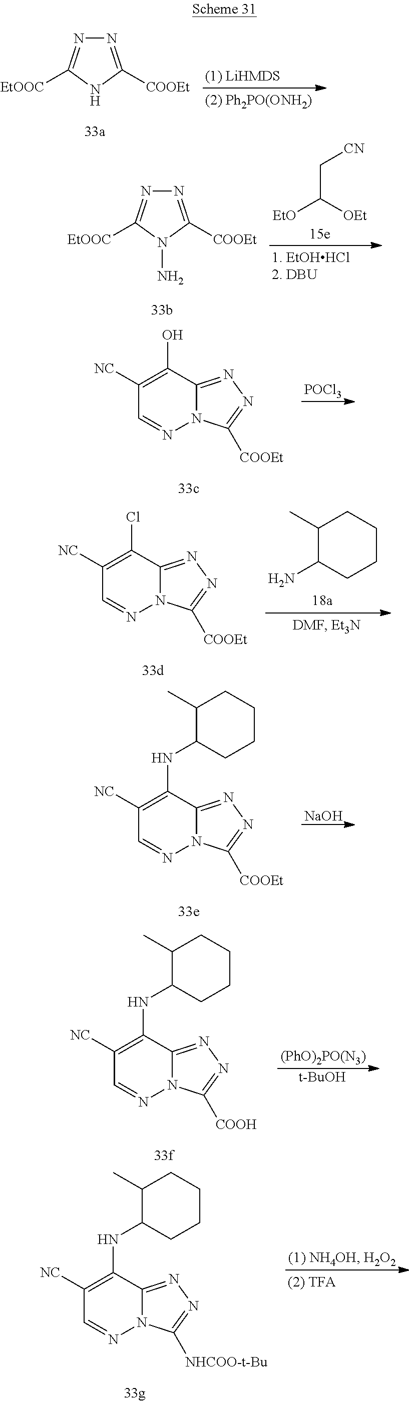

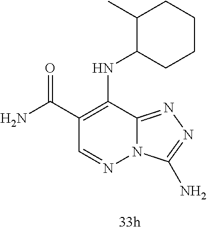

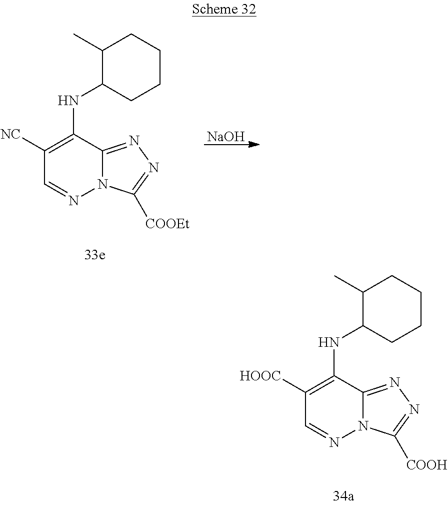

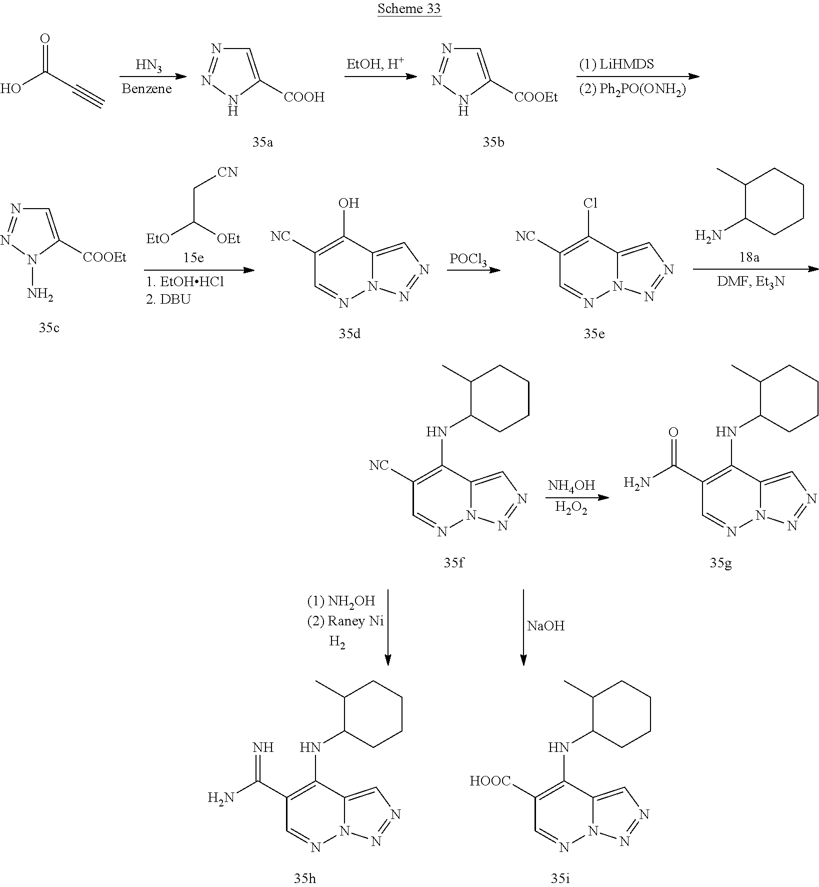

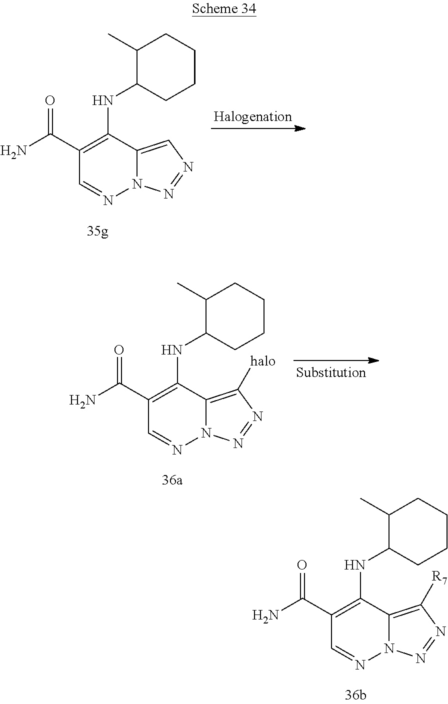

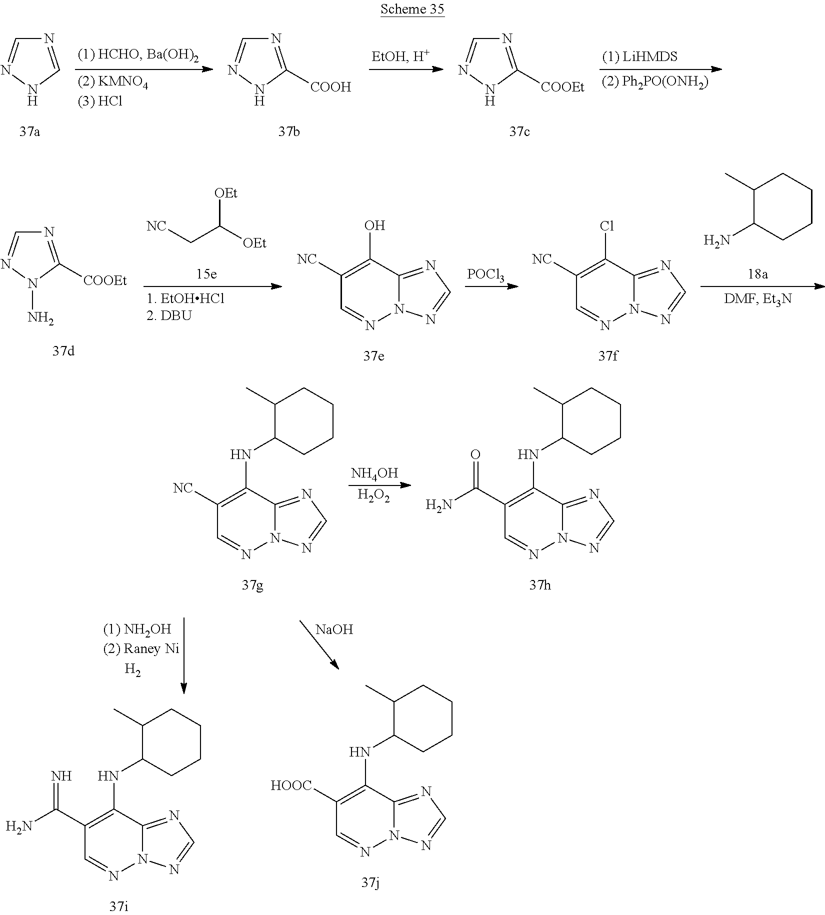

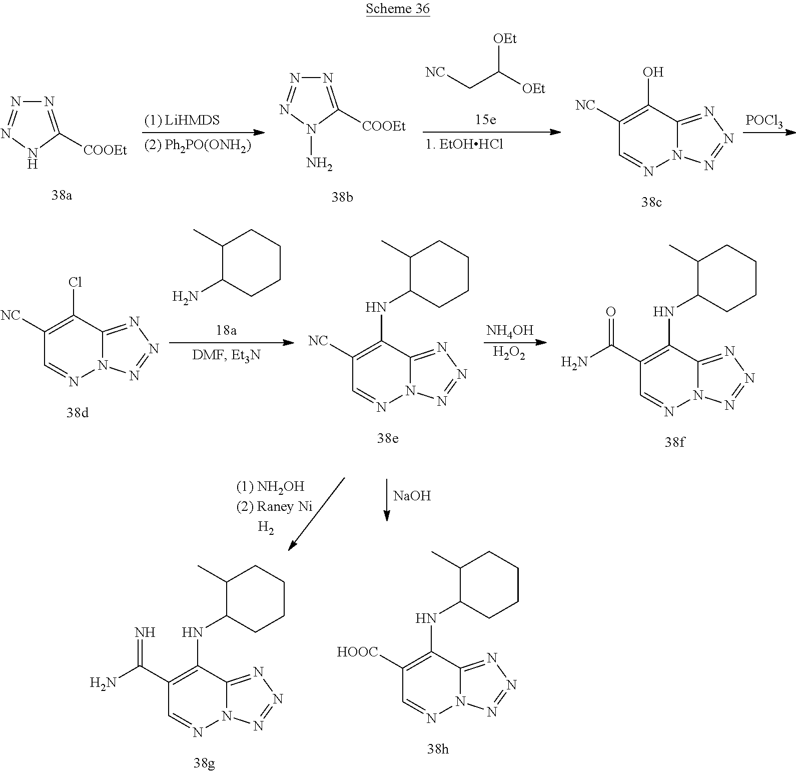

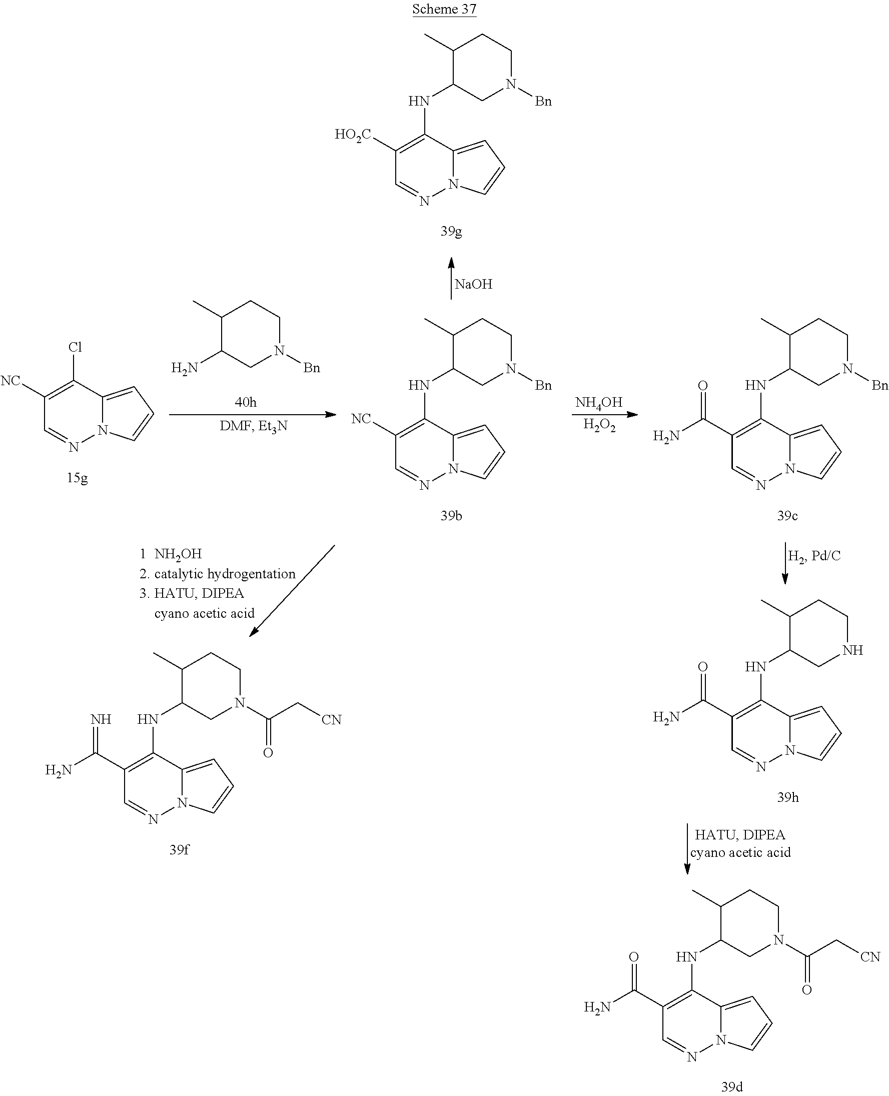

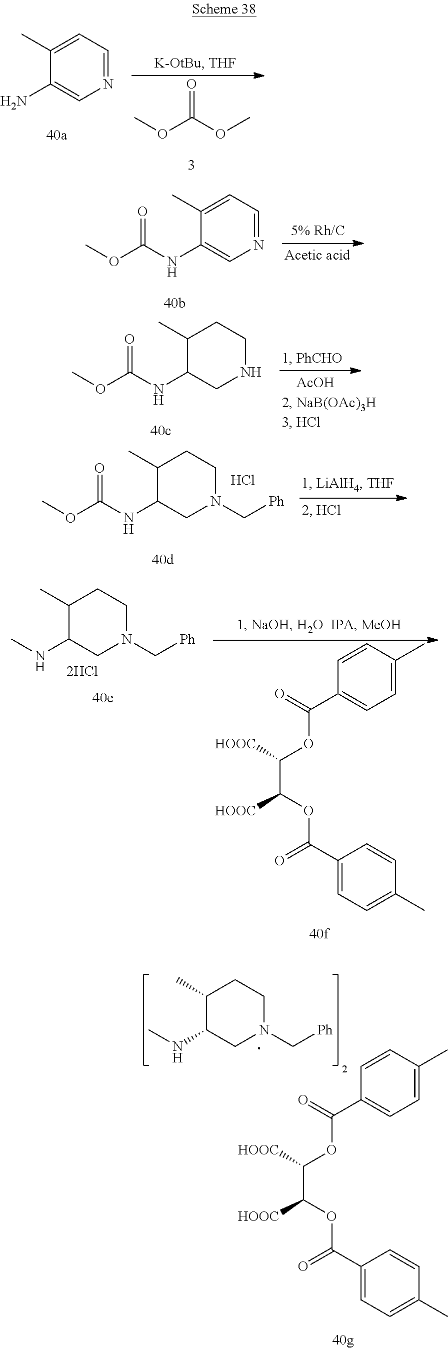

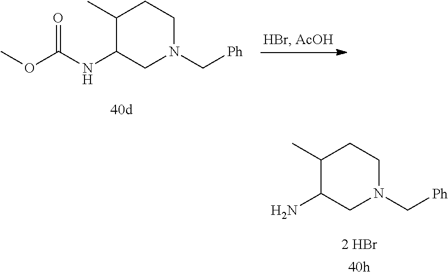

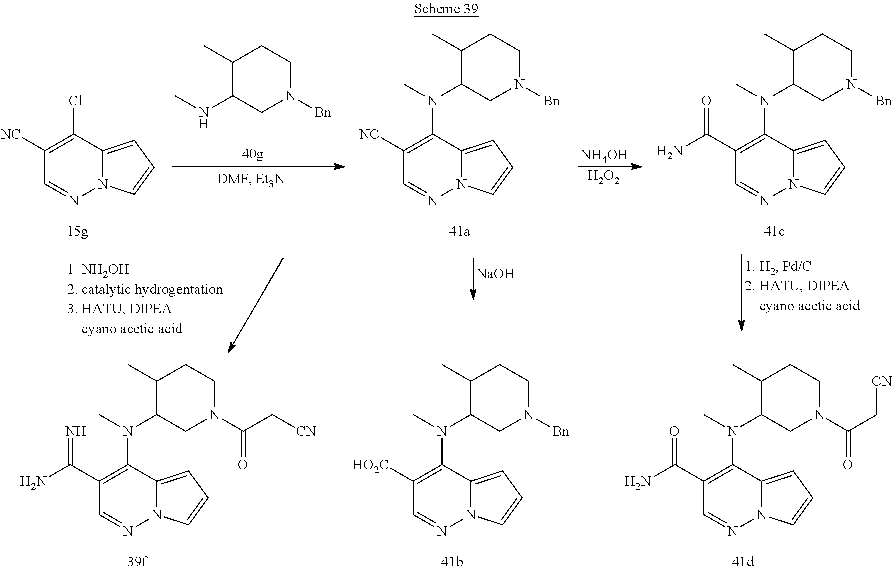

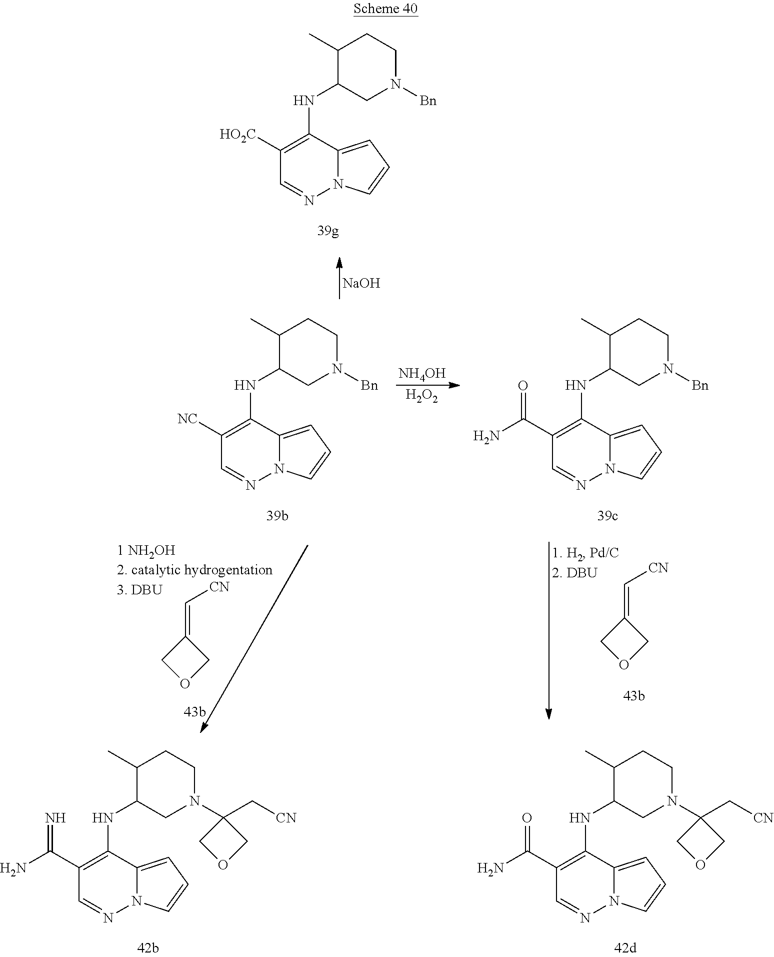

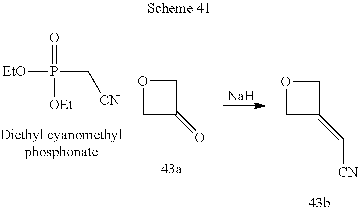

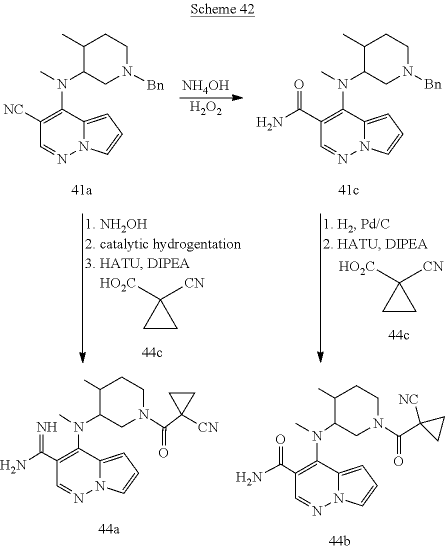

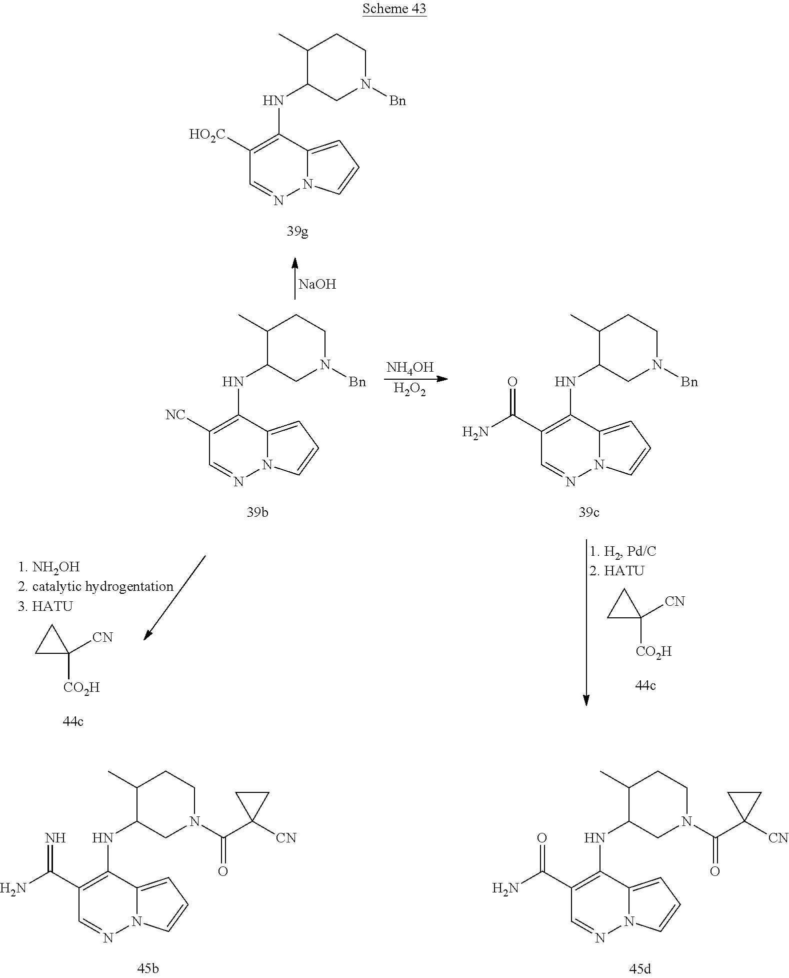

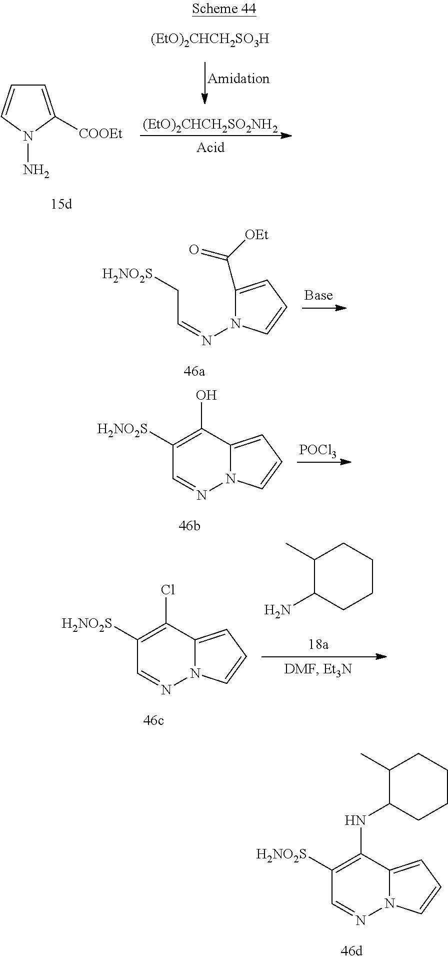

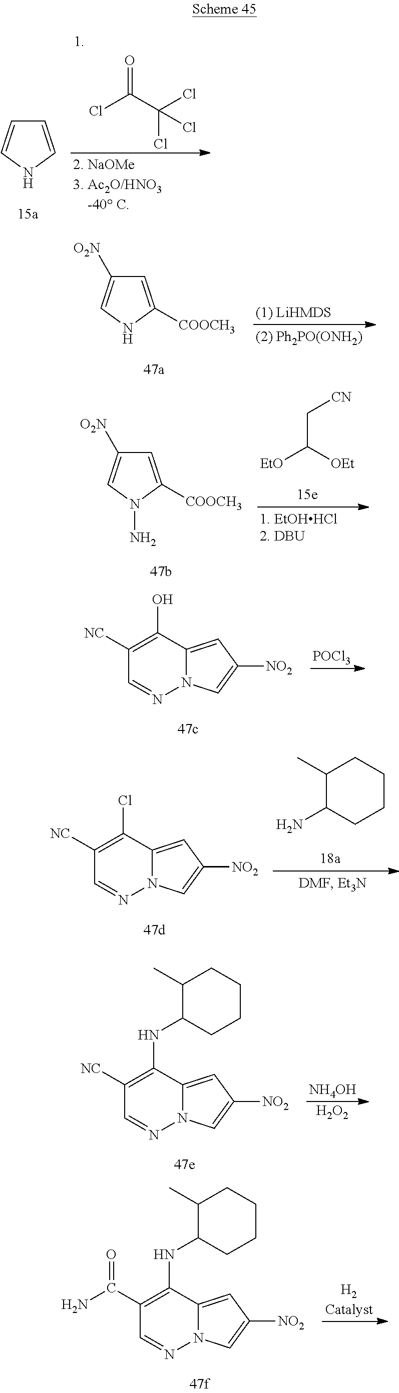

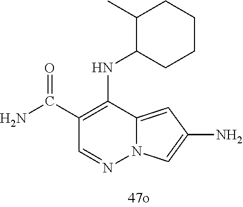

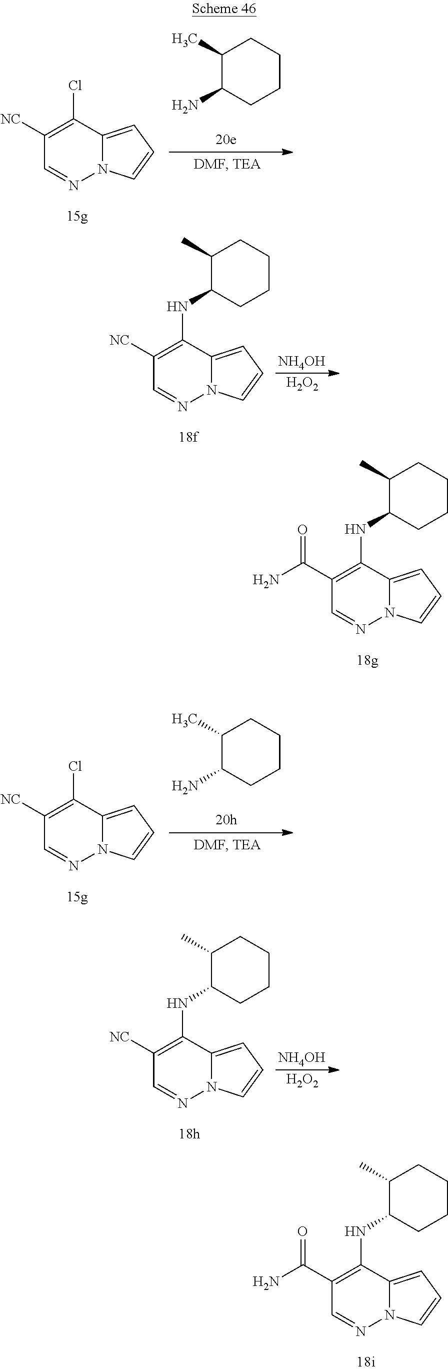

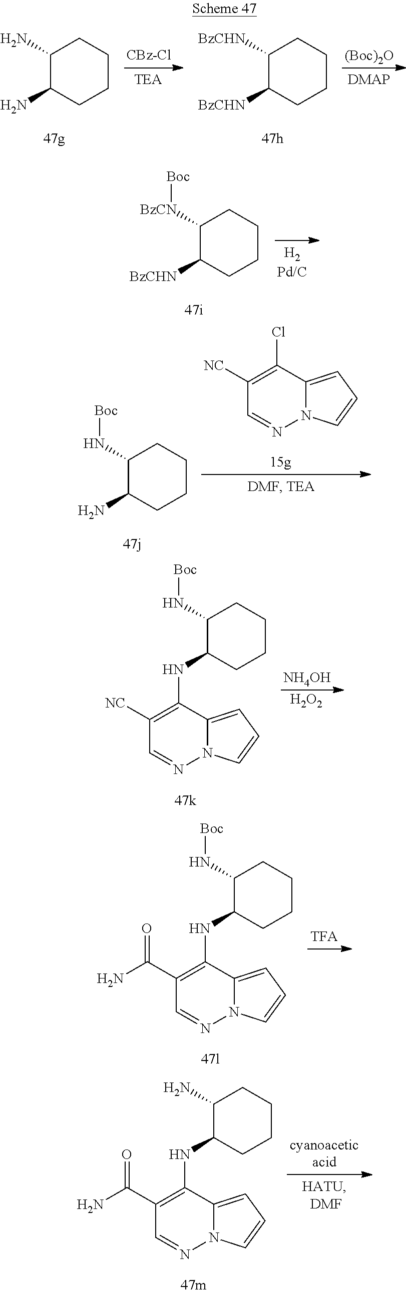

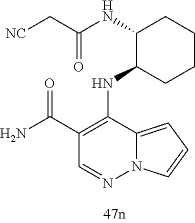

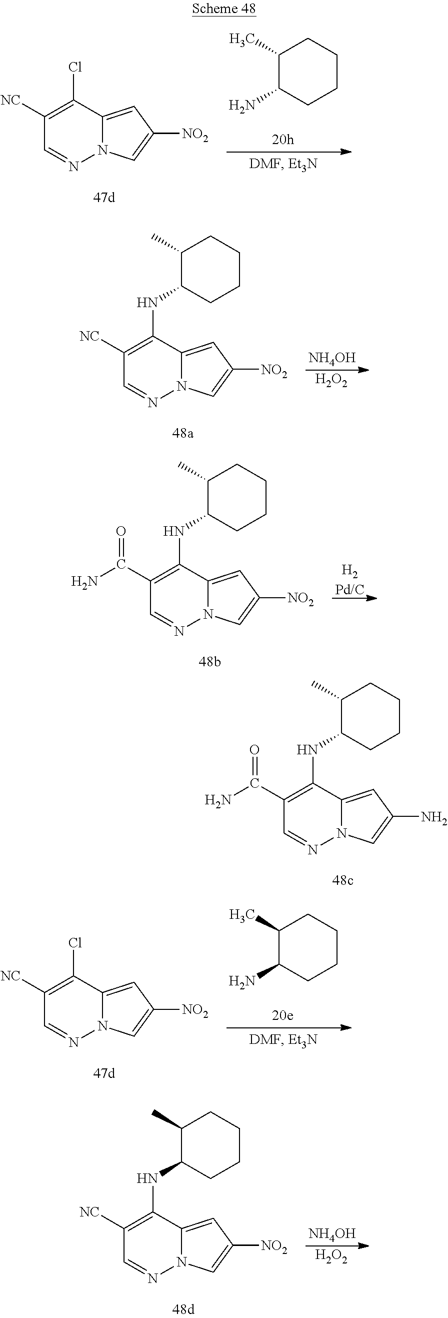

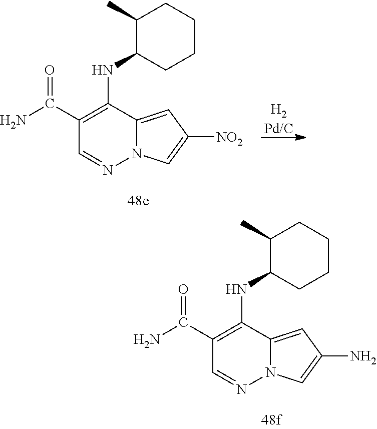

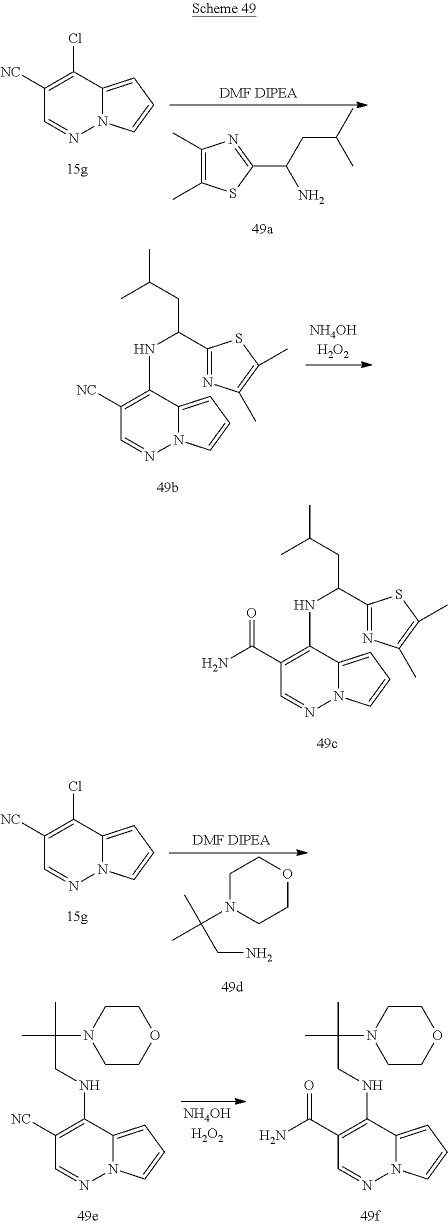

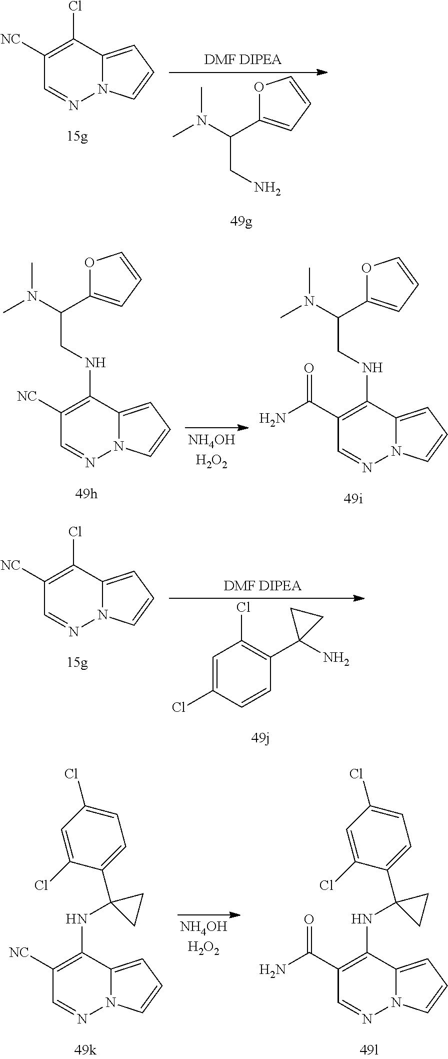

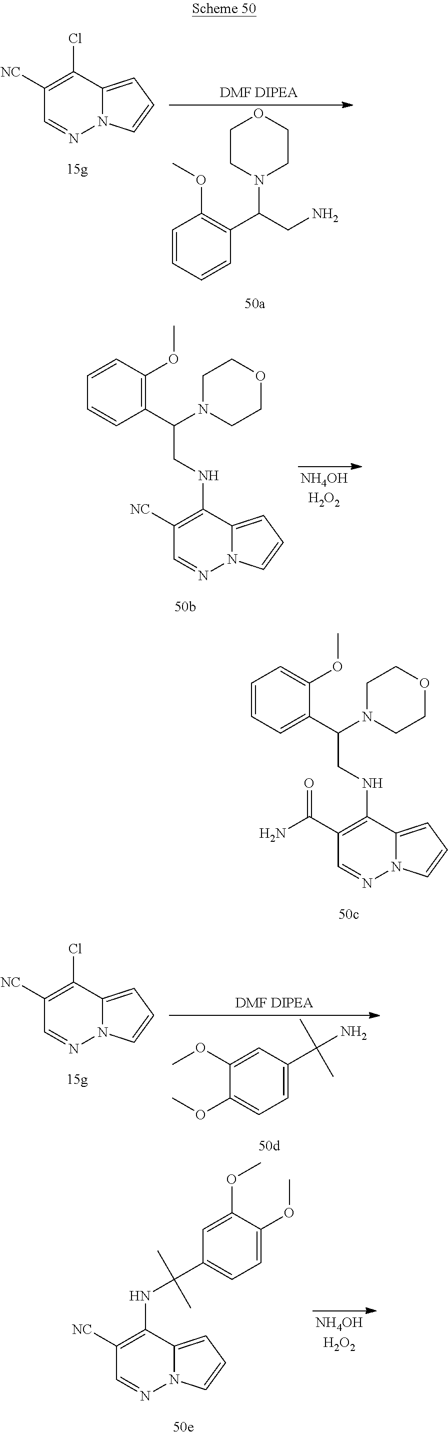

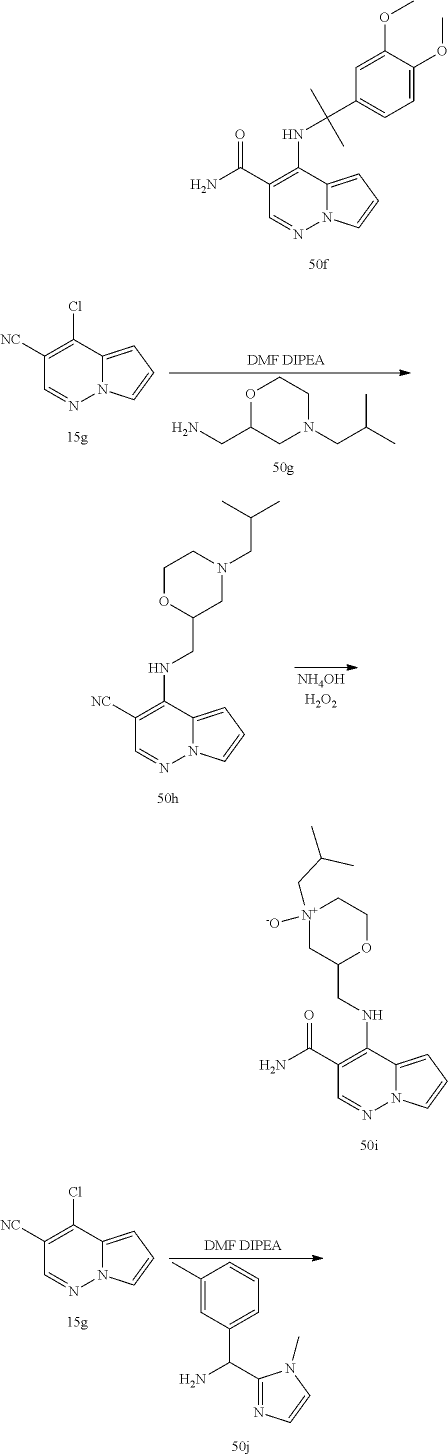

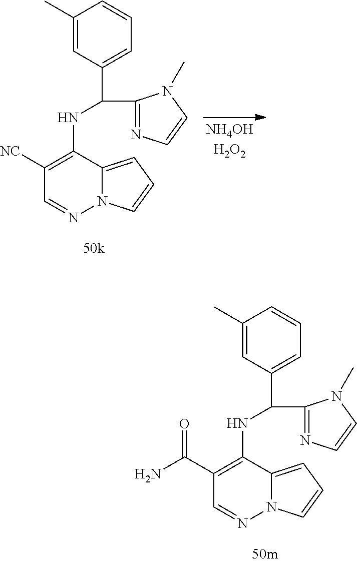

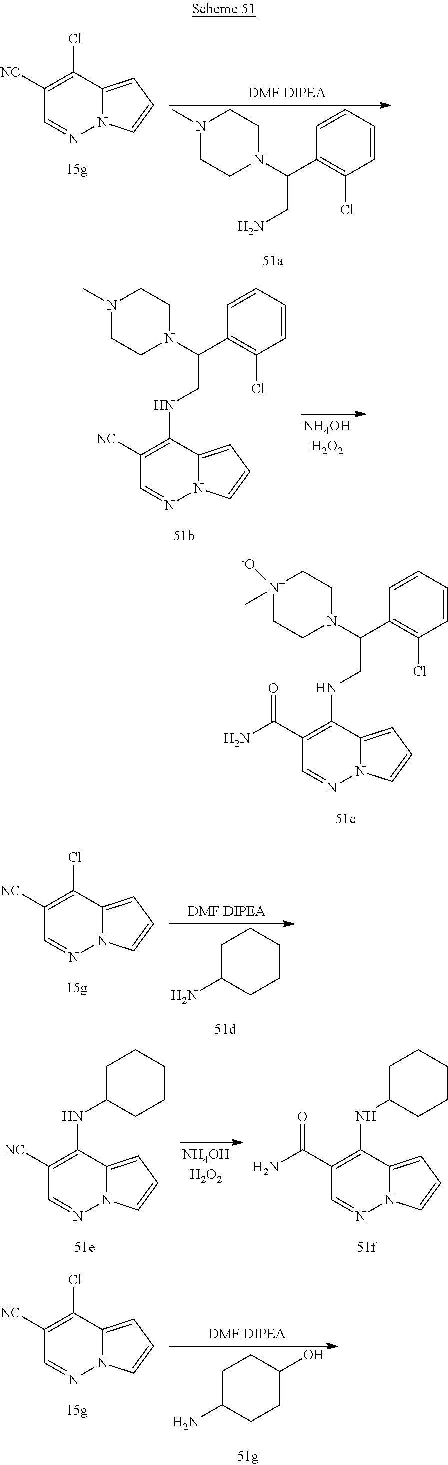

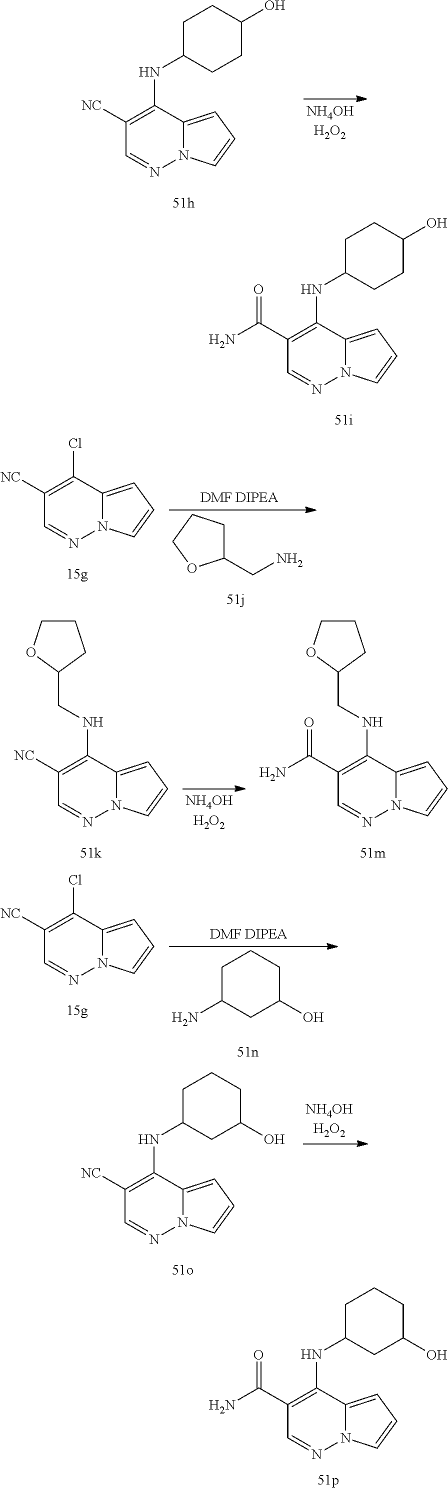

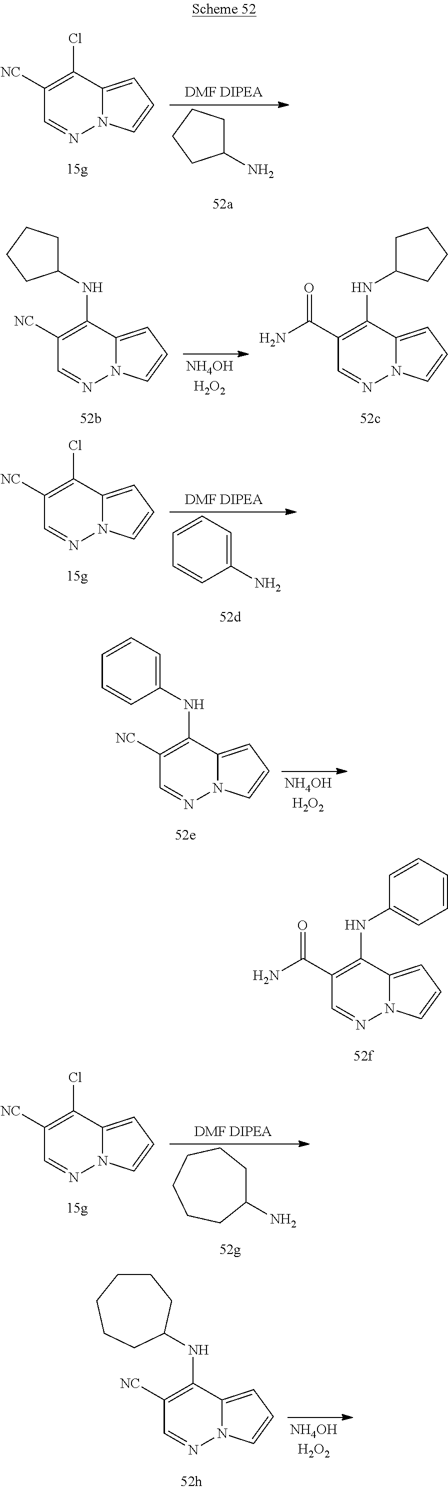

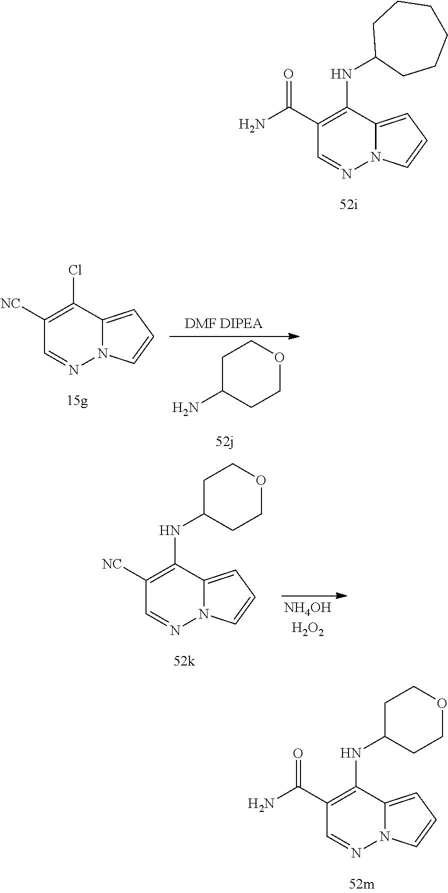

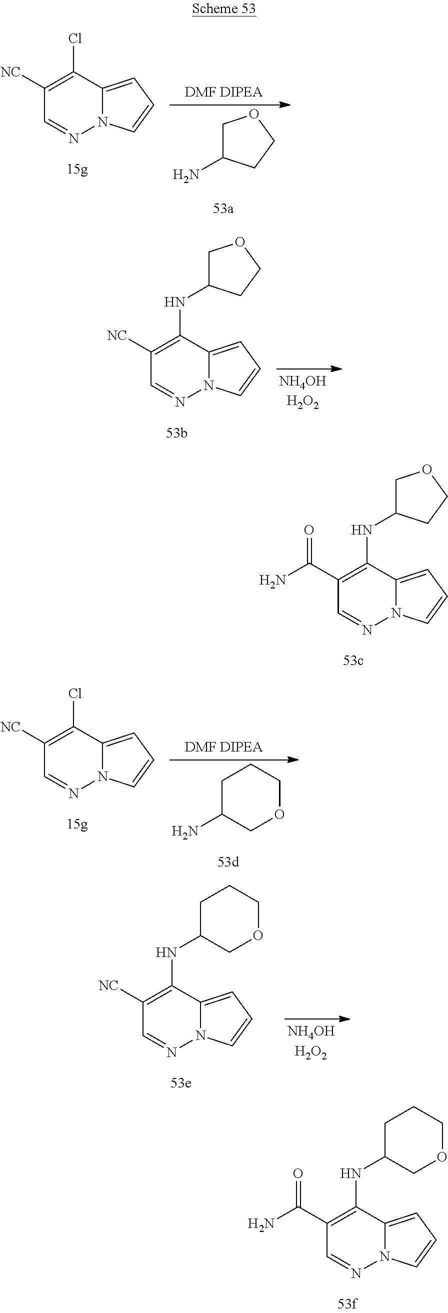

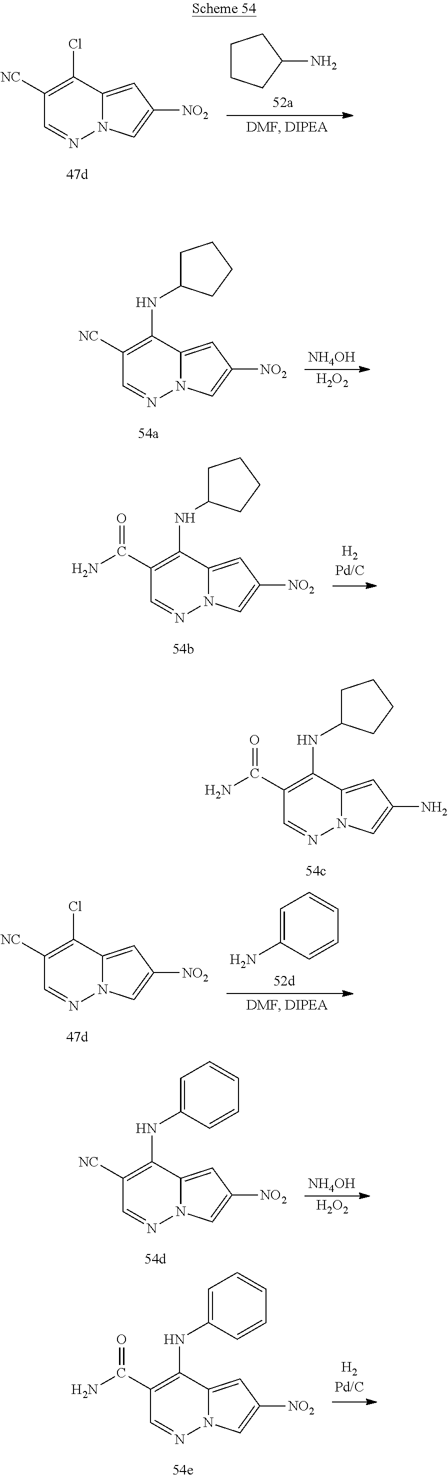

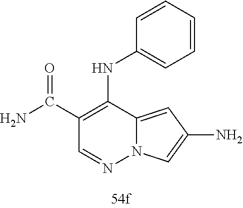

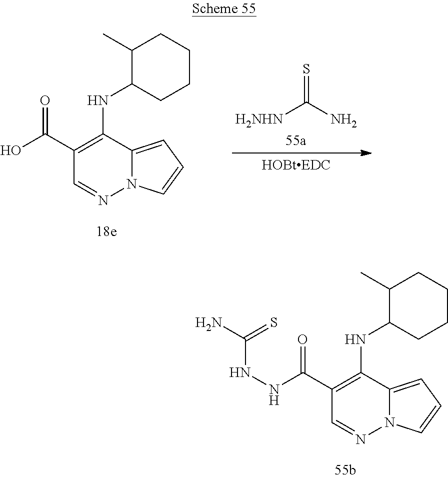

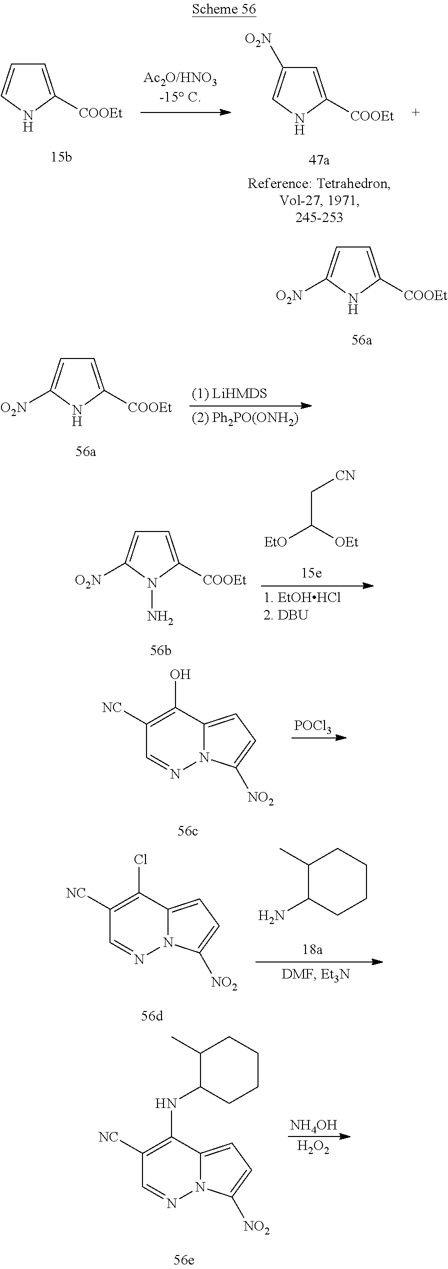

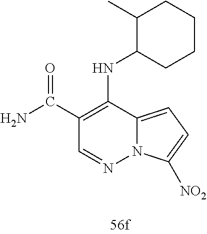

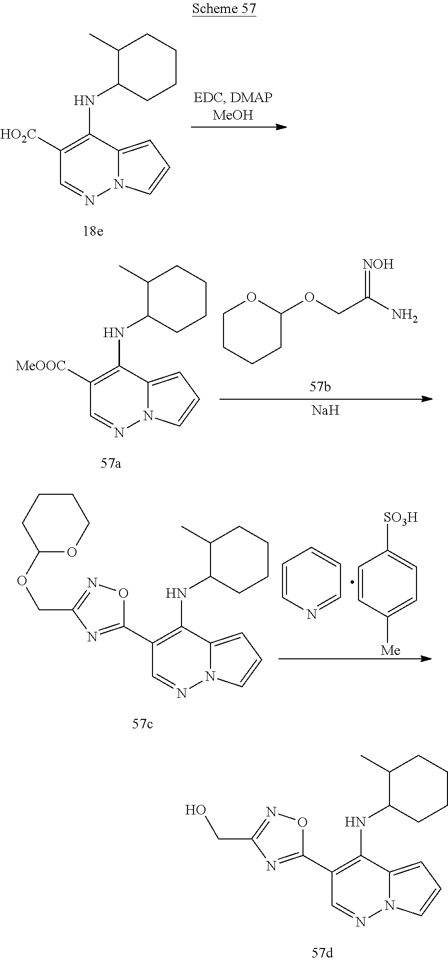

In one embodiment, the invention provides a method for preparing a salt of a compound of formula I, comprising reacting the compound of formula I with an acid under conditions suitable to provide the salt.

In one embodiment, the invention provides a method for preparing a pharmaceutical composition comprising a compound of formula I, or a pharmaceutically acceptable salt thereof, in combination with a pharmaceutically acceptable diluent or carrier, comprising combining the compound of formula I, or the pharmaceutically acceptable salt thereof, with the pharmaceutically acceptable diluent or carrier to provide the pharmaceutical composition.

The compounds of formula I can be formulated as pharmaceutical compositions and administered to a mammalian host, such as a human patient, in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful for immunosuppression. Accordingly, in one embodiment the invention also provides a composition comprising a compound of formula I, or a pharmaceutically acceptable salt thereof, at least one other therapeutic agent, and a pharmaceutically acceptable diluent or carrier. The invention also provides a kit comprising a compound of formula I, or a pharmaceutically acceptable salt thereof, at least one other therapeutic agent, packaging material, and instructions for administering the compound of formula I or the pharmaceutically acceptable salt thereof and the other therapeutic agent or agents to an animal to suppress an immune response in the animal.

Compounds of the invention may also be useful in the treatment of other diseases, conditions or disorders associated with the function of a kinase such as a Janus kinase (e.g. JAK1, JAK2 or TYK2) including the pathological activation of a kinase such as a Janus kinase (e.g. JAK1, JAK2 or TYK2). Accordingly, in one embodiment the invention provides a compound of formula I for the treatment of a kinase such as a Janus kinase (e.g. JAK1, JAK2 or TYK2) related disease, condition or disorder.

The ability of a compound of the invention to bind to JAK3 may be determined using pharmacological models which are well known to the art, or using Test A described below.

### Test A.

Inhibition constants (IC₅₀s) were determined against JAK3 (JH1domain-catalytic) kinase and other members of the JAK family. Assays were performed as described in Fabian et al. (2005) Nature Biotechnology, vol. 23, p. 329 and in Karaman et al. (2008) Nature Biotechnology, vol. 26, p. 127. Inhibition constants were determined using 11 point dose response curves which were performed in triplicate. Table 1 shown below lists compounds of the invention and their respective IC₅₀ values.

The ability of a compound of the invention to provide an immunomodulatory effect can also be determined using pharmacological models which are well known to the art. The ability of a compound of the invention to provide an anti-cancer effect can also be determined using pharmacological models which are well known to the art.

The invention will now be illustrated by the following non-limiting Examples.

### Example 1

### 4-(2-methylcyclohexylamino)pyrrolo[1,2-b]pyridazine-3-carboxamide (18c)

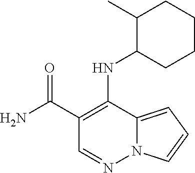

To a solution of 4-(2-methylcyclohexylamino)pyrrolo[1,2-b]pyridazine-3-carbonitrile 18b (167 mg, 0.66 mmol) in EtOH (16 mL) was added conc. NH₄OH (6 mL), followed by dropwise addition of H₂O₂ (0.27 mL, 2.64 mmol). The reaction mixture was stirred at room temperature for 14 h. The reaction mixture was concentrated to dryness and the residue obtained was purified by column chromatography (silica gel 30 g, eluting with hexanes/ethyl acetate, 1:0 to 1:1, product R_{f}=0.33 with hexanes/ethyl acetate=1:1) to furnish pure 4-(2-methylcyclohexylamino)pyrrolo[1,2-b]pyridazine-3-carboxamide (18c) (125 mg, 69%) as an off-white solid. ¹H NMR (300 MHz, DMSO-d₆): δ 10.99 (d, J=8.7, 1H), 8.20 (s, 1H), 7.65 (dd, J=1.5, 2.6 Hz, 1H), 6.87 (dd, J=1.5, 4.6 Hz, 1H), 6.65 (dd, J=2.7, 4.5 Hz, 1H), 4.37-4.27 (m, 1H), 1.97-1.24 (m, 9H), 0.90 (d, J=6.9 Hz, 3H); MS (ES⁺): 273.1 (M+H)⁺; IR (KBr pellet): 3448, 3185, 2929, 1620, 1562, 1352 cm⁻¹. Analysis, Calcd for C₁₅H₂₀N₄O: C, 66.15; H, 7.40; N, 20.57. Found: C, 66.12; H, 7.42; N, 20.54.

### Preparation of Intermediate Compound 18b.

### Step 1:

To a solution of ethyl pyrrole-2-carboxylate 15b (5 g, 98%, 35.21 mmol) in DMF (300 mL) cooled to −10° C. was added dropwise LiHMDS (1 M in THF, 42.3 mL) and stirred at −10° C. for 15 min. To the cold reaction mixture was added O-(diphenylphosphoryl)hydroxylamine 15e (15 g, 64.32 mmol) and stirred at RT for 16 h. The reaction mixture was diluted with ethyl acetate (800 mL) washed with water (2×400 mL), brine (200 mL), dried over MgSO₄ and filtered. The filtrate was concentrated in vacuo and the residue obtained was purified by column chromatography (silica gel 200 g, eluting with hexanes/ethyl acetate, 1:0 to 4:1, product R_{f}=˜0.46 in hexanes/ethyl acetate=4:1) to furnish ethyl 1-amino-1H-pyrrole-2-carboxylate (15d), (3.868 g, 71%) as a light yellow oil. ¹H NMR (300 MHz, DMSO-d₆): δ 7.01 (t, J=2.3 Hz, 1H), 6.70 (dd, J=2.0, 4.3 Hz, 1H), 6.26 (s, 2H), 5.97 (dd, J=2.6, 4.3 Hz, 1H), 4.22 (q, J=7.1 Hz, 2H), 1.27 (t, J=7.1 Hz, 3H).

### Step 2:

To a solution of ethyl 1-amino-1H-pyrrole-2-carboxylate (15d) (3.0 g, 19.46 mmol) in EtOH (100 mL) was added 3,3-diethoxypropanenitrile (25 mL, 95%, 158.23 mmol), 1N HCl (aq. 5 mL) and heated at reflux for 18 h. The reaction mixture was cooled to room temperature, treated with DBU (32.5 mL, 213.18 mmol), and stirred with heating at 80° C. for 1 h. The reaction mixture was concentrated in vacuo to remove most of EtOH. The residue obtained was diluted with EtOAc (300 mL), washed with water (200 mL, 150 mL). The combined aqueous solution was acidified with 4N HCl to pH=1 and extracted with chloroform (2×300 mL), chloroform/methanol (3:1, 200 mL). The combined extracts were dried over MgSO₄, filtered and the filtrate was concentrated in vacuo. The residue obtained was purified by column chromatography (silica gel 120 g, eluting with hexanes/ethyl acetate/MeOH, 1:1:0 to 2:2:1, product R_{f}=0.35 with hexanes/ethyl acetate/MeOH 2:2:1) to give 4-hydroxypyrrolo[1,2-b]pyridazine-3-carbonitrile (15f) (1.44 g, 47%) as a brown solid. ¹H NMR (300 MHz, DMSO-d₆): δ 8.16 (s, 1H), 7.90 (dd, J=1.6, 2.6 Hz, 1H), 7.08 (dd, J=1.6, 4.5 Hz, 1H), 6.80 (dd, J=2.6, 4.5 Hz, 1H); MS (ES⁻): 157.8 (M−H)¹.

### Step 3:

To a solution of 4-hydroxypyrrolo[1,2-b]pyridazine-3-carbonitrile (151) (1.26 g, 7.91 mmol) in acetonitrile (40 mL) was added benzyltriethylammonium chloride (3.68 g, 98%, 15.83 mmol) and N,N-diethylaniline (1.6 mL, 12.50 mmol). The mixture was heated to 80° C. followed by the addition of POCl₃ (4.4 mL, 47.59 mmol). The reaction mixture was stirred at 80° C. for 1 h and then concentrated to dryness. The residue obtained was dissolved in chloroform (400 mL), washed with 1N NaHCO₃ (200 mL), water (200 mL), brine (100 mL), dried over MgSO₄ and filtered. The filtrate was concentrated in vacuo and the residue obtained was purified by column chromatography (silica gel 50 g, eluting with hexanes/ethyl acetate, 1:0 to 6:1, product R_{f}=0.57 with hexanes/ethyl acetate 6:1) to 4-chloropyrrolo[1,2-b]pyridazine-3-carbonitrile (15g) (1.075 g, 77%, yellow solid). ¹H NMR (300 MHz, DMSO-d₆): δ 8.57 (s, 1H), 8.31 (dd, J=1.5, 2.6 Hz, 1H), 7.22-7.18 (m, 1H), 7.13 (dd, J=1.5, 4.6 Hz, 1H); Analysis: Calcd for C₈H₄ClN₃: C, 54.11; H, 2.27; N, 23.66. Found: C, 54.13; H, 2.21; N, 23.70.

### Step 4:

To a solution of 4-chloropyrrolo[1,2-b]pyridazine-3-carbonitrile (15g) (300 mg, 1.69 mmol) in DMF (40 mL) was added racemic 2-methylcyclohexanamine HCl salt (18a) (700 mg, 4.68 mmol), triethylamine (1.7 mL, 12.20 mmol) and stirred at RT for 15 h. The reaction mixture was diluted with EtOAc (300 mL) and washed with water (2×150 mL), brine (100 mL), dried over MgSO₄ and filtered. The filtrate was concentrated in vacuo and the residue obtained was purified by column chromatography (silica gel 30 g, eluting with hexanes/ethyl acetate, 1:0 to 6:1, product R_{f}=0.46 with hexanes/ethyl acetate 6:1) to afford 4-(2-methylcyclohexylamino)pyrrolo[1,2-b]pyridazine-3-carbonitrile (18b) (356 mg, 83%) as a yellow solid. ¹H NMR (300 MHz, DMSO-d₆): δ 7.90 (s, 1H), 7.70 (dd, J=1.6, 2.6 Hz, 1H), 7.34 (s, 1H), 7.32 (dd, J=1.6, 4.5 Hz, 1H), 6.68 (dd, J=2.7, 4.4 Hz, 1H), 4.53-4.27 (m, 1H), 2.34-2.19 (m, 1H), 1.89-1.33 (m, 8H), 0.92 (d, J=7.1 Hz, 3H); MS (ES⁻): 253.0 (M−H)⁻; Analysis: Calcd for C₁₅H₁₈N₄: C, 70.84; H, 7.13; N, 22.03. Found: C, 70.80; H, 7.21; N, 22.07.

Preparation of Intermediate Racemic Compound 18a.

To a cold solution (ice water) of trans-2-methylcyclohexanol (20a) (25 g, 218 mmol) in dichloromethane (500 mL) containing catalytic amount of DMAP was added dropwise methanesulfonyl chloride (34 mL, 436 mmol) followed by triethylamine (61 mL, 436 mmol). The reaction mixture was stirred at room temperature overnight and quenched with water (500 mL). The aqueous layer was separated and extracted with dichloromethane (2×200 mL). The combined organic layers were washed with water (200 mL), brine (200 mL), dried over MgSO₄, filtered and concentrated in vacuo to dryness to furnish 2-methylcyclohexy methanesulfonate as light brown oil, which was used as such for next step. ¹H NMR (300 MHz, DMSO) δ 4.19 (td, J=4.3, 10.2, 1H), 3.15 (s, 3H), 2.17-2.07 (m, 1H), 1.77-1.66 (m, 2H), 1.61-1.09 (m, 6H), 0.97 (d, J=6.5, 3H).

To a solution of 2-methylcyclohexy methanesulfonate in DMF (200 mL) was added sodium azide (71.5 g, 1100 mmol). The resulting mixture was heated in oil bath at 100° C. overnight. The reaction was allowed to cool to room temperature and diluted with water (2000 mL). The reaction mixture was extracted with ether (2×400 mL). The combined ether layers were washed with water (3×2000 mL), dried over MgSO4, filtered and concentrated in vacuo to remove ether to furnish 1-azido-2-methylcyclohexane (25 g, 84%) as light brown oil, which was pure enough to be used for next step. ¹H NMR (300 MHz, DMSO) δ 3.84-3.74 (m, 1H), 1.85-1.75 (m, 1H), 1.75-1.63 (m, 1H), 1.61-1.51 (m, 2H), 1.45-1.35 (m, 3H), 1.26 (dt, J=7.1, 17.6, 2H), 0.89 (d, J=6.8, 3H).

To a solution of 1-azido-2-methylcyclohexane (12 g, 86.4 mmol) in methanol (100 mL) was added Pd/C (10% on carbon, 2 g). The resulting mixture was hydrogenated on a parr shaker for 2 days (60 psi). The catalyst was removed by filtration through a pad of Celite. To the filtrate was added conc. HCl (7.2 mL) and stirred at room temperature for 30 min. The reaction mixture was concentrated in vacuum to dryness and the residue obtained was triturated with ether. The solid obtained was collected by filtration washed with ether and dried under vacuum at 35° C. overnight to afford 2-methylcyclohexanamine (18a) (6 g, 46.6%) as white solid. ¹H NMR (300 MHz, DMSO) δ 8.12 (s, 3H), 3.14 (s, 1H), 1.99 (s, 1H), 1.62 (t, J=15.3, 3H), 1.46 (s, 3H), 1.31 (s, 2H), 0.91 (d, J=7.1, 3H). MS (ES+) 114.3 (100%, M+1).

### Example 2

### 4-(2-methylcyclohexylamino)-7-(2,2,2-trifluoroacetamido)pyrrolo[1,2-b]pyridazine-3-carboxamide (21h)

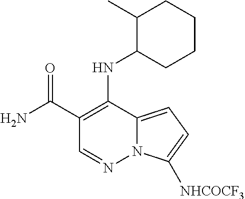

To a solution of tert-butyl 3-carbamoyl-4-(2-methylcyclohexylamino)pyrrolo[1,2-b]pyridazin-7-ylcarbamate 21g (22 mg, 0.057 mmol) in dichloromethane (4 mL) was added TFA (0.4 mL, 5.39 mmol) and stirred at room temperature for 22 h. The reaction mixture was concentrated in vacuo and the residue obtained was purified by flash column chromatography [(silica gel, 30 g eluting with hexanes/ethyl acetate/methanol, 1:1:0 to 1:1:0.04, (R_{f}=0.67 with hexanes/ethyl acetate/methanol=1:1:0.04)] to give 4-(2-methylcyclohexylamino)-7-(2,2,2-trifluoroacetamido)pyrrolo[1,2-b]pyridazine-3-carboxamide 21 h (10 mg, 61%) as a purple solid. ¹H NMR (300 MHz, DMSO-d₆): δ 11.47 (s, 1H), 11.04 (d, J=8.8 Hz, 1H), 8.29 (s, 1H), 6.94 (d, J=4.9 Hz, 1H), 6.76 (d, J=4.9 Hz, 1H), 4.40-4.27 (m, 1H), 2.00-1.15 (m, 9H), 0.91 (d, J=6.8 Hz, 3H); MS (ES−) 382.0.

### Preparation of Intermediate Compound 21g.

### Step 1:

To an ice cooled solution of DMF (24.5 mL, 316.43 mmol) in dichloromethane (70 mL) was added POCl₃ (29 mL, 313.63 mmol) followed by dropwise addition of a solution of ethyl pyrrole-2-carboxylate (15b) (40 g, 98%, 281.71 mmol) in dichloromethane (70 mL). The reaction mixture was stirred at 0° C. for 1 h and then refluxed for 3 h. The reaction was cooled to room temperature and diluted with ethyl acetate (250 mL); water (300 mL). The aqueous layer was separated and extracted with ethyl acetate (3×150 mL). The combined ethyl acetate layers were washed with aqueous 1 M NaHCO₃ (3×100 mL), dried over MgSO₄, filtered and concentrated in vacuum. The residue obtained was purified by column chromatography (silica gel, 450 g eluting with hexanes/ethyl acetate, 1:0 to 2:1, R_{f}=0.54 with hexanes/ethyl acetate=2:1) to give ethyl 5-formyl-1H-pyrrole-2-carboxylate (22b) (20.2 g, 43%) as a yellow solid. ¹H NMR (300 MHz, DMSO-d₆): δ 13.04 (bs, 1H), 9.71 (s, 1H), 6.97 (d, J=3.9 Hz, 1H), 6.88 (d, J=3.9 Hz, 1H), 4.30 (q, J=7.1 Hz, 2H), 1.31 (t, J=7.1 Hz, 3H); MS (ES⁻): 166.1 (M−H)⁻.

A solution of ethyl 5-formyl-1H-pyrrole-2-carboxylate (22b) (15 g, 89.73 mmol) in acetone (750 mL) was treated with a solution of KMnO₄ (28.36 g, 179.46 mmol) in a mixture of acetone (375 mL) and water (375 mL) over a period of 2 h followed by stirring at room temperature for 24 h. The reaction mixture was poured into a solution of Na₂SO₃ (63 g) in 1M HCl (1 L) and extracted with chloroform (1 L, 0.5 L, 0.5 L). The combined organic extracts were washed with water (1 L) and brine (0.5 L), dried over MgSO₄, filtered and concentrated in vacuum to give 5-(ethoxycarbonyl)-1H-pyrrole-2-carboxylic acid (22c) (14.09 g) as an off-white solid. It was used as such for next step; MS (ES⁻): 182.0 (M−H)⁻.

A solution of crude 5-(ethoxycarbonyl)-1H-pyrrole-2-carboxylic acid (22c) (14 g) in EtOH (500 mL) was treated with conc. H₂SO₄ (2 mL) and refluxed for 14 h. Additional conc. H₂SO₄ (5 mL) was added and the reaction mixture was refluxed for additional 22 h. The reaction was cooled to room temperature, neutralized with aq. 6N NaOH, and concentrated in vacuum to dryness. To the residue obtained was added ethyl acetate (500 mL) water (300 mL). The aqueous phase was separated and extracted with ethyl acetate (200 mL). The combined ethyl acetate layers was washed with brine (200 mL), dried over MgSO₄, filtered and concentrated in vacuum. The residue obtained was purified by column chromatography (silica gel, 200 g eluting with hexanes/ethyl acetate, 1:0 to 4:1, R_{f}=0.53 with hexanes/ethyl acetate=4:1) to give diethyl 1H-pyrrole-2,5-dicarboxylate 21a (8.135 g, 44%) as a white solid; ¹H NMR (300 MHz, DMSO-d₆): δ 12.67 (bs, 1H), 6.80 (s, 2H), 4.26 (q, J=7.1 Hz, 4H), 1.29 (t, J=7.1 Hz, 6H).

### Step 2:

A solution of diethyl 1H-pyrrole-2,5-dicarboxylate 21a (8.135 g, 38.52 mmol) in DMF (350 mL) cooled to −10° C. was added LiHMDS (1 M in THF, 46.5 mL) and stirred at −10° C. for 15 min. The reaction mixture was treated with O-(diphenylphosphoryl)hydroxylamine (17.3 g, 74.19 mmol) at −10° C. and stirred at room temperature for 17 h. The reaction mixture was diluted with ethyl acetate (800 mL) and washed with water (2×400 mL), brine (200 mL), dried over MgSO₄, filtered and concentrated in vacuum. The residue obtained was purified by column chromatography (silica gel, 200 g eluting with hexanes/ethyl acetate, 1:0 to 4:1, R_{f}=0.38 with hexanes/ethyl acetate=5:1) to give diethyl 1-amino-1H-pyrrole-2,5-dicarboxylate 21b (8.29 g, 95%) as a yellow solid; ¹H NMR (300 MHz, DMSO-d₆): δ 7.25 (s, 2H), 6.68 (s, 2H), 4.28 (q, J=7.1 Hz, 4H), 1.29 (t, J=7.1 Hz, 6H); MS (ES⁺): 227.1 (M+H)⁺.

### Step 3:

To a solution of diethyl 1-amino-1H-pyrrole-2,5-dicarboxylate 21b (3.0 g, 13.26 mmol) in EtOH (90 mL) was added 3,3-diethoxypropanenitrile (18 mL, 95%, 113.93 mmol), HCl (aqueous 1 N, 3.5 mL) and heated at reflux for 15 h. The reaction mixture was cooled to room temperature added DBU (24 mL, 157.43 mmol) and stirred at 80° C. for 1 h. The reaction mixture was concentrated in vacuum to remove ethanol. The residue obtained was diluted with EtOAc (200 mL) and extracted with water (200 mL, 150 mL). The aqueous layer was combined and acidified with 4N aqueous HCl to pH=1. The aqueous layer was with chloroform/methanol (3:1, 300 mL, 2×200 mL). The organic layers were combined dried over MgSO₄, filtered and concentrated in vacuum. The residue obtained was purified by column chromatography (silica gel, 120 g eluting with hexanes/ethyl acetate/MeOH, 1:1:0 to 2:2:1, R_{f}=0.39 with hexanes/ethyl acetate/MeOH=2:2:1) to give ethyl 3-cyano-4-hydroxypyrrolo[1,2-b]pyridazine-7-carboxylate 21c (1.379 g, 45%) as a yellow solid); ¹H NMR (300 MHz, DMSO-d₆): δ 7.95 (s, 1H), 7.07 (d, J=4.5 Hz, 1H), 6.60 (d, J=4.5 Hz, 1H), 4.24 (q, J=7.1 Hz, 2H), 1.28 (t, J=7.1 Hz, 3H); MS (ES⁻): 230.4 (M−H)⁻.

### Step 4:

To a solution of ethyl 3-cyano-4-hydroxypyrrolo[1,2-b]pyridazine-7-carboxylate 21c, (1.3 g, 5.62 mmol) in acetonitrile (40 mL) was added benzyltriethylammonium chloride (2.62 g, 98%, 11.39 mmol), N,N-dimethylaniline (1.15 mL, 8.04 mmol) and heated to 80° C. To the hot solution was added dropwise POCl₃ (3.2 mL, 34.61 mmol) and stirred at 80° C. for 1 h. The reaction mixture was concentrated to dryness and the residue obtained was dissolved in chloroform (300 mL). The chloroform layer was washed with 1N NaHCO₃ (150 mL), water (150 mL), brine (100 mL), dried over MgSO₄, filtered and concentrated in vacuum. The residue obtained was purified by column chromatography (silica gel, 120 g eluting with hexanes/ethyl acetate, 1:0 to 3:1, R_{f}=0.44 with hexanes/ethyl acetate=3:1) to give ethyl 4-chloro-3-cyanopyrrolo[1,2-b]pyridazine-7-carboxylate 21d (806 mg, 57%) as a yellow solid; ¹H NMR (300 MHz, DMSO-d₆): δ 8.84 (s, 1H), 7.71 (d, J=4.9 Hz, 1H), 7.19 (J=4.9 Hz, 1H), 4.36 (q, J=7.1 Hz, 2H), 1.33 (t, J=7.1 Hz, 3H).

### Step 5:

To a solution of ethyl 4-chloro-3-cyanopyrrolo[1,2-b]pyridazine-7-carboxylate 21d (347 mg, 1.39 mmol) in DMF (30 mL) was added 2-methylcyclohexanamine HCl salt 18a (550 mg, 3.68 mmol), triethylamine (1.4 mL, 10.04 mmol) and stirred at room temperature overnight. The reaction mixture was diluted with EtOAc (300 mL) and washed with water (2×150 mL), brine (100 mL), dried over MgSO₄, filtered and concentrated in vacuum. The residue obtained was purified by column chromatography (silica gel, 30 g, eluting with hexanes/ethyl acetate, 1:0 to 3:1, R_{f}=0.37 with hexanes/ethyl acetate=3:1) to afford ethyl 3-cyano-4-(2-methylcyclohexylamino)pyrrolo[1,2-b]pyridazine-7-carboxylate 21e (305 mg, 67%, yellow solid); ¹H NMR (300 MHz, DMSO-d₆): δ 8.16 (s, 1H), 7.62 (d, J=8.7 Hz, 1H), 7.45 (d, J=4.9 Hz, 1H), 7.32 (d, J=4.9 Hz, 1H), 4.46-4.35 (m, 1H), 4.28 (q, J=7.1 Hz, 2H), 2.33-2.44 (m, 1H), 1.90-1.20 (m, 8H), 1.30 (t, J=7.1 Hz, 3H), 0.92 (d, J=7.1 Hz, 3H); MS (ES⁻): 325.0 (M−H)⁻.

### Step 6:

To a solution of ethyl 3-cyano-4-(2-methylcyclohexylamino)pyrrolo[1,2-b]pyridazine-7-carboxylate 21e (419 mg, 1.28 mmol) in EtOH (30 mL) was added conc. NH₄OH (11.5 mL), followed by H₂O₂ (0.53 mL, 5.19 mmol) and stirred at RT for 12 h. The reaction mixture was concentrated in vacuum to dryness and to the residue obtained was added 30 mL of EtOH, 30 mL of water, and 6 mL of 6N aq. NaOH and stirred at room temperature for 5 h. The reaction mixture was acidified with conc. HCl followed and concentrated in vacuum to remove EtOH. The solid obtained was collected by filtration washed with water and dried in vacuum to give 3-carbamoyl-4-(2-methylcyclohexylamino)pyrrolo[1,2-b]pyridazine-7-carboxylic acid 21f (322 mg, 80%, light-brown solid); ¹H NMR (300 MHz, DMSO-d₆): δ 12.85 (s, 1H), 11.12 (d, J=8.9 Hz, 1H), 8.47 (s, 1H), 7.30 (d, J=5.1 Hz, 1H), 7.01 (d, J=5.1 Hz, 1H), 4.40-4.30 (m, 1H), 2.00-1.20 (m, 9H), 0.90 (d, J=6.8 Hz, 3H).

### Step 7:

To a solution of 3-carbamoyl-4-(2-methylcyclohexylamino)pyrrolo[1,2-b]pyridazine-7-carboxylic acid 21f (40 mg, 0.13 mmol) in t-BuOH (4 mL) was added triethylamine (0.06 mL, 0.43 mmol), diphenyl phosphoryl azide (0.06 mL, 97%, 0.27 mmol) and heated at reflux for 5 h. The reaction mixture was concentrated in vacuum to dryness and the residue obtained dissolved in chloroform (75 mL). The chloroform layer was washed with water (30 mL), dried over MgSO₄, filtered and concentrated in vacuum. The residue obtained was purified by column chromatography (silica gel, 30 g eluting with hexanes/ethyl acetate, 1:0 to 2:1, R_{f}=0.33 with hexanes/ethyl acetate=2:1) to give tert-butyl 3-carbamoyl-4-(2-methylcyclohexylamino)pyrrolo[1,2-b]pyridazin-7-ylcarbamate 21 g (25 mg, 50%, dark-green solid); ¹H NMR (300 MHz, DMSO-d₆): δ 10.98 (d, J=8.9 Hz, 1H), 8.85 (s, 1H), 8.21 (s, 1H), 6.83 (d, J=4.9 Hz, 1H), 6.56 (d, J=4.9 Hz, 1H), 4.35-4.25 (m, 1H), 2.00-1.20 (m, 9H), 1.46 (s, 9H), 0.89 (d, J=7.0 Hz, 3H).

### Example 3

### 4-(4-methylpiperidin-3-ylamino)pyrrolo[1,2-b]pyridazine-3-carboxamide

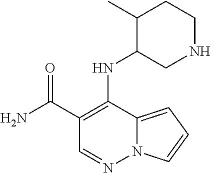

A solution of 4-(1-benzyl-4-methylpiperidin-3-ylamino)pyrrolo[1,2-b]pyridazine-3-carboxamide (39c) (0.38 g, 1.05 mmol) in methanol (20 mL) was subjected to hydrogenolysis in the presence of 10 wt % Pd/C (150 mg) under hydrogen atmosphere at 60 psi at room temperature for 3 h. The reaction mixture was filtered through Celite, and the filtrate was concentrated in vacuum. The residue obtained was purified by flash column chromatography (silica gel 12 g, eluting with chloroform in CMA-80 0-100% to give 4-(4-methylpiperidin-3-ylamino)pyrrolo[1,2-b]pyridazine-3-carboxamide (39h) (0.045 g, 16%) as a white solid; ¹HNMR (300 MHz, DMSO) δ 10.97 (s, 1H), 8.20 (s, 1H), 7.64 (bs, 3H), 7.64 (s, 1H), 6.85 (s, 1H), 6.63 (s, 1H), 4.25 (m, 1H), 2.92 (m, 2H), 2.76 (m, 1H), 1.92 (m, 2H), 1.45 (m, 2H), 0.89 (d, J=6.7, 3H). MS (ES+) 274.1 (M+1).

### Preparation of Intermediate Compound 39c.

### Step 1:

To methyl 1-benzyl-4-methylpiperidin-3-ylcarbamate (40d) was added HBr in acetic acid (5 ml, 33% HBr) and stirred at room temperature for 3 days. The reaction mixture was concentrated in vacuum to dryness to furnish 1-benzyl-4-methylpiperidin-3-amine (40h) (1.1 g, 66%) as a orange solid. ¹H NMR (300 MHz, DMSO) δ 10.27 (bs, 1H), 8.23 (bs, 3H), 7.62 (m, 2H), 7.53-7.40 (m, 3H), 4.54 (s, 2H), 3.71 (m, 1H), 3.61 (m, 2H), 3.16 (m, 2H), 2.34 (m, 1H), 2.09 (m, 1H), 1.75 (m, J=14.3, 1H), 1.05 (d, J=7.0, 3H); MS (ES+) 205.2 (M+1).

### Step 2:

To a solution of 4-chloropyrrolo[1,2-b]pyridazine-3-carbonitrile (15g) (619 mg, 3.5 mmol) in DMF (10 mL) was added racemic 1-benzyl-4-methylpiperidin-3-amine (40h) (1.1 g, 2.85 mmol), diisopropylethylamine (3.1 mL, 17.5 mmol) and heated at 80° C. for 15 h. The reaction mixture was diluted with EtOAc (20 mL) and washed with water (2×20 mL), brine (100 mL), dried over MgSO₄ and filtered. The filtrate was concentrated in vacuo and the residue obtained was purified by column chromatography (silica gel 24 g, eluting with hexanes/ethyl acetate 0 to 100%) to furnish 4-(1-benzyl-4-methylpiperidin-3-ylamino)pyrrolo[1,2-b]pyridazine-3-carbonitrile (39b) (748 mg, 62%) as a off white solid. ¹H NMR (300 MHz, DMSO) δ 7.95 (s, 1H), 7.77 (s, 1H), 7.45-7.13 (m, 6H), 7.09-6.81 (bs, 1H), 6.74 (dd, J=2.7, 4.5, 1H), 4.57 (m, 1H), 3.54 (dd, J=13.2, 30.6, 2H), 2.76 (m, 2H), 2.39 (m, 1H), 2.31-2.13 (m, 1H), 1.99 (m, 1H), 1.60 (m, 2H), 0.91 (d, J=6.6, 3H); MS (ES+) 346.1 (M+1); Analysis calcd: C, 73.02; H, 6.71; N, 20.27. Found C, 73.09; H, 6.68; N, 20.19.

### Step 3:

To a solution of 4-(1-benzyl-4-methylpiperidin-3-ylamino)pyrrolo[1,2-b]pyridazine-3-carbonitrile (39b) (586 mg, 1.69 mmol) in EtOH (50 mL) was added conc. NH₄OH (20 mL), followed by dropwise addition of H₂O₂ (1 mL). The reaction mixture was stirred at room temperature for 14 h. The reaction mixture was concentrated to dryness and the residue obtained was purified by column chromatography (silica gel 24 g, eluting with hexanes/ethyl acetate 0 to 100%) to furnish pure of 4-(1-benzyl-4-methylpiperidin-3-ylamino)pyrrolo[1,2-b]pyridazine-3-carboxamide (39c) as a green oil. ¹HNMR (300 MHz, DMSO) δ 12.16-11.73 (bs, 1H), 11.02 (d, J=9.7, 1H), 8.21 (s, 1H), 7.60 (dd, J=1.5, 2.6, 1H), 7.44-7.09 (m, 6H), 6.85 (d, J=3.2, 1H), 6.57 (dd, J=2.7, 4.5, 1H), 4.43 (m, 1H), 3.49 (d, J=6.0, 2H), 2.80 (m, 2H), 2.29 (m, 1H), 1.91 (m, 2H), 1.56 (m, 2H), 0.87 (d, J=6.7, 3H); MS (ES+) 364.1 (M+1). HPLC [Zorbax SBC3, 3.0×150 mm, 5 μm with a ZGC SBC3, 2.1×12.5 mm guard cartridge, “A” buffer=(98% of 0.1 M ammonium acetate in 2% acetonitrile); “B” buffer=100% acetonitrile, UV absorbance; Rt=18.766, 85.73%].

### Example 4

### 4-(1-(2-cyanoacetyl)-4-methylpiperidin-3-ylamino)pyrrolo[1,2-b]pyridazine-3

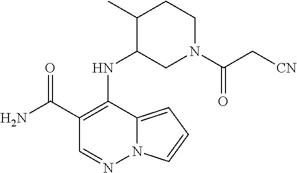

To a solution of 4-(4-methylpiperidin-3-ylamino)pyrrolo[1,2-b]pyridazine-3-carboxamide (39h) (0.33 mmol) in dimethylformamide (2 mL) was added cyanoacetic acid (0.03 g, 0.363 mmol), diisopropylethyl amine (0.213 g, 1.65 mmol) and cooled to −10° C. To this mixture (2-(7-Aza-1H-benzotriazole-1-yl)-1,1,3,3-tetramethyluronium hexafluorophosphate) (HATU, 0.15 g, 0.39 mmol) was added and stirred below 10° C. for 1 h. The reaction mixture was quenched with water (15 mL) and extracted with ethyl acetate (3×50 mL). The organic layers were combined washed with water (2×15 mL), brine (10 mL), dried and concentrated in vacuo. The residue obtained was purified by flash column chromatography [silica gel 12 g, eluting with 0 to 100% ethyl acetate/methanol (9:1) in hexane] to afford 4-(1-(2-cyanoacetyl)-4-methylpiperidin-3-ylamino)pyrrolo[1,2-b]pyridazine-3-carboxamide (39d) (52 mg, 46%) as a light green solid; ¹HNMR (300 MHz, DMSO, 360K) δ 10.66 (s, 1H), 8.20 (s, 1H), 7.63 (s, 1H), 7.10 (s, 2H), 6.91 (s, 1H), 6.66 (s, 1H), 4.36 (m, 1H), 4.08 (m, 1H), 3.80 (m, 3H), 3.19 (m, 2H), 2.04 (m, 1H), 1.41 (m, 2H), 0.94 (d, J=6.7, 3H); MS (ES+) 363.1 (M+23); HPLC [Zorbax SBC3, 3.0×150 mm, 5 μm with a ZGC SBC3, 2.1×12.5 mm guard cartridge, “A” buffer=(98% of 0.1 M ammonium acetate in 2% acetonitrile); “B” buffer=100% acetonitrile, UV absorbance; Rt=14.78 (97.39%)].

### Example 5

### 4-(2-methylcyclohexylamino)pyrrolo[1,2-b]pyridazine-3-carboxylic acid (18e)

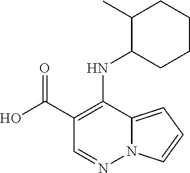

To a solution of 4-(2-methylcyclohexylamino)pyrrolo[1,2-b]pyridazine-3-carbonitrile (18b) (118 mg, 0.66 mmol) in EtOH (9.0 mL) was added 20 N NaOH (6 mL) and heated at reflux for 14 h. The reaction mixture was cooled to room temperature, diluted with water (10 mL) and acidified with conc. HCl. The solid obtained was collected by filtration, washed with water and dried under vacuum to give 4-(2-Methylcyclohexylamino)pyrrolo[1,2-b]pyridazine-3-carboxylic acid (18e) (121 mg, 96%) as an off-white solid; mp 195.1° C.; ¹H NMR (300 MHz, DMSO-d₆): δ 12.71-12.31 (m, 1H), 10.06 (d, J=8.3 Hz, 1H), 8.18 (s, 1H), 7.71 (dd, J=1.5, 2.6 Hz, 1H), 6.97 (dd, J=4.8, 1.4 Hz, 1H), 6.69 (dd, J=2.7, 4.5 Hz, 1H), 4.42-4.32 (m, 1H), 2.03-1.29 (m, 9H), 0.91 (d, J=6.9 Hz, 3H); MS (ES⁻): 272.0 (M−H)⁻.

### Example 6

### 4-(((3R,4R)-1-benzyl-4-methylpiperidin-3-yl)(methyl)amino)pyrrolo[1,2-b]pyridazine-3-carbonitrile (41a)

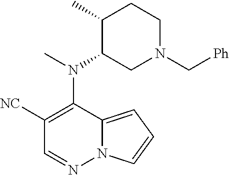

To a solution of 4-chloropyrrolo[1,2-b]pyridazine-3-carbonitrile (15g) (708 mg, 4 mmol) in DMF (10 mL) was added (3R,4R)-1-benzyl-N,4-dimethylpiperidin-3-amine (40g) (2.3 g, 2.8 mmol, prepared by the method described in WO2010/014930) and DIPEA (3.5 mL, 20 mmol) and stirred at 80° C. for 15 h. The reaction mixture was diluted with EtOAc (300 mL), washed with water (2×150 mL), brine (100 mL) and dried over MgSO₄. After filtration, the filtrate was concentrated and purified by flash column chromatography to afford 4-(((3R,4R)-1-Benzyl-4-methylpiperidin-3-yl)(methypamino)pyrrolo[1,2-b]pyridazine-3-carbonitrile (41a) (316 mg, 22%) as a white foam; ¹H NMR (300 MHz, DMSO-d₆) δ 7.96 (s, 1H), 7.80 (dd, J=1.5, 2.7 Hz, 1H), 7.33 (m, 4H), 7.25 (dd, J=4.6, 6.8 Hz, 1H), 6.86 (d, J=3.2 Hz, 1H), 6.77 (dd, J=2.7, 4.6 Hz, 1H), 4.45 (m, 1H), 3.78 (s, 3H), 3.33 (s, 1H), 3.20 (d, J=12.1 Hz, 1H), 2.83 (m, 1H), 2.65 (dd, J=3.9, 12.2 Hz, 1H), 2.16-1.88 (m, 3H), 1.86-1.53 (m, 2H), 0.93 (d, J=6.9 Hz, 3H). MS (ES⁺): 360.1 (M+1).

### Example 7

### 4-((1R,2S)-2-methylcyclohexylamino)pyrrolo[1,2-b]pyridazine-3-carboxamide (18g)

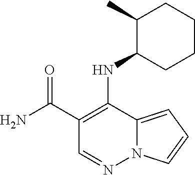

To a solution of 4-((1R,2S)-2-methylcyclohexylamino)pyrrolo[1,2-b]pyridazine-3-carbonitrile (18f) (83 mg, 0.33 mmol) in EtOH (8 mL) was added conc. NH₄OH (3 mL), followed by dropwise addition of H₂O₂ (0.14 mL, 1.37 mmol). The reaction mixture was stirred at room temperature for 13 h and concentrated in vacuum to dryness. The residue obtained was purified by flash column chromatography [silica gel 12 g, eluting with hexanes/ethyl acetate, 1:0 to 1:1, (R_{f}=0.33 with hexanes/ethyl acetate=1:1)] to furnish 4-((1R,2S)-2-methylcyclohexylamino)pyrrolo[1,2-b]pyridazine-3-carboxamide (18g) (38 mg, 42%) as a light blue solid; MP: 158.6° C.; ¹H NMR (300 MHz, DMSO) δ 10.99 (d, J=8.8 Hz, 1H), 8.20 (s, 1H), 7.65 (dd, J=2.7, 1.5 Hz, 1H), 6.87 (dd, J=4.8, 1.5 Hz, 1H), 6.65 (dd, J=4.6, 2.6 Hz, 1H), 4.38-4.26 (m, 1H), 2.00-1.24 (m, 9H), 0.90 (d, J=7.1 Hz, 3H). MS (ES+) 273.14 (M+1); [α]_{D}: −110.59 [CHCl₃, 0.17]; Analysis: Calcd for C₁₅H₂₀N₄O:C, 66.15; H, 7.40; N, 20.57. Found: C, 66.49; H, 7.63; N, 19.48.

### Preparation of Intermediate Compound 18f.

### Step 1: Preparation of Intermediate Compound 20e

To a solution of 2-methylcyclohexane (20b) (Aldrich, 56.53 g, 504 mmol) and (R)-1-phenylethanamine (61.39 g, 504 mmol) in benzene (750 mL) was added 4-methylbenzenesulfonic acid hydrate (0.96 g, 5.04 mmol) and heated at reflux using a dean stark apparatus for 72 h. The reaction was cooled to room temperature and neutralized with solid NaHCO₃ (2.1 g, 25.2 mmol). The reaction mixture was filtered through celite and the filtrate concentrated in vacuum to furnish (1R_{z}Z)—N-(2-methylcyclohexylidene)-1-phenylethanamine (20c) (108.7 g) as a colorless oil, which was used as such for next step.

To a solution of (R_{z} Z)—N—((S)-2-methylcyclohexylidene)-1-phenylethanamine (20c) (10 g) dissolved in EtOH (60 mL) was added Ra—Ni (3 g) and hydrogenated at 60 psi for 24 h. The catalyst was removed by filtration through celite and filtrate concentrated in vacuo to give 7.5 g of product which was treated with 17 mL of 4M HCl in dioxane. The product was concentrated to dryness to give (1R,2S)-2-Methyl-N—((R)-1-phenylethyl)cyclohexanamine (20d) (4.53 g, 51.2%) as an off-white solid after drying; mp 196.0° C. ¹H NMR (300 MHz, DMSO) δ 9.53 (s, 1H), 9.11 (s, 1H), 7.74 (d, J=6.4 Hz, 2H), 7.58-7.31 (m, 3H), 4.42 (s, 1H), 2.72 (s, 1H), 2.22 (s, 1H), 1.75 (s, 1H), 1.63 (d, J=6.7 Hz, 3H), 1.58 (s, 1H), 1.55-1.44 (m, 2H), 1.36-1.05 (m, 4H), 1.02 (d, J=7.0 Hz, 3H). MS (ES+) 218.3 (M+1). Optical rotation: [α]=+55.56 (c=1.26, EtOH). Analysis; Calcd for C₁₅H₂₃N.HCl: C, 70.98; H, 9.53; N, 5.52; Cl, 13.97. Found: C, 70.91; H, 9.61; N, 5.57; Cl, 13.79.

To a solution of (1R,2S)-2-Methyl-N—((R)-1-phenylethyl)cyclohexanamine hydrochloride (20d) (3.99 g) in EtOH (45 mL) was added Pd/C (10%) (750 mg) and hydrogenated at 50 psi for 24 h. The catalyst was removed by filtration through celite and filtrate concentrated in vacuum to give 2.3 g of white solid, which was recrystallized from EtOH/ether, to give (1R,2S)-2-Methylcyclohexanamine hydrochloride (20e) (1.35 g, 51.4%) as an off-white solid; mp 241.9° C.; ¹H NMR (300 MHz, DMSO) δ 8.13 (s, 3H), 3.20-3.08 (m, 1H), 1.99 (m, 1H), 1.63 (m, 3H), 1.44 (m, 3H), 1.31 (m, 2H), 0.92 (d, J=7.1 Hz, 3H). MS (ES+) 114.3 (M+1); Optical rotation: [α]=+7.97 (c=1.18, EtOH); Analysis: Calcd for C₇H₁₅N.HCl: C, 56.18; H, 10.78; N, 9.36; Cl, 23.69. Found: C, 56.06; H, 10.98; N, 9.21; Cl, 23.47.

### Step 2:

To a solution of 4-chloropyrrolo[1,2-b]pyridazine-3-carbonitrile (15g) (80 mg, 0.45 mmol) in DMF (10 mL) was added (1R,2S)-2-methylcyclohexanamine Hydrochloride (20e) (180 mg, 1.20 mmol), triethylamine (0.51 mL, 3.66 mmol) and stirred at room temperature for 15 h. The reaction mixture was diluted with EtOAc (100 mL), washed with water (2×50 mL), brine (50 mL), dried over MgSO₄, filtrated and the concentrated in vacuum. The residue was purified by flash column chromatography [silica gel, 30 g eluting with hexanes/ethyl acetate, 1:0 to 6:1 (R_{f}=0.46 hexanes/ethyl acetate=6:1)] to afford 4-((1R,2S)-2-methylcyclohexylamino)pyrrolo[1,2-b]pyridazine-3-carboxamide (18f) (0.105 g, 92%) as a light green oil; ¹H NMR (300 MHz, DMSO-d₆): δ 7.90 (s, 1H), 7.70 (dd, J=1.6, 2.6 Hz, 1H), 7.34 (s, 1H), 7.32 (dd, J=1.6, 4.5 Hz, 1H), 6.68 (dd, J=2.7, 4.4 Hz, 1H), 4.46-4.33 (m, 1H), 2.32-2.19 (m, 1H), 1.88-1.33 (m, 8H), 0.91 (d, J=7.1 Hz, 3H); MS (ES⁻): 253.0 (M−1).

### Example 8

### 4-((1S,2R)-2-methylcyclohexylamino)pyrrolo[1,2-b]pyridazine-3-carboxamide (18i)

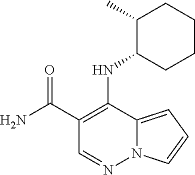

To a solution of 4-((1S,2R)-2-methylcyclohexylamino)pyrrolo[1,2-b]pyridazine-3-carbonitrile (18h) (105 mg, 0.41 mmol) in EtOH (10 mL) was added conc. NH₄OH (4 mL), followed by dropwise addition of H₂O₂ (0.18 mL, 1.76 mmol). The reaction mixture was stirred at room temperature for 19 h and concentrated in vacuum to dryness. The residue obtained was purified by flash column chromatography [silica gel 12 g, eluting with hexanes/ethyl acetate, 1:0 to 1:1, (R_{f}=0.33 with hexanes/ethyl acetate=1:1)] to furnish 4-((1S,2R)-2-methylcyclohexylamino)pyrrolo[1,2-b]pyridazine-3-carboxamide (18i) (50 mg, 45%) as a light blue solid; MP: 154.7° C.; ¹H NMR (300 MHz, DMSO) δ 10.99 (d, J=8.8 Hz, 1H), 8.20 (s, 1H), 7.65 (dd, J=2.7, 1.5 Hz, 1H), 6.87 (dd, J=4.8, 1.5 Hz, 1H), 6.65 (dd, J=4.6, 2.6 Hz, 1H), 4.38-4.26 (m, 1H), 2.00-1.24 (m, 9H), 0.90 (d, J=7.1 Hz, 3H). MS (ES+) 273.14 (M+1); [α]_{D}: +117.65 [CHCl₃, 0.17]; Analysis: Calcd for C₁₅H₂₀N₄O: C, 66.15; H, 7.40; N, 20.57. Found: C, 66.48; H, 7.78; N, 19.30.

### Preparation of Intermediate Compound 18h

### Step 1: Preparation of Intermediate Compound 20h

To a solution of 2-methylcyclohexane (20b) (Aldrich, 17.12 g, 153 mmol) and (S)-1-phenylethanamine (18.5 g, 153 mmol) in benzene (225 mL) was added 4-methylbenzenesulfonic acid hydrate (0.29 g, 1.53 mmol) and heated at reflux using a dean stark apparatus for 72 h. The reaction was cooled to room temperature and neutralized with solid NaHCO₃ (0.4 g, 7.65 mmol). The reaction mixture was filtered through Celite and the filtrate concentrated in vacuo to furnish (1S,Z)—N-(2-methylcyclohexylidene)-1-phenylethanamine (201) (32.1 g) as a colorless oil, which was used as such for next step.

A solution of (S,Z)—N—((S)-2-methylcyclohexylidene)-1-phenylethanamine (201) (32.5 g) was dissolved in EtOH (200 mL) and Ra—Ni (10 g) was added. The slurry was hydrogenated at 60 psi for 24 h. The catalyst was removed by filtration through Celite and the filtrate concentrated in vacuo and the product treated with 57 mL of 4M HCl in dioxane. The product was concentrated to dryness to give a residue which was recrystallized from EtOH/ether to give (1S,2R)-2-Methyl-N—((S)-1-phenylethyl)cyclohexanamine (20g) (16.5 g, 43.1%) as an off-white solid; mp 294.1° C.; ¹H NMR (300 MHz, DMSO δ 9.45 (s, 1H), 9.04 (s, 1H), 7.72 (m, 2H), 7.52-7.35 (m, 3H), 4.42 (m, 1H), 2.73 (m, 1H), 2.22 (m, 1H), 1.73 (m, 1H), 1.65 (m, 1H), 1.62 (d, J=6.7 Hz, 3H), 1.59-1.43 (m, 2H), 1.35-1.04 (m, 4H), 1.01 (d, J=7.0 Hz, 3H). MS (ES+): 218.3, (M+1); [α]_{D}=−52.75, (c, 1.365, EtOH); Analysis: Calcd for C₁₅H₂₃N.HCl: C, 70.98; H, 9.53; N, 5.52; Cl, 13.97. Found: C, 71.21; H, 9.60; N, 5.52; Cl, 14.00.

To a solution of (1S,2R)-2-methyl-N—((S)-1-phenylethyl)cyclohexanamine hydrochloride (20 g) (16 g) in EtOH (200 mL) was added Pd/C (10%) (3.2 g) and hydrogenated at 50 psi for 24 h. The catalyst was removed by filtration through Celite and the filtrate concentrated in vacuo to give product as a white solid, which was recrystallized from EtOH/ether, to give (1S,2R)-2-Methylcyclohexanamine (20h) (6.46 g, 68.5%) as an off-white solid; mp 241.4° C.; ¹H NMR (300 MHz, DMSO) δ 8.05 (s, 3H), 3.14 (m, 1H), 1.98 (m, 1H), 1.62 (m, 3H), 1.44 (m 3H), 1.31 (m, 2H), 0.92 (d, J=7.5, 3H). MS (ES+): 114.3 (M+1); [α]_{D}=−7.36, (c, 1.25, EtOH); Analysis: Calcd for C₇H₁₅N.HCl: C, 56.18; H, 10.78; N, 9.36; Cl, 23.69. Found: C, 55.84; H, 10.8; N, 9.31; Cl, 24.06.

### Step 2:

To a solution of 4-chloropyrrolo[1,2-b]pyridazine-3-carbonitrile (15g) (80 mg, 0.45 mmol) in DMF (10 mL) was added (1S,2R)-2-methylcyclohexanamine HCl salt (20h) (180 mg, 1.20 mmol), triethylamine (0.51 mL, 3.66 mmol) and stirred at room temperature for 13 h. The reaction mixture was diluted with EtOAc (100 mL), washed with water (2×50 mL), brine (50 mL), dried over MgSO₄, filtrated and the concentrated in vacuum. The residue was purified by flash column chromatography [silica gel, 30 g eluting with hexanes/ethyl acetate, 1:0 to 6:1 (R_{f}=0.46 hexanes/ethyl acetate=6:1)] to afford 4-((1S,2R)-2-methylcyclohexylamino)pyrrolo[1,2-b]pyridazine-3-carbonitrile (18h) (122 mg) as a colorless oil; ¹H NMR (300 MHz, DMSO-d₆): δ 7.90 (s, 1H), 7.70 (dd, J=1.6, 2.6 Hz, 1H), 7.34 (s, 1H), 7.32 (dd, J=1.6, 4.5 Hz, 1H), 6.68 (dd, J=2.7, 4.4 Hz, 1H), 4.45-4.33 (m, 1H), 2.32-2.20 (m, 1H), 1.88-1.30 (m, 8H), 0.92 (d, J=7.1 Hz, 3H); MS (ES⁻): 252.9 (M−1).

### Example 9

### tert-butyl (1R,2R)-2-(3-cyanopyrrolo[1,2-b]pyridazin-4-ylamino)cyclohexyl carbamate (47k)

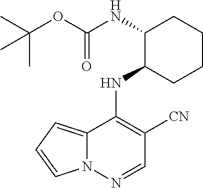

To a solution of 4-chloropyrrolo[1,2-b]pyridazine-3-carbonitrile (15g) (420 mg, 2.37 mmol) in DMF (40 mL) was added tert-butyl (1R,2R)-2-aminocyclohexylcarbamate (47j) (600 mg, 2.80 mmol), triethylamine (1.3 mL, 9.33 mmol) and stirred at room temperature for 16 h. The reaction mixture was diluted with EtOAc (300 mL), washed with water (2×150 mL), brine (100 mL), dried over MgSO₄, filtrated and the concentrated in vacuum. The residue was purified by flash column chromatography [silica gel, 24 g eluting with hexanes/ethyl acetate, 1:0 to 6:1, (R_{f}=0.38 with hexanes/ethyl acetate=6:1)] to afford tert-butyl (1R,2R)-2-(3-cyanopyrrolo[1,2-b]pyridazin-4-ylamino)cyclohexyl carbamate (47k) (440 mg, 54%) as a white solid. ¹HNMR (300 MHz, DMSO-d₆): δ 7.90 (s, 1H), 7.67 (dd, J=2.7, 1.4 Hz, 1H), 7.57 (bs, 1H), 7.06 (d, J=8.3 Hz, 1H), 6.93 (d, J=4.4, 1.4 Hz, 1H), 6.66 (dd, J=4.3, 2.7 Hz, 1H), 4.12-3.96 (m, 1H), 3.64-3.50 (m, 1H), 2.20-2.08 (m, 1H), 1.92-1.82 (m, 1H), 1.76-1.64 (m, 2H), 1.34-1.17 (m, 4H), 1.24 (s, 9H); MS (ES⁻) 354.4 (M−1); Analysis: Calcd for C₁₉H₂₅N₅O₂: C, 64.20; H, 7.09; N, 19.70. Found: C, 64.47; H, 7.32; N, 19.61.

### Preparation of Intermediate Compound 47j

To a solution of (1R,2R)-1,2-diaminocyclohexane (47g) (0.697 g, 6.1 mmol) and benzyloxycarbonyl Chloride (1.7 mL, 15.25 mmol) in CH₂Cl₂ (10 mL) at 0° C. was added triethylamine (2.55 mL, 18.3 mmol) dropwise. The reaction mixture was stirred for 15 min at 0° C., and it was allowed to warm to room temperature. The reaction mixture was stirred 2 h at room temperature, diluted with CH₂Cl₂ and washed with brine. The organic phase was dried and concentrated to give 2,2′-(1R,2R)-cyclohexane-1,2-diylbis(azan-1-yl-1-ylidene)bis(1-phenylethanone) (47h) (2.18 g) as a white solid, which was used as such in next step without further purification.

To a solution of 2,2′-(1R,2R)-cyclohexane-1,2-diylbis(azan-1-yl-1-ylidene)bis(1-phenylethanone) (47h) (2.18 g) in THF (10 mL) was added N,N-dimethyl-4-aminopyridine (149 mg, 1.22 mmol) followed by di-tert-butyldicarbonate (2.67 g, 12.2 mmol), and stirred at room temperature for 1 day. Extractive workup with EtOAc and purification by column chromatography (silica gel, eluting with 0-50% hexane:EtOAc) afforded mono Boc protected 2,2′-(1R,2R)-cyclohexane-1,2-diylbis(azan-1-yl-1-ylidene)bis(1-phenylethanone) (47i) (1.14 g, 42%) as a white solid. ¹H NMR (300 MHz, CDCl₃) δ 7.33 (m, 10H), 5.18 (m, 2H), 5.05 (d, J=5.0, 2H), 4.78 (m, 1H), 4.12 (m, 1H), 3.92 (m, 1H), 2.12 (m, 2H), 1.81-1.73 (m, 2H), 1.39 (s, 9H), 1.26 (m, 4H).

To a solution of mono Boc protected 2,2′-(1R,2R)-cyclohexane-1,2-diylbis(azan-1-yl-1-ylidene)bis(1-phenylethanone) (47i) (1.14 g, 2.4 mmol) in ethanol (20 mL) was added Pd/C (10%, 100 mg) and hydrogenated at 60 psi for 3 h. The reaction mixture was filtered through Celite, and the filtrate was concentrated to give tert-butyl (1R,2R)-2-aminocyclohexylcarbamate (47j) (0.53 g, 100%) as a white solid. A small portion of tent-Butyl (1R,2R)-2-aminocyclohexylcarbamate was recrystallized from CH₂Cl₂-hexane to give an analytically pure sample as an off-white solid; mp 116.6° C.; ¹H NMR (300 MHz, MeOD) δ 3.07 (td, J=3.9, 10.7 Hz, 1H), 2.38 (td, J=3.9, 10.4 Hz, 1H), 1.90 (t, J=12.6 Hz, 2H), 1.70 (dt, J=7.2, 18.1 Hz, 2H), 1.44 (s, 9H), 1.35-1.08 (m, 4H); ¹³C NMR (300 MHz, MeOD) δ 158.50, 80.00, 55.41, 34.98, 33.63, 28.78, 26.32, 26.07; MS ES (+) 215.3 (M+1); ES (−) 213.30 (M−1); [α]=−37.80 (0.545, MeOH); Analysis: Calcd for C₁₁H₂₂N₂O₂: C, 61.65; H, 10.35; N, 13.07. Found: C, 61.87; H, 10.43; N, 12.80.

### Example 10

### tert-butyl (1R,2R)-2-(3-carbamoylpyrrolo[1,2-b]pyridazin-4-ylamino)cyclohexyl carbamate (47l)

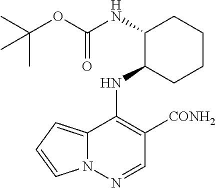

To a solution of tert-butyl (1R,2R)-2-(3-cyanopyrrolo[1,2-b]pyridazin-4-ylamino)cyclohexyl carbamate (47k) (428 mg, 1.2 mmol) in EtOH (30 mL) was added conc. NH₄OH (11 mL), followed by dropwise addition of 35% aqueous H₂O₂ (0.43 mL, 4.87 mmol). The reaction mixture was stirred at room temperature for 19 h and concentrated in vacuum to dryness. The residue obtained was purified by flash column chromatography [silica gel 12 g, eluting with hexanes/ethyl acetate, 1:0 to 1:1, (R_{f}=0.2 with hexanes/ethyl acetate=1:1)] to furnish tert-butyl (1R,2R)-2-(3-carbamoylpyrrolo[1,2-b]pyridazin-4-ylamino)cyclohexyl carbamate (47l) (209 mg, ¹HNMR (300 MHz, DMSO-d₆): δ 8.08 (s, 1H), 7.55 (dd, J=1.5, 2.7 Hz, 1H), 6.91 (dd, J=1.5, 4.6 Hz, 1H), 6.67 (dd, J=2.7, 4.6 Hz, 1H), 4.14-4.00 (m, 1H), 3.56-3.40 (m, 1H), 2.34-2.22 (m, 1H), 2.01-1.93 (m, 1H), 1.86-1.72 (m, 2H), 1.52-1.36 (m, 4H), 1.32 (s, 9H); MS (ES⁺) 396.1 (M+Na).

### Example 11

### 4-((1R,2R)-2-aminocyclohexylamino)pyrrolo[1,2-b]pyridazine-3-carboxamide

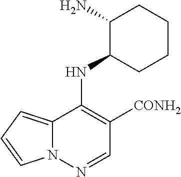

To solution of tert-butyl (1R,2R)-2-(3-carbamoylpyrrolo[1,2-b]pyridazin-4-ylamino)cyclohexyl carbamate (47l) (0.196 g, 0.52 mmol) in dichloromethane (6 mL) was added trifluoroacetic acid (2 mL, 26 mmol) and stirred at room temperature for 2 h. The reaction mixture was concentrated in vacuo and residue obtained was purified by flash column chromatography [silica gel 4 g, eluting with chloroforms/methanol, 1:0 to 3:2, (R_{f}=0.21 with chloroforms/methanol=3:2)] to furnish 4-((1R,2R)-2 aminocyclohexylamino)pyrrolo[1,2-b]pyridazine-3-carboxamide (47m) (86 mg, 35%) as a brown solid; ¹HNMR (300 MHz, DMSO-d₆): δ 10.65 (d, J=8.6 Hz, 1H), 8.27 (s, 1H), 8.01 (bs, 3H), 7.74 (dd, J=1.4, 2.6 Hz, 1H), 6.92 (dd, J=1.4, 4.6 Hz, 1H), 6.74 (dd, J=2.7, 4.5 Hz, 1H), 4.24-4.08 (m, 1H), 3.28-3.12 (m, 2H), 2.10-1.98 (m, 2H), 1.78-1.64 (m, 2H), 1.52-1.30 (s, 4H); MS (ES+): 274.1 (M+1).

### Example 12

### 4-((1R,2R)-2-(2-cyanoacetamido)cyclohexylamino)pyrrolo[1,2-b]pyridazine-3

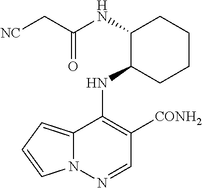

To a ice cold solution of 4-((1R,2R)-2 aminocyclohexylamino)pyrrolo[1,2-b]pyridazine-3-carboxamide (47m) (66 mg, 0.26 mmol) in DMF (4 mL) was added DIPEA (0.09 mL, 0.52 mmol) followed by cyano acetic acid (0.021 g, 0.24 mmol) and HATU (0.092 g, 0.24 mmol) and allowed to warm to room temperature. The reaction mixture was diluted with water (75 mL) and extracted with chloroform (100 mL). The organic layer was dried and concentrated under vacuum. The residue obtained was purified by flash column chromatography [silica gel, 4 g, eluting with chloroform/methanol, 1:0 to 10:1, (R_{f}=0.32 with chloroform/methanol=10:1)] to furnish 4-((1R,2R)-2-(2-cyanoacetamido)cyclohexylamino)pyrrolo[1,2-b]pyridazine-3-carboxamide (47n) (30 mg, 37%) as an off white solid; ¹HNMR (300 MHz, DMSO-d₆) δ 10.78 (d, J=8.3 Hz, 1H), 8.32 (d, J=8.0, 1H), 8.19 (s, 1H), 7.67 (dd, J=1.4, 2.6 Hz, 1H), 6.85 (dd, J=1.4, 4.5 Hz, 1H), 6.68 (dd, J=2.7, 4.5 Hz, 1H), 4.10-3.96 (m, 1H), 3.78-3.66 (m, 1H), 3.63-3.42 (m, 2H), 2.24-2.10 (m, 1H), 1.96-1.82 (m, 1H), 1.74-1.62 (m, 2H), 1.52-1.28 (m, 4H); IR (KBr, cm⁻¹): 3450, 2925, 1658, 1619, 1458; MS (ES⁺): 341.1 (M+1).

### Example 13

### 4-((1S,2R)-2-methylcyclohexylamino)-6-nitropyrrolo[1,2-b]pyridazine-3-carbonitrile (48a)

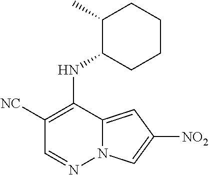

To a solution of 4-chloro-6-nitropyrrolo[1,2-b]pyridazine-3-carbonitrile (47d) (180 mg, 0.81 mmol) in DMF (20 mL) was added (1S,2R)-2-methylcyclohexanamine hydrochloride (20h) (320 mg, 2.14 mmol) triethylamine (0.90 mL, 6.46 mmol) and stirred at room temperature overnight. The reaction mixture was diluted with EtOAc (150 mL), washed with water (2×75 mL), brine (50 mL), dried over MgSO₄ filtered and concentrated in vacuum to dryness. The residue obtained was purified by flash column chromatography [silica gel 12 g, eluting with hexanes/ethyl acetate, 1:0 to 5:1, (R_{f}=0.46 with hexanes/ethyl acetate=5:1)] to afford 4-((1S,2R)-2-methylcyclohexylamino)-6-nitropyrrolo[1,2-b]pyridazine-3-carbonitrile (48a) (239 mg, 99%) as a yellow solid; ¹H NMR (300 MHz, DMSO-d₆): δ 8.68 (d, J=1.8 Hz, 1H), 8.18 (s, 1H), 8.16 (d, J=1.8 Hz, 1H), 7.97 (d, J=8.1 Hz, 1H), 4.48-4.36 (m, 1H), 2.34-2.22 (m, 1H), 1.91-1.29 (m, 8H), 0.93 (d, J=7.1 Hz, 3H); MS (ES⁻): 298.0 (M−1).

### Preparation of Intermediate Compound 47d

A stirred solution of 2,2,2-trichloro-1-(1H-pyrrol-2-yl)ethanone [20 g, 94.14 mmol, Prepared from pyrrole using the procedure from Organic Syntheses, Coll. Vol. 6, p. 618 (1988); Vol. 51, p. 100 (1971)] and Ac₂O (110 mL) was cooled to −40° C. and treated dropwise with 70% nitric acid (8.24 mL, 128.16 mmol) over 2 h. After completion of addition, the mixture was warmed to room temperature over 2 h and then cooled back down to −40° C. Sufficient ice-water was added to precipitate crude 2,2,2-trichloro-1-(4-nitro-1H-pyrrol-2-yl)ethanone. The residue was filtered and washing with ice-water, dried and purified by flash column chromatography on silica gel (hexanes:ethyl acetate 1:0 to 5:2, R_{f}=0.54 with hexanes:ethyl acetate 5:2) to give 2,2,2-trichloro-1-(4-nitro-1H-pyrrol-2-yl)ethanone (12.5 g, 52%) as a solid; ¹H NMR (300 MHz, DMSO-d₆): δ=13.67 (s, 1H), 8.40 (d, J=1.5 Hz, 1H), 7.71 (d, J=1.52, 1H).

To a solution of 2,2,2-trichloro-1-(4-nitro-1H-pyrrol-2-yl)ethanone (12.47 g, 48.43 mmol) in methanol (26 mL) at room temperature was added MeONa (17 mL, 25% w/w, 74.29 mmol). The mixture was stirred for 2 h, then quenched with aqueous H₂SO₄ (3 M, 26 mL) and cooled to 0° C. Ice-water was added to precipitate methyl 4-nitro-1H-pyrrole-2-carboxylate (47a) (8.07 g, 98%) as a solid; ¹H NMR: (DMSO-d₆, 300 MHz): δ=13.19 (s, 1H), 8.07 (d, J=1.68, 1H), 7.31 (d, J=1.65, 1H), 3.83 (s, 3H).

To a solution of methyl 4-nitro-1H-pyrrole-2-carboxylate (47a) (1.0 g, 5.88 mmol) in DMF (50 mL) cooled to −10° C. was added LiHMDS (1 M in THF, 7.1 mL) and stirred at −10° C. for 15 min. To the cold reaction mixture was added O-(diphenylphosphoryl)hydroxylamine 15e (1.8 g, 7.72 mmol) and stirred at room temperature for 20 h. The reaction mixture was diluted with ethyl acetate (200 mL) washed with water (2×100 mL), brine (100 mL), dried over MgSO₄ and filtered. The filtrate was concentrated in vacuo and the residue obtained was purified by column chromatography [silica gel 30 g, eluting with chloroform/methanol, 1:0 to 100:1, (R_{f}=0.59 with chloroform/methanol=100:1)] to furnish methyl 1-amino-4-nitro-1H-pyrrole-2-carboxylate (47b) (437 mg, 40%) as a white solid; ¹H NMR (300 MHz, DMSO-d₆): δ 8.08 (d, J=2.3, 1H), 7.26 (d, J=2.3, 1H), 6.72 (s, 2H), 3.82 (s, 3H); MS (ES⁻): 219.9 (M+Cl); Analysis: Calcd for C₆H₇N₃O₄:C, 38.92; H, 3.81; N, 22.70. Found: C, 39.13; H, 3.75; N, 22.66.

To a solution of methyl 1-amino-4-nitro-1H-pyrrole-2-carboxylate (47b) (417 mg, 2.25 mmol) in EtOH (12 mL) was added 3,3-diethoxypropanenitrile (2.9 mL, 95%, 18.36 mmol), 1N HCl (aq. 0.6 mL) and heated at reflux for 15 h. The reaction mixture was cooled to room temperature, treated with DBU (3.8 mL, 24.90 mmol), and stirred at 80° C. for 1 h. The reaction mixture was concentrated in vacuo to remove most of EtOH. The residue obtained was diluted with EtOAc (75 mL), washed with water (50 mL, 30 mL). The combined aqueous solution was acidified with 4N HCl to pH=1 and extracted with chloroform/methanol (3:1, 4×100 mL). The combined extracts were dried over MgSO_{4, filtered} and the filtrate was concentrated in vacuo. The residue obtained was purified by column chromatography [silica gel 120 g, eluting with chloroform/methanol, 1:0 to 4:1, (R_{f}=0.46 with chloroform/methanol=4:1)] to give 4-hydroxy-6-nitropyrrolo[1,2-b]pyridazine-3-carbonitrile (47c) (343 mg) as a brown-purple gum; ¹H NMR (300 MHz, DMSO-d₆): δ 9.58 (s, 1H), 8.21 (d, J=2.2 Hz, 1H), 7.87 (s, 1H), 6.93 (d, J=2.2 Hz, 1H); MS (ES⁻): 203.0 (M−1).

To a solution of 4-hydroxy-6-nitropyrrolo[1,2-b]pyridazine-3-carbonitrile (47c) (320 mg) in acetonitrile (8 mL) was added benzyltriethylammonium chloride (mg, 98%, 3.15 mmol) and N,N-diethylaniline (0.32 mL, 2.50 mmol). The mixture was heated to 80° C. followed by the addition of POCl₃ (0.88 mL, 9.52 mmol). The reaction mixture was stirred at 80° C. for 1 h and then concentrated to dryness. The residue obtained was dissolved in chloroform (200 mL), washed with 1N NaHCO₃ (100 mL), water (100 mL), brine (50 mL), dried over MgSO₄ and filtered. The filtrate was concentrated in vacuo and the residue obtained was purified by column chromatography [silica gel 30 g, eluting with hexanes/ethyl acetate, 1:0 to 5:1, (R_{f}=0.45 with hexanes/ethyl acetate 5:1)] to afford 4-chloro-6-nitropyrrolo[1,2-b]pyridazine-3-carbonitrile (47d) (95 mg, 20% for two steps) as a yellow solid; ¹H NMR (300 MHz, DMSO-d₆): δ 9.26 (d, J=1.9 Hz, 1H), 8.84 (s, 1H), 7.75 (d, J=1.9 Hz, 1H).

### Example 14

### 4-((1S,2R)-2-methylcyclohexylamino)-6-nitropyrrolo[1,2-b]pyridazine-3-carboxamide (48b)

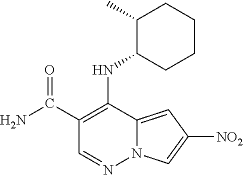

To a solution of 4-((1S,2R)-2-methylcyclohexylamino)-6-nitropyrrolo[1,2-b]pyridazine-3-carbonitrile (48a) (219 mg, 0.73 mmol) in EtOH (18 mL) was added conc. NH₄OH (7 mL), followed by dropwise addition of H₂O₂ (0.27 mL, 35%, 3.06 mmol). The reaction mixture was stirred at room temperature for 16 h and concentrated in vacuum to dryness. The residue obtained was purified by flash column chromatography [silica gel 4 g, eluting with hexanes/ethyl acetate, 1:0 to 2:1, (R_{f}=0.27 with hexanes/ethyl acetate=2:1)] to furnish 4-((1S,2R)-2-methylcyclohexylamino)-6-nitropyrrolo[1,2-b]pyridazine-3-carboxamide (48b) (178 mg, 77%) as a yellow solid; ¹H NMR (300 MHz, DMSO-d₆): δ 11.36 (d, J=8.6 Hz, 1H), 8.62 (d, J=1.9 Hz, 1H), 8.42 (s, 1H), 7.46 (d, J=1.9 Hz, 1H), 4.42-4.32 (m, 1H), 1.97-1.31 (m, 9H), 0.89 (d, J=6.9 Hz, 3H); MS (ES₋): 315.7 (M−1).

### Example 15

### 6-amino-4-((1S,2R)-2-methylcyclohexylamino)pyrrolo[1,2-b]pyridazine-3-carboxamide (48c)

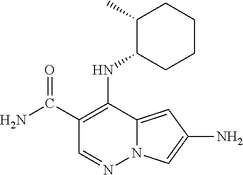

A solution of 4-((1S,2R)-2-methylcyclohexylamino)-6-nitropyrrolo[1,2-b]pyridazine-3-carboxamide (48b) (145 mg) in EtOH/ethyl acetate (30 mL/10 mL) was added Pd/C (10%, 60 mg) and hydrogenated at ˜50 psi for 5 h. The reaction mixture was filtered through celite to remove catalyst and concentrated in vacuum. The residue obtained was purified by flash column chromatography (silica gel 4 g, eluting with chloroform with 10% acetic acid/methanol=1:0 to 92:8) to give 6-amino-4-((1S,2R)-2-methylcyclohexylamino)pyrrolo[1,2-b]pyridazine-3-carboxamide (48c) (58 mg, 44%) as a yellow solid; ¹HNMR (300 MHz, DMSO-d₆): δ 10.54 (d, J=8.6 Hz, 1H), 8.02 (s, 1H), 7.03 (d, J=1.8 Hz, 1H), 6.21 (d, J=1.8 Hz, 1H), 4.24-4.12 (m, 1H), 1.85-1.30 (m, 9H), 0.89 (d, J=6.9 Hz, 3H); MS (ES⁺): 310.1 (M+Na).

### Example 16

### 4-((1R,2S)-2-methylcyclohexylamino)-6-nitropyrrolo[1,2-b]pyridazine-3-carbonitrile (48d)

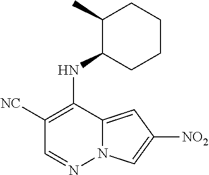

To a solution of 4-chloro-6-nitropyrrolo[1,2-b]pyridazine-3-carbonitrile (47d) (180 mg, 0.81 mmol) in DMF (20 mL) was added (1R,2S)-2-methylcyclohexanamine hydrochloride (20e) (320 mg, 2.14 mmol) triethylamine (0.90 mL, 6.46 mmol) and stirred at room temperature overnight. The reaction mixture was diluted with EtOAc (150 mL), washed with water (2×75 mL), brine (50 mL), dried over MgSO₄ filtered and concentrated in vacuum to dryness. The residue obtained was purified by flash column chromatography [silica gel 12 g, eluting with hexanes/ethyl acetate, 1:0 to 5:1, (R_{f}=0.46 with hexanes/ethyl acetate=5:1)] to afford 4-((1R,2S)-2-methylcyclohexylamino)-6-nitropyrrolo[1,2-b]pyridazine-3-carbonitrile (48d) (228 mg, 94%) as a yellow solid; ¹H NMR (300 MHz, DMSO-d₆): δ 8.68 (d, J=2.0 Hz, 1H), 8.18 (s, 1H), 8.16 (d, J=1.9 Hz, 1H), 7.97 (d, J=7.9 Hz, 1H), 4.48-4.36 (m, 1H), 2.34-2.22 (m, 1H), 1.91-1.29 (m, 8H), 0.93 (d, J=7.1 Hz, 3H); MS (ES⁻): 297.9 (M−1).

### Example 17

### 4-((1R,2S)-2-methylcyclohexylamino)-6-nitropyrrolo[1,2-b]pyridazine-3

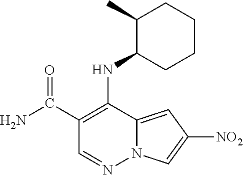

To a solution of 4-((1R,2S)-2-methylcyclohexylamino)-6-nitropyrrolo[1,2-b]pyridazine-3-carbonitrile (48d) (208 mg, 0.69 mmol) in EtOH (16 mL) was added conc. NH₄OH (6 mL), followed by dropwise addition of H₂O₂ (0.25 mL, 35%, 2.83 mmol). The reaction mixture was stirred at room temperature for 16 h and concentrated in vacuum to dryness. The residue obtained was purified by flash column chromatography [silica gel 4 g, eluting with hexanes/ethyl acetate, 1:0 to 2:1, (R_{f}=0.27 with hexanes/ethyl acetate=2:1)] to furnish 4-((1R,2S)-2-methylcyclohexylamino)-6-nitropyrrolo[1,2-b]pyridazine-3-carboxamide (48e) (144 mg, 66%) as a yellow solid; ¹H NMR (300 MHz, DMSO): δ 11.36 (d, J=8.9 Hz, 1H), 8.62 (d, J=1.9 Hz, 1H), 8.42 (s, 1H), 7.89 (bs, 1H), 7.46 (d, J=1.9 Hz, 1H), 7.28 (bs, 1H), 4.42-4.32 (m, 1H), 1.96-1.33 (m, 9H), 0.89 (d, J=6.9 Hz, 3H); MS (ES⁻): 315.9 (M−1).

### Example 18

### 6-amino-4-((1R,2S)-2-methylcyclohexylamino)pyrrolo[1,2-b]pyridazine-3-carboxamide (48f)

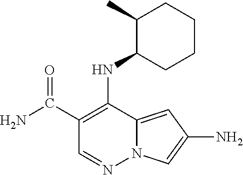

A solution of 4-((1R,2S)-2-methylcyclohexylamino)-6-nitropyrrolo[1,2-b]pyridazine-3-carboxamide (48e) (74 mg, 0.23 mmol) in EtOH/ethyl acetate (15 mL/5 mL) was added Pd/C (10%, 30 mg) and hydrogenated at ˜50 psi for 5 h. The reaction mixture was filtered through celite to remove catalyst and concentrated in vacuum. The residue obtained was purified by flash column chromatography (silica gel 4 g, eluting with chloroform with 10% acetic acid/methanol=1:0 to 92:8) to give 6-amino-4-((1R,2S)-2-methylcyclohexylamino)pyrrolo[1,2-b]pyridazine-3-carboxamide (481) (54 mg, 62%) as a light brown solid; ¹HNMR (300 MHz, DMSO-d₆): δ 10.54 (d, J=8.8, 1H), 8.02 (s, 1H), 7.03 (d, J=1.8 Hz, 1H), 6.21 (d, J=1.8 Hz, 1H), 4.24-4.12 (m, 1H), 1.87-1.27 (m, 9H), 0.89 (d, J=6.9 Hz, 3H); MS (ES⁺): 288.1 (M+1) [α]_{D}=−77.60 (c 0.235, MeOH).

### Example 19

### 4-(1-(4,5-Dimethylthiazol-2-yl)-3-methylbutylamino)pyrrolo[1,2-b]pyridazine-3-carbonitrile (49b)

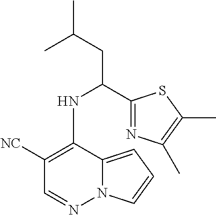

To a solution of 4-chloropyrrolo[1,2-b]pyridazine-3-carbonitrile (15g) (0.190 g, 1.070 mmol) in DMF (2.5 mL) was added at room temperature 1-(4,5-dimethylthiazol-2-yl)-3-methylbutan-1-amine (49a) (OTAVA 1044264, 0.25 g, 1.26 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-(1-(4,5-Dimethylthiazol-2-yl)-3-methylbutylamino)pyrrolo[1,2-b]pyridazine-3-carbonitrile (49b) as a white semisolid, which was crystallized from ether/hexane to furnish (0.208 g, 57%) as a white, crystalline solid; MP 137.9° C.; ¹HNMR (300 MHz, DMSO) δ 8.24 (d, J=9.2, 1H), 7.95 (s, 1H), 7.77 (dd, J=1.6, 2.6, 1H), 7.31 (s, 1H), 6.73 (dd, J=2.7, 4.4, 1H), 5.84 (m, 1H), 2.28 (s, 3H), 2.23 (s, 3H), 2.12 (m, 1H), 1.92 (m, 1H), 1.78 (m, 1H), 0.96 (t, J=6.5, 6H); MS (ES+) 340.1 (M+1), 362.0 (M+Na), 701.0 (2M+Na), (ES−) 337.9 (m−1), 373.9 (M+Cl); Analysis: Calcd for C₁₈H₂₁N₅S: C, 62.85; H, 6.30; N, 20.36; S, 9.32. Found: C, 63.03; H, 6.46; N, 20.33; S, 9.58

### Example 20

### 4-(1-(4,5-dimethylthiazol-2-yl)-3-methylbutylamino)pyrrolo[1,2-b]pyridazine-3-carboxamide (49c)

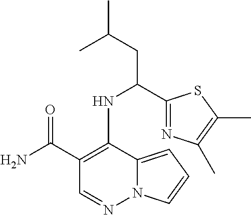

To a solution of 4-(1-(4,5-dimethylthiazol-2-yl)-3-methylbutylamino)pyrrolo[1,2-b]pyridazine-3-carbonitrile (49b) (0.136 g, 0.4 mmol) in EtOH (15 mL) was added concentrated NH₄OH (4 mL), followed by dropwise addition of H₂O₂ (0.2 mL, 1.6 mmol) and stirred at room temperature for 14 h. The reaction mixture was concentrated to dryness in vacuum. The residue obtained was purified by flash column chromatography (silica gel 4 g, eluting with 0-100% ethyl acetate in hexanes) to furnish a white semisolid, which was crystallized from ether/hexane to furnish 4-(1-(4,5-dimethylthiazol-2-yl)-3-methylbutylamino)pyrrolo[1,2-b]pyridazine-3-carboxamide (49c) (0.068 g, 0.190 mmol, 47.5%) as a white solid; ¹H NMR (300 MHz, DMSO) 11.21 (d, J=7.6, 1H), 8.28 (s, 1H), 8.05-7.74 (bs, 1H), 7.69 (dd, J=1.5, 2.6, 1H), 7.52-6.99 (bs, 1H), 6.77 (dd, J=1.5, 4.7, 1H), 6.62 (dd, J=2.7, 4.6, 1H), 5.44 (s, 1H), 2.24 (s, 6H), 1.81 (d, J=4.9, 3H), 0.95 (d, J=6.1, 3H), 0.87 (d, J=6.1, 3H); MS (ES−) 356.4 (M−1); Analysis: Calcd for C₁₆H₂₁N₅O: C, 60.48; H, 6.49; N, 19.59. Found: C, 60.15; H, 6.50; N, 19.38.

### Example 21

### 4-(2-methyl-2-morpholinopropylamino)pyrrolo[1,2-b]pyridazine-3-carboxamide (49e)

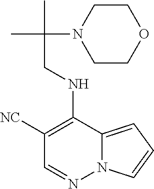

To a solution of 4-chloropyrrolo[1,2-b]pyridazine-3-carbonitrile (15g) (0.177 g, 0.997 mmol) in DMF (2.5 mL) was added at room temperature 2-methyl-2-morpholinopropan-1-amine (49d) (OTAVA 7020410146, 0.25 g, 1.580 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-(2-methyl-2-morpholinopropylamino)pyrrolo[1,2-b]pyridazine-3-carbonitrile (49e) as a white semisolid, which was crystallized from ether/hexane to furnish (0.238 g, 79.7%) white, crystalline solid; MP 178.8° C.; ¹H NMR (300 MHz, DMSO) δ 7.98 (s, 1H), 7.81 (dd, J=1.5, 2.6, 1H), 7.08 (d, J=4.6, 2H), 6.76 (dd, J=2.7, 4.5, 1H), 3.71 (d, J=4.5, 2H), 3.64 (d, J=4.2, 4H), 3.33 (s, 4H), 1.10 (s, 6H); MS (ES+) 300.1, (ES−) 298.0 (M−1).

### Example 22

### 4-(2-methyl-2-morpholinopropylamino)pyrrolo[1,2-b]pyridazine-3-carboxamide (49f)

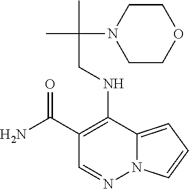

To a solution of 4-(2-methyl-2-morpholinopropylamino)pyrrolo[1,2-b]pyridazine-3-carbonitrile (49e) (0.120 g, 0.4 mmol) in EtOH (15 mL) was added concentrated NH₄OH (4 mL), followed by dropwise addition of H₂O₂ (0.2 mL, 1.6 mmol) and stirred at room temperature for 14 h. The reaction mixture was concentrated to dryness in vacuum. The residue obtained was purified by flash column chromatography (silica gel 4 g, eluting with 0-100% ethyl acetate in hexanes) to furnish a white semisolid, which was crystallized from ether/hexane to furnish 4-(2-methyl-2-morpholinopropylamino)pyrrolo[1,2-b]pyridazine-3-carboxamide (49f) (0.026 g, 0.083 mmol, 20.7%) as a white solid; ¹H NMR (300 MHz, DMSO) d 10.74 (s, 1H), 8.16 (s, 1H), 7.64 (dd, J=1.5, 2.6, 1H), 7.56-7.03 (bs, 2H), 6.99 (dd, J=1.5, 4.5, 1H), 6.62 (dd, J=2.7, 4.5, 1H), 3.71 (d, J=4.2, 2H), 3.62 (s, 4H), 2.49-2.44 (m, 4H), 1.07 (s, 6H); MS (ES+) 340.1 (M+Na), (ES−) 316.0 (M−1).

### Example 23

### 4-(2-(dimethylamino)-2-(furan-2-yl)ethylamino)pyrrolo[1,2-b]pyridazine-3-carbonitrile (49h)

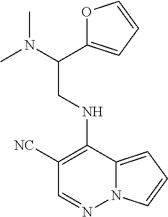

To a solution of 4-chloropyrrolo[1,2-b]pyridazine-3-carbonitrile (15g) (0.177 g, 0.997 mmol) in DMF (2.5 mL) was added at room temperature 1-(furan-2-yl)-N1,N1-dimethylethane-1,2-diamine (49g) (OTAVA 7020410165, 0.25 g, 1.62 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-(2-(dimethylamino)-2-(furan-2-yl)ethylamino)pyrrolo[1,2-b]pyridazine-3-carbonitrile (49h) as a white semisolid, which was crystallized from ether/hexane to furnish (0.253 g, 86%) white, crystalline solid; MP 106.9° C.; ¹H NMR (300 MHz, DMSO) δ 7.94 (s, 1H), 7.76-7.71 (m, 1H), 7.65 (dd, J=0.7, 1.8, 2H), 7.04 (dd, J=1.6, 4.5, 1H), 6.70 (dd, J=2.7, 4.4, 1H), 6.44 (dd, J=1.8, 3.2, 1H), 6.39 (d, J=3.0, 1H), 4.09 (m, 3H), 2.16 (s, 6H); MS (ES+) 588.9 (2M), (ES−) 329.9 (M+Cl); Analysis: Calcd for C₁₆H₁₇N₅O: C, 65.07; H, 5.80; N, 23.71. Found: C, 65.23; H, 5.98; N, 23.64.

### Example 24

### 4-(2-(dimethylamino)-2-(furan-2-yl)ethylamino)pyrrolo[1,2-b]pyridazine-3-carboxamide (49i)

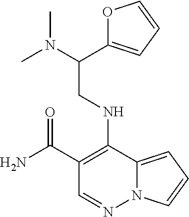

To a solution of 4-(2-(dimethylamino)-2-(furan-2-yl)ethylamino)pyrrolo[1,2-b]pyridazine-3-carbonitrile (49h) (0.114 g, 0.386 mmol) in EtOH (15 mL) was added concentrated NH₄OH (4 mL), followed by dropwise addition of H₂O₂ (0.18 mL, 1.56 mmol) and stirred at room temperature for 14 h. The reaction mixture was concentrated to dryness in vacuum. The residue obtained was purified by flash column chromatography (silica gel 4 g, eluting with 0-100% ethyl acetate in hexanes) to furnish 4-(2-(dimethylamino)-2-(furan-2-yl)ethylamino)pyrrolo[1,2-b]pyridazine-3-carboxamide (49i) as a white semisolid, which was crystallized from ether/hexane to furnish (0.045 g, 37.2%) as an olive colored solid; ¹H NMR (300 MHz, DMSO) δ 10.69 (s, 1H), 8.17 (s, 1H), 7.73-7.63 (m, 2H), 6.98 (dd, J=1.5, 4.6, 1H), 7.58-6.86 (bs, 2H), 6.66 (dd, J=2.7, 4.5, 1H), 6.47 (d, J=1.6, 2H), 4.01 (m, 3H), 2.17 (s, 6H); MS (ES+) 314.1 (M+1).

### Example 25

### 4-(1-(2,4-dichlorophenyl)cyclopropylamino)pyrrolo[1,2-b]pyridazine-3-carbonitrile (49k)

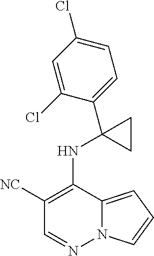

To a solution of 4-chloropyrrolo[1,2-b]pyridazine-3-carbonitrile (15g) (0.187 g, 1.05 mmol) in DMF (2.5 mL) was added at room temperature 1-(2,4-dichlorophenyl)cyclopropanamine (49j) (OTAVA 1059458, 0.25 g, 1.05 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish a white semisolid, which was crystallized from ether/hexane to furnish 4-(1-(2,4-dichlorophenyl)cyclopropylamino)pyrrolo[1,2-b]pyridazine-3-carbonitrile (49k) (0.196 g, 54.4%) as a white, crystalline solid; MP 207.7° C.; ¹H NMR (300 MHz, DMSO) δ 8.40 (s, 1H), 7.90 (s, 1H), 7.86 (d, J=8.5, 1H), 7.73 (dd, J=1.6, 2.6, 1H), 7.55 (d, J=2.2, 1H), 7.43 (dd, J=2.2, 8.5, 1H), 7.27 (dd, J=1.6, 4.5, 1H), 6.72 (dd, J=2.7, 4.5, 1H), 1.68 (s, 2H), 1.51 (s, 21-1); MS (ES−) 376.6 (M±Cl); Analysis: Calcd for C₁₇H₁₂Cl₂N₄: C, 59.49; H, 3.52; N, 16.32. Found: C, 59.73; H, 3.41; N, 16.28.

### Example 26

### 4-(1-(2,4-dichlorophenyl)cyclopropylamino)pyrrolo[1,2-b]pyridazine-3-carboxamide (49l)

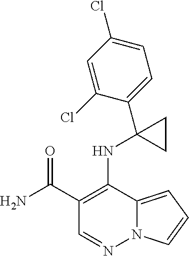

To a solution of 4-(1-(2,4-dichlorophenyl)cyclopropylamino)pyrrolo[1,2-b]pyridazine-3-carbonitrile (49k) (0.092 g, 0.286 mmol) in EtOH (13 mL) was added concentrated NH₄OH (3 mL), followed by dropwise addition of H₂O₂ (0.13 mL, 1.072 mmol) and stirred at room temperature for 22 h. The reaction mixture was concentrated to dryness in vacuum. The residue obtained was purified by flash column chromatography (silica gel 4 g, eluting with 0-100% ethyl acetate in hexanes) to furnish off-white semisolid, which was crystallized from ether/hexane to furnish 4-(1-(2,4-dichlorophenyl)cyclopropylamino)pyrrolo[1,2-b]pyridazine-3-carboxamide (49l) (0.019 g, 19.8%) as a white solid; ¹H NMR (300 MHz, DMSO) δ 11.58 (s, 1H), 8.16 (s, 1H), 7.81 (d, J=8.5, 1H), 7.65 (dd, J=1.5, 2.6, 1H), 7.55 (d, J=2.2, 1H), 7.39 (dd, J=1.6, 4.6, 1H), 7.32 (dd, J=2.2, 8.4, 1H), 7.26-7.00 (m, 1H), 6.68 (dd, J=2.6, 4.5, 1H), 1.55 (s, 2H), 1.46 (s, 2H); MS (ES−) 360.4 (M−1);

### Example 27

### 4-(2-(2-methoxyphenyl)-2-morpholinoethylamino)pyrrolo[1,2-b]pyridazine-3-carbonitrile (50b)

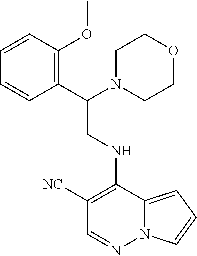

To a solution of 4-chloropyrrolo[1,2-b]pyridazine-3-carbonitrile (15g) (0.177 g, 0.997 mmol) in DMF (2.5 mL) was added at room temperature 2-(2-methoxyphenyl)-2-morpholinoethanamine (50a) (OTAVA 7020410260, 0.25 g, 1.058 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-(2-(2-methoxyphenyl)-2-morpholinoethylamino)pyrrolo[1,2-b]pyridazine-3-carbonitrile (50b) as a white semisolid, which was crystallized from ether/hexane to furnish (0.281 g, 68.86%) white, crystalline solid; MP 156.9° C.; ¹H NMR (300 MHz, DMSO) δ 7.92 (s, 1H), 7.72 (s, 1H), 7.57-7.45 (m, 1H), 7.29 (dd, J=7.5, 16.6, 2H), 6.98 (d, J=7.6, 3H), 6.71-6.66 (m, 1H), 4.44 (d, J=5.6, 2H), 4.39-4.30 (m, 1H), 3.84 (s, 1H), 3.65 (s, 3H), 3.52 (s, 5H), 2.75-2.33 (s, 2H), MS (ES+) 378.0 (M+1); (ES−) 376.1 (M−1); Analysis: Calcd for C₂₁H₂₃N₅O₂: C, 66.83; H, 6.14; N, 18.55. Found: C, 67.08; H, 6.29; N, 18.39.

### Example 28

### 4-(2-(2-methoxyphenyl)-2-morpholinoethylamino)pyrrolo[1,2-b]pyridazine-3-carboxamide (50c)

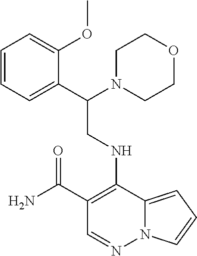

To a solution of 4-(2-(2-methoxyphenyl)-2-morpholinoethylamino)pyrrolo[1,2-b]pyridazine-3-carbonitrile (50b) (0.123 g, 0.3 mmol) in EtOH (15 mL) was added concentrated NH₄OH (4 mL), followed by dropwise addition of H₂O₂ (0.2 mL, 1.6 mmol) and stirred at room temperature for 14 h. The reaction mixture was concentrated to dryness in vacuum. The residue obtained was purified by flash column chromatography (silica gel 4 g, eluting with 0-100% ethyl acetate in hexanes) to furnish 4-(2-(2-methoxyphenyl)-2-morpholinoethylamino)pyrrolo[1,2-b]pyridazine-3-carboxamide (50c), which was crystallized from ether/hexane to furnish (0.033 g, 27.8%) as an olive colored solid. ¹H NMR (300 MHz, DMSO) δ 10.83 (s, 1H), 8.15 (s, 1H), 7.64 (dd, J=1.5, 2.6, 1H), 7.51 (d, J=6.1, 1H), 7.46-7.07 (bs, 2H), 7.27 (t, J=7.0, 1H), 7.03 (d, J=7.6, 1H), 6.94 (dd, J=6.0, 13.3, 2H), 6.61 (dd, J=2.7, 4.5, 1H), 4.25 (m, 1H), 4.11 (m, 1H), 4.03 (m, 1H), 3.78 (s, 3H), 3.60 (m, 4H), 2.44 (m, 2H), 2.36 (m, 2H); MS (ES−) 393.5 (M−1); 430.0 (M+Cl); Analysis: Calcd for C₂₁H₂₅N₅O₃: C, 63.78; H, 6.37; N, 17.71. Found: C, 63.50; H, 6.39; N, 17.51.

### Example 29

### 4-(2-(3,4-dimethoxyphenyl)propan-2-ylamino)pyrrolo[1,2-b]pyridazine-3-carbonitrile (50e)

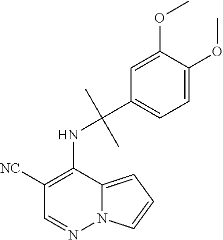

To a solution of 4-chloropyrrolo[1,2-b]pyridazine-3-carbonitrile (15g) (0.177 g, 1.0 mmol) in DMF (2.5 mL) was added at room temperature to 2-(3,4-dimethoxyphenyl)propan-2-amine (50d) (0.25 g, 1.08 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-(2-(3,4-dimethoxyphenyl)propan-2-ylamino)pyrrolo[1,2-b]pyridazine-3-carbonitrile (50e) which was crystallized from ether/hexane to furnish (0.160 g, 47.7%) as a reddish brown solid; ¹HNMR (300 MHz, DMSO) δ 7.81 (s, 1H), 7.71 (dd, J=1.6, 2.6, 1H), 7.27 (s, 1H), 6.97-6.93 (m, 2H), 6.89-6.83 (m, 2H), 6.67 (dd, J=2.7, 4.5, 1H), 3.70 (d, J=14.5, 6H), 1.82 (s, 6H). MS (ES−) 371.3 (M+Cl).

### Example 30

### 4-(2-(3,4-dimethoxyphenyl)propan-2-ylamino)pyrrolo[1,2-b]pyridazine-3-carboxamide (50f)

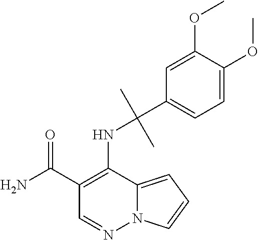

To a solution of 4-(2-(3,4-dimethoxyphenyl)propan-2-ylamino)pyrrolo[1,2-b]pyridazine-3-carbonitrile (50e) (118 mg, 0.352 mmol) in ethanol (15 mL) was added at room temperature ammonium hydroxide (4 mL), hydrogen peroxide (0.2 mL) and stirred at room temperature overnight. The reaction was concentrated in vacuum, and the residue obtained was purified by flash column chromatography (silica gel 12 g, eluting with 0-100% (9:1) ethyl acetate/methanol in hexanes) to furnish 4-(2-(3,4-dimethoxyphenyl)propan-2-ylamino)pyrrolo[1,2-b]pyridazine-3-carboxamide (501) as a dark green semisolid, which was crystallized from ether/hexane to furnish (0.016 g, 13.5%) as a greenish brown solid; ¹HNMR (300 MHz, DMSO) δ 8.57-8.08 (bs, 1H), 8.03 (s, 1H), 7.62-7.18 (m, 1H), 6.96 (d, J=4.4, 2H), 6.75 (d, J=8.5, 1H), 6.63 (dd, J=3.3, 12.6, 2H), 6.50 (dd, J=2.2, 8.4, 1H), 3.67 (s, 3H), 3.60 (s, 3H), 1.75 (s, 6H). MS (ES+) 355.0 (M+1), (ES−) 389.1 (M+Cl).

### Example 31

### 4-((4-isobutylmorpholin-2-yl)methylamino)pyrrolo[1,2-b]pyridazine-3-carbonitrile (50h)

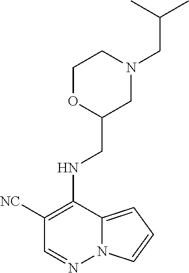

To a solution of 4-chloropyrrolo[1,2-b]pyridazine-3-carbonitrile (15g) (0.177 g, 1.0 mmol) in DMF (2.5 mL) was added at room temperature to (4-isobutylmorpholin-2-yl)methanamine (50g) (Ottava 1044939, 0.25 g, 1.02 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-((4-isobutylmorpholin-2-yl)methylamino)pyrrolo[1,2-b]pyridazine-3-carbonitrile (50h) (0.248 g, 79%) as a white solid; ¹H NMR (300 MHz, DMSO) δ 8.16 (s, 1H), 7.91 (s, 1H), 7.71 (dd, J=1.6, 2.6, 1H), 7.11 (dd, J=1.6, 4.5, 1H), 6.68 (dd, J=2.7, 4.4, 1H), 3.82 (m, 3H), 3.64 (m, 1H), 3.50 (t, J=10.0, 1H), 2.85 (d, J=11.1, 1H), 2.63 (d, J=10.6, 1H), 2.03 (m, 3H), 1.78 (m, 2H), 0.86 (s, 3H), 0.84 (s, 3H); IR (KBr) 2200 cm⁻¹; MS (ES+) 314.1 (M+1) (ES−) 312.0 (M−1); Analysis. Calcd for C₁₇H₂₃N₅O: C, 65.15; H, 7.40; N, 22.35. Found: C, 65.46, H, 7.61; N, 22.60.

### Example 32

### 2-((3-carbamoylpyrrolo[1,2-b]pyridazin-4-ylamino)methyl)-4-isobutylmorpholine 4-oxide (50i)

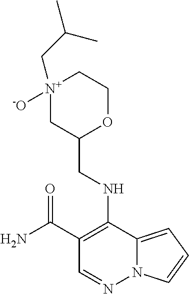

To a solution of 4-((4-isobutylmorpholin-2-yl)methylamino)pyrrolo[1,2-b]pyridazine-3-carbonitrile (50h) (0.130 g, 0.4 mmol) in EtOH (15 mL) was added concentrated NH₄OH (4 mL), followed by dropwise addition of H₂O₂ (0.2 mL, 1.6 mmol) and stirred at room temperature for 14 h. The reaction mixture was concentrated to dryness in vacuum. The residue obtained was purified by flash column chromatography (silica gel 4 g, eluting with 0-100% ethyl acetate in hexanes) to furnish a white semisolid, which was crystallized from ether/hexane to furnish 2-((3-carbamoylpyrrolo[1,2-b]pyridazin-4-ylamino)methyl)-4-isobutylmorpholine 4-oxide (50i) (0.052 g, 0.15 mmol, 37.4%) as a blue solid; ¹H NMR (300 MHz, DMSO) δ 10.72 (s, 1H), 8.20 (s, 1H), 7.69 (dd, J=1.5, 2.6, 1H), 6.97 (d, J=3.1, 1H), 6.66 (dd, J=2.7, 4.5, 1H), 4.47 (m, 1H), 4.24 (d, J=9.9, 1H), 3.88 (m, 1H), 3.84-3.65 (m, 2H), 3.30 (m, 1H), 3.07 (dd, J=7.2, 26.6, 4H), 2.85 (d, J=11.6, 1H), 2.38 (s, 1H), 1.04 (d, J=1.7, 3H), 1.02 (d, J=1.7, 3H); MS 370.1 (M+Na), 695.2 (2M+1), 717.1 (2M+Na), (ES−) 346.2 (M−1); Analysis Calcd for: C₁₇H₂₅N₅O₃.0.5H₂O: C, 57.29; H, 7.35; N, 19.65. Found: C, 57.58; H, 7.72; N, 19.58.

### Example 33

### 4-((1-methyl-1H-imidazol-2-yl)(m-tolyl)methylamino)pyrrolo[1,2-b]pyridazine-3-carbonitrile (50k)

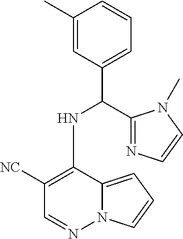

To a solution of 4-chloropyrrolo[1,2-b]pyridazine-3-carbonitrile (15g) (0.177 g, 1.0 mmol) in DMF (2.5 mL) was added at room temperature (1-methyl-1H-imidazol-2-yl)(m-tolyl)methanamine (50j) (Ottava 1156352, 0.25 g, 0.91 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-((1-methyl-1H-imidazol-2-yl)(m-tolyl)methylamino)pyrrolo[1,2-b]pyridazine-3-carbonitrile (50k) (0.243 g, 71%) as a off white solid; ¹H NMR (300 MHz, DMSO) δ 8.32 (d, J=7.7, 1H), 7.93 (s, 1H), 7.75 (dd, J=1.6, 2.6, 1H), 7.37 (dd, J=1.5, 4.5, 1H), 7.27 (t, J=7.5, 1H), 7.21-7.09 (m, 4H), 6.86 (d, J=1.1, 1H), 6.76 (s, 2H), 3.51 (s, 3H), 2.28 (s, 3H); IR (KBr) 2197 cm⁻¹; MS (ES−) 342.4 (M−1); Analysis: Calcd for C₂₀H₁₈N₆: C, 69.25; H, 5.38; N, 24.23. Found: C, 69.64; H, 5.37; N, 24.27.

### Example 34

### 4-((1-methyl-1H-imidazol-2-yl)(m-tolyl)methylamino)pyrrolo[1,2-b]pyridazine-3-carboxamide (50m)

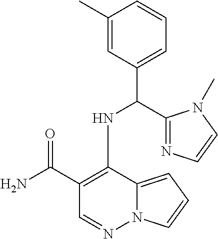

To a solution of 4-((1-methyl-1H-imidazol-2-yl)(m-tolyl)methylamino)pyrrolo[1,2-b]pyridazine-3-carbonitrile (50k) (0.136 g, 0.4 mmol) in EtOH (15 mL) was added concentrated NH₄OH (4 mL), followed by dropwise addition of H₂O₂ (0.2 mL, 1.6 mmol) and stirred at room temperature for 14 h. A combination of hexane and ether were used to induce crystallization and the product was filtered, washed with EtOH and ether, and dried to furnish 4-((1-methyl-1H-imidazol-2-yl)(m-tolyl)methylamino)pyrrolo[1,2-b]pyridazine-3-carboxamide (50m) as a blue solid (0.085 g, 58.96%); ¹H NMR (300 MHz, DMSO) δ 11.44 (d, J=8.0, 1H), 8.25 (s, 1H), 7.65 (dd, J=1.5, 2.6, 1H), 7.27-7.19 (m, 3H), 7.09 (d, J=1.1, 2H), 6.92 (dd, J=1.4, 4.7, 1H), 6.80 (d, J=1.1, 1H), 6.62 (dd, J=3.4, 7.8, 2H), 3.63 (s, 3H), 2.27 (s, 3H); MS (ES+) 361.1 (M+1), 721.1 (2M+1); 742.1 (2M+Na), (ES−) 358.6 (M−1); Analysis; Calcd for: C₂₀H₂₀N₆O.0.25H₂O: C, 65.83; H, 5.66; N, 23.03. Found C, 65.94; H, 5.63; N, 23.00.

### Example 35

### 4-(2-(2-chlorophenyl)-2-(4-methylpiperazin-1-yl)ethylamino)pyrrolo[1,2-b]pyridazine-3-carbonitrile (51b)

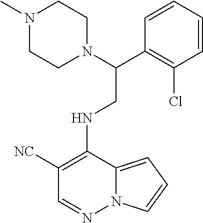

To a solution of 4-chloropyrrolo[1,2-b]pyridazine-3-carbonitrile (15g) (0.177 g, 1.0 mmol) in DMF (2.5 mL) was added at room temperature 2-(2-chlorophenyl)-2-(4-methylpiperazin-1-yl)ethanamine (51a) (Ottava 7020410288, 0.25 g, 1.0 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-(2-(2-chlorophenyl)-2-(4-methylpiperazin-1-yl)ethylamino)pyrrolo[1,2-b]pyridazine-3-carbonitrile (51b) (0.366 g, 93%) as a off white solid; ¹H NMR (300 MHz, DMSO) δ 7.92 (s, 1H), 7.72 (s, 1H), 7.61-7.55 (m, 1H), 7.52 (d, J=7.7, 1H), 7.36 (m, 3H), 6.95 (s, 1H), 6.71-6.65 (m, 1H), 4.56 (m, 1H), 4.37 (m, 1H), 3.94 (m, 1H), 3.35 (m, 4H), 3.33-3.32 (m, 4H), 2.27 (s, 3H); MS (ES+) 395.0 (M+1), (ES−) 392.8 (M−1); IR (KBr) 2206 cm⁻¹.

### Example 36

### 4-(2-(3-carbamoylpyrrolo[1,2-b]pyridazin-4-ylamino)-1-(2-chlorophenyl)ethyl)-1-methylpiperazine 1-oxide (51c)

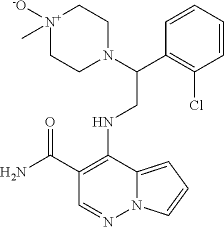

To a solution of 4-(2-(2-chlorophenyl)-2-(4-methylpiperazin-1-yl)ethylamino)pyrrolo[1,2-b]pyridazine-3-carbonitrile (51b) (0.167 g, 0.4 mmol) in EtOH (15 mL) was added concentrated NH₄OH (4 mL), followed by dropwise addition of H₂O₂ (0.2 mL, 1.6 mmol) and stirred at room temperature for 14 h. The reaction mixture was concentrated to dryness in vacuum. The residue obtained was purified by flash column chromatography (silica gel 4 g, eluting with 0-100% ethyl acetate in hexanes) to furnish a blue semisolid, which was crystallized from ether/hexane to furnish 4-(2-(3-carbamoylpyrrolo[1,2-b]pyridazin-4-ylamino)-1-(2-chlorophenyl)ethyl)-1-methylpiperazine 1-oxide (51c) (0.022 g, 13.3%) as a blue solid; ¹H NMR (300 MHz, DMSO) δ 10.80 (s, 1H), 8.17 (s, 1H), 7.68 (m, 2H), 7.50 (d, J=9.3, 1H), 7.36 (m, 2H), 6.95 (m, 1H), 6.63 (m, 1H), 4.45 (m, 1H), 4.30-4.03 (m, 2H), 3.31-3.27 (m, 2H), 3.04 (s, 3H), 3.01-2.59 (m, 6H); MS (ES+) 429.02 (M+1), 857.09 (2M+1), (ES−) 427.1.

### Example 37

### 4-(cyclohexylamino)pyrrolo[1,2-b]pyridazine-3-carbonitrile (51e)

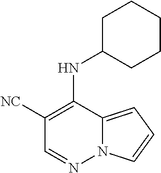

To a solution of 4-chloropyrrolo[1,2-b]pyridazine-3-carbonitrile (15g) (0.15 g, 0.84 mmol) in DMF (2.5 mL) was added at room temperature cyclohexylamine (51d) (0.2 mL, 1.68 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-(cyclohexylamino)pyrrolo[1,2-b]pyridazine-3-carbonitrile (51e) (0.172 g, 85%) as a white solid; ¹HNMR (300 MHz, DMSO) δ 7.89 (s, 1H), 7.68 (m, 2H), 7.17 (dd, J=1.6, 4.5, 1H), 6.67 (dd, J=2.7, 4.3, 1H), 4.20 (m, 1H), 2.01 (m, 2H), 1.79 (m, 2H), 1.64 (m, 1H), 1.52-1.30 (m, 4H), 1.17 (m, 1H); IR (KBr) 2190 cm⁻¹; MS (ES+) 241.1 (M+1), (ES−) 239.0 (M−1); Analysis: Calculated for C₁₄H₁₆N₄: C, 56.73; H, 7.14; N, 19.46. Found: C, 56.49; H, 6.85; N, 19.18.

### Example 38

### 4-(cyclohexylamino)pyrrolo[1,2-b]pyridazine-3-carboxamide (51f)

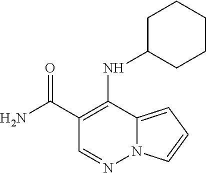

To a solution of 4-(cyclohexylamino)pyrrolo[1,2-b]pyridazine-3-carbonitrile (51e) (110 mg, 0.48 mmol) in ethanol (15 mL) was added at room temperature ammonium hydroxide (4 mL), hydrogen peroxide (0.2 mL) and stirred at room temperature overnight. The reaction was concentrated in vacuum, and the residue obtained was purified by flash column chromatography (silica gel 12 g, eluting with 0-100% (9:1) ethyl acetate/methanol in hexanes) to furnish 4-(cyclohexylamino)pyrrolo[1,2-b]pyridazine-3-carboxamide (51f) (0.070 g, 59%) as a blue solid; ¹HNMR (300 MHz, DMSO) δ 10.78 (d, J=8.0, 1H), 8.19 (s, 1H), 7.66 (s, 1H), 7.63-6.93 (bs, 2H), 6.83 (d, J=3.2, 1H), 6.72-6.62 (m, 1H), 4.07 (m, 1H), 1.99 (m, 2H), 1.68 (m, 2H), 1.62-1.52 (m, 1H), 1.51-1.23 (m, 5H); MS (ES+) 259.1 (M+1), (ES−) 257.3 (M−1); Analysis: Calcd for C₁₄H₁₈N₄O: C, 65.09; H, 7.02; N, 21.69. Found: C, 64.55; H, 7.16; N, 21.34.

### Example 39

### 4-(4-hydroxycyclohexylamino)pyrrolo[1,2-b]pyridazine-3-carbonitrile (51h)

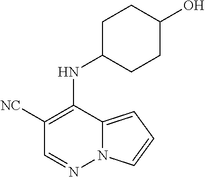

To a solution of 4-chloropyrrolo[1,2-b]pyridazine-3-carbonitrile (15g) (0.15 g, 0.84 mmol) in DMF (2.5 mL) was added at room temperature trans-4-aminocyclohexanol (51g) (194 mgs, 1.68 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-(4-hydroxycyclohexylamino)pyrrolo[1,2-b]pyridazine-3-carbonitrile (51h) (0.173 g, 80%) as a white solid; ¹HNMR (300 MHz, DMSO) δ 7.90 (s, 1H), 7.68 (dd, J=1.6, 2.6, 1H), 7.63 (d, 1H), 7.15 (dd, J=1.6, 4.5, 1H), 6.66 (dd, J=2.7, 4.4, 1H), 4.63 (d, J=4.8, 1H), 4.18 (m, 1H), 3.42 (m, 1H), 1.97 (m, 2H), 1.88 (m, 2H), 1.52 (m, 2H), 1.28 (m, 2H); IR (KBr) 2199 cm⁻¹; MS (ES+) 257.1 (M+1), 279.1 (M+Na), MS (ES−) 255.4 (M−1); Analysis: Calcd for C₁₄H₁₆N₄O: C, 65.61; H, 6.29; N, 21.86. Found: C, 65.60; H, 6.49; N, 21.84.

### Example 40

### 4-(4-hydroxycyclohexylamino)pyrrolo[1,2-b]pyridazine-3-carboxamide (51i)

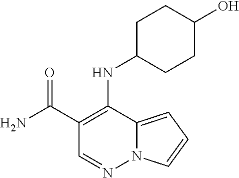

To a solution of 4-(4-hydroxycyclohexylamino)pyrrolo[1,2-b]pyridazine-3-carbonitrile (51h) (110 mg, 0.48 mmol) in ethanol (15 mL) was added at room temperature ammonium hydroxide (4 mL), hydrogen peroxide (0.2 mL) and stirred at room temperature overnight. The reaction was concentrated in vacuum, and the residue obtained was purified by flash column chromatography (silica gel 12 g, eluting with 0-100% (9:1) ethyl acetate/methanol in hexanes) to furnish 4-(4-hydroxycyclohexylamino)pyrrolo[1,2-b]pyridazine-3-carboxamide (51i) (0.092 g, 78%) as a blue solid; MP 192.2° C.; ¹HNMR (300 MHz, DMSO) δ 10.71 (d, J=8.2, 1H), 8.19 (s, 1H), 7.66 (s, 1H), 7.62-6.92 (m, 2H), 6.84 (s, 1H), 6.68 (d, J=2.6, 1H), 4.63 (d, J=4.0, 1H), 4.02 (m, 1H), 3.51 (m, 1H), 2.08 (m, 2H), 1.83 (m, 2H), 1.40 (m, 4H); MS (ES+) 275.1 (M+1), MS (ES−) 272.7 (M−1); Analysis: Calcd for C₁₄H₁₈N₄O₂.0.75H₂O: C, 58.42; H, 6.83; N, 19.47. Found: C, 58.72; H, 6.96; N, 19.28.

### Example 41

### 4-((tetrahydrofuran-2-yl)methylamino)pyrrolo[1,2-b]pyridazine-3-carbonitrile (51k)

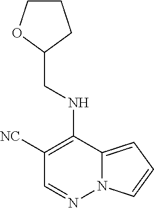

To a solution of 4-chloropyrrolo[1,2-b]pyridazine-3-carbonitrile (15g) (0.15 g, 0.84 mmol) in DMF (2 mL) was added at room temperature (tetrahydrofuran-2-yl)methanamine (51j) (Aldrich™, 0.26 mL, 2.52 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-((tetrahydrofuran-2-yl)methylamino)pyrrolo[1,2-b]pyridazine-3-carbonitrile (51k) as a white semisolid, which was crystallized from ether/hexane to furnish (0.183 g, 90%) tan solid; MP 101.8° C.; ¹HNMR (300 MHz, DMSO) δ 8.15 (s, 1H), 7.90 (s, 1H), 7.70 (dd, J=1.6, 2.6, 1H), 7.11 (dd, J=1.6, 4.5, 1H), 6.68 (dd, J=2.7, 4.4, 1H), 4.22-4.09 (m, 1H), 3.88-3.62 (m, 4H), 2.09-1.95 (m, 1H), 1.94-1.77 (m, 2H), 1.61 (m 1H); IR (KBr) 2195 cm⁻¹; MS (ES+) 265.1 (M+Na); (ES−) 241.0 (M−1); Analysis: Calcd for C₁₃H₁₄N₄O: C, 64.45; H, 5.82; N, 23.13. Found: C, 64.64; H, 5.87; N, 23.05.

### Example 42

### 4-((tetrahydrofuran-2-yl)methylamino)pyrrolo[1,2-b]pyridazine-3-carboxamide (51m)

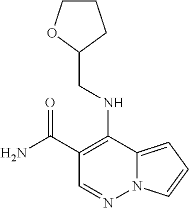

To a solution of 4-((tetrahydrofuran-2-yl)methylamino)pyrrolo[1,2-b]pyridazine-3-carbonitrile (51k) (126 mg, 0.52 mmol) in ethanol (15 mL) was added at room temperature ammonium hydroxide (4 mL), hydrogen peroxide (0.2 mL) and stirred at room temperature overnight. The reaction was concentrated in vacuum, and the residue obtained was purified by flash column chromatography (silica gel 12 g, eluting with 0-100% (9:1) ethyl acetate/methanol in hexanes) to furnish 4-((tetrahydrofuran-2-yl)methylamino)pyrrolo[1,2-b]pyridazine-3-carboxamide (51m) (0.073 g, 54%) as a light green solid; MP 120° C.; ¹HNMR (300 MHz, DMSO) δ 10.65 (s, 1H), 8.19 (s, 1H), 7.67 (dd, J=1.5, 2.6, 1H), 7.61-7.04 (bs, 2H), 6.98 (dd, J=1.6, 4.6, 1H), 6.64 (dd, J=2.7, 4.5, 1H), 4.10 (m, 1H), 3.86 (m, 2H), 3.79-3.65 (m, 2H), 2.09-1.79 (m, 3H), 1.75-1.61 (m, 1H); MS (ES+) 543.1 (M+Na); (ES−) 259.3 (M−1); Analysis: Calcd for C₁₃H₁₆N₄O₂.0.5H₂O: C, 57.98; H, 6.36; N, 20.81. Found: C, 57.99; H, 6.36; N, 20.75.

### Example 43

### 4-(cyclopentylamino)pyrrolo[1,2-b]pyridazine-3-carbonitrile (52b)

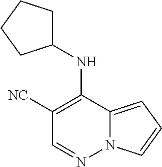

To a solution of 4-chloropyrrolo[1,2-b]pyridazine-3-carbonitrile (15g) (0.15 g, 0.84 mmol) in DMF (2 mL) was added at room temperature cyclopentylamine (0.25 mL, 2.52 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-(cyclopentylamino)pyrrolo[1,2-b]pyridazine-3-carbonitrile (52b) as a white semisolid, which was crystallized from ether/hexane to furnish (0.164 g, 86.3%) white solid; MP 102.9° C.; ¹HNMR (300 MHz, DMSO) δ 7.91 (s, 1H), 7.68 (dd, J=1.7, 2.7, 2H), 7.20 (dd, J=1.6, 4.5, 1H), 6.67 (dd, J=2.7, 4.3, 1H), 4.64 (m, 1H), 2.05 (m, 2H), 1.76 (m, 4H), 1.59 (m, 2H); IR (KBr) 2198 cm⁻¹; MS (ES−) 225.0 (M−1); Analysis: Calcd for C₁₃H₁₄N₄: C, 69.00; H, 6.24; N, 24.76. Found: C, 69.00; H, 6.26; N, 24.70.

### Example 44

### 4-(cyclopentylamino)pyrrolo[1,2-b]pyridazine-3-carboxamide (52c)

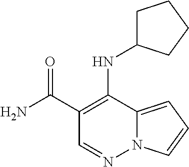

To a solution of 4-(cyclopentylamino)pyrrolo[1,2-b]pyridazine-3-carbonitrile (52b) (0.106 g, 0.468 mmol) in ethanol (15 mL) was added at room temperature ammonium hydroxide (4 mL), hydrogen peroxide (0.2 mL) and stirred at room temperature overnight. The reaction was concentrated in vacuum, and the residue obtained was purified by flash column chromatography (silica gel 12 g, eluting with 0-100% ethyl acetate in hexanes) to furnish 4-(cyclopentylamino)pyrrolo[1,2-b]pyridazine-3-carboxamide (52c) (0.51 g, 44.9%) as a light blue solid; ¹HNMR (300 MHz, DMSO) δ 10.78 (d, J=7.5, 1H), 8.19 (s, 1H), 7.66 (dd, J=1.6, 2.6, 1H), 7.60-7.05 (bs, 2H), 6.95 (dd, J=1.5, 4.6, 1H), 6.66 (dd, J=2.7, 4.5, 1H), 4.57 (m, 1H), 2.06 (m, 2H), 1.78-1.52 (m, 6H); MS (ES+) 245.2 (M+1); (ES−) 243.0 (M−1); Analysis: Calcd for C₁₃H₁₆N₄O.0.25H₂O: C, 62.76; H, 6.68; N, 22.52. Found: C, 62.83, H, 6.49; N, 22.44.

### Example 45

### 4-(phenylamino)pyrrolo[1,2-b]pyridazine-3-carbonitrile (52e)

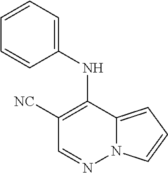

To a solution of 4-chloropyrrolo[1,2-b]pyridazine-3-carbonitrile (15g) (0.15 g, 0.84 mmol) in DMF (2 mL) was added at room temperature aniline (52d) (0.25 mL, 2.52 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-(phenylamino)pyrrolo[1,2-b]pyridazine-3-carbonitrile (52e) as a yellow semisolid, which was crystallized from ether/hexane to furnish (0.157 g, 79.8%) light yellow solid; MP 163.5° C.; ¹HNMR (300 MHz, DMSO) δ 9.90 (s, 1H), 7.99 (s, 1H), 7.81 (dd, J=1.7, 2.6, 1H), 7.50-7.40 (m, 2H), 7.39-7.30 (m, 3H), 6.77 (dd, J=1.6, 4.5, 1H), 6.72 (dd, J=2.7, 4.4, 1H); IR (KBr) 2202 cm⁻¹; MS (ES+) 235.1 (M+1); 233.0 (M−1); Analysis: Calcd for C₁₄H₁₀N₄: C, 71.78; H, 4.30; N, 23.92. Found: C, 71.84; H, 4.26; N, 23.94.

### Example 46

### 4-(phenylamino)pyrrolo[1,2-b]pyridazine-3-carboxamide (52f)

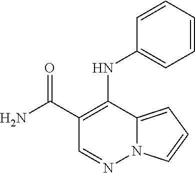

To a solution of 4-(phenylamino)pyrrolo[1,2-b]pyridazine-3-carbonitrile (52e) (0.113 g, 0.482 mmol) in ethanol (15 mL) was added at room temperature ammonium hydroxide (4 mL), hydrogen peroxide (0.2 mL) and stirred at room temperature overnight. The reaction was concentrated in vacuum, and the residue obtained was purified by flash column chromatography (silica gel 12 g, eluting with 0-100% ethyl acetate in hexanes) to furnish 4-(phenylamino)pyrrolo[1,2-b]pyridazine-3-carboxamide (52f) as a light brown solid (0.54 g, 44.4%); MP 247.2° C. ¹HNMR (300 MHz, DMSO) δ 11.98 (s, 1H), 8.39 (s, 1H), 7.96 (s, 1H), 7.66 (dd, J=1.6, 2.6, 1H), 7.49-7.29 (m, 6H), 6.45 (dd, J=2.7, 4.5, 1H), 5.39 (dd, J=1.6, 4.5, 1H); MS (ES+) 253.1 (M+1); (ES−) 251.4 (M−1).

### Example 47

### 4-(cycloheptylamino)pyrrolo[1,2-b]pyridazine-3-carbonitrile (52h)

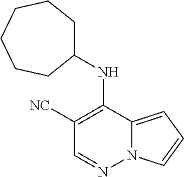

To a solution of 4-chloropyrrolo[1,2-b]pyridazine-3-carbonitrile (15g) (0.15 g, 0.84 mmol) in DMF (2 mL) was added at room temperature cycloheptylamine (0.32 mL, 2.52 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-(cycloheptylamino)pyrrolo[1,2-b]pyridazine-3-carbonitrile (52h) as a white semisolid, which was crystallized from ether/hexane to furnish (0.190 g, 88.9%) white solid; MP 108.0° C.; ¹HNMR (300 MHz, DMSO) d 7.89 (s, 1H), 7.67 (m, 2H), 7.18 (s, 1H), 6.66 (s, 1H), 4.41 (m, 1H), 1.99 (m, 2H), 1.71 (m, 4H), 1.56 (m, 6H); IR (KBr) 2201 cm⁻¹; MS (ES+) 255.2, (ES−) 253.0 (M−1); Analysis: Calcd for C₁₅H₁₈N₄: C, 70.84; H, 7.13; N, 22.03. Found: C, 70.83; H, 7.18; N, 21.94

### Example 48

### 4-(cycloheptylamino)pyrrolo[1,2-b]pyridazine-3-carboxamide (52i)

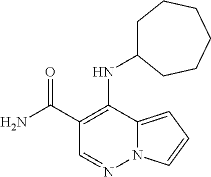

To a solution of 4-(cycloheptylamino)pyrrolo[1,2-b]pyridazine-3-carbonitrile (52h) (0.113 g, 0.444 mmol) in ethanol (15 mL) was added at room temperature ammonium hydroxide (4 mL), hydrogen peroxide (0.2 mL) and stirred at room temperature overnight. The reaction was concentrated in vacuum, and the residue obtained was purified by flash column chromatography (silica gel 12 g, eluting with 0-100% ethyl acetate in hexanes) to furnish 4-(cycloheptylamino)pyrrolo[1,2-b]pyridazine-3-carboxamide (52i) as a dark blue solid (0.066 g, 54.6%); MP 279.2° C.; ¹HNMR (300 MHz, DMSO) δ 10.80 (d, J=8.3, 1H), 8.19 (s, 1H), 7.66 (dd, J=1.5, 2.6, 1H), 7.62-6.91 (m, 2H), 6.86 (dd, J=1.5, 4.6, 1H), 6.67 (dd, J=2.7, 4.5, 1H), 4.28 (m, 1H), 2.01 (m, 2H), 1.59 (m, 10H); MS (ES+) 273.2 (M+1); 271.0 (M−1).

### Example 49

### 4-(tetrahydro-2H-pyran-4-ylamino)pyrrolo[1,2-b]pyridazine-3-carbonitrile (52k)

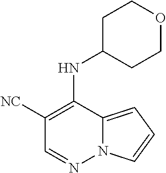

To a solution of 4-chloropyrrolo[1,2-b]pyridazine-3-carbonitrile (15g) (0.15 g, 0.84 mmol) in DMF (2 mL) was added at room temperature tetrahydro-2H-pyran-4-amine (52j) (0.25 mgs, 2.52 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-(tetrahydro-2H-pyran-4-ylamino)pyrrolo[1,2-b]pyridazine-3-carbonitrile (52k) (0.172 g, 85%) as a light yellow solid; ¹HNMR (300 MHz, DMSO) δ7.92 (s, 1H), 7.76-7.68 (m, 2H), 7.17 (dd, J=1.6, 4.5, 1H), 6.69 (dd, J=2.7, 4.4, 1H), 4.51-4.36 (m, 1H), 3.95 (dd, J=3.4, 11.4, 2H), 3.45-3.35 (m, 2H), 1.96 (d, J=10.3, 2H), 1.83-1.63 (m, 2H). IR (KBr) 2194 cm⁻¹; MS (ES−) 241.0 (M−1); 277.3 (M+Cl).

### Example 50

### 4-(tetrahydro-2H-pyran-4-ylamino)pyrrolo[1,2-b]pyridazine-3-carboxamide (52m)

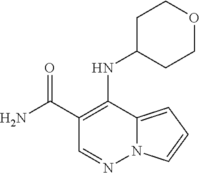

To a solution of 4-(tetrahydro-2H-pyran-4-ylamino)pyrrolo[1,2-b]pyridazine-3-carbonitrile (52k) (0.130 g, 0.54 mmol) in ethanol (15 mL) was added at room temperature ammonium hydroxide (4 mL), hydrogen peroxide (0.2 mL) and stirred at room temperature overnight. The reaction was concentrated in vacuum, and the residue obtained was purified by flash column chromatography (silica gel 12 g, eluting with 0-100% ethyl acetate in hexanes) to furnish 4-(tetrahydro-2H-pyran-4-ylamino)pyrrolo[1,2-b]pyridazine-3-carboxamide (52m) (0.085 g, 61%) as a olive colored solid. ¹H NMR (300 MHz, DMSO) d 10.83 (d, J=8.1, 1H), 8.22 (s, 1H), 7.68 (dd, J=1.5, 2.6, 1H), 6.91 (dd, J=1.5, 4.7, 1H), 6.68 (dd, J=2.7, 4.5, 1H), 4.32 (s, 1H), 3.84 (d, J=11.8, 2H), 3.57 (t, J=9.7, 2H), 2.08-1.96 (m, 2H), 1.52 (d, J=9.5, 2H); MS (ES+) 261.1 (M+1) 283.1 (M+Na), (ES−) 259.0 (M−1); Analysis: Calcd for C₁₃H₁₆N₄O₂: C, 59.99; H, 6.20; N, 21.52. Found: C, 59.99; H, 6.19; N, 21.37.

### Example 51

### 4-(tetrahydrofuran-3-ylamino)pyrrolo[1,2-b]pyridazine-3-carbonitrile (53b)

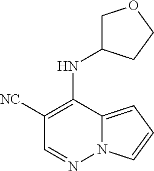

To a solution of 4-chloropyrrolo[1,2-b]pyridazine-3-carbonitrile (15g) (0.15 g, 0.84 mmol) in DMF (2 mL) was added at room temperature tetrahydrofuran-3-amine (53a) (0.22 mgs, 2.52 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-(tetrahydrofuran-3-ylamino)pyrrolo[1,2-b]pyridazine-3-carbonitrile (53b) (0.175 g, 91%) as a tan solid; ¹H NMR (300 MHz, DMSO) d 7.95 (s, 1H), 7.89 (d, J=7.0, 1H), 7.71 (dd, J=1.6, 2.6, 1H), 7.24 (dd, J=1.6, 4.5, 1H), 6.69 (dd, J=2.7, 4.4, 1H), 4.86 (dt, J=3.6, 11.1, 1H), 4.01-3.83 (m, 3H), 3.76 (td, J=5.8, 8.3, 1H), 2.39-2.23 (m, 1H), 2.15 (m, 1H); IR (KBr) 2194 cm⁻¹; MS (ES−) 227.0 (M−1) 262.9 (M+Cl); Analysis: Calcd for C₁₂H₁₂N₄O.0.25H₂O: C, 61.92; H, 5.41; N, 24.07. Found: C, 62.05; H, 5.23; N, 24.01.

### Example 52

### 4-(tetrahydrofuran-3-ylamino)pyrrolo[1,2-b]pyridazine-3-carboxamide (53c)

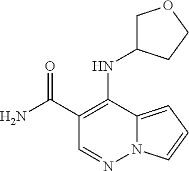

To a solution of 4-(tetrahydrofuran-3-ylamino)pyrrolo[1,2-b]pyridazine-3-carbonitrile (53b) (0.125 g, 0.55 mmol) in ethanol (15 mL) was added at room temperature ammonium hydroxide (4 mL), hydrogen peroxide (0.2 mL) and stirred at room temperature overnight. The reaction was concentrated in vacuum, and the residue obtained was purified by flash column chromatography (silica gel 12 g, eluting with 0-100% ethyl acetate in hexanes) to furnish 4-(tetrahydrofuran-3-ylamino)pyrrolo[1,2-b]pyridazine-3-carboxamide (53c) (0.068 g, 50%) as a light yellow colored solid; ¹H NMR (300 MHz, DMSO) d 10.88 (d, J=7.3, 1H), 8.22 (s, 1H), 7.70 (dd, J=1.5, 2.6, 1H), 6.94 (dd, J=1.5, 4.6, 1H), 6.68 (dd, J=2.7, 4.5, 1H), 4.85 (m, 1H), 3.96-3.85 (m, 2H), 3.79 (m, 1H), 3.70 (d, J=9.3, 1H), 2.38 (m, 1H), 1.95-1.82 (m, 1H); MS (ES−) 244.7 (M−1); 281.5 (M+Cl); Analysis: Calcd for C₁₂H₁₄N₄O₂: C, 58.53; H, 5.73; N, 22.75. Found: C, 58.22; H, 5.73; N, 22.47.

### Example 53

### 4-(tetrahydro-2H-pyran-3-ylamino)pyrrolo[1,2-b]pyridazine-3-carbonitrile

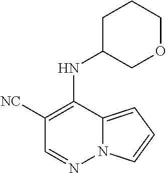

To a solution of 4-chloropyrrolo[1,2-b]pyridazine-3-carbonitrile (15g) (0.15 g, 0.84 mmol) in DMF (2 mL) was added at room temperature tetrahydro-2H-pyran-3-amine hydrochloride (53d) (0.25 mgs, 1.82 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-(tetrahydro-2H-pyran-3-ylamino)pyrrolo[1,2-b]pyridazine-3-carbonitrile (53e) (0.223 g, 92%) as a light yellow solid; ¹H NMR (300 MHz, DMSO) d 7.93 (s, 1H), 7.72 (dd, J=1.6, 2.7, 1H), 7.54 (d, J=8.0, 1H), 7.16 (dd, J=1.6, 4.5, 1H), 6.70 (dd, J=2.7, 4.4, 1H), 4.37 (m, 1H), 3.99 (d, J=10.8, 1H), 3.81 (d, J=11.2, 1H), 3.37 (m, 1H), 3.30 (m, 1H), 2.12 (m, 1H), 1.69 (m, 3H); IR 2194 cm⁻¹; MS (ES+) 243.1 (M+1); (ES−) 241.0 (M−1); Analysis: Calcd for C₁₃H₁₄N₄O: C, 64.45; H, 5.82; N, 23.13. Found: C, 64.36; H, 5.95; N, 23.20

### Example 54

### 4-(tetrahydro-2H-pyran-3-ylamino)pyrrolo[1,2-b]pyridazine-3-carboxamide

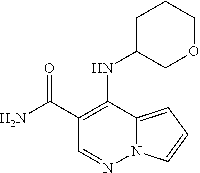

To a solution of 4-(tetrahydro-2H-pyran-3-ylamino)pyrrolo[1,2-b]pyridazine-3-carbonitrile (53e) (0.162 g, 0.67 mmol) in ethanol (15 mL) was added at room temperature ammonium hydroxide (4 mL), hydrogen peroxide (0.2 mL) and stirred at room temperature overnight. The reaction was concentrated in vacuum, and the residue obtained was purified by flash column chromatography (silica gel 12 g, eluting with 0-100% ethyl acetate in hexanes) to furnish 4-(tetrahydro-2H-pyran-3-ylamino)pyrrolo[1,2-b]pyridazine-3-carboxamide (530 (0.041 g, 24%) as a blue colored solid; ¹H NMR (300 MHz, DMSO) δ 10.89 (d, J=8.5, 1H), 8.21 (s, 1H), 7.68 (dd, J=1.5, 2.6, 1H), 6.85 (d, J=3.2, 1H), 6.68 (dd, J=2.7, 4.5, 1H), 4.24 (m, 1H), 3.85 (d, J=11.3, 1H), 3.60 (m, 2H), 3.55-3.42 (m, 1H), 2.03 (m, 1H), 1.73 (m, 2H), 1.65-1.50 (m, 1H); MS (ES+) 261.1 (M+1); 283.1 (M+Na); 543.0 (2M+Na), (ES−) 258.9 (M−1).

### Example 55

### 4-(cyclopentylamino)-6-nitropyrrolo[1,2-b]pyridazine-3-carbonitrile (54a)

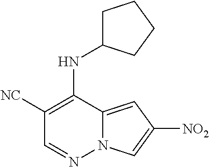

To a solution of 4-chloro-6-nitropyrrolo[1,2-b]pyridazine-3-carbonitrile (47d) (0.111 g, 0.5 mmol) in DMF (2 mL) was added at room temperature cyclopentanamine (52a) (0.12 mL, 0.6 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-(cyclopentylamino)-6-nitropyrrolo[1,2-b]pyridazine-3-carbonitrile (54a) (0.106 g, 78%) as a yellow solid. ¹HNMR (300 MHz, DMSO) δ 8.67 (s, 1H), 8.21 (s, 1H), 8.18 (s, 1H), 8.00 (s, 1′-1), 4.65 (m, 1H), 2.04 (m, 2H), 1.77 (m, 4H), 1.61 (m, 2H); IR (KBr) 2211 cm⁻¹; MS (ES−) 269.9 (M−1); Analysis: Calcd for C₁₃H₁₃N₅O₂: C, 57.56; H, 4.83; N, 25.82. Found: C, 57.77; H, 4.97; N, 25.52.

### Example 56

### 4-(cyclopentylamino)-6-nitropyrrolo[1,2-b]pyridazine-3-carboxamide (54b)

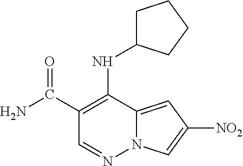

To a solution of 4-(cyclopentylamino)-6-nitropyrrolo[1,2-b]pyridazine-3-carbonitrile (54a) (85 mg, 0.31 mmol) in ethanol (15 mL) was added at room temperature ammonium hydroxide (4 mL), hydrogen peroxide (0.2 mL) and stirred at room temperature overnight. The reaction was concentrated in vacuum, and the residue obtained was purified by flash column chromatography (silica gel 12 g, eluting with 0-100% (9:1) ethyl acetate/methanol in hexanes) to furnish 4-(cyclopentylamino)-6-nitropyrrolo[1,2-b]pyridazine-3-carboxamide (54b) (0.062 g, 69%) as a yellow solid; ¹HNMR (300 MHz, DMSO) δ 11.17-11.06 (m, 1H), 10.45-10.05 (bs, 2H), 8.63 (d, J=1.9, 1H), 8.40 (s, 1H), 7.52 (d, J=2.0, 1H), 4.71-4.56 (m, 1H), 2.14-2.01 (m, 2H), 1.70 (s, 4H), 1.67-1.61 (m, 1H), 1.61-1.56 (m, 1H); MS (ES+) 290.1 (M+1), (ES−) 288.3 (M−1).

### Example 57

### 6-amino-4-(cyclopentylamino)pyrrolo[1,2-b]pyridazine-3-carboxamide (54c)

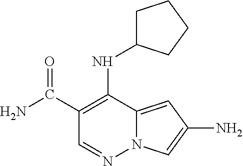

To a solution of 4-(cyclopentylamino)-6-nitropyrrolo[1,2-b]pyridazine-3-carboxamide (54b) (0.088 g, 0.3 mmol) in ethanol (20 mL) and ethyl acetate (20 mL) was added 10 wt % Pd/C (50 mg) and hydrogenated at 60 psi for 5 h. The reaction mixture was filtered through Celite, and the filtrate was concentrated in vacuum. The residue obtained was purified twice by flash column chromatography (silica gel 4 g, eluting with 1% acetic acid in chloroform and methanol 0-10%) to give 6-amino-4-(cyclopentylamino)pyrrolo[1,2-b]pyridazine-3-carboxamide (54c) (0.008 g, 10%) as a yellow solid; ¹HNMR (300 MHz, DMSO) δ 10.37 (d, 1H), 8.00 (s, 1H), 7.77-7.11 (m, 2H), 7.04 (d, J=1.9, 1H), 6.30 (d, J=1.9, 1H), 4.42 (m, 3H), 2.02 (m, 2H), 1.69 (m, 4H), 1.61-1.51 (m, 2H); ¹HNMR (300 MHz, DMSO/D₂O) δ 10.28 (d, 1H), 8.00 (s, 1H), 7.09 (d, J=1.8, 1H), 6.35 (s, 1H), 4.53-4.39 (m, 1H), 2.03 (m, 2H), 1.69 (m, 4H), 1.61-1.53 (m, 2H). MS (ES+) 260.2 (M+1), MS (ES−) 258.3 (M−1).

### Example 58

### 6-nitro-4-(phenylamino)pyrrolo[1,2-b]pyridazine-3-carbonitrile (54d)

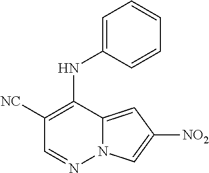

To a solution of 4-chloro-6-nitropyrrolo[1,2-b]pyridazine-3-carbonitrile (47d) (0.111 g, 0.5 mmol) in DMF (2 mL) was added at room temperature aniline (52d) (0.137 mL, 0.75 mmol), DIPEA (0.87 mL, 5 mmol) and stirred at 50° C. overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 6-nitro-4-(phenylamino)pyrrolo[1,2-b]pyridazine-3-carbonitrile (54d) (0.137 g, 98%) as a yellow solid; ¹HNMR (300 MHz, DMSO) δ 10.40 (s, 1H), 8.77 (s, 1H), 8.23 (s, 1H), 7.62-7.52 (m, 1H), 7.46 (d, J=7.1, 2H), 7.39 (s, 3H); IR (KBr) 2212 cm⁻¹; MS (ES−) 277.9 (M−1).

### Example 59

### 6-nitro-4-(phenylamino)pyrrolo[1,2-b]pyridazine-3-carboxamide (54e)

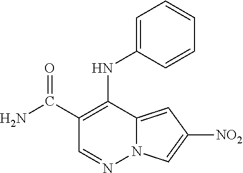

To a solution of 6-nitro-4-(phenylamino)pyrrolo[1,2-b]pyridazine-3-carbonitrile (54d) (117 mg, 0.42 mmol) in ethanol (15 mL) was added at room temperature ammonium hydroxide (4 mL), hydrogen peroxide (0.2 mL) and stirred at room temperature overnight. The reaction was concentrated in vacuum, and the residue obtained was purified by flash column chromatography (silica gel 12 g, eluting with 0-100% (9:1) ethyl acetate/methanol in hexanes) to furnish 6-nitro-4-(phenylamino)pyrrolo[1,2-b]pyridazine-3-carboxamide (54e) (0.085 g, 68%) as a dark yellow solid; ¹HNMR (300 MHz, DMSO) δ 12.22 (s, 1H), 8.63 (s, 1H), 8.59 (s, 1H), 8.23-8.05 (m, 1H), 7.67-7.57 (m, 1H), 7.51 (m, 3H), 7.42 (m, 2H), 5.79 (d, J=2.0, 1H); MS (ES−) 295.9 (M−1); Analysis: Calcd for C₁₄H₁₁N₅O₃.H₂O: C, 53.33; H, 4.16; N, 22.21. Found: C, 53.38; H, 3.78; N, 22.43.

### Example 60

### 6-amino-4-(phenylamino)pyrrolo[1,2-b]pyridazine-3-carboxamide (54f)

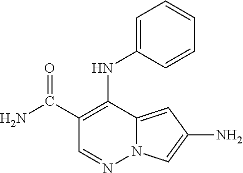

To a solution of 6-nitro-4-(phenylamino)pyrrolo[1,2-b]pyridazine-3-carboxamide (54e) (0.085 g, 0.29 mmol) in ethanol (20 mL) and ethyl acetate (20 mL) was added 10 wt % Pd/C (50 mg) and hydrogenated at 60 psi for 5 h. The reaction mixture was filtered through Celite, and the filtrate was concentrated in vacuum. The residue obtained was purified twice by flash column chromatography (silica gel 4 g, eluting with 1% acetic acid in chloroform and methanol 0-10%) to give 6-amino-4-(phenylamino)pyrrolo[1,2-b]pyridazine-3-carboxamide (54f) (0.03 g, 38%) as a yellow solid; ¹HNMR (300 MHz, DMSO) δ 11.54 (s, 1H), 8.20 (s, 1H), 8.06-7.66 (m, 1H), 7.39 (m, 2H), 7.28 (m, 1H), 7.21 (m, 2H), 7.07 (d, J=1.9, 1H), 4.43 (s, 2H); ¹HNMR (300 MHz, DMSO/D₂O) δ 8.19 (s, 1H), 7.41 (m, 3H), 7.32 (m, 1H), 7.22 (m, 2H), 7.14 (s, 1H); MS (ES+) 268.1 (M+1), MS (ES−) 266.0 (M−1).

### Example 61

### 4-(2-methylcyclohexylamino)pyrrolo[1,2-b]pyridazine-3,7-dicarboxamide (21i)

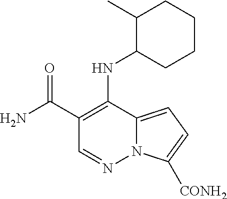

To a solution of 3-carbamoyl-4-(2-methylcyclohexylamino)pyrrolo[1,2-b]pyridazine-7-carboxylic acid (21f) (100 mg, 0.32 mmol) in DMF (3 mL) cooled with ice water was added 2-(1H-7-azabenzotriazol-1-yl)-1,1,3,3-tetramethyl uronium hexafluorophosphate methanaminium (HATU, 210 mg, 0.55 mmol), N,N-diisopropylethylamine (0.8 mL, 3.33 mmol), ammonium chloride (89 mg, 1.66 mmol) and stirred at room temperature for 16 h. The reaction mixture was diluted with EtOAc (75 mL) and washed with water (2×40 mL), brine (40 mL), dried over MgSO₄ and filtered. The filtrate was concentrated in vacuum and the residue obtained was purified by flash column chromatography [silica gel 4 g, eluting with hexanes/10% methanol in ethyl acetate, 1:0 to 1:1, (R_{f}=0.36 with hexanes/ethyl acetate/methanol=1:1:0.1)] to afford 4-(2-methylcyclohexylamino)pyrrolo[1,2-b]pyridazine-3,7-dicarboxamide (21i) (54 mg, 54%, off-white solid); ¹HNMR (300 MHz, DMSO) δ 11.08 (d, J=8.6 Hz, 1H), 8.62 (s, 1H), 8.44 (s, 1H), 7.76 (s, 1H), 7.24 (d, J=4.9 Hz, 1H), 6.99 (d, J=5.0 Hz, 1H), 4.40-4.30 (m, 1H), 1.98-1.25 (m, 9H), 0.90 (d, J=6.9 Hz, 3H); IR (KBr, cm⁻¹): 3380, 3215, 2929, 1652, 1619, 1439; MS (ES−): 314.1 (M−1).

### Example 62

### N-hydroxy-4-(2-methylcyclohexylamino)pyrrolo[1,2-b]pyridazine-3-carboximidamide (18d)

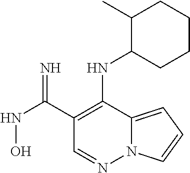

To a solution of 4-(2-methylcyclohexylamino)pyrrolo[1,2-b]pyridazine-3-carbonitrile (18b) (0.36 g, 1.41 mmol) in ethanol (30 mL) was added 50% aqueous solution of NH₂OH (2.6 mL, 42.6 mmol) and heated at reflux for 5 h. The reaction mixture was concentrated in vacuum and the residue obtained was purified by flash column chromatography (silica gel 12 g, eluting with 0-50% ethyl acetate in hexanes) to furnish N-hydroxy-4-(2-methylcyclohexylamino)pyrrolo[1,2-b]pyridazine-3-carboximidamide (18d) (0.3 g, 74%) as a off-white solid: ¹HNMR (300 MHz, DMSO) δ 9.67 (d, J=8.8, 1H), 9.61 (s, 1H), 8.03 (s, 1H), 7.60 (dd, J=1.5, 2.6, 1H), 6.75 (d, J=3.1, 1H), 6.63 (dd, J=2.7, 4.4, 1H), 5.89 (s, 2H), 4.34 (s, 1H), 1.80 (s, 2H), 1.73-1.22 (m, 7H), 0.90 (d, J=6.9, 3H); MS (ES+) 288.14 (M+1).

### Example 63

### 4-(2-methylcyclohexylamino)pyrrolo[1,2-b]pyridazine-3-carboximidamide

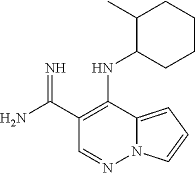

To a solution of N-hydroxy-4-(2-methylcyclohexylamino)pyrrolo[1,2-b]pyridazine-3-carboximidamide (18d) (0.2 g, 0.7 mmol) in ethanol (15 ml) was added wet Raney Nickel (10 mL) and hydrogenated at 50 psi overnight. The catalyst was removed by filtration through celite and the filtrate was concentrated in vacuum. The residue obtained was purified by flash column chromatography (silica gel 12 g, eluting with 0-100% CMA-80 in chloroform) to furnish 4-(2-methylcyclohexylamino)pyrrolo[1,2-b]pyridazine-3-carboximidamide (180 (0.019 g, 10%) as a white solid: ¹H NMR NMR (300 MHz, DMSO) δ 12.83-12.57 (m, 1H), 8.07 (s, 1H), 7.57 (dd, J=1.6, 2.6, 1H), 6.95-6.82 (m, 1H), 6.78 (d, J=3.1, 1H), 6.60 (dd, J=2.7, 4.5, 1H), 6.14 (s, 2H), 4.34 (s, 1H), 1.86 (s, 2H), 1.73-1.19 (m, 7H), 0.89 (d, J=6.9, 3H). MS (ES+) 272.2 (M+1); Analysis: Calcd for: C₁₅H₂₁N₅: C, 66.39; H, 7.80; N, 25.81. Found: C, 66.07; H, 7.85; N, 25.47.

### Example 64

### 4-(3-hydroxycyclohexylamino)pyrrolo[1,2-b]pyridazine-3-carbonitrile (51o)

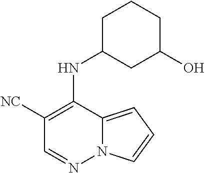

To a solution of 4-chloropyrrolo[1,2-b]pyridazine-3-carbonitrile (15g) (0.15 g, 0.84 mmol) in DMF (2.5 mL) was added at room temperature 3-aminocyclohexanol (51n) (194 mgs, 1.68 mmol), DIPEA (0.87 mL, 5 mmol) and stirred at room temperature overnight. The reaction was quenched with water (10 mL) and extracted with ethyl acetate (10 mL). The aqueous layer was separated and extracted with ethyl acetate (2×10 mL). The organic layers were combined washed with water (2×10 ml), brine (10 mL), dried, filtered and concentrated in vacuum. The residue obtained was purified by flash column chromatography (silica gel 12 g, eluting with 0-100% ethyl acetate in hexanes) to furnish 4-(3-hydroxycyclohexylamino)pyrrolo[1,2-b]pyridazine-3-carbonitrile (51o) (0.105 g, 46%) as a white solid: ¹H NMR (300 MHz, DMSO) δ 7.90 (s, 1H), 7.78 (d, J=8.5 Hz, 1H), 7.69 (dd, J=2.6, 1.6 Hz, 1H), 7.12 (dd, J=4.4, 1.6 Hz, 1H), 6.68 (dd, J=4.4, 2.7 Hz, 1H), 4.88 (d, J=4.3 Hz, 1H), 4.25 (m, 1H), 3.54 (m, 1H), 2.17 (m, 1H), 1.92 (m, 1H), 1.76 (d, J=13.3 Hz, 2H), 1.54-1.24 (m, 3H), 1.16 (dd, J=14.6, 10.6 Hz, 1H). MS (ES+) 536.3 (2M+Na), MS (ES−) 291.0 (M+Cl).

### Example 65

### 4-(3-hydroxycyclohexylamino)pyrrolo[1,2-b]pyridazine-3-carboxamide (51p)

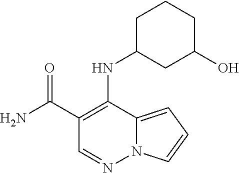

To a solution of 4-(3-hydroxycyclohexylamino)pyrrolo[1,2-b]pyridazine-3-carbonitrile (51o) (100 mg, 0.39 mmol) in ethanol (15 mL) was added at room temperature ammonium hydroxide (4 mL), hydrogen peroxide (0.2 mL) and stirred at room temperature overnight. The reaction was concentrated in vacuum, and the residue obtained was purified by flash column chromatography (silica gel 12 g, eluting with 0-100% (9:1) ethyl acetate/methanol in hexanes) to furnish 4-(3-hydroxycyclohexylamino)pyrrolo[1,2-b]pyridazine-3-carboxamide (51p) (0.016 g, 15%) as a brown solid. ¹HNMR (300 MHz, DMSO) δ 10.75 (s, 1H), 8.19 (s, 1H), 7.65 (s, 1H), 7.56-7.03 (m, 1H), 6.98 (s, 1H), 6.67 (d, J=2.7, 1H), 4.70 (d, J=3.7, 1H), 4.48-4.39 (m, 1H), 3.97-3.90 (m, 1H), 2.01-1.83 (m, 2H), 1.83-1.65 (m, 1H), 1.52 (s, 5H). MS (ES+) 275.2 (M+1), 297.1 (M+23), 571.1 (2M+Na); (ES−) 273.4 (M−1), 308.9 (M+Cl), 547.3 (2M−1).

### Example 66

### 2-(4-(2-methylcyclohexylamino)pyrrolo[1,2-b]pyridazine-3-carbonyl) hydrazine-carbothioamide (55b)

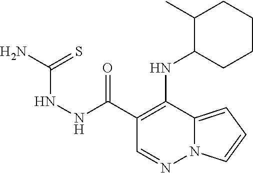

To a solution of 4-(2-methylcyclohexylamino)pyrrolo[1,2-b]pyridazine-3-carboxylic acid (18e) (700 mg, 2.56 mmol) in DMF (28 ml) was added thiosemicarbazide (55a) (336 mg, 3.65 mmol), and 1-hyxroxybenzotrizole (HOBt, 490 mg, 3.63 mmol) and cooled with ice/water. To the cold mixture was added N-(3-dimethylaminopropyl)-N′-ethylcarbodiimide hydrochloride (EDCI.HCl, 700 mg, 3.65 mmol) and stirred at 0° C. for 2 h followed at room temperature for 15 h. The reaction mixture was diluted with chloroform (240 mL) and methanol (80 mL), washed with water (150 mL), dried over MgSO4 and filtered. The filtrate was concentrated and the residue obtained was purified by flash column chromatography [silica gel 25 g, eluting with chloroform/methanol, 1:0 to 95:5, (R_{f}=0.31 with chloroform/methanol=20:1)] to give 2-(4-(2-methylcyclohexylamino)pyrrolo[1,2-b]pyridazine-3-carbonyphydrazine-carbothioamide 2-(4-(2-methylcyclohexylamino)pyrrolo[1,2-b]pyridazine-3-carbonyl)hydrazine-carbothioamide (55b) (342 mg, 39%) as a off-white solid; ¹H NMR (300 MHz, DMSO-d₆) δ 10.47 (d, J=8.7 Hz, 1H), 10.03 (s, 1H), 9.18 (s, 1H), 8.25 (s, 1H), 7.85 (s, 1H), 7.71-7.66 (m, 2H), 6.93 (dd, J=4.5, 1.5 Hz, 1H), 6.67 (dd, J=2.7, 4.5 Hz, 1H), 4.36 (s, 1H), 2.00-1.30 (m, 9H), 0.91 (d, J=6.9 Hz, 3H); MS (ES⁺): 347.1 (M+1); Analysis: Calcd for: C₁₆H₂₂N₆OS.0.25H₂O: C, 54.76; H, 6.46; N, 23.95; S, 9.14. Found: C, 54.78; H, 6.24; N, 24.19; S, 8.91.

### Example 67

### Racemic 4-(2-methylcyclohexylamino)-6-nitropyrrolo[1,2-b]pyridazine-3-carbonitrile (47e)

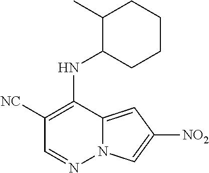

To a solution of 4-chloro-6-nitropyrrolo[1,2-b]pyridazine-3-carbonitrile (47d) (90 mg, 0.4 mmol) in DMF (10 mL) was added racemic 2-methylcyclohexanamine hydrochloride (18a) (160 mg, 1.07 mmol) triethylamine (0.45 mL, 3.23 mmol) and stirred at room temperature overnight. The reaction mixture was diluted with EtOAc (150 mL), washed with water (2×75 mL), brine (50 mL), dried over MgSO₄ filtered and concentrated in vacuum to dryness. The residue obtained was purified by flash column chromatography [silica gel 30 g, eluting with hexanes/ethyl acetate, 1:0 to 5:1, (R_{f}=0.46 with hexanes/ethyl acetate=5:1)] to afford racemic 4-(2-methylcyclohexylamino)-6-nitropyrrolo[1,2-b]pyridazine-3-carbonitrilee (47e) (110 mg, 92%) as a yellow solid; ¹H NMR (300 MHz, DMSO-d₆): δ 8.68 (d, J=2.0 Hz, 1H), 8.18 (s, 1H), 8.16 (d, J=1.9 Hz, 1H), 7.97 (d, J=8.1 Hz, 1H), 4.48-4.36 (m, 1H), 2.34-2.22 (m, 1H), 1.91-1.29 (m, 8H), 0.93 (d, J=7.1 Hz, 3H); MS (ES⁻): 298.1 (M−1).

### Example 68

### Racemic 4-(2-methylcyclohexylamino)-6-nitropyrrolo[1,2-b]pyridazine-3-carboxamide (47f)

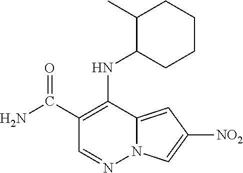

To a solution of racemic 4-(2-methylcyclohexylamino)-6-nitropyrrolo[1,2-b]pyridazine-3-carbonitrilee (47e) (100 mg, 0.33 mmol) in EtOH (8 mL) was added conc. NH₄OH (3 mL), followed by dropwise addition of H₂O₂ (0.14 mL, 35%, 1.37 mmol). The reaction mixture was stirred at room temperature for 4 h and concentrated in vacuum to dryness to furnish racemic 4-(2-methylcyclohexylamino)-6-nitropyrrolo[1,2-b]pyridazine-3-carboxamide (47f) (96 mg) as a yellow solid, which was pure enough to be used as such in next step; ¹H NMR (300 MHz, DMSO-d₆): δ 11.36 (d, J=8.6 Hz, 1H), 8.62 (d, J=1.9 Hz, 1H), 8.42 (s, 1H), 7.46 (d, J=1.9 Hz, 1H), 4.42-4.32 (m, 1H), 1.97-1.31 (m, 9H), 0.89 (d, J=6.9 Hz, 3H); MS (ES⁺): 318.2 (M+1).

### Example 69

### Racemic 6-amino-4-(2-methylcyclohexylamino)pyrrolo[1,2-b]pyridazine-3-carboxamide (47o)

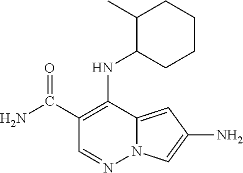

A solution of racemic 4-(2-methylcyclohexylamino)-6-nitropyrrolo[1,2-b]pyridazine-3-carboxamide (47f) (50 mg) in EtOH/ethyl acetate (12 mL/4 mL) was added Pd/C (10%, 30 mg) and hydrogenated at ˜50 psi for 5.5 h. The reaction mixture was filtered through celite to remove catalyst and concentrated in vacuum. The residue obtained was purified by flash column chromatography (silica gel 4 g, eluting with chloroform/methanol=1:0 to 9:1) to give racemic 6-amino-4-(2-methylcyclohexylamino)pyrrolo[1,2-b]pyridazine-3-carboxamide (47o) (10 mg, 20%) as a brown solid; ¹HNMR (300 MHz, DMSO-d₆): δ 10.57 (d, J=9.1 Hz, 1H), 8.03 (s, 1H), 7.07 (d, J=1.8 Hz, 1H), 6.25 (d, J=1.8 Hz, 1H), 4.24-4.10 (m, 1H), 1.99-1.18 (m, 9H), 0.89 (d, J=6.9 Hz, 3H); IR (KBr) MS (ES⁺): 288.2 (M+1).

### Example 70

### Racemic methyl 4-(2-methylcyclohexylamino)pyrrolo[1,2-b]pyridazine-3-carboxylate (57a)

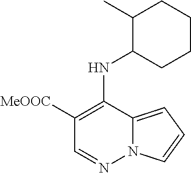

To a cooled (ice/water) solution of racemic 4-(2-Methylcyclohexylamino)pyrrolo[1,2-b]pyridazine-3-carboxylic acid (18e) (100 mg, 0.37 mmol) in DMF (3 mL) was added 4-dimethylaminopyridine (20 mg, 0.16 mmol), methanol (0.15 mL, 3.70 mmol) followed by N-(3-dimethylaminopropyl)-N′-ethylcarbodiimide hydrochloride (140 mg, 0.73 mmol). The reaction mixture was stirred at 0° C. for 2 h, allowed to warm to room temperature and continued stirring for 14 h. The reaction mixture was diluted with ethyl acetate (75 mL), washed with water (2×30 mL), brine (30 mL), dried, filtered and concentrated in vacuum. The residue obtained was purified by flash column chromatography [silica gel 4 g, eluting with hexanes/ethyl acetate, 1:0 to 9:1 (R_{f}=0.54 with hexanes/ethyl acetate=9:1)] to give methyl racemic 4-(2-methylcyclohexylamino)pyrrolo[1,2-b]pyridazine-3-carboxylate (57a) (77 mg, 72%) as a solid; ¹H NMR (300 MHz, DMSO-d₆): δ 9.76 (d, J=8.9 Hz, 1H), 8.21 (s, 1H), 7.75 (dd, J=1.5, 2.7 Hz, 1H), 7.02 (dd, J=1.5, 4.7 Hz, 1H), 6.71 (dd, J=2.7, 4.5 Hz, 1H), 4.35-4.46 (m, 1H), 3.80 (s, 3H), 2.01-1.34 (m, 9H), 0.91 (d, J=6.9 Hz, 3H); MS (ES⁺): 288.2 (M+1).

### Example 71

### Racemic N-(2-methylcyclohexyl)-3-(3-((tetrahydro-2H-pyran-2-yloxy)methyl)-1,2,4-oxadiazol-5-yl)pyrrolo[1,2-b]pyridazin-4-amine (57c)

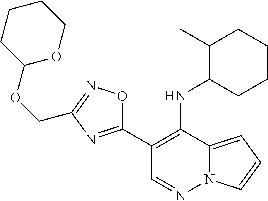

To a solution of N′-hydroxy-2-(tetrahydro-2H-pyran-2-yloxy)acetimidamide (57b) (85 mg, 0.49 mmol, prepared according to literature procedure given in Showell, G. A.; Gibbons, T. L.; Kneen, C. O.; MacLeod, A. M.; Merchant, K.; Saunders, J.; Freedman, S. B.; Patel, S.; Baker, R. J. Med. Chem. 1991, 34, 1086-1094) in THF (4 mL) was added NaH (60% in mineral oil, 22 mg, 0.55 mmol) and 4A molecular sieves (370 mg). The reaction mixture was heated at 50° C. for 1 h. To the anion formed was added a solution of racemic methyl 4-(2-methylcyclohexylamino)pyrrolo[1,2-b]pyridazine-3-carboxylate (57a) (70 mg, 0.24 mmol) in THF (2 mL) and heated at reflux for 19 h. The reaction mixture was cooled to room temperature quenched with water (30 mL) and extracted with dichloromethane (2×50 mL). The organic layers were combined dried, filtered and concentrated in vacuum to dryness. The residue obtained was purified by flash column chromatography [silica gel 4 g, eluting with hexanes/ethyl acetate, 1:0 to 9:1 (R_{f}=0.44 with hexanes/ethyl acetate=9:1)] to give racemic N-(2-methylcyclohexyl)-3-(3-((tetrahydro-2H-pyran-2-yloxy)methyl)-1,2,4-oxadiazol-5-yl)pyrrolo[1,2-b]pyridazin-4-amine (57c) (16 mg, 16%) as a solid; ¹H NMR (300 MHz, DMSO-d₆): δ 9.44 (d, J=8.8 Hz, 1H), 8.36 (s, 1H), 7.84 (dd, J=1.4, 2.7 Hz, 1H), 7.13 (dd, J=1.4, 4.6 Hz, 1H), 6.78 (dd, J=2.7, 4.6 Hz, 1H), 4.85-4.80 (m, 1H), 4.80-4.63 (m, 2H), 4.53 (s, 1H), 3.86-3.70 (m, 1H), 3.55-3.45 (m, 1H), 2.03-1.09 (m, 15H), 0.95 (d, J=6.9 Hz, 3H); MS (ES⁺): 412.13 (M+1).

### Example 72

### Racemic (5-(4-(2-methylcyclohexylamino)pyrrolo[1,2-b]pyridazin-3-yl)-1,2,4-oxadiazol-3-yl)methanol (57d)

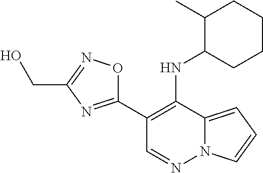

To a solution of racemic N-(2-methylcyclohexyl)-3-(3-((tetrahydro-2H-pyran-2-yloxy)methyl)-1,2,4-oxadiazol-5-yl)pyrrolo[1,2-b]pyridazin-4-amine (57c) (14 mg, 0.034 mmol) in ethanol (2 ml) was added pyridinium p-toluene sulfonate (1 mg, 0.004 mmol) and stirred at 55° C. for 2 h. Additional pyridinium p-toluene sulfonate (1 mg, 0.004 mmol) was added to the reaction mixture and continued heating at 55° C. until hydrolysis was complete. The reaction mixture was concentrated in vacuum to dryness and the residue was dissolved dichloromethane (50 mL). The organic layer was washed with water (25 ml), dried, filtered and concentrated in vacuum to dryness. The residue obtained was purified by flash column chromatography [silica gel 4 g, eluting with hexanes/ethyl acetate, 1:0 to 4:1 (R_{f}=0.32 with hexanes/ethyl acetate=4:1)] to give racemic (5-(4-(2-methylcyclohexylamino)pyrrolo[1,2-b]pyridazin-3-yl)-1,2,4-oxadiazol-3-yl)methanol (57d) (6.0 mg, 54%) as a solid; ¹H NMR (300 MHz, DMSO-d₆): δ 9.48 (d, J=8.5 Hz, 1H), 8.35 (s, 1H), 7.83 (dd, J=1.5, 2.7 Hz, 1H), 7.11 (dd, J=1.4, 4.7 Hz, 1H), 6.77 (dd, J=2.7, 4.6 Hz, 1H), 5.69 (t, J=6.0 Hz, 1H), 4.62 (d, J=5.9 Hz, 2H), 4.55-4.45 (m, 1H), 2.10-1.10 (m, 9H), 0.95 (d, J=6.9 Hz, 3H); MS (ES⁺): 328.14 (M+1).

### Example 73

The following illustrate representative pharmaceutical dosage forms, containing a compound of formula I (‘Compound X’), for therapeutic or prophylactic use in humans.

| (i) Tablet 1 | mg/tablet |
|---|---|
| Compound X = | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X = | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X = | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X = (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| Compound X = (free acid form) | 10.0 |
| Monobasic sodium phosphate | 0.3 |
| Dibasic sodium phosphate | 1.1 |
| Polyethylene glycol 400 | 200.0 |
| 01N Sodium hydroxide solution | q.s. |
| (pH adjustment to 7.0-7.5) | |
| Water for injection | q.s. ad 1 mL |

| (v) Aerosol | mg/can |
|---|---|
| Compound X = | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

TABLE I
Activity for Representative Compounds of
the Invention for JAK Family of Enzymes

| Compound | Activity | Compound | Activity |
|---|---|---|---|
| 18c | IC₅₀ < 5 uM | 48d | IC₅₀ < 5 uM |
| 18b | IC₅₀ < 5 uM | 39h | IC₅₀ < 5 uM |
| 21f | IC₅₀ > 10 uM | 54b | IC₅₀ < 5 uM |
| 41a | IC₅₀ > 10 uM | 54e | IC₅₀ < 5 uM |
| 39b | IC₅₀ > 10 uM | 48b | IC₅₀ < 5 uM |
| 39d | IC₅₀ < 5 uM | 21i | IC₅₀ < 5 uM |
| 18g | IC₅₀ < 5 uM | 51e | IC₅₀ < 5 uM |
| 18i | IC₅₀ < 5 uM | 51f | IC₅₀ < 5 uM |
| 47k | IC₅₀ > 10 uM | 51i | IC₅₀ < 5 uM |
| 47m | IC₅₀ < 10 uM | 54c | IC₅₀ < 5 uM |
| 47n | IC₅₀ < 5 uM | 51m | IC₅₀ < 5 uM |
| 48c | IC₅₀ < 5 uM | 52c | IC₅₀ < 5 uM |
| 49c | IC₅₀ < 5 uM | 52f | IC₅₀ < 5 uM |
| 49f | IC₅₀ < 5 uM | 52i | IC₅₀ < 5 uM |
| 49i | IC₅₀ < 5 uM | 52m | IC₅₀ < 5 uM |
| 49l | IC₅₀ < 5 uM | 53c | IC₅₀ < 5 uM |
| 50c | IC₅₀ < 10 uM | 53f | IC₅₀ < 5 uM |
| 48f | IC₅₀ < 5 uM | 47l | IC₅₀ < 10 uM |
| 50i | IC₅₀ > 10 uM | 54f | IC₅₀ < 5 uM |
| 50m | IC₅₀ < 5 uM | 51h | IC₅₀ < 5 uM |
| 51c | IC₅₀ < 5 uM | | |

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A compound of formula I: 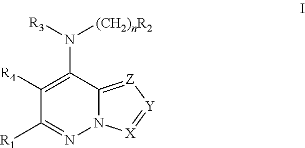
wherein
X is N or CR₅;
Y is N or CR₆;
Z is N or CR₇;
n is 0 or 1;
R₁ is H, F, Br, I, (C₂-C₁₀)alkyl, cycloalkyl, alkenyl, alkynyl, aryl, heteroaryl, heterocycle, NO₂, —CN, —OH, —OR_{d}, —NR_{b}R_{c}, N₃, SH, —SR_{d}, —C(O)Rₐ, —C(O)ORₐ, —C(O)NR_{b}R_{c}, —C(═NR_{b})NR_{b}R_{c}, —NR_{b}COR_{d}, —NR_{b}C(O)OR_{d}, —NR_{b}S(O)₂R_{d}, —NR_{b}CONR_{b}R_{c}, —OC(O)NR_{b}R_{c}, —S(O)R_{d}, —S(O)NR_{b}R_{c}, —S(O)₂R_{d}, —S(O)₂OH, or —S(O)₂NR_{b}R_{c}; wherein any aryl or heteroaryl of R₁ may be optionally substituted with one or more Rₑ groups; and wherein any alkyl, cycloalkyl, alkenyl, alkynyl or heterocycle of R₁ may be optionally substituted with one or groups selected from Rₑ, oxo and ═NOR_{z};
R₂ is alkyl, cycloalkyl, heterocycle, heteroaryl, aryl, —Oalkyl or a bridged ring group; wherein any aryl or heteroaryl of R₂ may be optionally substituted with one or more R_{f} groups; and wherein any alkyl, —Oalkyl, cycloalkyl, heterocycle or bridged ring group of R₂ may be optionally substituted with one or more groups selected from R_{f}, oxo and ═NOR_{z};
R₃ is H, —CN, —C(O)alkyl, —C(O)alkenyl, —C(O)alkynyl, —C(O)cycloalkyl, —C(O)aryl, —C(═O)C(═O)NHlower alkyl, —CONR_{g}Rₕ, alkyl, alkenyl, heterocycle, or heteroaryl; wherein any —C(O)aryl or heteroaryl of R₃ may be optionally substituted with one or more Rᵢ groups; and wherein any alkyl, alkenyl, —C(O)alkyl, —C(O)alkenyl, —C(O)alkynyl, —C(O)cycloalkyl, heterocycle or —C(═O)C(═O)NHlower alkyl of R₃ may be optionally substituted with one or more groups selected from oxo and ═NOR_{z};
R₄ is halogen, alkyl, cycloalkyl, alkenyl, alkynyl, aryl, heteroaryl, heterocycle, NO₂, —CN, OH, —ORₙ, —NRₖRₘ, N₃, —SH, —SRₙ, —C(O)alkyl, —C(O)alkenyl, —C(O)alkynyl, —C(O)cycloalkyl, —C(O)aryl, —C(O)heteroaryl, —C(O)heterocycle, —C(O)OR_{J}, —C(O)NRₖRₘ, —C(═NRₖ)NRₖRₘ, —NRₖCORₙ, —NRₖC(O)ORₙ, —NRₖS(O)₂Rₙ, —NRₖCONRₖRₘ, —OC(O)NRₖRₘ, —S(O)Rₙ, —S(O)NRₖRₘ, —S(O)₂Rₙ, —S(O)₂OH, —S(O)₂NRₖRₘ, —C(═O)NHNHC(═S)NH₂, —C(═NH)NHOH or —C(═O)C(═O)NHlower alkyl; wherein any aryl, heteroaryl, C(O)aryl or —C(O)heteroaryl of R₄ may be optionally substituted with one or more Rₚ groups and wherein any alkyl, cycloalkyl, alkenyl, alkynyl, heterocycle, C(O)alkyl, —C(O)alkenyl, —C(O)alkynyl, —C(O)cycloalkyl, —C(O)heterocycle or —C(═O)C(═O)NHlower alkyl of R₄ may be optionally substituted with one or more groups selected from Rₚ, oxo and ═NOR_{z};
R₅ is H, OH, NO₂, CO₂H, —NR_{q}Rᵣ, —NHC(O)CF₃, —CONR_{q}Rᵣ, halogen or lower alkyl; which lower alkyl is optionally substituted with one or more Rₛ groups;
R₆ is H, OH, NO₂, CO₂H, —NR_{q}Rᵣ, halogen, —CONR_{q}Rᵣ, alkenyl or lower alkyl; which lower alkyl or alkenyl is optionally substituted with one or more Rₛ groups;
R₇ is H, OH, NO₂, CO₂H, —NR_{q}Rᵣ, —CONR_{q}Rᵣ, or halogen;
each Rₐ is independently selected from H, alkyl, alkenyl, alkynyl, cycloalkyl, heterocycle, heteroaryl and aryl;
R_{b} and R_{c} are each independently selected from H, alkyl, alkenyl, alkynyl, cycloalkyl, heterocycle and heteroaryl; or R_{b} and R_{c} together with the nitrogen to which they are attached form a pyrrolidino, piperidino, piperazino, azetidino, morpholino, or thiomorpholino ring;
each R_{d} is independently selected from alkyl, alkenyl, alkynyl, cycloalkyl, heterocycle, heteroaryl and aryl;
each Rₑ is independently selected from halogen, aryl, heteroaryl, heterocycle, R_{z}, OH, —CN, —OR_{z}, —Oaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z}; wherein any aryl, —Oaryl, —Saryl, —S(O)aryl, —S(O)₂aryl, —NHCOaryl, or NHS(O)₂aryl of Rₑ may be optionally substituted with one or more R_{y} groups;
each R_{f} is independently selected from halogen, aryl, heteroaryl, heterocycle, R_{z}, OH, —CN, —OR_{z}, —Oaryl, —Oheterocycle, —Oheteroaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2}, —C(O)heterocycle, —C(O)heteroaryl and —C(O)C(O)R_{z};
wherein any aryl, heteroaryl, —Oaryl, —Oheteroaryl, —Saryl, —Sheteroaryl, —S(O)heteroaryl, —S(O)₂aryl, —S(O)₂heteroaryl, —NHCOaryl, —NHCOheteroaryl, —NHS(O)₂aryl or —C(O)heteroaryl of R_{f} may be optionally substituted with one or more R_{y} groups; and wherein any heterocycle or —C(O)heterocycle of R_{f} may be optionally substituted with one or more groups selected from R_{y}, oxo and ═NOR_{z};
R_{g} and Rₕ are each independently selected from H, alkyl, alkenyl, alkynyl, cycloalkyl, heterocycle and heteroaryl; or R_{g} and Rₕ together with the nitrogen to which they are attached form a pyrrolidino, piperidino, piperazino, azetidino, morpholino, or thiomorpholino ring;
each Rᵢ is independently selected from halogen, aryl, heteroaryl, heterocycle, R_{z}, OH, —CN, —OR_{z}, —Oaryl, —OC(O)R_{z}, —OC(O)NR_{z1}R_{z2}, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z}; wherein any aryl, —Oaryl, —Saryl, —Sheteroaryl, —S(O)aryl, —S(O)₂aryl, —NHCOaryl, or —NHS(O)₂aryl, of Rᵢ may be optionally substituted with one or more R_{y} groups;
Rⱼ is H, alkyl, alkenyl, alkynyl, cycloalkyl, heterocycle, heteroaryl or aryl;
Rₖ and Rₘ are each independently selected from H, alkyl, alkenyl, alkynyl, cycloalkyl, heterocycle and heteroaryl; or Rₖ and Rₘ together with the nitrogen to which they are attached form a pyrrolidino, piperidino, piperazino, azetidino, morpholino, or thiomorpholino ring;
each Rₙ is independently selected from alkyl, alkenyl, alkynyl, cycloalkyl, heterocycle, heteroaryl and aryl;
each Rₚ is independently selected from halogen, aryl, heteroaryl, heterocycle, R_{z}, OH, —CN, —OR_{z}, —Oaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z}; wherein any aryl, —Oaryl, —Saryl, —S(O)aryl, —S(O)₂aryl, —NHCOaryl, —NHCOheteroaryl, —NHCO₂R_{z}, —NHCONR_{z1}R_{z2} or —NHS(O)₂aryl, of Rₚ may be optionally substituted with one or more R_{y} groups;
R_{q} and Rᵣ are each independently selected from H, alkyl, alkenyl, alkynyl, cycloalkyl, heterocycle and heteroaryl; or R_{q} and Rᵣ together with the nitrogen to which they are attached form a pyrrolidino, piperidino, piperazino, azetidino, morpholino, or thiomorpholino ring;
each Rₛ is independently selected from halogen, aryl, heteroaryl, heterocycle, R_{z}, OH, —CN, —OR_{z}, —Oaryl, —OC(O)R_{z}, —OC(O)NR_{z1}R_{z2}, oxo, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, ═NOR_{z}, —CHO, —C(O)R_{z}, —C(O)OH, —C(O)OR_{z}, —C(O)NR_{z1}R_{z2} and —C(O)C(O)R_{z}; wherein any aryl, Oaryl, —Saryl, —S(O)aryl, —S(O)₂aryl, —NHCOaryl or —NHS(O)₂aryl of Rₛ may be optionally substituted with one or more R_{y} groups;
each R_{z} is independently lower alkyl or lower cycloalkyl; wherein any lower alkyl or lower cycloalkyl of R_{z} may be optionally substituted with one or more groups selected from halogen, —CN, OH, —Olower alkyl, —NHlower alkyl, —C(O)NHlower alkyl, —C(O)N(lower alkyl)₂, aryl, heterocycle, —Oheterocycle and heteroaryl; wherein any aryl, heteroaryl or heterocycle of R_{z} may be optionally substituted with one or more lower alkyl;
R_{z1} and R_{z2} are each independently selected from H, lower alkyl, alkenyl, alkynyl, lower cycloalkyl, heterocycle and heteroaryl; wherein any lower alkyl or lower cycloalkyl of R_{z1} and R_{z2} may be optionally substituted with one or more Rₜ groups; or R_{z1} and R_{z2} together with the nitrogen to which they are attached form a cyclic amino;
each Rₜ is independently selected from halogen, —CN, OH, —Olower alkyl, —NHlower alkyl, —C(O)NHlower alkyl, —C(O)N(lower alkyl)₂, heterocycle and heteroaryl; wherein any heterocycle of Rₜ may be substituted with one or more lower alkyl; and
each R_{y} is independently halogen, aryl, R_{z}, OH, —CN, OR_{z}, —Oaryl, —Oheteroaryl, —OC(O)R_{z}, —OC(O)NR_{z1}R_{z2}, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, CHO, —C(O)R_{z}, —C(O)OH, —C(O)OR_{z}, —C(O)NR_{z1}R_{z2}, —C(O)C(O)R_{z}, heterocycle or heteroaryl;
or a salt thereof.

2. The compound of claim 1, wherein X is CR₅.

3. The compound of claim 2, wherein R₅ is H, OH, NO₂, CO₂H, —NR_{q}Rᵣ, or CONH₂.

4. The compound of claim 3, wherein R₅ is H.

5. (canceled)

6. The compound of claim 1, wherein Y is CR₆.

7. The compound of claim 6, wherein R₆ is H, OH, NO₂, halogen or NH₂.

8. The compound of claim 6, wherein R₆ is H, NO₂ or NH₂.

9. (canceled)

10. The compound of claim 1, wherein Z is CR₇.

11. The compound of claim 10, wherein R₇ is H.

12. (canceled)

13. The compound of claim 1, wherein Y and Z are each CH.

14. The compound of claim 1, wherein n is 0.

15. The compound of claim 1, wherein R₁ is H.

16. (canceled)

17. (canceled)

18. The compound of claim 1, wherein R₃ is H.

19. (canceled)

20. The compound of claim 1, wherein R₄ is —C(O)NRₖRₘ, —C(O)ORⱼ or —CN.

21. The compound of claim 1, wherein R₄ is —C(O)NRₖRₘ.

22. The compound of claim 1, wherein R₄ is —C(O)NH₂.

23. (canceled)

24. (canceled)

25. The compound of claim 1, wherein R₄ is —CN.

26-29. (canceled)

30. The compound of claim 1, wherein R₂ is alkyl, cycloalkyl, heterocycle or aryl; wherein any aryl of R₂ may be optionally substituted with one or more R_{f} groups; and wherein any alkyl, cycloalkyl or heterocycle of R₂ may be optionally substituted with one or more groups selected from R_{f}, oxo and ═NOR_{z}.

31. The compound of claim 30, wherein R₂ is alkyl; wherein any alkyl of R₂ is substituted with one or more R_{f} groups.

32. (canceled)

33. The compound of claim 1, wherein R₂ is aryl; wherein any aryl of R₂ may be optionally substituted with one or more R_{f} groups.

34. (canceled)

35. The compound of claim 1, wherein R₂ is cycloalkyl or heterocycle; wherein any cycloalkyl or heterocycle of R₂ may be optionally substituted with one or more groups selected from R_{f} and oxo.

36. The compound of claim 1, wherein R₂ is cyclopropyl, cyclopentyl, cyclohexyl, cycloheptyl, tetrahydropyranyl, tetrahydrofuranyl or piperidinyl; wherein any cyclopropyl, cyclopentyl, cyclohexyl, cycloheptyl, tetrahydropyranyl, tetrahydrofuranyl or piperidinyl of R₂ may be optionally substituted with one or more groups selected from R_{f} and oxo.

37-49. (canceled)

50. The compound of claim 1, wherein R₂ is:

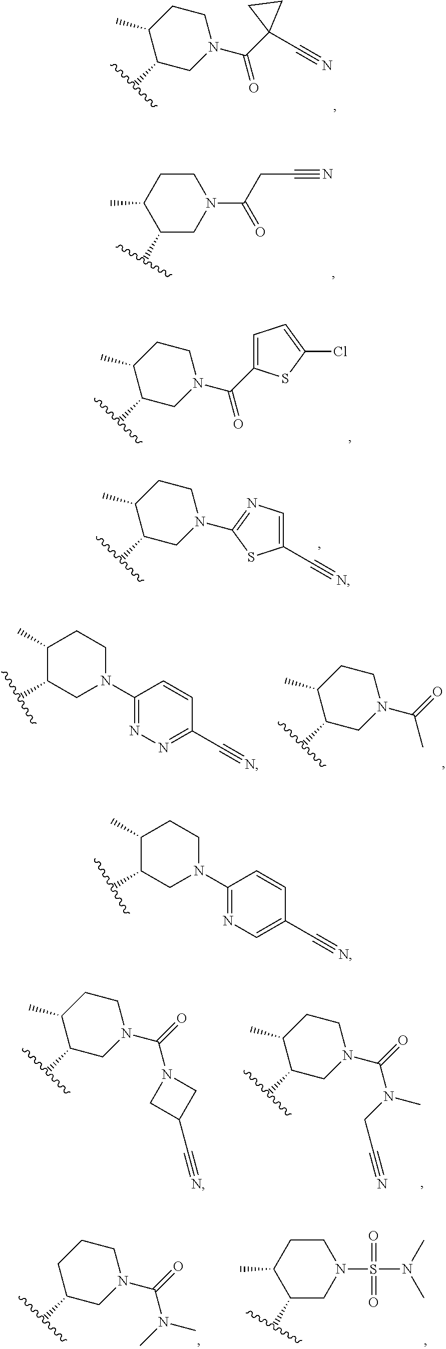

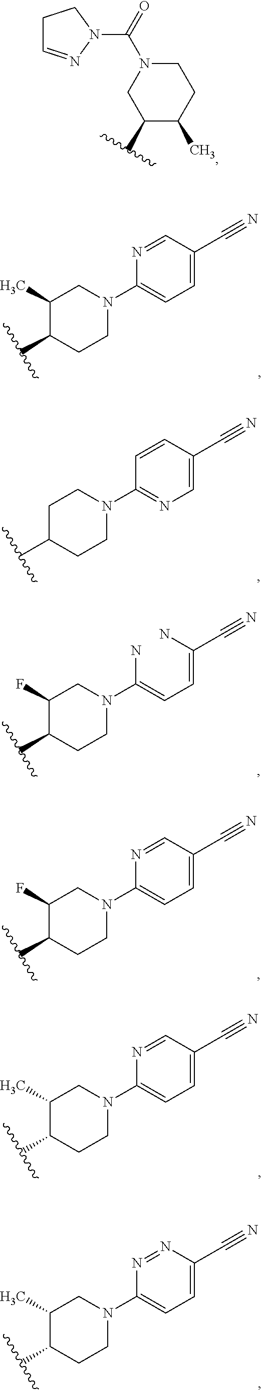

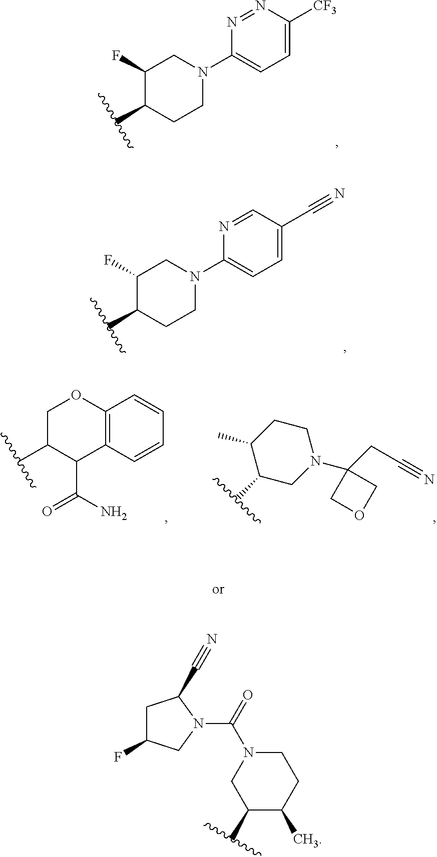

51. The compound of claim 1, wherein R₂ is:

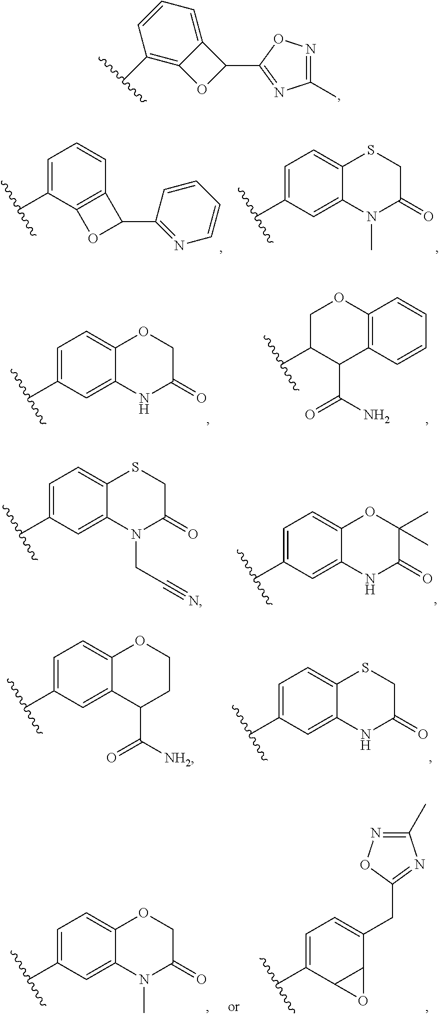

52. The compound of claim 1, wherein R₂ is:

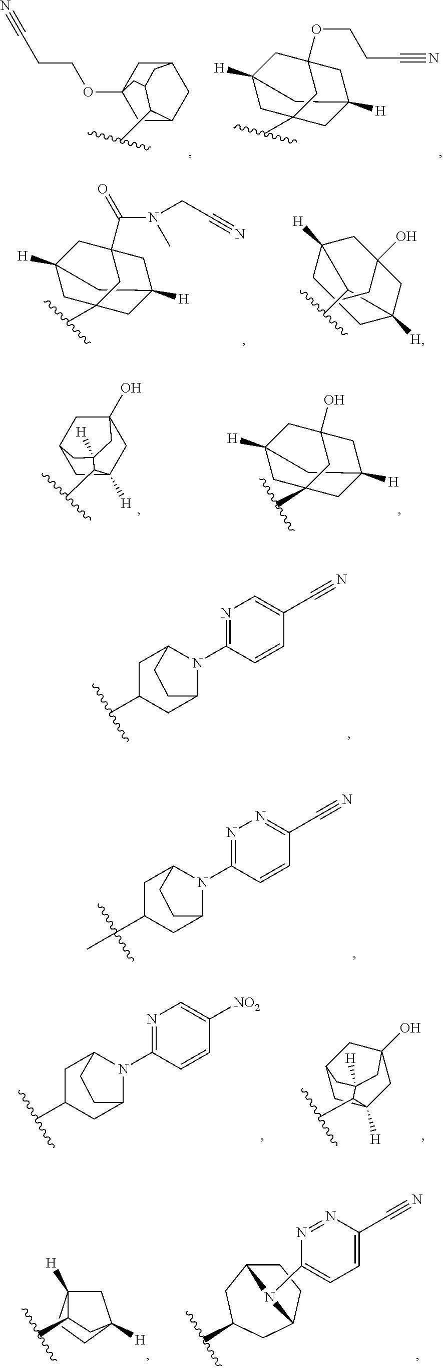

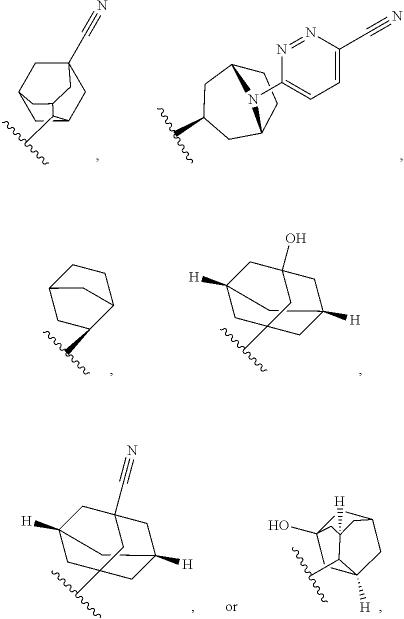

53. The compound of claim 1, wherein R₂ is:

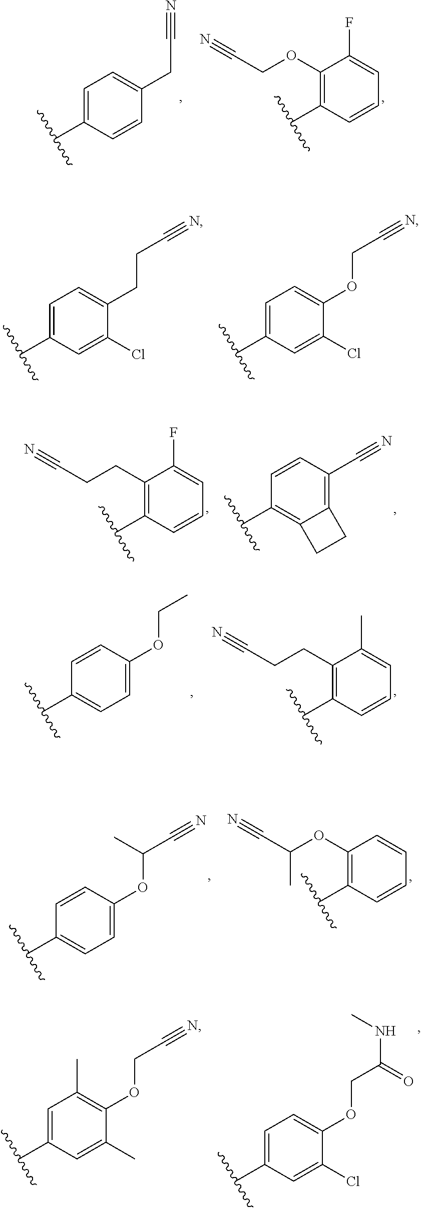

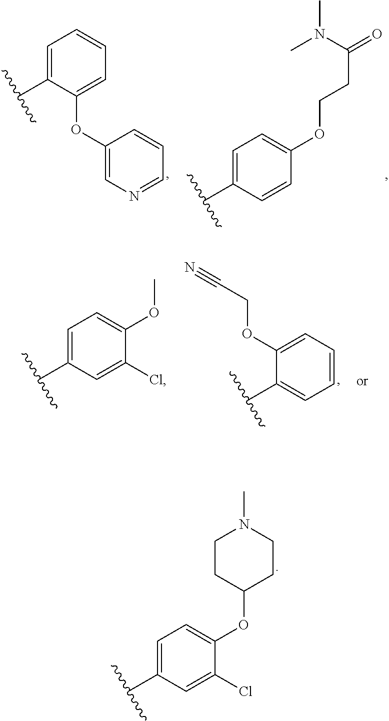

54. The compound of claim 1, wherein R₂ is:

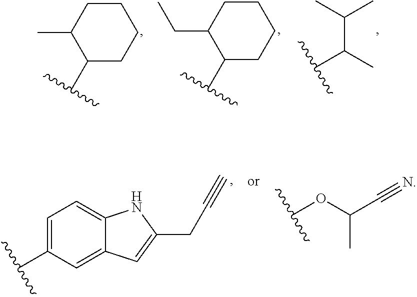

55. The compound of claim 1, wherein R₂ is:

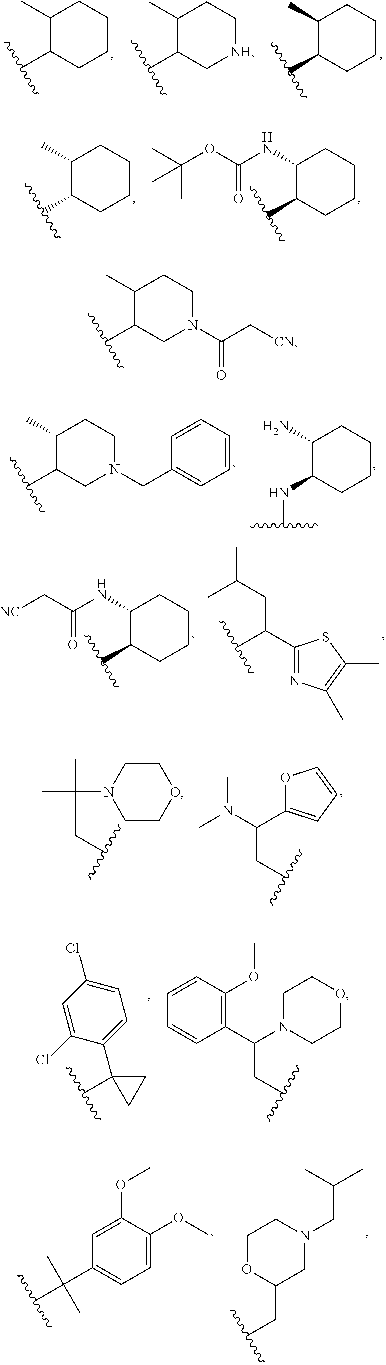

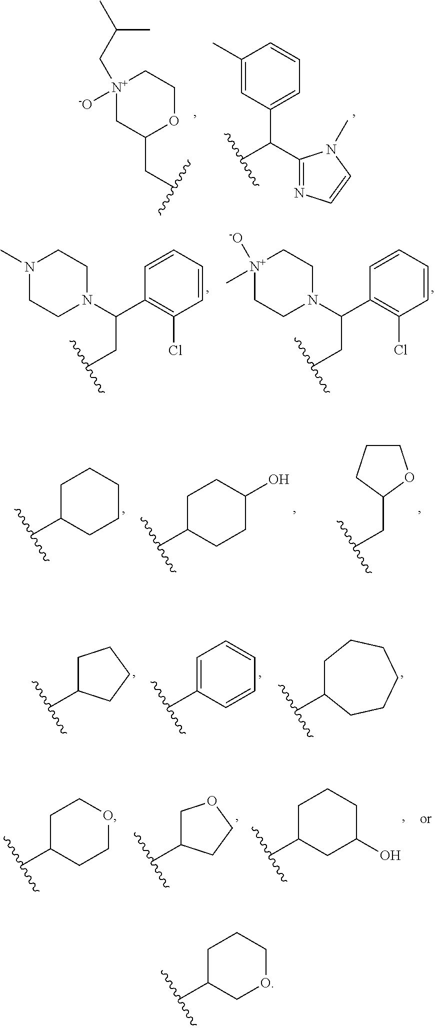

56. A compound selected from the group consisting of:
4-(2-methylcyclohexylamino)pyrrolo[1,2-b]pyridazine-3-carboxamide;
7-amino-4-(2-methylcyclohexylamino)pyrrolo[1,2-b]pyridazine-3-carboxamide;
4-(4-methylpiperidin-3-ylamino)pyrrolo[1,2-b]pyridazine-3-carboxamide;
4-(1-(2-cyanoacetyl)-4-methylpiperidin-3-ylamino)pyrrolo[1,2-b]pyridazine-3-carboxamide;
4-(2-methylcyclohexylamino)pyrrolo[1,2-b]pyridazine-3-carboxylic acid;
4-(((3R,4R)-1-benzyl-4-methylpiperidin-3-yl)(methyl)amino)pyrrolo[1,2-b]pyridazine-3-carbonitrile;
4-((1R,2S)-2-methylcyclohexylamino)pyrrolo[1,2-b]pyridazine-3-carboxamide;
4-((1S,2R)-2-methylcyclohexylamino)pyrrolo[1,2-b]pyridazine-3-carboxamide;
tert-butyl (1R,2R)-2-(3-cyanopyrrolo[1,2-b]pyridazin-4-ylamino)cyclohexyl carbamate;
tert-butyl (1R,2R)-2-(3-carbamoylpyrrolo[1,2-b]pyridazin-4-ylamino)cyclohexyl carbamate;
4-((1R,2R)-2-aminocyclohexylamino)pyrrolo[1,2-b]pyridazine-3-carboxamide;
4-((1R,2R)-2-(2-cyanoacetamido)cyclohexylamino)pyrrolo[1,2-b]pyridazine-3-carboxamide;
4-((1S,2R)-2-methylcyclohexylamino)-7-nitropyrrolo[1,2-b]pyridazine-3-carbonitrile;
4-((1S,2R)-2-methylcyclohexylamino)-7-nitropyrrolo[1,2-b]pyridazine-3-carboxamide;
7-amino-4-((1S,2R)-2-methylcyclohexylamino)pyrrolo[1,2-b]pyridazine-3-carboxamide;
4-((1R,2S)-2-methylcyclohexylamino)-7-nitropyrrolo[1,2-b]pyridazine-3-carbonitrile;
4-((1R,2S)-2-methylcyclohexylamino)-7-nitropyrrolo[1,2-b]pyridazine-3-carboxamide;
7-amino-4-((1R,2S)-2-methylcyclohexylamino)pyrrolo[1,2-b]pyridazine-3-carboxamide;
4-(1-(4,5-Dimethylthiazol-2-yl)-3-methylbutylamino)pyrrolo[1,2-b]pyridazine-3-carbonitrile;
4-(1-(4,5-dimethylthiazol-2-yl)-3-methylbutylamino)pyrrolo[1,2-b]pyridazine-3-carboxamide;
4-(2-methyl-2-morpholinopropylamino)pyrrolo[1,2-b]pyridazine-3-carbonitrile;
4-(2-methyl-2-morpholinopropylamino)pyrrolo[1,2-b]pyridazine-3-carboxamide;
4-(2-(dimethylamino)-2-(furan-2-yl)ethylamino)pyrrolo[1,2-b]pyridazine-3-carbonitrile;
4-(2-(dimethylamino)-2-(furan-2-yl)ethylamino)pyrrolo[1,2-b]pyridazine-3-carboxamide;
4-(1-(2,4-dichlorophenyl)cyclopropylamino)pyrrolo[1,2-b]pyridazine-3-carbonitrile;
4-(1-(2,4-dichlorophenyl)cyclopropylamino)pyrrolo[1,2-b]pyridazine-3-carboxamide;
4-(2-(2-methoxyphenyl)-2-morpholinoethylamino)pyrrolo[1,2-b]pyridazine-3-carbonitrile;
4-(2-(2-methoxyphenyl)-2-morpholinoethylamino)pyrrolo[1,2-b]pyridazine-3-carboxamide;
4-(2-(3,4-dimethoxyphenyl)propan-2-ylamino)pyrrolo[1,2-b]pyridazine-3-carbonitrile;
4-(2-(3,4-dimethoxyphenyl)propan-2-ylamino)pyrrolo[1,2-b]pyridazine-3-carboxamide;
4-((4-isobutylmorpholin-2-yl)methylamino)pyrrolo[1,2-b]pyridazine-3-carbonitrile;
2-((3-carbamoylpyrrolo[1,2-b]pyridazin-4-ylamino)methyl)-4-isobutylmorpholine 4-oxide;
4-((1-methyl-1H-imidazol-2-yl)(m-tolyl)methylamino)pyrrolo[1,2-b]pyridazine-3-carbonitrile;
4-((1-methyl-1H-imidazol-2-yl)(m-tolyl)methylamino)pyrrolo[1,2-b]pyridazine-3-carboxamide;
4-(2-(2-chlorophenyl)-2-(4-methylpiperazin-1-yl)ethylamino)pyrrolo[1,2-b]pyridazine-3-carbonitrile;
4-(2-(3-carbamoylpyrrolo[1,2-b]pyridazin-4-ylamino)-1-(2-chlorophenyl)ethyl)-1-methylpiperazine-1-oxide;
4-(cyclohexylamino)pyrrolo[1,2-b]pyridazine-3-carbonitrile;
4-(cyclohexylamino)pyrrolo[1,2-b]pyridazine-3-carboxamide;
4-(4-hydroxycyclohexylamino)pyrrolo[1,2-b]pyridazine-3-carbonitrile;
4-(4-hydroxycyclohexylamino)pyrrolo[1,2-b]pyridazine-3-carboxamide;
4-((tetrahydrofuran-2-yl)methylamino)pyrrolo[1,2-b]pyridazine-3-carbonitrile;
4-((tetrahydrofuran-2-yl)methylamino)pyrrolo[1,2-b]pyridazine-3-carboxamide;
4-(cyclopentylamino)pyrrolo[1,2-b]pyridazine-3-carbonitrile;
4-(cyclopentylamino)pyrrolo[1,2-b]pyridazine-3-carboxamide;
4-(phenylamino)pyrrolo[1,2-b]pyridazine-3-carbonitrile;
4-(phenylamino)pyrrolo[1,2-b]pyridazine-3-carboxamide;
4-(cycloheptylamino)pyrrolo[1,2-b]pyridazine-3-carbonitrile;
4-(cycloheptylamino)pyrrolo[1,2-b]pyridazine-3-carboxamide;
4-(tetrahydro-2H-pyran-4-ylamino)pyrrolo[1,2-b]pyridazine-3-carbonitrile;
4-(tetrahydro-2H-pyran-4-ylamino)pyrrolo[1,2-b]pyridazine-3-carboxamide;
4-(tetrahydrofuran-3-ylamino)pyrrolo[1,2-b]pyridazine-3-carbonitrile;
4-(tetrahydrofuran-3-ylamino)pyrrolo[1,2-b]pyridazine-3-carboxamide;
4-(tetrahydro-2H-pyran-3-ylamino)pyrrolo[1,2-b]pyridazine-3-carbonitrile;
4-(tetrahydro-2H-pyran-3-ylamino)pyrrolo[1,2-b]pyridazine-3-carboxamide;
4-(cyclopentylamino)-7-nitropyrrolo[1,2-b]pyridazine-3-carbonitrile;
4-(cyclopentylamino)-7-nitropyrrolo[1,2-b]pyridazine-3-carboxamide;
7-amino-4-(cyclopentylamino)pyrrolo[1,2-b]pyridazine-3-carboxamide;
7-nitro-4-(phenylamino)pyrrolo[1,2-b]pyridazine-3-carbonitrile;
7-nitro-4-(phenylamino)pyrrolo[1,2-b]pyridazine-3-carboxamide;
7-amino-4-(phenylamino)pyrrolo[1,2-b]pyridazine-3-carboxamide;
4-(2-methylcyclohexylamino)pyrrolo[1,2-b]pyridazine-3,7-dicarboxamide;
N-hydroxy-4-(2-methylcyclohexylamino)pyrrolo[1,2-b]pyridazine-3-carboximidamide;
4-(2-methylcyclohexylamino)pyrrolo[1,2-b]pyridazine-3-carboximidamide;
4-(3-hydroxycyclohexylamino)pyrrolo[1,2-b]pyridazine-3-carbonitrile;
4-(3-hydroxycyclohexylamino)pyrrolo[1,2-b]pyridazine-3-carboxamide;
2-(4-(2-methylcyclohexylamino)pyrrolo[1,2-b]pyridazine-3-carbonyl)hydrazine-carbothioamide;
4-(2-methylcyclohexylamino)-7-(2,2,2-trifluoroacetamido)pyrrolo[1,2-b]pyridazine-3-carboxamide;
4-((1S,2R)-2-methylcyclohexylamino)-6-nitropyrrolo[1,2-b]pyridazine-3-carbonitrile;
4-((1S,2R)-2-methylcyclohexylamino)-6-nitropyrrolo[1,2-b]pyridazine-3-carboxamide;
6-amino-4-((1S,2R)-2-methylcyclohexylamino)pyrrolo[1,2-b]pyridazine-3-carboxamide;
4-((1R,2S)-2-methylcyclohexylamino)-6-nitropyrrolo[1,2-b]pyridazine-3-carbonitrile;
4-((1R,2S)-2-methylcyclohexylamino)-6-nitropyrrolo[1,2-b]pyridazine-3-carboxamide;
6-amino-4-((1R,2S)-2-methylcyclohexylamino)pyrrolo[1,2-b]pyridazine-3-carboxamide;
4-(cyclopentylamino)-6-nitropyrrolo[1,2-b]pyridazine-3-carbonitrile;
4-(cyclopentylamino)-6-nitropyrrolo[1,2-b]pyridazine-3-carboxamide;
6-amino-4-(cyclopentylamino)pyrrolo[1,2-b]pyridazine-3-carboxamide;
6-nitro-4-(phenylamino)pyrrolo[1,2-b]pyridazine-3-carbonitrile;
6-nitro-4-(phenylamino)pyrrolo[1,2-b]pyridazine-3-carboxamide;
6-amino-4-(phenylamino)pyrrolo[1,2-b]pyridazine-3-carboxamide;
methyl 4-(2-methylcyclohexylamino)pyrrolo[1,2-b]pyridazine-3-carboxylate;
N-(2-methylcyclohexyl)-3-(3-((tetrahydro-2H-pyran-2-yloxy)methyl)-1,2,4-oxadiazol-5-yl)pyrrolo[1,2-b]pyridazin-4-amine;
(5-(4-(2-methylcyclohexylamino)pyrrolo[1,2-b]pyridazin-3-yl)-1,2,4-oxadiazol-3-yl)methanol methyl 4-((1S,2R)-2-methylcyclohexylamino)pyrrolo[1,2-b]pyridazine-3-carboxylate;
N-((1S,2R)-2-methylcyclohexyl)-3-(3-((tetrahydro-2H-pyran-2-yloxy)methyl)-1,2,4-oxadiazol-5-yl)pyrrolo[1,2-b]pyridazin-4-amine;
(5-(4-((1S,2R)-2-methylcyclohexylamino)pyrrolo[1,2-b]pyridazin-3-yl)-1,2,4-oxadiazol-3-yl)methanol;
methyl 4-((1R,2S)-2-methylcyclohexylamino)pyrrolo[1,2-b]pyridazine-3-carboxylate;
N-((1R,2S)-2-methylcyclohexyl)-3-(3-((tetrahydro-2H-pyran-2-yloxy)methyl)-1,2,4-oxadiazol-5-yl)pyrrolo[1,2-b]pyridazin-4-amine; and
(5-(4-((1R,2S)-2-methylcyclohexylamino)pyrrolo[1,2-b]pyridazin-3-yl)-1,2,4-oxadiazol-3-yl)methanol;
or a salt thereof.

57. A pharmaceutical composition comprising a compound of claim 1, or a pharmaceutically acceptable salt thereof, in combination with a pharmaceutically acceptable diluent or carrier.

58. (canceled)

59. A method for treating a disease or condition associated with pathologic JAK activation in a mammal, comprising administering to a mammal in need thereof an effective amount of a compound of claim 1, or a pharmaceutically acceptable salt thereof.

60. (canceled)

61. (canceled)

62. The method of claim 59, wherein the disease or condition associated with pathologic JAK activation is cancer.

63. (canceled)

64. A method for suppressing an immune response in a mammal, comprising administering to a mammal in need thereof an effective amount of a compound of claim 1, or a pharmaceutically acceptable salt thereof.

65. (canceled)

66. (canceled)
